SEQUENCE LISTING (62 sequences)

SEQ ID NO: 1 - DNA, 4140 bp, Unknown
  Description of Unknown: ORF encoding Sp. Cas9 sequence
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 2 - DNA, 4140 bp, Unknown
  Description of Unknown: ORF encoding Sp. Cas9 sequence
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 3 - RNA, 4140 nt, Unknown
  Description of Unknown: ORF encoding Sp. Cas9 sequence
auggacaagaaguacagcaucggccuggacaucggcacgaacagcguuggcugggcugugaucacggacgaguacaagguucccucaaagaaguucaaggugcugggcaacacggaccggcacagcaucaagaagaaucucaucggugcacugcuguucgacagcggugagacggccgaagccacgcggcugaagcggacggcccgccggcgguacacgcggcggaagaaccggaucugcuaccugcaggagaucuucagcaacgagauggccaagguggacgacagcuucuuccaccggcuggaggagagcuuccugguggaggaggacaagaagcacgagcggcaccccaucuucggcaacaucguggacgaagucgccuaccacgagaaguaccccaccaucuaccaccugcggaagaagcugguggacucgacugacaaggccgaccugcggcugaucuaccuggcacuggcccacaugauaaaguuccggggccacuuccugaucgagggcgaccugaacccugacaacagcgacguggacaagcuguucauccagcuggugcagaccuacaaccagcuguucgaggagaaccccaucaacgccagcggcguggacgccaaggccauccucagcgcccgccucagcaagagccggcggcuggagaaucucaucgcccagcuuccaggugagaagaagaaugggcuguucggcaaucucaucgcacucagccugggccugacucccaacuucaagagcaacuucgaccuggccgaggacgccaagcugcagcucagcaaggacaccuacgacgacgaccuggacaaucuccuggcccagaucggcgaccaguacgccgaccuguuccuggcugccaagaaucucagcgacgccauccugcucagcgacauccugcgggugaacacagagaucacgaaggccccccucagcgccagcaugauaaagcgguacgacgagcaccaccaggaccugacgcugcugaaggcacuggugcggcagcagcuuccagagaaguacaaggagaucuucuucgaccagagcaagaauggguacgccggguacaucgacgguggugccagccaggaggaguucuacaaguucaucaagcccauccuggagaagauggacggcacagaggagcugcuggugaagcugaacagggaggaccugcugcggaagcagcggacguucgacaaugggagcaucccccaccagauccaccugggugagcugcacgccauccugcggcggcaggaggacuucuaccccuuccugaaggacaacagggagaagaucgagaagauccugacguuccggauccccuacuacguuggcccccuggcccgcggcaacagccgguucgccuggaugacgcggaagagcgaggagacgaucacucccuggaacuucgaggaagucguggacaagggugccagcgcccagagcuucaucgagcggaugacgaacuucgacaagaaucuuccaaacgagaaggugcuuccaaagcacagccugcuguacgaguacuucacgguguacaacgagcugacgaaggugaaguacgugacagagggcaugcggaagcccgccuuccucagcggugagcagaagaaggccaucguggaccugcuguucaagacgaaccggaaggugacggugaagcagcugaaggaggacuacuucaagaagaucgagugcuucgacagcguggagaucagcggcguggaggaccgguucaacgccagccugggcaccuaccacgaccugcugaagaucaucaaggacaaggacuuccuggacaacgaggagaacgaggacauccuggaggacaucgugcugacgcugacgcuguucgaggacagggagaugauagaggagcggcugaagaccuacgcccaccuguucgacgacaaggugaugaagcagcugaagcggcggcgguacacgggcuggggccggcucagccggaagcugaucaaugggauccgagacaagcagagcggcaagacgauccuggacuuccugaagagcgacggcuucgccaaccggaacuucaugcagcugauccacgacgacagccugacguucaaggaggacauccagaaggcccaggucagcggccagggcgacagccugcacgagcacaucgccaaucucgccgggagccccgccaucaagaaggggauccugcagacggugaaggugguggacgagcuggugaaggugaugggccggcacaagccagagaacaucgugaucgagauggccagggagaaccagacgacucaaaaggggcagaagaacagcagggagcggaugaagcggaucgaggagggcaucaaggagcugggcagccagauccugaaggagcaccccguggagaacacucaacugcagaacgagaagcuguaccuguacuaccugcagaaugggcgagacauguacguggaccaggagcuggacaucaaccggcucagcgacuacgacguggaccacaucguuccccagagcuuccugaaggacgacagcaucgacaacaaggugcugacgcggagcgacaagaaccggggcaagagcgacaacguucccucagaggaagucgugaagaagaugaagaacuacuggcggcagcugcugaacgccaagcugaucacucaacggaaguucgacaaucucacgaaggccgagcgggguggccucagcgagcuggacaaggccggguucaucaagcggcagcugguggagacgcggcagaucacgaagcacguggcccagauccuggacagccggaugaacacgaaguacgacgagaacgacaagcugaucagggaagucaaggugaucacgcugaagagcaagcuggucagcgacuuccggaaggacuuccaguucuacaaggugagggagaucaacaacuaccaccacgcccacgacgccuaccugaacgcugugguuggcacggcacugaucaagaaguaccccaagcuggagagcgaguucguguacggcgacuacaagguguacgacgugcggaagaugauagccaagagcgagcaggagaucggcaaggccacggccaaguacuucuucuacagcaacaucaugaacuucuucaagacagagaucacgcuggccaauggugagauccggaagcggccccugaucgagacgaauggugagacgggugagaucgugugggacaaggggcgagacuucgccacggugcggaaggugcucagcaugccccaggugaacaucgugaagaagacagaaguccagacggguggcuucagcaaggagagcauccuuccaaagcggaacagcgacaagcugaucgcccgcaagaaggacugggaccccaagaaguacgguggcuucgacagccccaccguggccuacagcgugcuggugguggccaagguggagaaggggaagagcaagaagcugaagagcgugaaggagcugcugggcaucacgaucauggagcggagcagcuucgagaagaaccccaucgacuuccuggaagccaagggguacaaggaagucaagaaggaccugaucaucaagcuuccaaaguacagccuguucgagcuggagaaugggcggaagcggaugcuggccagcgccggugagcugcagaaggggaacgagcuggcacuucccucaaaguacgugaacuuccuguaccuggccagccacuacgagaagcugaaggggagcccagaggacaacgagcagaagcagcuguucguggagcagcacaagcacuaccuggacgagaucaucgagcagaucagcgaguucagcaagcgggugauccuggccgacgccaaucucgacaaggugcucagcgccuacaacaagcaccgagacaagcccaucagggagcaggccgagaacaucauccaccuguucacgcugacgaaucucggugcccccgcugccuucaaguacuucgacacgacgaucgaccggaagcgguacacgucgacuaaggaaguccuggacgccacgcugauccaccagagcaucacgggccuguacgagacgcggaucgaccucagccagcuggguggcgacggugguggcagccccaagaagaagcggaagguguag

SEQ ID NO: 4 - RNA, 4140 nt, Unknown
  Description of Unknown: ORF encoding Sp. Cas9 sequence
auggacaagaaguacagcaucggccucgacaucggcaccaacagcgucggcugggccgucaucaccgacgaguacaagguccccagcaagaaguucaagguccucggcaacaccgaccgccacagcaucaagaagaaccucaucggcgcccuccucuucgacagcggcgagaccgccgaggccacccgccucaagcgcaccgcccgccgccgcuacacccgccgcaagaaccgcaucugcuaccuccaggagaucuucagcaacgagauggccaaggucgacgacagcuucuuccaccgccucgaggagagcuuccucgucgaggaggacaagaagcacgagcgccaccccaucuucggcaacaucgucgacgaggucgccuaccacgagaaguaccccaccaucuaccaccuccgcaagaagcucgucgacagcaccgacaaggccgaccuccgccucaucuaccucgcccucgcccacaugaucaaguuccgcggccacuuccucaucgagggcgaccucaaccccgacaacagcgacgucgacaagcucuucauccagcucguccagaccuacaaccagcucuucgaggagaaccccaucaacgccagcggcgucgacgccaaggccauccucagcgcccgccucagcaagagccgccgccucgagaaccucaucgcccagcuccccggcgagaagaagaacggccucuucggcaaccucaucgcccucagccucggccucacccccaacuucaagagcaacuucgaccucgccgaggacgccaagcuccagcucagcaaggacaccuacgacgacgaccucgacaaccuccucgcccagaucggcgaccaguacgccgaccucuuccucgccgccaagaaccucagcgacgccauccuccucagcgacauccuccgcgucaacaccgagaucaccaaggccccccucagcgccagcaugaucaagcgcuacgacgagcaccaccaggaccucacccuccucaaggcccucguccgccagcagcuccccgagaaguacaaggagaucuucuucgaccagagcaagaacggcuacgccggcuacaucgacggcggcgccagccaggaggaguucuacaaguucaucaagcccauccucgagaagauggacggcaccgaggagcuccucgucaagcucaaccgcgaggaccuccuccgcaagcagcgcaccuucgacaacggcagcaucccccaccagauccaccucggcgagcuccacgccauccuccgccgccaggaggacuucuaccccuuccucaaggacaaccgcgagaagaucgagaagauccucaccuuccgcauccccuacuacgucggcccccucgcccgcggcaacagccgcuucgccuggaugacccgcaagagcgaggagaccaucacccccuggaacuucgaggaggucgucgacaagggcgccagcgcccagagcuucaucgagcgcaugaccaacuucgacaagaaccuccccaacgagaagguccuccccaagcacagccuccucuacgaguacuucaccgucuacaacgagcucaccaaggucaaguacgucaccgagggcaugcgcaagcccgccuuccucagcggcgagcagaagaaggccaucgucgaccuccucuucaagaccaaccgcaaggucaccgucaagcagcucaaggaggacuacuucaagaagaucgagugcuucgacagcgucgagaucagcggcgucgaggaccgcuucaacgccagccucggcaccuaccacgaccuccucaagaucaucaaggacaaggacuuccucgacaacgaggagaacgaggacauccucgaggacaucguccucacccucacccucuucgaggaccgcgagaugaucgaggagcgccucaagaccuacgcccaccucuucgacgacaaggucaugaagcagcucaagcgccgccgcuacaccggcuggggccgccucagccgcaagcucaucaacggcauccgcgacaagcagagcggcaagaccauccucgacuuccucaagagcgacggcuucgccaaccgcaacuucaugcagcucauccacgacgacagccucaccuucaaggaggacauccagaaggcccaggucagcggccagggcgacagccuccacgagcacaucgccaaccucgccggcagccccgccaucaagaagggcauccuccagaccgucaaggucgucgacgagcucgucaaggucaugggccgccacaagcccgagaacaucgucaucgagauggcccgcgagaaccagaccacccagaagggccagaagaacagccgcgagcgcaugaagcgcaucgaggagggcaucaaggagcucggcagccagauccucaaggagcaccccgucgagaacacccagcuccagaacgagaagcucuaccucuacuaccuccagaacggccgcgacauguacgucgaccaggagcucgacaucaaccgccucagcgacuacgacgucgaccacaucgucccccagagcuuccucaaggacgacagcaucgacaacaagguccucacccgcagcgacaagaaccgcggcaagagcgacaacguccccagcgaggaggucgucaagaagaugaagaacuacuggcgccagcuccucaacgccaagcucaucacccagcgcaaguucgacaaccucaccaaggccgagcgcggcggccucagcgagcucgacaaggccggcuucaucaagcgccagcucgucgagacccgccagaucaccaagcacgucgcccagauccucgacagccgcaugaacaccaaguacgacgagaacgacaagcucauccgcgaggucaaggucaucacccucaagagcaagcucgucagcgacuuccgcaaggacuuccaguucuacaagguccgcgagaucaacaacuaccaccacgcccacgacgccuaccucaacgccgucgucggcaccgcccucaucaagaaguaccccaagcucgagagcgaguucgucuacggcgacuacaaggucuacgacguccgcaagaugaucgccaagagcgagcaggagaucggcaaggccaccgccaaguacuucuucuacagcaacaucaugaacuucuucaagaccgagaucacccucgccaacggcgagauccgcaagcgcccccucaucgagaccaacggcgagaccggcgagaucgucugggacaagggccgcgacuucgccaccguccgcaagguccucagcaugccccaggucaacaucgucaagaagaccgagguccagaccggcggcuucagcaaggagagcauccuccccaagcgcaacagcgacaagcucaucgcccgcaagaaggacugggaccccaagaaguacggcggcuucgacagccccaccgucgccuacagcguccucgucgucgccaaggucgagaagggcaagagcaagaagcucaagagcgucaaggagcuccucggcaucaccaucauggagcgcagcagcuucgagaagaaccccaucgacuuccucgaggccaagggcuacaaggaggucaagaaggaccucaucaucaagcuccccaaguacagccucuucgagcucgagaacggccgcaagcgcaugcucgccagcgccggcgagcuccagaagggcaacgagcucgcccuccccagcaaguacgucaacuuccucuaccucgccagccacuacgagaagcucaagggcagccccgaggacaacgagcagaagcagcucuucgucgagcagcacaagcacuaccucgacgagaucaucgagcagaucagcgaguucagcaagcgcgucauccucgccgacgccaaccucgacaagguccucagcgccuacaacaagcaccgcgacaagcccauccgcgagcaggccgagaacaucauccaccucuucacccucaccaaccucggcgcccccgccgccuucaaguacuucgacaccaccaucgaccgcaagcgcuacaccagcaccaaggagguccucgacgccacccucauccaccagagcaucaccggccucuacgagacccgcaucgaccucagccagcucggcggcgacggcggcggcagccccaagaagaagcgcaaggucuag

SEQ ID NO: 5 - DNA, 4140 bp, Unknown
  Description of Unknown: ORF encoding Sp. Cas9 sequence
atggacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacggcggcggcagccccaagaagaagcggaaggtgtga

SEQ ID NO: 6 - DNA, 4140 bp, Unknown
  Description of Unknown: ORF encoding Sp. Cas9 nickase sequence
atggacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 7 - DNA, 4140 bp, Unknown
  Description of Unknown: ORF encoding Sp. Cas9 nickase sequence
atggacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacggcggcggcagccccaagaagaagcggaaggtgtga

SEQ ID NO: 8 - RNA, 4511 nt, Unknown
  Description of Unknown: mRNA encoding Sp. Cas9 sequence
gggucccgcagucggcguccagcggcucugcuuguucgugugugugucguugcaggccuuauucggauccgccaccauggacaagaaguacagcaucggacuggacaucggaacaaacagcgucggaugggcagucaucacagacgaauacaaggucccgagcaagaaguucaagguccugggaaacacagacagacacagcaucaagaagaaccugaucggagcacugcuguucgacagcggagaaacagcagaagcaacaagacugaagagaacagcaagaagaagauacacaagaagaaagaacagaaucugcuaccugcaggaaaucuucagcaacgaaauggcaaaggucgacgacagcuucuuccacagacuggaagaaagcuuccuggucgaagaagacaagaagcacgaaagacacccgaucuucggaaacaucgucgacgaagucgcauaccacgaaaaguacccgacaaucuaccaccugagaaagaagcuggucgacagcacagacaaggcagaccugagacugaucuaccuggcacuggcacacaugaucaaguucagaggacacuuccugaucgaaggagaccugaacccggacaacagcgacgucgacaagcuguucauccagcugguccagacauacaaccagcuguucgaagaaaacccgaucaacgcaagcggagucgacgcaaaggcaauccugagcgcaagacugagcaagagcagaagacuggaaaaccugaucgcacagcugccgggagaaaagaagaacggacuguucggaaaccugaucgcacugagccugggacugacaccgaacuucaagagcaacuucgaccuggcagaagacgcaaagcugcagcugagcaaggacacauacgacgacgaccuggacaaccugcuggcacagaucggagaccaguacgcagaccuguuccuggcagcaaagaaccugagcgacgcaauccugcugagcgacauccugagagucaacacagaaaucacaaaggcaccgcugagcgcaagcaugaucaagagauacgacgaacaccaccaggaccugacacugcugaaggcacuggucagacagcagcugccggaaaaguacaaggaaaucuucuucgaccagagcaagaacggauacgcaggauacaucgacggaggagcaagccaggaagaauucuacaaguucaucaagccgauccuggaaaagauggacggaacagaagaacugcuggucaagcugaacagagaagaccugcugagaaagcagagaacauucgacaacggaagcaucccgcaccagauccaccugggagaacugcacgcaauccugagaagacaggaagacuucuacccguuccugaaggacaacagagaaaagaucgaaaagauccugacauucagaaucccguacuacgucggaccgcuggcaagaggaaacagcagauucgcauggaugacaagaaagagcgaagaaacaaucacaccguggaacuucgaagaagucgucgacaagggagcaagcgcacagagcuucaucgaaagaaugacaaacuucgacaagaaccugccgaacgaaaagguccugccgaagcacagccugcuguacgaauacuucacagucuacaacgaacugacaaaggucaaguacgucacagaaggaaugagaaagccggcauuccugagcggagaacagaagaaggcaaucgucgaccugcuguucaagacaaacagaaaggucacagucaagcagcugaaggaagacuacuucaagaagaucgaaugcuucgacagcgucgaaaucagcggagucgaagacagauucaacgcaagccugggaacauaccacgaccugcugaagaucaucaaggacaaggacuuccuggacaacgaagaaaacgaagacauccuggaagacaucguccugacacugacacuguucgaagacagagaaaugaucgaagaaagacugaagacauacgcacaccuguucgacgacaaggucaugaagcagcugaagagaagaagauacacaggauggggaagacugagcagaaagcugaucaacggaaucagagacaagcagagcggaaagacaauccuggacuuccugaagagcgacggauucgcaaacagaaacuucaugcagcugauccacgacgacagccugacauucaaggaagacauccagaaggcacaggucagcggacagggagacagccugcacgaacacaucgcaaaccuggcaggaagcccggcaaucaagaagggaauccugcagacagucaaggucgucgacgaacuggucaaggucaugggaagacacaagccggaaaacaucgucaucgaaauggcaagagaaaaccagacaacacagaagggacagaagaacagcagagaaagaaugaagagaaucgaagaaggaaucaaggaacugggaagccagauccugaaggaacacccggucgaaaacacacagcugcagaacgaaaagcuguaccuguacuaccugcagaacggaagagacauguacgucgaccaggaacuggacaucaacagacugagcgacuacgacgucgaccacaucgucccgcagagcuuccugaaggacgacagcaucgacaacaagguccugacaagaagcgacaagaacagaggaaagagcgacaacgucccgagcgaagaagucgucaagaagaugaagaacuacuggagacagcugcugaacgcaaagcugaucacacagagaaaguucgacaaccugacaaaggcagagagaggaggacugagcgaacuggacaaggcaggauucaucaagagacagcuggucgaaacaagacagaucacaaagcacgucgcacagauccuggacagcagaaugaacacaaaguacgacgaaaacgacaagcugaucagagaagucaaggucaucacacugaagagcaagcuggucagcgacuucagaaaggacuuccaguucuacaaggucagagaaaucaacaacuaccaccacgcacacgacgcauaccugaacgcagucgucggaacagcacugaucaagaaguacccgaagcuggaaagcgaauucgucuacggagacuacaaggucuacgacgucagaaagaugaucgcaaagagcgaacaggaaaucggaaaggcaacagcaaaguacuucuucuacagcaacaucaugaacuucuucaagacagaaaucacacuggcaaacggagaaaucagaaagagaccgcugaucgaaacaaacggagaaacaggagaaaucgucugggacaagggaagagacuucgcaacagucagaaagguccugagcaugccgcaggucaacaucgucaagaagacagaaguccagacaggaggauucagcaaggaaagcauccugccgaagagaaacagcgacaagcugaucgcaagaaagaaggacugggacccgaagaaguacggaggauucgacagcccgacagucgcauacagcguccuggucgucgcaaaggucgaaaagggaaagagcaagaagcugaagagcgucaaggaacugcugggaaucacaaucauggaaagaagcagcuucgaaaagaacccgaucgacuuccuggaagcaaagggauacaaggaagucaagaaggaccugaucaucaagcugccgaaguacagccuguucgaacuggaaaacggaagaaagagaaugcuggcaagcgcaggagaacugcagaagggaaacgaacuggcacugccgagcaaguacgucaacuuccuguaccuggcaagccacuacgaaaagcugaagggaagcccggaagacaacgaacagaagcagcuguucgucgaacagcacaagcacuaccuggacgaaaucaucgaacagaucagcgaauucagcaagagagucauccuggcagacgcaaaccuggacaagguccugagcgcauacaacaagcacagagacaagccgaucagagaacaggcagaaaacaucauccaccuguucacacugacaaaccugggagcaccggcagcauucaaguacuucgacacaacaaucgacagaaagagauacacaagcacaaaggaaguccuggacgcaacacugauccaccagagcaucacaggacuguacgaaacaagaaucgaccugagccagcugggaggagacggaggaggaagcccgaagaagaagagaaaggucuagcuagccaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - RNA, 4418 nt, Unknown
  Description of Unknown: mRNA encoding Sp. Cas9 sequence
gggaagcucagaauaaacgcucaacuuuggccggaucugccaccauggacaagaaguacuccaucggccuggacaucggcaccaacuccgugggcugggccgugaucaccgacgaguacaaggugcccuccaagaaguucaaggugcugggcaacaccgaccggcacuccaucaagaagaaccugaucggcgcccugcuguucgacuccggcgagaccgccgaggccacccggcugaagcggaccgcccggcggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgagguggccuaccacgagaaguaccccaccaucuaccaccugcggaagaagcugguggacuccaccgacaaggccgaccugcggcugaucuaccuggcccuggcccacaugaucaaguuccggggccacuuccugaucgagggcgaccugaaccccgacaacuccgacguggacaagcuguucauccagcuggugcagaccuacaaccagcuguucgaggagaaccccaucaacgccuccggcguggacgccaaggccauccuguccgcccggcuguccaagucccggcggcuggagaaccugaucgcccagcugcccggcgagaagaagaacggccuguucggcaaccugaucgcccugucccugggccugacccccaacuucaaguccaacuucgaccuggccgaggacgccaagcugcagcuguccaaggacaccuacgacgacgaccuggacaaccugcuggcccagaucggcgaccaguacgccgaccuguuccuggccgccaagaaccuguccgacgccauccugcuguccgacauccugcgggugaacaccgagaucaccaaggccccccuguccgccuccaugaucaagcgguacgacgagcaccaccaggaccugacccugcugaaggcccuggugcggcagcagcugcccgagaaguacaaggagaucuucuucgaccaguccaagaacggcuacgccggcuacaucgacggcggcgccucccaggaggaguucuacaaguucaucaagcccauccuggagaagauggacggcaccgaggagcugcuggugaagcugaaccgggaggaccugcugcggaagcagcggaccuucgacaacggcuccaucccccaccagauccaccugggcgagcugcacgccauccugcggcggcaggaggacuucuaccccuuccugaaggacaaccgggagaagaucgagaagauccugaccuuccggauccccuacuacgugggcccccuggcccggggcaacucccgguucgccuggaugacccggaaguccgaggagaccaucacccccuggaacuucgaggaggugguggacaagggcgccuccgcccaguccuucaucgagcggaugaccaacuucgacaagaaccugcccaacgagaaggugcugcccaagcacucccugcuguacgaguacuucaccguguacaacgagcugaccaaggugaaguacgugaccgagggcaugcggaagcccgccuuccuguccggcgagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggaccgggagaugaucgaggagcggcugaagaccuacgcccaccuguucgacgacaaggugaugaagcagcugaagcggcggcgguacaccggcuggggccggcugucccggaagcugaucaacggcauccgggacaagcaguccggcaagaccauccuggacuuccugaaguccgacggcuucgccaaccggaacuucaugcagcugauccacgacgacucccugaccuucaaggaggacauccagaaggcccagguguccggccagggcgacucccugcacgagcacaucgccaaccuggccggcucccccgccaucaagaagggcauccugcagaccgugaaggugguggacgagcuggugaaggugaugggccggcacaagcccgagaacaucgugaucgagauggcccgggagaaccagaccacccagaagggccagaagaacucccgggagcggaugaagcggaucgaggagggcaucaaggagcugggcucccagauccugaaggagcaccccguggagaacacccagcugcagaacgagaagcuguaccuguacuaccugcagaacggccgggacauguacguggaccaggagcuggacaucaaccggcuguccgacuacgacguggaccacaucgugccccaguccuuccugaaggacgacuccaucgacaacaaggugcugacccgguccgacaagaaccggggcaaguccgacaacgugcccuccgaggagguggugaagaagaugaagaacuacuggcggcagcugcugaacgccaagcugaucacccagcggaaguucgacaaccugaccaaggccgagcggggcggccuguccgagcuggacaaggccggcuucaucaagcggcagcugguggagacccggcagaucaccaagcacguggcccagauccuggacucccggaugaacaccaaguacgacgagaacgacaagcugauccgggaggugaaggugaucacccugaaguccaagcugguguccgacuuccggaaggacuuccaguucuacaaggugcgggagaucaacaacuaccaccacgcccacgacgccuaccugaacgccguggugggcaccgcccugaucaagaaguaccccaagcuggaguccgaguucguguacggcgacuacaagguguacgacgugcggaagaugaucgccaaguccgagcaggagaucggcaaggccaccgccaaguacuucuucuacuccaacaucaugaacuucuucaagaccgagaucacccuggccaacggcgagauccggaagcggccccugaucgagaccaacggcgagaccggcgagaucgugugggacaagggccgggacuucgccaccgugcggaaggugcuguccaugccccaggugaacaucgugaagaagaccgaggugcagaccggcggcuucuccaaggaguccauccugcccaagcggaacuccgacaagcugaucgcccggaagaaggacugggaccccaagaaguacggcggcuucgacucccccaccguggccuacuccgugcuggugguggccaagguggagaagggcaaguccaagaagcugaaguccgugaaggagcugcugggcaucaccaucauggagcgguccuccuucgagaagaaccccaucgacuuccuggaggccaagggcuacaaggaggugaagaaggaccugaucaucaagcugcccaaguacucccuguucgagcuggagaacggccggaagcggaugcuggccuccgccggcgagcugcagaagggcaacgagcuggcccugcccuccaaguacgugaacuuccuguaccuggccucccacuacgagaagcugaagggcucccccgaggacaacgagcagaagcagcuguucguggagcagcacaagcacuaccuggacgagaucaucgagcagaucuccgaguucuccaagcgggugauccuggccgacgccaaccuggacaaggugcuguccgccuacaacaagcaccgggacaagcccauccgggagcaggccgagaacaucauccaccuguucacccugaccaaccugggcgcccccgccgccuucaaguacuucgacaccaccaucgaccggaagcgguacaccuccaccaaggaggugcuggacgccacccugauccaccaguccaucaccggccuguacgagacccggaucgaccugucccagcugggcggcgacggcggcggcucccccaagaagaagcggaaggugugacuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - RNA, 4416 nt, Unknown
  Description of Unknown: mRNA encoding Sp. Cas9 sequence
gggaagcucagaauaaacgcucaacuuuggccggaucugccaccauggacaagaaguacagcaucggccuggacaucggcaccaacagcgugggcugggccgugaucaccgacgaguacaaggugcccagcaagaaguucaaggugcugggcaacaccgaccggcacagcaucaagaagaaccugaucggcgcccugcuguucgacagcggcgagaccgccgaggccacccggcugaagcggaccgcccggcggcgguacacccggcggaagaaccggaucugcuaccugcaggagaucuucagcaacgagauggccaagguggacgacagcuucuuccaccggcuggaggagagcuuccugguggaggaggacaagaagcacgagcggcaccccaucuucggcaacaucguggacgagguggccuaccacgagaaguaccccaccaucuaccaccugcggaagaagcugguggacagcaccgacaaggccgaccugcggcugaucuaccuggcccuggcccacaugaucaaguuccggggccacuuccugaucgagggcgaccugaaccccgacaacagcgacguggacaagcuguucauccagcuggugcagaccuacaaccagcuguucgaggagaaccccaucaacgccagcggcguggacgccaaggccauccugagcgcccggcugagcaagagccggcggcuggagaaccugaucgcccagcugcccggcgagaagaagaacggccuguucggcaaccugaucgcccugagccugggccugacccccaacuucaagagcaacuucgaccuggccgaggacgccaagcugcagcugagcaaggacaccuacgacgacgaccuggacaaccugcuggcccagaucggcgaccaguacgccgaccuguuccuggccgccaagaaccugagcgacgccauccugcugagcgacauccugcgggugaacaccgagaucaccaaggccccccugagcgccagcaugaucaagcgguacgacgagcaccaccaggaccugacccugcugaaggcccuggugcggcagcagcugcccgagaaguacaaggagaucuucuucgaccagagcaagaacggcuacgccggcuacaucgacggcggcgccagccaggaggaguucuacaaguucaucaagcccauccuggagaagauggacggcaccgaggagcugcuggugaagcugaaccgggaggaccugcugcggaagcagcggaccuucgacaacggcagcaucccccaccagauccaccugggcgagcugcacgccauccugcggcggcaggaggacuucuaccccuuccugaaggacaaccgggagaagaucgagaagauccugaccuuccggauccccuacuacgugggcccccuggcccggggcaacagccgguucgccuggaugacccggaagagcgaggagaccaucacccccuggaacuucgaggaggugguggacaagggcgccagcgcccagagcuucaucgagcggaugaccaacuucgacaagaaccugcccaacgagaaggugcugcccaagcacagccugcuguacgaguacuucaccguguacaacgagcugaccaaggugaaguacgugaccgagggcaugcggaagcccgccuuccugagcggcgagcagaagaaggccaucguggaccugcuguucaagaccaaccggaaggugaccgugaagcagcugaaggaggacuacuucaagaagaucgagugcuucgacagcguggagaucagcggcguggaggaccgguucaacgccagccugggcaccuaccacgaccugcugaagaucaucaaggacaaggacuuccuggacaacgaggagaacgaggacauccuggaggacaucgugcugacccugacccuguucgaggaccgggagaugaucgaggagcggcugaagaccuacgcccaccuguucgacgacaaggugaugaagcagcugaagcggcggcgguacaccggcuggggccggcugagccggaagcugaucaacggcauccgggacaagcagagcggcaagaccauccuggacuuccugaagagcgacggcuucgccaaccggaacuucaugcagcugauccacgacgacagccugaccuucaaggaggacauccagaaggcccaggugagcggccagggcgacagccugcacgagcacaucgccaaccuggccggcagccccgccaucaagaagggcauccugcagaccgugaaggugguggacgagcuggugaaggugaugggccggcacaagcccgagaacaucgugaucgagauggcccgggagaaccagaccacccagaagggccagaagaacagccgggagcggaugaagcggaucgaggagggcaucaaggagcugggcagccagauccugaaggagcaccccguggagaacacccagcugcagaacgagaagcuguaccuguacuaccugcagaacggccgggacauguacguggaccaggagcuggacaucaaccggcugagcgacuacgacguggaccacaucgugccccagagcuuccugaaggacgacagcaucgacaacaaggugcugacccggagcgacaagaaccggggcaagagcgacaacgugcccagcgaggagguggugaagaagaugaagaacuacuggcggcagcugcugaacgccaagcugaucacccagcggaaguucgacaaccugaccaaggccgagcggggcggccugagcgagcuggacaaggccggcuucaucaagcggcagcugguggagacccggcagaucaccaagcacguggcccagauccuggacagccggaugaacaccaaguacgacgagaacgacaagcugauccgggaggugaaggugaucacccugaagagcaagcuggugagcgacuuccggaaggacuuccaguucuacaaggugcgggagaucaacaacuaccaccacgcccacgacgccuaccugaacgccguggugggcaccgcccugaucaagaaguaccccaagcuggagagcgaguucguguacggcgacuacaagguguacgacgugcggaagaugaucgccaagagcgagcaggagaucggcaaggccaccgccaaguacuucuucuacagcaacaucaugaacuucuucaagaccgagaucacccuggccaacggcgagauccggaagcggccccugaucgagaccaacggcgagaccggcgagaucgugugggacaagggccgggacuucgccaccgugcggaaggugcugagcaugccccaggugaacaucgugaagaagaccgaggugcagaccggcggcuucagcaaggagagcauccugcccaagcggaacagcgacaagcugaucgcccggaagaaggacugggaccccaagaaguacggcggcuucgacagccccaccguggccuacagcgugcuggugguggccaagguggagaagggcaagagcaagaagcugaagagcgugaaggagcugcugggcaucaccaucauggagcggagcagcuucgagaagaaccccaucgacuuccuggaggccaagggcuacaaggaggugaagaaggaccugaucaucaagcugcccaaguacagccuguucgagcuggagaacggccggaagcggaugcuggccagcgccggcgagcugcagaagggcaacgagcuggcccugcccagcaaguacgugaacuuccuguaccuggccagccacuacgagaagcugaagggcagccccgaggacaacgagcagaagcagcuguucguggagcagcacaagcacuaccuggacgagaucaucgagcagaucagcgaguucagcaagcgggugauccuggccgacgccaaccuggacaaggugcugagcgccuacaacaagcaccgggacaagcccauccgggagcaggccgagaacaucauccaccuguucacccugaccaaccugggcgcccccgccgccuucaaguacuucgacaccaccaucgaccggaagcgguacaccagcaccaaggaggugcuggacgccacccugauccaccagagcaucaccggccuguacgagacccggaucgaccugagccagcugggcggcgacggcggcggcagccccaagaagaagcggaaggugugacuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - RNA, 4420 nt, Unknown
  Description of Unknown: mRNA encoding Sp. Cas9 sequence
gggaagcucagaauaaacgcucaacuuuggccggaucugccaccauggacaagaaguacagcaucggccuggacaucggcacgaacagcguuggcugggcugugaucacggacgaguacaagguucccucaaagaaguucaaggugcugggcaacacggaccggcacagcaucaagaagaaucucaucggugcacugcuguucgacagcggugagacggccgaagccacgcggcugaagcggacggcccgccggcgguacacgcggcggaagaaccggaucugcuaccugcaggagaucuucagcaacgagauggccaagguggacgacagcuucuuccaccggcuggaggagagcuuccugguggaggaggacaagaagcacgagcggcaccccaucuucggcaacaucguggacgaagucgccuaccacgagaaguaccccaccaucuaccaccugcggaagaagcugguggacucgacugacaaggccgaccugcggcugaucuaccuggcacuggcccacaugauaaaguuccggggccacuuccugaucgagggcgaccugaacccugacaacagcgacguggacaagcuguucauccagcuggugcagaccuacaaccagcuguucgaggagaaccccaucaacgccagcggcguggacgccaaggccauccucagcgcccgccucagcaagagccggcggcuggagaaucucaucgcccagcuuccaggugagaagaagaaugggcuguucggcaaucucaucgcacucagccugggccugacucccaacuucaagagcaacuucgaccuggccgaggacgccaagcugcagcucagcaaggacaccuacgacgacgaccuggacaaucuccuggcccagaucggcgaccaguacgccgaccuguuccuggcugccaagaaucucagcgacgccauccugcucagcgacauccugcgggugaacacagagaucacgaaggccccccucagcgccagcaugauaaagcgguacgacgagcaccaccaggaccugacgcugcugaaggcacuggugcggcagcagcuuccagagaaguacaaggagaucuucuucgaccagagcaagaauggguacgccggguacaucgacgguggugccagccaggaggaguucuacaaguucaucaagcccauccuggagaagauggacggcacagaggagcugcuggugaagcugaacagggaggaccugcugcggaagcagcggacguucgacaaugggagcaucccccaccagauccaccugggugagcugcacgccauccugcggcggcaggaggacuucuaccccuuccugaaggacaacagggagaagaucgagaagauccugacguuccggauccccuacuacguuggcccccuggcccgcggcaacagccgguucgccuggaugacgcggaagagcgaggagacgaucacucccuggaacuucgaggaagucguggacaagggugccagcgcccagagcuucaucgagcggaugacgaacuucgacaagaaucuuccaaacgagaaggugcuuccaaagcacagccugcuguacgaguacuucacgguguacaacgagcugacgaaggugaaguacgugacagagggcaugcggaagcccgccuuccucagcggugagcagaagaaggccaucguggaccugcuguucaagacgaaccggaaggugacggugaagcagcugaaggaggacuacuucaagaagaucgagugcuucgacagcguggagaucagcggcguggaggaccgguucaacgccagccugggcaccuaccacgaccugcugaagaucaucaaggacaaggacuuccuggacaacgaggagaacgaggacauccuggaggacaucgugcugacgcugacgcuguucgaggacagggagaugauagaggagcggcugaagaccuacgcccaccuguucgacgacaaggugaugaagcagcugaagcggcggcgguacacgggcuggggccggcucagccggaagcugaucaaugggauccgagacaagcagagcggcaagacgauccuggacuuccugaagagcgacggcuucgccaaccggaacuucaugcagcugauccacgacgacagccugacguucaaggaggacauccagaaggcccaggucagcggccagggcgacagccugcacgagcacaucgccaaucucgccgggagccccgccaucaagaaggggauccugcagacggugaaggugguggacgagcuggugaaggugaugggccggcacaagccagagaacaucgugaucgagauggccagggagaaccagacgacucaaaaggggcagaagaacagcagggagcggaugaagcggaucgaggagggcaucaaggagcugggcagccagauccugaaggagcaccccguggagaacacucaacugcagaacgagaagcuguaccuguacuaccugcagaaugggcgagacauguacguggaccaggagcuggacaucaaccggcucagcgacuacgacguggaccacaucguuccccagagcuuccugaaggacgacagcaucgacaacaaggugcugacgcggagcgacaagaaccggggcaagagcgacaacguucccucagaggaagucgugaagaagaugaagaacuacuggcggcagcugcugaacgccaagcugaucacucaacggaaguucgacaaucucacgaaggccgagcgggguggccucagcgagcuggacaaggccggguucaucaagcggcagcugguggagacgcggcagaucacgaagcacguggcccagauccuggacagccggaugaacacgaaguacgacgagaacgacaagcugaucagggaagucaaggugaucacgcugaagagcaagcuggucagcgacuuccggaaggacuuccaguucuacaaggugagggagaucaacaacuaccaccacgcccacgacgccuaccugaacgcugugguuggcacggcacugaucaagaaguaccccaagcuggagagcgaguucguguacggcgacuacaagguguacgacgugcggaagaugauagccaagagcgagcaggagaucggcaaggccacggccaaguacuucuucuacagcaacaucaugaacuucuucaagacagagaucacgcuggccaauggugagauccggaagcggccccugaucgagacgaauggugagacgggugagaucgugugggacaaggggcgagacuucgccacggugcggaaggugcucagcaugccccaggugaacaucgugaagaagacagaaguccagacggguggcuucagcaaggagagcauccuuccaaagcggaacagcgacaagcugaucgcccgcaagaaggacugggaccccaagaaguacgguggcuucgacagccccaccguggccuacagcgugcuggugguggccaagguggagaaggggaagagcaagaagcugaagagcgugaaggagcugcugggcaucacgaucauggagcggagcagcuucgagaagaaccccaucgacuuccuggaagccaagggguacaaggaagucaagaaggaccugaucaucaagcuuccaaaguacagccuguucgagcuggagaaugggcggaagcggaugcuggccagcgccggugagcugcagaaggggaacgagcuggcacuucccucaaaguacgugaacuuccuguaccuggccagccacuacgagaagcugaaggggagcccagaggacaacgagcagaagcagcuguucguggagcagcacaagcacuaccuggacgagaucaucgagcagaucagcgaguucagcaagcgggugauccuggccgacgccaaucucgacaaggugcucagcgccuacaacaagcaccgagacaagcccaucagggagcaggccgagaacaucauccaccuguucacgcugacgaaucucggugcccccgcugccuucaaguacuucgacacgacgaucgaccggaagcgguacacgucgacuaaggaaguccuggacgccacgcugauccaccagagcaucacgggccuguacgagacgcggaucgaccucagccagcuggguggcgacggugguggcagccccaagaagaagcggaagguguagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - RNA, 4429 nt, Unknown
  Description of Unknown: mRNA encoding Sp. Cas9 sequence
gggaagcucagaauaaacgcucaacuuuggccggaucugccaccauggacaagaaguacagcaucggccucgacaucggcaccaacagcgucggcugggccgucaucaccgacgaguacaagguccccagcaagaaguucaagguccucggcaacaccgaccgccacagcaucaagaagaaccucaucggcgcccuccucuucgacagcggcgagaccgccgaggccacccgccucaagcgcaccgcccgccgccgcuacacccgccgcaagaaccgcaucugcuaccuccaggagaucuucagcaacgagauggccaaggucgacgacagcuucuuccaccgccucgaggagagcuuccucgucgaggaggacaagaagcacgagcgccaccccaucuucggcaacaucgucgacgaggucgccuaccacgagaaguaccccaccaucuaccaccuccgcaagaagcucgucgacagcaccgacaaggccgaccuccgccucaucuaccucgcccucgcccacaugaucaaguuccgcggccacuuccucaucgagggcgaccucaaccccgacaacagcgacgucgacaagcucuucauccagcucguccagaccuacaaccagcucuucgaggagaaccccaucaacgccagcggcgucgacgccaaggccauccucagcgcccgccucagcaagagccgccgccucgagaaccucaucgcccagcuccccggcgagaagaagaacggccucuucggcaaccucaucgcccucagccucggccucacccccaacuucaagagcaacuucgaccucgccgaggacgccaagcuccagcucagcaaggacaccuacgacgacgaccucgacaaccuccucgcccagaucggcgaccaguacgccgaccucuuccucgccgccaagaaccucagcgacgccauccuccucagcgacauccuccgcgucaacaccgagaucaccaaggccccccucagcgccagcaugaucaagcgcuacgacgagcaccaccaggaccucacccuccucaaggcccucguccgccagcagcuccccgagaaguacaaggagaucuucuucgaccagagcaagaacggcuacgccggcuacaucgacggcggcgccagccaggaggaguucuacaaguucaucaagcccauccucgagaagauggacggcaccgaggagcuccucgucaagcucaaccgcgaggaccuccuccgcaagcagcgcaccuucgacaacggcagcaucccccaccagauccaccucggcgagcuccacgccauccuccgccgccaggaggacuucuaccccuuccucaaggacaaccgcgagaagaucgagaagauccucaccuuccgcauccccuacuacgucggcccccucgcccgcggcaacagccgcuucgccuggaugacccgcaagagcgaggagaccaucacccccuggaacuucgaggaggucgucgacaagggcgccagcgcccagagcuucaucgagcgcaugaccaacuucgacaagaaccuccccaacgagaagguccuccccaagcacagccuccucuacgaguacuucaccgucuacaacgagcucaccaaggucaaguacgucaccgagggcaugcgcaagcccgccuuccucagcggcgagcagaagaaggccaucgucgaccuccucuucaagaccaaccgcaaggucaccgucaagcagcucaaggaggacuacuucaagaagaucgagugcuucgacagcgucgagaucagcggcgucgaggaccgcuucaacgccagccucggcaccuaccacgaccuccucaagaucaucaaggacaaggacuuccucgacaacgaggagaacgaggacauccucgaggacaucguccucacccucacccucuucgaggaccgcgagaugaucgaggagcgccucaagaccuacgcccaccucuucgacgacaaggucaugaagcagcucaagcgccgccgcuacaccggcuggggccgccucagccgcaagcucaucaacggcauccgcgacaagcagagcggcaagaccauccucgacuuccucaagagcgacggcuucgccaaccgcaacuucaugcagcucauccacgacgacagccucaccuucaaggaggacauccagaaggcccaggucagcggccagggcgacagccuccacgagcacaucgccaaccucgccggcagccccgccaucaagaagggcauccuccagaccgucaaggucgucgacgagcucgucaaggucaugggccgccacaagcccgagaacaucgucaucgagauggcccgcgagaaccagaccacccagaagggccagaagaacagccgcgagcgcaugaagcgcaucgaggagggcaucaaggagcucggcagccagauccucaaggagcaccccgucgagaacacccagcuccagaacgagaagcucuaccucuacuaccuccagaacggccgcgacauguacgucgaccaggagcucgacaucaaccgccucagcgacuacgacgucgaccacaucgucccccagagcuuccucaaggacgacagcaucgacaacaagguccucacccgcagcgacaagaaccgcggcaagagcgacaacguccccagcgaggaggucgucaagaagaugaagaacuacuggcgccagcuccucaacgccaagcucaucacccagcgcaaguucgacaaccucaccaaggccgagcgcggcggccucagcgagcucgacaaggccggcuucaucaagcgccagcucgucgagacccgccagaucaccaagcacgucgcccagauccucgacagccgcaugaacaccaaguacgacgagaacgacaagcucauccgcgaggucaaggucaucacccucaagagcaagcucgucagcgacuuccgcaaggacuuccaguucuacaagguccgcgagaucaacaacuaccaccacgcccacgacgccuaccucaacgccgucgucggcaccgcccucaucaagaaguaccccaagcucgagagcgaguucgucuacggcgacuacaaggucuacgacguccgcaagaugaucgccaagagcgagcaggagaucggcaaggccaccgccaaguacuucuucuacagcaacaucaugaacuucuucaagaccgagaucacccucgccaacggcgagauccgcaagcgcccccucaucgagaccaacggcgagaccggcgagaucgucugggacaagggccgcgacuucgccaccguccgcaagguccucagcaugccccaggucaacaucgucaagaagaccgagguccagaccggcggcuucagcaaggagagcauccuccccaagcgcaacagcgacaagcucaucgcccgcaagaaggacugggaccccaagaaguacggcggcuucgacagccccaccgucgccuacagcguccucgucgucgccaaggucgagaagggcaagagcaagaagcucaagagcgucaaggagcuccucggcaucaccaucauggagcgcagcagcuucgagaagaaccccaucgacuuccucgaggccaagggcuacaaggaggucaagaaggaccucaucaucaagcuccccaaguacagccucuucgagcucgagaacggccgcaagcgcaugcucgccagcgccggcgagcuccagaagggcaacgagcucgcccuccccagcaaguacgucaacuuccucuaccucgccagccacuacgagaagcucaagggcagccccgaggacaacgagcagaagcagcucuucgucgagcagcacaagcacuaccucgacgagaucaucgagcagaucagcgaguucagcaagcgcgucauccucgccgacgccaaccucgacaagguccucagcgccuacaacaagcaccgcgacaagcccauccgcgagcaggccgagaacaucauccaccucuucacccucaccaaccucggcgcccccgccgccuucaaguacuucgacaccaccaucgaccgcaagcgcuacaccagcaccaaggagguccucgacgccacccucauccaccagagcaucaccggccucuacgagacccgcaucgaccucagccagcucggcggcgacggcggcggcagccccaagaagaagcgcaaggucuagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - protein, 1379 aa, Unknown
  Description of Unknown: Amino acid sequence for Sp. Cas9 sequence
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 14 - protein, 1379 aa, Unknown
  Description of Unknown: Amino acid sequence Sp. Cas9 nickase sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 15 - RNA, 20 nt, Artificial Sequence
  Description of Artificial Sequence: Synthetic oligonucleotide
aaaggcugcugaugacaccu

SEQ ID NO: 16 - RNA, 100 nt, Artificial Sequence
  Description of Artificial Sequence: Synthetic polynucleotide
aaaggcugcugaugacaccuguuuuagagcuagaaauagcaaguuaaaauaaggcuaguccguuaucaacuugaaaaaguggcaccgagucggugcuuuu

SEQ ID NO: 17 - skipped

SEQ ID NO: 18 - RNA, 100 nt, Artificial Sequence
  Description of Artificial Sequence: Synthetic polynucleotide
  modified_base (1)..(20): a, c, u, g, unknown or other
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 19 - RNA, 100 nt, Artificial Sequence; identical to SEQ ID NO: 18
  Description of Artificial Sequence: Synthetic polynucleotide
  modified_base (1)..(20): a, c, u, g, unknown or other

SEQ ID NO: 20 - RNA, 88 nt, Artificial Sequence
  Description of Artificial Sequence: Synthetic oligonucleotide
  modified_base (1)..(20): a, c, u, g, unknown or other
nnnnnnnnnnnnnnnnnnnnguuuuagagcuagaaauagcaaguuaaaauaaggcuaguccguuaucaacuuggcaccgagucggugc

SEQ ID NO: 21 - RNA, 88 nt, Artificial Sequence; identical to SEQ ID NO: 20
  Description of Artificial Sequence: Synthetic oligonucleotide
  modified_base (1)..(20): a, c, u, g, unknown or other

SEQ ID NO: 22 - RNA, 90 nt, Artificial Sequence
  Description of Artificial Sequence: Synthetic oligonucleotide
  modified_base (1)..(20): a, c, u, g, unknown or other
nnnnnnnnnnnnnnnnnnnnguuuuagagcuagaaauagcaaguuaaaauaaggcuaguccguuaucacgaaagggcaccgagucggugc

SEQ ID NO: 23 - RNA, 90 nt, Artificial Sequence; identical to SEQ ID NO: 22
  Description of Artificial Sequence: Synthetic oligonucleotide
  modified_base (1)..(20): a, c, u, g, unknown or other

SEQ ID NO: 24 - RNA, 89 nt, Artificial Sequence
  Description of Artificial Sequence: Synthetic oligonucleotide
  modified_base (1)..(20): a, c, u, g, unknown or other
nnnnnnnnnnnnnnnnnnnnguuuuagagcuagaaauagcaaguuaaaauaaggcuaguccguuaucaaaaauggcaccgagucggugc

SEQ ID NO: 25 - RNA, 89 nt, Artificial Sequence; identical to SEQ ID NO: 24
  Description of Artificial Sequence: Synthetic oligonucleotide
  modified_base (1)..(20): a, c, u, g, unknown or other

SEQ ID NO: 26 - RNA, 88 nt, Artificial Sequence
  Description of Artificial Sequence: Synthetic oligonucleotide
  modified_base (1)..(20): a, c, u, g, unknown or other
nnnnnnnnnnnnnnnnnnnnguuuuagagcuagaaauagcaaguuaaaauaaggcuaguccguuaucacaagggcaccgagucggugc

SEQ ID NO: 27 - RNA, 88 nt, Artificial Sequence; identical to SEQ ID NO: 26
  Description of Artificial Sequence: Synthetic oligonucleotide
  modified_base (1)..(20): a, c, u, g, unknown or other

SEQ ID NO: 28 - RNA, 88 nt, Artificial Sequence
  Description of Artificial Sequence: Synthetic oligonucleotide
  modified_base (1)..(20): a, c, u, g, unknown or other
nnnnnnnnnnnnnnnnnnnnguuuuagagcuagaaauagcaaguuaaaauaaggcuaguccguuaucaaaauggcaccgagucggugc

SEQ ID NO: 29 - RNA, 88 nt, Artificial Sequence; identical to SEQ ID NO: 28
  Description of Artificial Sequence: Synthetic oligonucleotide
  modified_base (1)..(20): a, c, u, g, unknown or other

SEQ ID NO: 30 - RNA, 87 nt, Artificial Sequence
  Description of Artificial Sequence: Synthetic oligonucleotide
  modified_base (1)..(20): a, c, u, g, unknown or other
nnnnnnnnnnnnnnnnnnnnguuuuagagcgcgaagcgcaaguuaaaauaaggcuaguccguuaucaaaauggcaccgagucggugc

SEQ ID NO: 31 - RNA, 87 nt, Artificial Sequence; identical to SEQ ID NO: 30
  Description of Artificial Sequence: Synthetic oligonucleotide
  modified_base (1)..(20): a, c, u, g, unknown or other

SEQ ID NO: 32 - RNA, 80 nt, Artificial Sequence
  Description of Artificial Sequence: Synthetic oligonucleotide
guuuuagagcuagaaauagcaaguuaaaauaaggcuaguccguuaucaacuugaaaaaguggcaccgagucggugcuuuu

SEQ ID NO: 33 - RNA, 22 nt, Artificial Sequence
  Description of Artificial Sequence: Synthetic oligonucleotide
guuuuagagcuaugcuguuuug

SEQ ID NO: 34 - RNA, 100 nt, Artificial Sequence
  Description of Artificial Sequence: Synthetic polynucleotide
acacaaauaccaguccagcaguuuuagagcuagaaauagcaaguuaaaauaaggcuaguccguuaucaacuugaaaaaguggcaccgagucggugcuuuu

SEQ ID NO: 35 - RNA, 100 nt, Artificial Sequence
  Description of Artificial Sequence: Synthetic polynucleotide
acacaaauaccaguccagcgguuuuagagcuagaaauagcaaguuaaaauaaggcuaguccguuaucaacuugaaaaaguggcaccgagucggugcuuuu

SEQ ID NO: 36 - RNA, 4143 nt, Unknown
  Description of Unknown: ORF encoding Sp. Cas9 sequence
auggauaagaaguacucaaucgggcuggauaucggaacuaauuccguggguugggcagugaucacggaugaauacaaagugccguccaagaaguucaagguccuggggaacaccgauagacacagcaucaagaaaaaucucaucggagcccugcuguuugacuccggcgaaaccgcagaagcgacccggcucaaacguaccgcgaggcgacgcuacacccggcggaagaaucgcaucugcuaucugcaagagaucuuuucgaacgaaauggcaaaggucgacgacagcuucuuccaccgccuggaagaaucuuuccugguggaggaggacaagaagcaugaacggcauccuaucuuuggaaacaucgucgacgaaguggcguaccacgaaaaguacccgaccaucuaccaucugcggaagaaguugguugacucaacugacaaggccgaccucagauugaucuacuuggcccucgcccauaugaucaaauuccgcggacacuuccugaucgaaggcgaucugaacccugauaacuccgacguggauaagcuuuucauucaacuggugcagaccuacaaccaacuguucgaagaaaacccaaucaaugcuagcggcgucgaugccaaggccauccuguccgcccggcugucgaagucgcggcgccucgaaaaccugaucgcacagcugccgggagagaaaaagaacggacuuuucggcaacuugaucgcucucucacugggacucacucccaauuucaaguccaauuuugaccuggccgaggacgcgaagcugcaacucucaaaggacaccuacgacgacgacuuggacaauuugcuggcacaaauuggcgaucaguacgcggaucuguuccuugccgcuaagaaccuuucggacgcaaucuugcuguccgauauccugcgcgugaacaccgaaauaaccaaagcgccgcuuagcgccucgaugauuaagcgguacgacgagcaucaccaggaucucacgcugcucaaagcgcucgugagacagcaacugccugaaaaguacaaggagaucuucuucgaccaguccaagaauggguacgcaggguacaucgauggaggcgcuagccaggaagaguucuauaaguucaucaagccaauccuggaaaagauggacggaaccgaagaacugcuggucaagcugaacagggaggaucugcuccggaaacagagaaccuuugacaacggauccauuccccaccagauccaucugggugagcugcacgccaucuugcggcgccaggaggacuuuuacccauuccucaaggacaaccgggaaaagaucgagaaaauucugacguuccgcaucccguauuacgugggcccacuggcgcgcggcaauucgcgcuucgcguggaugacuagaaaaucagaggaaaccaucacuccuuggaauuucgaggaaguuguggauaagggagcuucggcacaaagcuucaucgaacgaaugaccaacuucgacaagaaucucccaaacgagaaggugcuuccuaagcacagccuccuuuacgaauacuucacugucuacaacgaacugacuaaagugaaauacguuacugaaggaaugaggaagccggccuuucuguccggagaacagaagaaagcaauugucgaucugcuguucaagaccaaccgcaaggugaccgucaagcagcuuaaagaggacuacuucaagaagaucgaguguuucgacucaguggaaaucagcgggguggaggacagauucaacgcuucgcugggaaccuaucaugaucuccugaagaucaucaaggacaaggacuuccuugacaacgaggagaacgaggacauccuggaagauaucguccugaccuugacccuuuucgaggaucgcgagaugaucgaggagaggcuuaagaccuacgcucaucucuucgacgauaaggucaugaaacaacucaagcgccgccgguacacugguuggggccgccucucccgcaagcugaucaacgguauucgcgauaaacagagcgguaaaacuauccuggauuuccucaaaucggauggcuucgcuaaucguaacuucaugcaauugauccacgacgacagccugaccuuuaaggaggacauccaaaaagcacaaguguccggacagggagacucacuccaugaacacaucgcgaaucuggccgguucgccggcgauuaagaagggaauucugcaaacugugaagguggucgacgagcuggugaaggucaugggacggcacaaaccggagaauaucgugauugaaauggcccgagaaaaccagacuacccagaagggccagaaaaacucccgcgaaaggaugaagcggaucgaagaaggaaucaaggagcugggcagccagauccugaaagagcacccgguggaaaacacgcagcugcagaacgagaagcucuaccuguacuauuugcaaaauggacgggacauguacguggaccaagagcuggacaucaaucgguugucugauuacgacguggaccacaucguuccacaguccuuucugaaggaugacucgaucgauaacaagguguugacucgcagcgacaagaacagagggaagucagauaaugugccaucggaggaggucgugaagaagaugaagaauuacuggcggcagcuccugaaugcgaagcugauuacccagagaaaguuugacaaucucacuaaagccgagcgcggcggacucucagagcuggauaaggcuggauucaucaaacggcagcuggucgagacucggcagauuaccaagcacguggcgcagaucuuggacucccgcaugaacacuaaauacgacgagaacgauaagcucauccgggaagugaaggugauuacccugaaaagcaaacuugugucggacuuucggaaggacuuucaguuuuacaaagugagagaaaucaacaacuaccaucacgcgcaugacgcauaccucaacgcuguggucgguaccgcccugaucaaaaaguacccuaaacuugaaucggaguuuguguacggagacuacaaggucuacgacgugaggaagaugauagccaaguccgaacaggaaaucgggaaagcaacugcgaaauacuucuuuuacucaaacaucaugaacuuuuucaagacugaaauuacgcuggccaauggagaaaucaggaagaggccacugaucgaaacuaacggagaaacgggcgaaaucgugugggacaagggcagggacuucgcaacuguucgcaaagugcucucuaugccgcaagucaauauugugaagaaaaccgaagugcaaaccggcggauuuucaaaggaaucgauccucccaaagagaaauagcgacaagcucauugcacgcaagaaagacugggacccgaagaaguacggaggauucgauucgccgacugucgcauacuccguccucgugguggccaagguggagaagggaaagagcaaaaagcucaaauccgucaaagagcugcuggggauuaccaucauggaacgauccucguucgagaagaacccgauugauuuccucgaggcgaaggguuacaaggaggugaagaaggaucugaucaucaaacuccccaaguacucacuguucgaacuggaaaauggucggaagcgcaugcuggcuucggccggagaacuccaaaaaggaaaugagcuggccuugccuagcaaguacgucaacuuccucuaucuugcuucgcacuacgaaaaacucaaagggucaccggaagauaacgaacagaagcagcuuuucguggagcagcacaagcauuaucuggaugaaaucaucgaacaaaucuccgaguuuucaaagcgcgugauccucgccgacgccaaccucgacaaaguccugucggccuacaauaagcauagagauaagccgaucagagaacaggccgagaacauuauccacuuguucacccugacuaaccugggagccccagccgccuucaaguacuucgauacuacuaucgaucgcaaaagauacacguccaccaaggaaguucuggacgcgacccugauccaccaaagcaucacuggacucuacgaaacuaggaucgaucugucgcagcuggguggcgauggcgguggaucuccgaaaaagaagagaaagguguaauga

SEQ ID NO: 37 - RNA, 13 nt, Artificial Sequence
  Description of Artificial Sequence: Synthetic oligonucleotide
gccgccrccaugg

SEQ ID NO: 38 - RNA, 100 nt, Artificial Sequence; identical to SEQ ID NO: 16
  Description of Artificial Sequence: Synthetic polynucleotide

SEQ ID NO: 39 - RNA, 100 nt, Artificial Sequence; identical to SEQ ID NO: 16
  Description of Artificial Sequence: Synthetic polynucleotide

SEQ ID NO: 40 - RNA, 100 nt, Artificial Sequence; identical to SEQ ID NO: 16
  Description of Artificial Sequence: Synthetic polynucleotide

SEQ ID NO: 41 - RNA, 100 nt, Artificial Sequence; identical to SEQ ID NO: 16
  Description of Artificial Sequence: Synthetic polynucleotide

SEQ ID NO: 42 - RNA, 91 nt, Artificial Sequence
  Description of Artificial Sequence: Synthetic oligonucleotide
acacaaauaccaguccagcgguuuuagagcuagaaauagcaaguuaaaauaaggcuaguccguuaucacgaaagggcaccgagucggugcu

SEQ ID NO: 43 - RNA, 88 nt, Artificial Sequence
  Description of Artificial Sequence: Synthetic oligonucleotide
aaaggcugcugaugacaccuguuuuagagcuagaaauagcaaguuaaaauaaggcuaguccguuaucaacuuggcaccgagucggugc

SEQ ID NO: 44 - RNA, 88 nt, Artificial Sequence; identical to SEQ ID NO: 43
  Description of Artificial Sequence: Synthetic oligonucleotide

SEQ ID NO: 45 - RNA, 90 nt, Artificial Sequence
  Description of Artificial Sequence: Synthetic oligonucleotide
aaaggcugcugaugacaccuguuuuagagcuagaaauagcaaguuaaaauaaggcuaguccguuaucacgaaagggcaccgagucggugc

SEQ ID NO: 46 - RNA, 90 nt, Artificial Sequence; identical to SEQ ID NO: 45
  Description of Artificial Sequence: Synthetic oligonucleotide

SEQ ID NO: 47 - RNA, 89 nt, Artificial Sequence
  Description of Artificial Sequence: Synthetic oligonucleotide
aaaggcugcugaugacaccuguuuuagagcuagaaauagcaaguuaaaauaaggcuaguccguuaucaaaaauggcaccgagucggugc

SEQ ID NO: 48 - RNA, 89 nt, Artificial Sequence; identical to SEQ ID NO: 47
  Description of Artificial Sequence: Synthetic oligonucleotide

SEQ ID NO: 49 - RNA, 88 nt, Artificial Sequence
  Description of Artificial Sequence: Synthetic oligonucleotide
aaaggcugcugaugacaccuguuuuagagcuagaaauagcaaguuaaaauaaggcuaguccguuaucacaagggcaccgagucggugc

SEQ ID NO: 50 - RNA, 88 nt, Artificial Sequence; identical to SEQ ID NO: 49
  Description of Artificial Sequence: Synthetic oligonucleotide

SEQ ID NO: 51 - RNA, 88 nt, Artificial Sequence
  Description of Artificial Sequence: Synthetic oligonucleotide
aaaggcugcugaugacaccuguuuuagagcuagaaauagcaaguuaaaauaaggcuaguccguuaucaaaauggcaccgagucggugc

SEQ ID NO: 52 - RNA, 88 nt, Artificial Sequence; identical to SEQ ID NO: 51
  Description of Artificial Sequence: Synthetic oligonucleotide

SEQ ID NO: 53 - RNA, 87 nt, Artificial Sequence
  Description of Artificial Sequence: Synthetic oligonucleotide
aaaggcugcugaugacaccuguuuuagagcgcgaagcgcaaguuaaaauaaggcuaguccguuaucaaaauggcaccgagucggugc

SEQ ID NO: 54 - RNA, 87 nt, Artificial Sequence; identical to SEQ ID NO: 53
  Description of Artificial Sequence: Synthetic oligonucleotide

SEQ ID NO: 55 - RNA, 91 nt, Artificial Sequence
  Description of Artificial Sequence: Synthetic oligonucleotide
  modified_base (1)..(20): a, c, u, g, unknown or other
nnnnnnnnnnnnnnnnnnnnguuuuagagcuagaaauagcaaguuaaaauaaggcuaguccguuaucacgaaagggcaccgagucggugcu

SEQ ID NO: 56 - RNA, 99 nt, Artificial Sequence
  Description of Artificial Sequence: Synthetic oligonucleotide
  modified_base (1)..(19): a, c, u, g, unknown or other
nnnnnnnnnnnnnnnnnnnguuuuagagcuagaaauagcaaguuaaaauaaggcuaguccguuaucaacuugaaaaaguggcaccgagucggugcuuuu

SEQ ID NO: 57 - RNA, 100 nt, Artificial Sequence; identical to SEQ ID NO: 18
  Description of Artificial Sequence: Synthetic polynucleotide
  modified_base (1)..(20): a, c, u, g, unknown or other

SEQ ID NO: 58 - RNA, 100 nt, Artificial Sequence
  Description of Artificial Sequence: Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 59 - DNA, 39 bp, Unknown
  Description of Unknown: TTR gene sequence
agacacaaataccagtccagcgaggcagagraggagcag

SEQ ID NO: 60 - DNA, 40 bp, Artificial Sequence
  Description of Artificial Sequence: Synthetic oligonucleotide
agacacaaataccagtccaagcgaggcagagraggagcag

SEQ ID NO: 61 - DNA, 40 bp, Artificial Sequence
  Description of Artificial Sequence: Synthetic oligonucleotide
  modified_base (20)..(20): a, c, t, g, unknown or other
agacacaaataccagtccangcgaggcagagraggagcag

SEQ ID NO: 62 - RNA, 79 nt, Artificial Sequence
  Description of Artificial Sequence: Synthetic oligonucleotide
uuuuagagcuagaaauagcaaguuaaaauaaggcuaguccguuaucaacuugaaaaaguggcaccgagucggugcuuuu

(19) United States
(12) Patent Application Publication
(10) Pub. No.: US 2025/0269062 A1
(43) Pub. Date: Aug. 28, 2025

(54) METHODS FOR IN VIVO EDITING OF A LIVER GENE

(71) Applicant: INTELLIA THERAPEUTICS, INC., Cambridge, MA (US)

(72) Inventors: David LEBWOHL, Cambridge, MA (US); Michael MAITLAND, Cambridge, MA (US); Mark STROH, Cambridge, MA (US); Yuanxin XU, Cambridge, MA (US)

(21) Appl. No.: 18/572,155

(22) PCT Filed: Jun. 22, 2022
(86) PCT No.: PCT/US2022/034454

§ 371 Date: Dec. 19, 2023

Related U.S. Application Data
(60) Provisional application No. 63/314,878, filed on Feb. 28, 2022.
(60) Provisional application No. 63/264,435, filed on Nov. 22, 2021.
(60) Provisional application No. 63/263,466, filed on Nov. 3, 2021.
(60) Provisional application No. 63/202,812, filed on Jun. 25, 2021.
(60) Provisional application No. 63/202,744, filed on Jun. 22, 2021.

(51) Int. Cl. A61K 48/00; C12N 9/22; C12N 15/11; C12N 15/88

(52) U.S. Cl. CPC A61K 48/0058; C12N 9/226; C12N 15/11; C12N 15/88; C12N 2310/20

(57) ABSTRACT

The first systemic administration of a CRISPR/Cas9-based therapeutic for in vivo editing in a clinical trial is described. Described herein are methods for in vivo editing of a liver gene by systemically administering a lipid nanoparticle composition comprising an mRNA encoding a Cas nuclease and a guide RNA that targets the gene. For example, disclosed herein are methods for in vivo editing of a transthyretin gene by systemically administering a lipid nanoparticle composition comprising an mRNA encoding a Cas nuclease and a guide RNA that targets the TTR gene. Assessment of biosafety metrics and clinical efficacy metrics, as well as methods of treatment, are also described herein.

### SEQUENCE LISTING

The instant application contains a Sequence Listing which has been submitted electronically in ASCII format and is hereby incorporated by reference in its entirety. Said ASCII copy, created on Sep. 9, 2022, is named 12793_0031-00304_SL.txt and is 113,976 bytes in size.

### BACKGROUND

This application is a U.S. national stage entry under 35 U.S.C. § 371 of international application number PCT/US2022/034454, filed Jun. 22, 2022, which designates the U.S. and claims the benefit of U.S. Provisional Application No. 63/202,744, filed on Jun. 22, 2021; U.S. Provisional Application No. 63/202,812, filed on Jun. 25, 2021; U.S. Provisional Application No. 63/263,466, filed on Nov. 3, 2021; U.S. Provisional Application No. 63/264,435, filed on Nov. 22, 2021; and U.S. Provisional Application No. 63/314,878, filed on Feb. 28, 2022, the contents of each of which are hereby incorporated by reference in their entireties.

Amyloidosis characterized by accumulation in tissues of amyloid fibrils composed of misfolded transthyretin (TTR) protein may be referred to as ATTR and is a progressive fatal disease (Marcoux et al., EMBO Mol Med 2015; Gertz et al., J Am Coll Cardiol 2015). ATTR can present with a wide spectrum of symptoms, and subjects with different classes of ATTR may have different characteristics and prognoses. Some classes of ATTR include familial amyloid polyneuropathy (FAP), familial amyloid cardiomyopathy (FAC), and wild-type TTR amyloidosis (wt-TTR amyloidosis). FAP commonly presents with sensorimotor neuropathy, while FAC and wt-TTR amyloidosis commonly present with congestive heart failure. ATTR amyloidosis may be acquired (wild-type ATTR amyloidosis; ATTRwt) and is a recognized cause of cardiomyopathy and heart failure (Gertz et al., J Am Coll Cardiol 2015). TTR amyloidosis may be hereditary (variant ATTR; ATTRv; hATTR) and triggered by over 100 pathogenic mutations in the TTR gene (Ando et al., Orphanet J Rare Dis 2013). ATTRv amyloidosis, thought to be present in approximately 50,000 individuals worldwide (Hawkins et al., Ann Med, 2015; Schmidt et al., Muscle Nerve, 2018), has an autosomal dominant pattern of inheritance and a clinical phenotype dominated by amyloid polyneuropathy or cardiomyopathy, with most subjects demonstrating a combination of the two (ATTR-PN-CM) (Dohrn et al., J Neurochem 2021). Following the onset of symptoms, ATTR amyloidosis is progressive, culminating in death within a median of 2-6 years after diagnosis in subjects with amyloid cardiomyopathy (Maurer et al., Circ Heart Fail, 2019) and 4-17 years after symptom onset in subjects with amyloid polyneuropathy in the absence of cardiomyopathy (Merlini et al., Neurol Ther 2020).

TTR is a protein produced by the TTR gene that normally functions to transport retinol and thyroxine throughout the body. TTR is predominantly synthesized in the liver, with small fractions being produced in the choroid plexus and retina. TTR normally circulates as a soluble tetrameric protein in the blood. Pathogenic variants of TTR, which may disrupt tetramer stability, can be encoded by mutant alleles of the TTR gene. Mutant TTR may result in misfolded TTR, which may generate amyloids (i.e., aggregates of misfolded TTR protein). In some cases, pathogenic variants of TTR can lead to amyloidosis, or disease resulting from build-up of amyloids. For example, misfolded TTR monomers can polymerize into amyloid fibrils within tissues, such as the peripheral nerves, heart, and gastrointestinal tract. Amyloid fibrils can also comprise wild-type TTR that has deposited on misfolded TTR.

Current treatments for ATTR amyloidosis rely on reducing ongoing amyloid formation via stabilization of the tetrameric form of TTR (diflunisal, tafamidis) (Maurer et al., NEJM 2018; Berk et al., Jama 2013) or via inhibition of TTR protein synthesis (inotersen, patisiran) through degradation of TTR mRNA (Benson et al., NEJM 2018; Adams et al., NEJM 2018). Such treatments produce symptom relief, functional improvement and prolonged survival (Adams et al., NEJM 2018; Adams et al., Lancet Neurol 2021; Solomon et al., Circulation 2019), but are limited by the requirement for lifelong administration to maintain TTR knockdown. More generally, existing gene editing approaches for many disorders produce short-term effects in gene expression but would require chronic administration to maintain the desired effects in gene expression. In the case of patisiran, chronic treatment is associated with premedication with glucocorticoids and antihistamines (Urits et al., Neurol Ther 2020). In addition, subjects receiving TTR-stabilizing agents experience disease progression (Lozeron et al., Eur J Neurol 2013). Inotersen is associated with serious side effects, including glomerulonephritis and decreased platelet count (Gertz et al., Expert Rev Clin Pharmacol 2019). More extensive TTR knockdown is associated with greater improvement in neuropathy endpoints in subjects with hATTR polyneuropathy (Adams et al., NEJM 2018). Enhancements in TTR reduction, including sustained knockdown, may translate to improved outcomes for subjects with ATTR amyloidosis. Thus, there remains an unmet need for gene editing therapies that are capable of producing long-lasting effects in gene expression, e.g., knockdown of TTR, without requiring chronic administration.

### SUMMARY

The present disclosure describes the first systemic administration of a CRISPR/Cas9-based therapeutic for in vivo editing in a clinical trial. In some embodiments, the present invention provides methods using a guide RNA with a Cas nuclease such as the CRISPR/Cas system to substantially reduce or knockdown expression of the TTR gene, thereby substantially reducing or eliminating the production of TTR protein associated with ATTR. The substantial reduction or elimination of the production of TTR protein associated with ATTR through alteration of the TTR gene can be a long-term reduction or elimination of serum TTR levels, such as a durable reduction of serum TTR. Additional embodiments include a lipid nanoparticle system for use in in vivo liver-targeted delivery of a CRISPR/Cas RNA components to a human subject, such as guide RNA and mRNA encoding a Cas nuclease, and methods of using the same.

In one aspect, provided herein is a method for treating amyloidosis associated with TTR (ATTR) in a human subject, comprising systemically administering to the human subject a LNP composition, wherein the LNP composition comprises an effective amount of i. an mRNA encoding a Cas nuclease, and ii. a guide RNA that targets the TTR gene, thereby treating ATTR, wherein the administration of the composition reduces serum TTR relative to baseline serum.

In one aspect, provided herein is a method for in vivo editing of the transthyretin (TTR) gene in a human subject having amyloidosis associated with TTR (ATTR), also known as transthyretin amyloidosis), comprising systemically administering to the human subject a lipid nano particle (LNP) composition, comprising i. an mRNA encoding a Cas nuclease, and ii. a guide RNA that targets the TTR gene, editing the gene at the site targeted by the guide RNA in a hepatocyte of the subject; wherein the administration of the composition results in a change in a level of a biosafety metric in the subject that is acceptable as compared to a baseline level of the biosafety metric.

In one aspect, provided herein is a method for in vivo editing of the transthyretin (TTR) gene in a human subject having amyloidosis associated with TTR (ATTR), comprising systemically administering to the human subject a LNP composition wherein the LNP composition comprises an effective amount of i. an mRNA encoding a Cas nuclease, and ii. a guide RNA that targets the TTR gene, and editing the TTR gene at the site targeted by the guide RNA in a hepatocyte of the subject; wherein the administration of the composition results in a clinically significant improvement in a level of a clinical metric in the subject as compared to a baseline level.

In one aspect, provided herein is a method for treating amyloidosis associated with TTR (ATTR) in a human subject, comprising systemically administering to the human subject a LNP composition, wherein the LNP comprises an effective amount of i. an mRNA encoding a Cas nuclease, and ii. a guide RNA that targets the TTR gene, thereby treating ATTR, wherein the administration of the composition results in a clinically significant improvement in a level of a clinical metric in the subject as compared to a baseline level of the clinical metric.

In one aspect, provided herein is a method for treating amyloidosis associated with TTR (ATTR) in a human subject, comprising systemically administering to the human subject a LNP composition, wherein the LNP composition comprises an effective amount of i. an mRNA encoding a Cas nuclease, and ii. a guide RNA that targets the TTR gene, thereby treating ATTR, wherein the mRNA encoding a Cas nuclease and the guide RNA that targets the TTR gene are administered at a combined dose of about 25 to about 100 mg.

In one aspect, provided herein is a method for in vivo editing of a gene in the liver of a human subject having a monogenic disorder, comprising systemically administering to the human subject a LNP composition, wherein the LNP composition comprises i. an mRNA encoding a Cas nuclease, and ii. a guide RNA that targets a gene in the liver, editing the gene at the site targeted by the guide RNA in a hepatocyte of the subject; wherein the administration of the composition results in a change in a level of a biosafety metric in the subject that is acceptable as compared to a baseline level of the biosafety metric. In some embodiments, the monogenic disorder is ATTR. In some embodiments, the gene is TTR.

In one aspect, provided herein is a method for treating a human subject having a monogenic disorder, comprising systemically administering to the human subject a LNP composition, wherein the LNP composition comprises an effective amount of: i. an mRNA encoding a Cas nuclease, and ii. a guide RNA that targets a gene in the liver; and editing the gene in the liver thereby treating the monogenic disorder, wherein the treatment is safe and well-tolerated. In some embodiments, the monogenic disorder is ATTR. In some embodiments, the gene is TTR.

In one aspect, provided herein is a method for treating a human subject having a monogenic disorder, comprising systemically administering to the human subject a LNP composition, wherein the LNP composition comprises an effective amount of i. an mRNA encoding a Cas nuclease, and ii. a guide RNA that targets a gene in the liver. The method further comprises determining a first level of a biosafety metric in the subject prior to administration, determining a second level of the biosafety metric in the subject a period of time after administration; and assessing the change between the first and the second level of the biosafety metric. In some embodiments, the change between the first and the second level of the biosafety metric is an acceptable change. In some embodiments, the monogenic disorder is ATTR. In some embodiments, the gene is TTR.

In any of the foregoing aspects and embodiments, the LNP comprises (9Z, 12Z)-3-((4,4-bis(octyloxy)butanoyl)oxy)-2-((((3-(diethylamino)propoxy)carbonyl)oxy)methyl)propyl octadeca-9, 12-dienoate.

In any of the foregoing aspects and embodiments, the LNP comprises a PEG lipid. In some embodiments, the PEG lipid comprises dimyristoylglycerol (DMG). In some embodiments, the PEG lipid comprises PEG-2k. In some embodiments, the PEG lipid is PEG-DMG2000.

In any of the foregoing aspects and embodiments, the LNP composition has an N/P ratio of about 5-7. In any of the foregoing aspects and embodiments, the N/P ratio of the LNP composition is about 4-6.

In any of the foregoing aspects and embodiments, the guide RNA and Cas nuclease are present in a ratio ranging from about 5:1 to about 1:5 by weight. In any of the foregoing aspects and embodiments the guide RNA and Cas nuclease are present in a ratio ranging from about 3:1 to about 1:3 by weight. In any of the foregoing aspects and embodiments the guide RNA and Cas nuclease are present in a ratio ranging from about 2:1 to about 1:2 by weight.

In any of the foregoing aspects and embodiments, the mRNA encodes a Class 2 Cas nuclease. In some embodiments, the mRNA encodes a Cas9 nuclease. In some embodiments, the mRNA encodes S. pyogenes Cas9. In some embodiments, the mRNA encoding the Cas nuclease is codon-optimized. In some embodiments, the mRNA comprises at least one modification.

In any of the foregoing aspects and embodiments, the guide RNA comprises at least one modification. In some embodiments, the at least one modification to the guide RNA includes a 2′-O-methyl modified nucleotide and/or a phosphorothioate bond between nucleotides.

In any of the foregoing aspects and embodiments, the ATTR is hereditary transthyretin amyloidosis. In any of the foregoing aspects and embodiments, the ATTR is wild-type transthyretin amyloidosis. In any of the foregoing aspects and embodiments, the ATTR is hereditary transthyretin amyloidosis with polyneuropathy. In any of the foregoing aspects and embodiments, the ATTR is hereditary transthyretin amyloidosis with cardiomyopathy. In any of the foregoing aspects and embodiments, the ATTR is wildtype transthyretin amyloidosis with cardiomyopathy, e.g., wherein the subject is classified under the New York Health Association (NYHA) classification as Class I, Class II, or Class III. In any of the foregoing aspects and embodiments, the subject has ATTRv-PN and/or ATTR-CM.

In any of the foregoing aspects and embodiments, the administration of the LNP composition results in a change in a level of a biosafety metric in the subject that is acceptable as compared to a baseline level of the biosafety metric.

In any of the foregoing aspects and embodiments, the biosafety metric is prothrombin. In any of the foregoing aspects and embodiments, the biosafety metric is activated partial thromboplastin time (aPTT). In any of the foregoing aspects and embodiments the biosafety metric is fibrinogen. In any of the foregoing aspects and embodiments the biosafety metric is alanine aminotransferase (ALT). In any of the foregoing aspects and embodiments the biosafety metric is aspartate aminotransferase (AST).

In any of the foregoing aspects and embodiments, the mRNA encoding a Cas nuclease and the guide RNA that targets the TTR gene are administered at a combined dose of about 0.3 mg/kg to about 2 mg/kg. In any of the foregoing aspects and embodiments, the mRNA encoding a Cas nuclease and the guide RNA that targets the TTR gene are administered at a combined dose of about 0.3 mg/kg to about 1 mg/kg. In any of the foregoing aspects and embodiments, the mRNA encoding a Cas nuclease and the guide RNA that targets the TTR gene are administered at a combined dose of about 0.3 mg/kg. In any of the foregoing aspects and embodiments, the mRNA encoding a Cas nuclease and the guide RNA that targets the TTR gene are administered at a combined dose of about 0.7 mg/kg. In any of the foregoing aspects and embodiments, the mRNA encoding a Cas nuclease and the guide RNA that targets the TTR gene are administered at a combined dose of about 1.0 mg/kg.

In any of the foregoing aspects and embodiments, the mRNA encoding a Cas nuclease and the guide RNA that targets the TTR gene are administered at a combined dose of about 5 mg to about 9 mg of total RNA. In any of the foregoing aspects and embodiments the mRNA encoding a Cas nuclease and the guide RNA that targets the TTR gene are administered at a combined dose of about 15 mg to about 27 mg of total RNA. In any of the foregoing aspects and embodiments, the mRNA encoding a Cas nuclease and the guide RNA that targets the TTR gene are administered at a combined dose of about 7 mg to about 9 mg of total RNA. In any of the foregoing aspects and embodiments, the mRNA encoding a Cas nuclease and the guide RNA that targets the TTR gene are administered at a combined dose of about 25 mg to about 27 mg of total RNA.

In any of the foregoing aspects and embodiments the mRNA encoding a Cas nuclease and the guide RNA that targets the TTR gene are administered at a combined dose of about 25 mg to about 150 mg of total RNA. In any of the foregoing aspects and embodiments, the mRNA encoding a Cas nuclease and the guide RNA that targets the TTR gene are administered at a combined dose of about 25 mg to about 100 mg of total RNA. In any of the foregoing aspects and embodiments, the mRNA encoding a Cas nuclease and the guide RNA that targets the TTR gene are administered at a combined dose of about 50 mg to about 90 mg of total RNA. In any of the foregoing aspects and embodiments, the mRNA encoding a Cas nuclease and the guide RNA that targets the TTR gene are administered at a combined dose of about 50 mg of total RNA.

In any of the foregoing aspects and embodiments, the mRNA encoding a Cas nuclease and the guide RNA that targets the TTR gene are administered at a combined dose of about 35 mg to 65 mg of total RNA. In any of the foregoing aspects and embodiments, the mRNA encoding a Cas nuclease and the guide RNA that targets the TTR gene are administered at a combined dose of about 40 mg of total RNA. In any of the foregoing aspects and embodiments, the mRNA encoding a Cas nuclease and the guide RNA that targets the TTR gene are administered at a combined dose of about 60 mg of total RNA. In any of the foregoing aspects and embodiments, the mRNA encoding a Cas nuclease and the guide RNA that targets the TTR gene are administered at a combined dose of about 70 mg of total RNA. In any of the foregoing aspects and embodiments, the mRNA encoding a Cas nuclease and the guide RNA that targets the TTR gene are administered at a combined dose of about 80 mg of total RNA. In any of the foregoing aspects and embodiments, the mRNA encoding a Cas nuclease and the guide RNA that targets the TTR gene are administered at a combined dose of about 90 mg of total RNA. In any of the foregoing aspects and embodiments, the mRNA encoding a Cas nuclease and the guide RNA that targets the TTR gene are administered at a combined dose of about 100 mg of total RNA.

In any of the foregoing aspects and embodiments, the clinical metric is serum TTR level.

In any of the foregoing aspects and embodiments, the clinical metric is serum prealbumin level.

In any of the foregoing aspects and embodiments, administration of the LNP composition reduces or knocks down expression of the TTR gene.

In any of the foregoing aspects and embodiments, administration of the LNP composition reduces or knocks down expression of the TTR gene by 60-70%, 70-80%, 80-90%, 90-95%, 95-98%, 98-99%, or 99-100% as compared to baseline before administration of the composition.

In any of the foregoing aspects and embodiments, administration of the LNP composition reduces or knocks down expression of the TTR gene by 80%, 81%, 82%, 83%, 84%, 85%, 86%, 87%, 88%, 89%, 90%, 91%, 92%, 93%, 94%, 95%, 96%, 97%, 98%, or 99% as compared to baseline before administration of the composition.

In any of the foregoing aspects and embodiments, administration of the LNP composition reduces TTR serum level in the subject by at least 60% as compared to serum TTR level before administration of the composition (e.g., baseline).

In any of the foregoing aspects and embodiments, administration of the LNP composition reduces TTR serum level in the subject by at least 70% as compared to serum TTR level before administration of the composition (e.g., baseline).

In any of the foregoing aspects and embodiments, administration of the LNP composition reduces TTR serum level in the subject by at least 80% as compared to serum TTR level before administration of the composition (e.g., baseline).

In any of the foregoing aspects and embodiments, administration of the LNP composition reduces TTR serum level in the subject by at least 84% as compared to serum TTR level before administration of the composition (e.g., baseline).

In any of the foregoing aspects and embodiments, administration of the LNP composition reduces TTR serum level in the subject by at least 90% as compared to serum TTR level before administration of the composition (e.g., baseline).

In any of the foregoing aspects and embodiments, administration of the LNP composition reduces TTR serum level in the subject by at least 95% as compared to serum TTR level before administration of the composition (e.g., baseline).

In any of the foregoing aspects and embodiments, administration of the LNP composition reduces TTR serum level in the subject by at least 96% as compared to serum TTR level before administration of the composition (e.g., baseline).

In any of the foregoing aspects and embodiments, administration of the LNP composition reduces TTR serum level in the subject by 60-70%, 70-80%, 80-90%, 90-95%, 95-98%, 98-99%, or 99-100% as compared to serum TTR level before administration of the composition (e.g., baseline).

In any of the foregoing aspects and embodiments, administration of the LNP composition reduces TTR serum level in the subject by any of the foregoing amounts at 7 days after administration of the LNP composition.

In any of the foregoing aspects and embodiments, administration of the LNP composition reduces TTR serum level in the subject by any of the foregoing amounts at 14 days after administration of the LNP composition.

In any of the foregoing aspects and embodiments, administration of the LNP composition reduces TTR serum level in the subject by any of the foregoing amounts at 28 days after administration of the LNP composition.

In any of the foregoing aspects and embodiments, administration of the LNP composition reduces serum prealbumin level in the subject by at least 60% as compared to serum prealbumin level before administration of the composition (e.g., baseline).

In any of the foregoing aspects and embodiments, administration of the LNP composition reduces serum prealbumin level in the subject by at least 70% as compared to serum prealbumin level before administration of the composition (e.g., baseline).

In any of the foregoing aspects and embodiments, administration of the LNP composition reduces serum prealbumin level in the subject by at least 80% as compared to serum prealbumin level before administration of the composition (e.g., baseline).

In any of the foregoing aspects and embodiments, administration of the LNP composition reduces serum prealbumin level in the subject by at least 84% as compared to serum prealbumin level before administration of the composition (e.g., baseline).

In any of the foregoing aspects and embodiments, administration of the LNP composition reduces serum prealbumin level in the subject by at least 90% as compared to serum prealbumin level before administration of the composition (e.g., baseline).

In any of the foregoing aspects and embodiments, administration of the LNP composition reduces serum prealbumin level in the subject by at least 95% as compared to serum prealbumin level before administration of the composition (e.g., baseline).

In any of the foregoing aspects and embodiments, administration of the LNP composition reduces serum prealbumin level in the subject by at least 96% as compared to serum prealbumin level before administration of the composition (e.g., baseline).

In any of the foregoing aspects and embodiments, administration of the LNP composition reduces serum prealbumin level in the subject by 60-70%, 70-80%, 80-90%, 90-95%, 95-98%, 98-99%, or 99-100% as compared to serum prealbumin level before administration of the composition (e.g., baseline).

In any of the foregoing aspects and embodiments, administration of the LNP composition reduces serum TTR levels to less than about 50 μg/mL. In any of the foregoing aspects and embodiments, administration of the LNP composition reduces serum TTR levels to less than about 40 μg/mL. In any of the foregoing aspects and embodiments, administration of the LNP composition reduces serum TTR levels to less than about 30 μg/mL. In any of the foregoing aspects and embodiments, administration of the LNP composition reduces serum TTR levels to less than about 20 μg/mL. In any of the foregoing aspects and embodiments, administration of the LNP composition reduces serum TTR levels to less than about 10 μg/mL.

In any of the foregoing aspects and embodiments, the LNP composition is also administered with a second therapeutic agent. In any of the foregoing aspects and embodiments, the second therapeutic agent is a stabilizer of the tetrameric form of TTR. In any of the foregoing aspects and embodiments, the second therapeutic is diflunisal or tafamidis.

In any of the foregoing aspects and embodiments, administration of the LNP composition reduces serum prealbumin level in the subject by any of the foregoing amounts at 14 days after administration of the LNP composition.

In any of the foregoing aspects and embodiments, administration of the LNP composition reduces serum prealbumin level in the subject by any of the foregoing amounts at 28 days after administration of the LNP composition.

In any of the foregoing aspects and embodiments, the method further comprises durably reducing expression of the gene, e.g. the TTR gene, after a single administration of the LNP composition.

In any of the foregoing aspects and embodiments, serum TTR level or serum prealbumin level at 28 days after administration of the LNP composition is durable.

In any of the foregoing aspects and embodiments, serum TTR level or serum prealbumin level at 28 days after administration of the LNP composition is durable, e.g., at 2 months, at 3 months, at 4 months, at 5 months, at 6 months, at 7 months, at 8 months, at 9 months, at 10 months, at 11 months, and/or at 12 months.

In one aspect, disclosed herein is a method for treating amyloidosis associated with TTR (ATTR) in a human subject, comprising administering to the subject an effective amount of a composition that reduces serum TTR level in the subject by at least 95% as compared to a baseline serum TTR level.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Summary of methods for NTLA-2001 LNP-based gene therapy. Panel A describes composition of LNP particle and the infusion therapy, panel B illustrates a proposed mechanism of gene therapy delivery, panel C describes CRISPR-Cas9 based gene editing of TTR gene.

FIG. 2: Primary human hepatocyte cell culture-based evaluation of NTLA-2001 editing of TTR gene as a function of sgRNA concentration. Primary metrics shown are TTR gene editing, TTR mRNA levels and TTR protein levels as a percentage compared to control.

FIG. 3A: In vivo evaluation of TTR editing frequency and gene editing pattern using Cyn-LNP in non-human primates. FIG. 3B discloses SEQ ID NOS 59-61, respectively, in order of appearance.

FIGS. 4A-C: Serum TTR protein concentration change (compared to control) in humans treated with NTLA-2001 as a function of time (data shown to day 28). Metrics shown are from both cohorts FIG. 4A and FIG. 4B.

FIG. 5: Potential off target sites of the sgRNA of NTLA-2001 as identified by Cas-OFFinder, GUIDE-seq, and SITE-Seq.

FIGS. 6A-B: On- and off-target gene editing frequency evaluated in primary human hepatocyte cell cultures treated with NTLA-2001.

FIG. 7: Summary figure describing method used to characterize gene mutations induced by NTLA-2001.

FIGS. 8A-B: Summary figures describing PCR methods used for high throughput sequencing.

FIG. 9: Evaluation of structural variants detected surrounding the TTR locus from NTLA-2001 treated human hepatocytes primary cells.

FIGS. 10A-B: Dose-dependent evaluation of NTLA-2001 editing in mouse models measured as a percentage of TTR gene editing in liver and serum protein levels.

FIG. 11: Evaluation of permanence of NTLA-2001 based editing of TTR gene measured by serum TTR levels after partial hepatic resection in mice.

FIG. 12: Serum RNA concentration as a function of time in Cyn-LNP treated non-human primates.

FIGS. 13A-B: Evaluation of TTR editing measured by percent gene editing (FIG. 13A) and serum TTR levels (FIG. 13B) in Cyn-LNP treated non-human primates.

FIG. 14: Correlation of TTR serum protein levels and percent gene editing in liver in Cyn-LNP treated non-human primates.

FIGS. 15A-E: Liver and coagulation parameters in NTLA-2001 treated subjects, measured as prothrombin time (PT; FIG. 15A), activated partial thromboplastin time (aPTT;

FIG. 15D), fibrinogen (FIG. 15B), alanine aminotransferase (ALT; FIG. 15C) and aspartate aminotransferase (AST; FIG. 15E).

FIG. 16: Plasma concentration of components of Cyn-LNP in non-human primates.

FIG. 17: NTLA-2001 treatment adverse events.

FIG. 18: Enrolled clinical trial subject characteristics.

FIGS. 19A-D: Clinical trial subject data for polyneuropathy dose escalation study.

FIG. 20: TTR reduction by dose. SE, standard error. (*) N=2 at Month 2. (1) N=5 at Month 2.

FIGS. 21A-F: Data for prothrombin time (PT; FIG. 21A), activated partial thromboplastin time (aPTT; FIG. 21B;), fibrinogen (FIG. 21C), alanine aminotransferase (ALT; FIG. 21D), aspartate aminotransferase (AST; FIG. 21E), and d-dimer (FIG. 21F). SE=standard error.

FIG. 22: NTLA-2001 treatment adverse events, including cohorts 3 and 4.

FIGS. 23A-B: Enrolled clinical trial subject demographics and baseline characteristics.

FIG. 24: Interim mean plasma concentration-time profiles of LP01 following single dose IV infusion of NTLA-2001. NTLA-2001 declines rapidly from peak and then exhibits a secondary peak and log-linear phase. Available LP01 PK data are depicted up to 48 hours post-dose.

FIG. 25: Interim mean (SE) observed (points) and model-predicted (line) day 28 TTR versus NTLA (AUC mg*h/mL), shows day 28 serum TTR decreases with increasing NTLA-2001 exposure. Predose TTR concentration data is depicted at AUC=0. Data are depicted at the mean of the distribution of individual observed TTR and AUC values at each indicated dose level.

FIG. 26: Interim model-predicted distribution of NTLA-2001 AUC (mg*h/mL) following 1.0 mg/kg and 80 mg by indicated weight quartile. Simulations identified NTLA-2001 80 mg as the fixed dose equivalent to 1.0 mg/kg.

FIG. 27: Minor, transient changes in AST and ALT levels observed post NTLA-2001 infusion.

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended embodiments.

Before describing the present teachings in detail, it is to be understood that the disclosure is not limited to specific compositions or process steps, as such may vary. It should be noted that, as used in this specification and the appended embodiments, the singular form “a”, “an” and “the” include plural references unless the context clearly dictates otherwise. Thus, for example, reference to “a conjugate” includes a plurality of conjugates and reference to “a cell” includes a plurality or population of cells and the like. As used herein, the term “include” and its grammatical variants are intended to be non-limiting, such that recitation of items in a list is not to the exclusion of other like items that can be substituted or added to the listed items.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise. The term “about”, when used before a list, modifies each member of the list. The term “about” or “approximately” means an acceptable error for a particular value as determined by one of ordinary skill in the art, which depends in part on how the value is measured or determined. In some embodiments of the invention, “about” includes ±10%, or optionally ±5% of the stated value.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 20 nucleotide nucleic acid molecule” means that 18, 19, or 20 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, ranges include both the upper and lower limit.

As used herein, it is understood that when the maximum amount of a value is represented by 100% (e.g., 100% inhibition or 100% encapsulation) that the value is limited by the method of detection. For example, 100% inhibition is understood as inhibition to a level below the level of detection of the assay, and 100% encapsulation is understood as no material intended for encapsulation can be detected outside the vesicles.

Unless stated otherwise, the following terms and phrases as used herein are intended to have the following meanings.

“mRNA” is used herein to refer to a polynucleotide comprising RNA or modified RNA that includes an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of a nucleic acid phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. In some embodiments, a polynucleotide is chemically synthesized or in vitro transcribed. A polynucleotide may be an mRNA, such as in vitro transcribed RNA comprising modified uridine. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. An RNA may comprise DNA or one or more deoxynucleosides or deoxynucleoside analogs. “Guide RNA”, “gRNA”, and “guide” are used herein interchangeably to refer to an RNA such as a crRNA (also known as CRISPR RNA), or the combination of a crRNA and a trRNA (also known as tracrRNA), which targets a Cas nuclease to a genomic location. Cognate guide RNA structures for Cas nucleases such as Cas9 nucleases are known in the art. The crRNA and trRNA sequences of a guide RNA may be associated as a single RNA molecule (single guide RNA, sgRNA) or as, e.g. separate RNA molecules (dual guide RNA, dgRNA). The trRNA may be a naturally-occurring sequence, or a trRNA sequence with modifications or variations compared to naturally-occurring sequences. Guide RNAs can include modified RNAs as described herein.

As used herein, a “guide sequence” refers to a sequence within a guide RNA that is complementary to a target sequence and functions to direct a guide RNA to a target sequence for binding or modification (e.g., cleavage) by a Cas nuclease. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. The guide sequence may be 18-25 or 18-20 nucleotides in length. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 18, 19, 20 or more base pairs. In some embodiments, the guide sequence and the target region may contain 0 or 1-4 mismatches where the guide sequence comprises at least 18, 19, 20 or more nucleotides. In some embodiments, the guide sequence and the target region may contain 1, 2, 3, or 4 mismatches where the guide sequence comprises 20 nucleotides.

Target sequences for Cas proteins include both the positive and negative strands of genomic DNA (i.e., the sequence given and the sequence's reverse compliment), as a nucleic acid substrate for a Cas protein is a double-stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, a “Cas nuclease” means a polypeptide or complex of polypeptides having RNA and DNA binding activity, or a DNA-binding subunit of such a complex, wherein the DNA binding activity is sequence-specific and depends on the sequence of a guide RNA. Exemplary Cas nucleases (and also “Cas protein”) include Cas cleavases/nickases. In some embodiments, the Cas nuclease cleaves one or two strands of the DNA. In some embodiments, the Cas nuclease is a nickase. In some embodiments, the Cas nuclease is a dsDNA cleavase. Cas nucleases include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity, such as a Cas9 nuclease or a Cpf1 nuclease. Class 2 Cas nucleases include Class 2 Cas cleavases and Class 2 Cas nickases (e.g., H840A, D10A, or N863A variants), which further have RNA-guided DNA cleavases or nickase activity. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9 (1.0) (e.g, K810A, K1003A, R1060A variants), and eSPCas9 (1.1) (e.g., K848A, K1003A, R1060A variants) proteins and modifications thereof. Cpf1 protein, Zetsche et al., Cell, 163:1-13 (2015), is homologous to Cas9, and contains a RuvC-like nuclease domain. Cpf1 sequences of Zetsche are incorporated by reference in their entirety. See, e.g., Zetsche, Tables S1 and S3. “Cas9” encompasses Spy Cas9, the variants of Cas9 listed herein, and equivalents thereof. See, e.g., Makarova et al., Nat Rev Microbiol, 13 (11): 722-36 (2015); Shmakov et al., Molecular Cell, 60:385-397 (2015). As used herein, delivery of a Cas nuclease (e.g., a Cas9 nuclease, or an S. pyogenes Cas9 nuclease) includes delivery of the polypeptide or mRNA. For example, the LNP composition described herein may comprise an mRNA encoding a Cas nuclease.

“Modified uridine” is used herein to refer to a nucleoside other than thymidine with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, i.e., a pseudouridine in which one or more non-proton substituents (e.g., alkyl, such as methyl) takes the place of a proton, e.g., N1-methyl pseudouridine. In some embodiments, a modified uridine is any of a substituted uridine, pseudouridine, or a substituted pseudouridine.

“Uridine position” as used herein refers to a position in a polynucleotide occupied by a uridine or a modified uridine. Thus, for example, a polynucleotide in which “100% of the uridine positions are modified uridines” contains a modified uridine at every position that would be a uridine in a conventional RNA (where all bases are standard A, U, C, or G bases) of the same sequence. Unless otherwise indicated, a U in a polynucleotide sequence of a sequence table or sequence listing in, or accompanying, this disclosure can be a uridine or a modified uridine.

As used herein, “treatment” refers to any administration or application of a therapeutic for disease or disorder in a subject, and includes inhibiting the disease, slowing progression of the disease, arresting its development, reversing progression of disease (e.g., reversing build up of amyloid fibrils), relieving one or more symptoms of the disease, curing the disease, improving one or more clinical metrics described herein, or preventing reoccurrence of one or more symptoms of the disease. In some embodiments, treatment of ATTR may comprise alleviating symptoms of ATTR. In some embodiments, treatment of ATTR may comprise a substantial reduction or knockdown expression of the TTR gene, e.g., a durable reduction by at least 95%, thereby substantially reducing or eliminating the production of TTR protein associated with ATTR.

As used herein, “amyloid” refers to abnormal aggregates of proteins or peptides that are normally soluble. Amyloids are insoluble, and amyloids can create proteinaceous deposits in organs and tissues. Proteins or peptides in amyloids may be misfolded into a form that allows many copies of the protein to stick together to form fibrils. While some forms of amyloid may have normal functions in the human body, “amyloids” as used herein refers to abnormal or pathologic aggregates of protein. Amyloids may comprise a single protein or peptide, such as TTR, or they may comprise multiple proteins or peptides, such as TTR and additional proteins.

As used herein, “amyloid fibrils” refers to insoluble fibers of amyloid that are resistant to degradation. Amyloid fibrils can produce symptoms based on the specific protein or peptide and the tissue and cell type in which it has aggregated.

As used herein, “amyloidosis” refers to a disease characterized by symptoms caused by deposition of amyloid or amyloid fibrils. Amyloidosis can affect numerous organs including the heart, kidney, liver, spleen, nervous system, and digestive track.

As used herein, “TTR” refers to transthyretin, which is the gene product of a TTR gene. TTR is also known in the art as CTS, CTS1, HEL111, HsT2651, PALB, prealbumin, TBPA, and ATTN. See, e.g., HGNC: HGNC: 12405 (https://www.ncbi.nlm.nih.gov/gene/7276).

As used herein, “ATTR,” “TTR-related amyloidosis,” “TTR amyloidosis,” “ATTR amyloidosis,” “amyloidosis associated with TTR,” or “transthyretin amyloidosis” refers to a condition resulting from misfolded TTR protein that accumulates as amyloid fibrils in multiple tissues (primarily nerve and muscle) leading to the predominant polyneuropathy (PN) and/or cardiomyopathy (CM) phenotypes of the illness. Symptoms of PN include numbness in the extremities due to peripheral neuropathy, dizziness, and gastrointestinal disturbances due to autonomic neuropathy. Symptoms of CM include shortness of breath and other symptoms of cardiac impairment, including congestive heart failure. Both phenotypes are associated with hereditary (familial) ATTR (ATTRv). ATTR-CM can result from mutation(s) in the TTR gene (ATTRv-CM) and/or wildtype TTR gene (ATTR-CM). Wild-type ATTR (ATTR-wt) is primarily associated with CM. A subject with ATTRv may present with a mixed clinical phenotype consisting of both neurologic and cardiac impairment.

As used herein, “hereditary ATTR” refers to ATTR that is associated with a mutation in the sequence of the TTR gene. Known mutations in the TTR gene associated with ATTR include those resulting in TTR with substitutions of T60A, V30M, V30A, V30G, V30L, V122I, V122A, or V122(−). Hereditary ATTR includes familial amyloid cardiomyopathy (“FAC”) characterized by restrictive cardiomyopathy, which is also known as hereditary transthyretin amyloidosis with cardiomyopathy (“ATTRv-CM”). Congestive heart failure is common in FAC. Average age of onset is approximately 60-70 years of age, with an estimated life expectancy of 4-5 years after diagnosis. Hereditary ATTR also includes familial amyloid polyneuropathy (“FAP”), also known as hereditary transthyretin amyloidosis with polyneuropathy (“ATTRv-PN”) characterized primarily by sensorimotor neuropathy. Autonomic neuropathy is common in FAP. While neuropathy is a primary feature, symptoms of FAP may also include cachexia, renal failure, and cardiac disease. Average age of onset of FAP is approximately 30-50 years of age, with an estimated life expectancy of 5-15 after diagnosis. As used herein, “hereditary ATTR” refers to ATTRv-PN and/or ATTRv-CM.

As used herein, “wild-type ATTR” and “ATTRwt” refer to ATTR not associated with a pathological TTR mutation such as T60A, V30M, V30A, V30G, V30L, V122I, V122A, or V122(−). ATTRwt has also been referred to as senile systemic amyloidosis. Onset typically occurs in men aged 60 or higher with the most common symptoms being congestive heart failure and abnormal heart rhythm such as atrial fibrillation. Additional symptoms include consequences of cardiac impairment such as shortness of breath, fatigue, dizziness, swelling (especially in the legs), nausea, angina, disrupted sleep, and weight loss. As used herein, “wild-type ATTR” refers to polyneuropathy and/or cardiomyopathy phenotypes of the illness, not associated with a TTR mutation.

In some embodiments, a human subject has been or concurrently is diagnosed with ATTR prior to treatment. In some embodiments, a human subject is diagnosed with ATTR based on genetic testing (e.g., a documented TTR mutation). In some embodiments, a human subject is diagnosed with ATTR based on a clinical diagnosis of sensorimotor peripheral neuropathy. In some embodiments, a human subject is diagnosed with ATTR based on a Neuropathy Impairment Score (NIS)≥5 and ≤130 prior to treatment. In some embodiments, a human subject is diagnosed with ATTR based on a documented tissue deposition of TTR amyloid by biopsy or by validated noninvasive imaging. In some embodiments, a human subject is diagnosed with ATTR based on a Polyneuropathy Disability (PND) score≤3b. In some embodiments, a human subject is diagnosed with ATTR amyloidosis with cardiomyopathy, classified as hereditary ATTR (ATTRv) amyloidosis with cardiomyopathy or wild-type cardiomyopathy (ATTRwt). In some embodiments, a human subject with ATTR-CM is classified under the New York Health Association (NYHA) classification as Class I or Class II. In some embodiments, a human subject with ATTR-CM is classified under the New York Health Association (NYHA) classification as Class III.

In some embodiments, a human subject has progression of symptoms (e.g., polyneuropathy symptoms) prior to treatment. In some embodiments, the human subject has an increase in Polyneuropathy Disability (PND) score≥1 point. In some embodiments, the human subject has an increase Familial Amyloid Polyneuropathy (FAP) stage≥1 point. In some embodiments, the human subject has an increase in Neuropathy Impairment Score (NIS)≥5 points. In some embodiments, the human subject has an increase in NIS-Lower Limb (LL)≥5 points. In some embodiments, the human subject has a decrease in Modified Body-Mass Index (mBMI)≥25 kg/m2×g/L. In some embodiments, the human subject has a decrease in 6-minute walk test≥30 meters. In some embodiments, the human subject has a decrease in 10-meter walk test>0.1 m/s. As used herein, “mutant TTR” refers to a gene product of TTR (i.e., the TTR protein) having a change in the amino acid sequence of TTR compared to the wildtype amino acid sequence of TTR. The human wild-type TTR sequence is available at NCBI Gene ID: 7276; Ensembl: Ensembl: ENSG000001 18271. Mutant forms of TTR associated with ATTR, e.g., in humans, include but are not limited to T60A, V30M, V30A, V30G, V30L, V122I, V122A, or V122(−), notated according to amino acid positions based on mature protein sequence, without the signal sequence (e.g., T60A is equivalent to T80A, also denoted as p.T80A).

As used herein, “knockdown” refers to a decrease in expression of a particular gene product (e.g., TTR), e.g., in a cell, population of cells, tissue, or organ, by gene editing. In some embodiments, gene editing can be assessed by sequence, e.g., next generation sequencing (NGS). Expression may be decreased by at least 60%, 65%, 70%, 75%, 80%, 85%, 90%, 95%, or to below the level of detection of the assay as compared to a suitable control, e.g., the subject's baseline or prior to treatment. Methods for measuring knockdown of mRNA are known and include sequencing of mRNA isolated from a tissue or cell population of interest. Knockdown of a protein can be measured by detecting the amount of the protein from a tissue, cell population, or fluid of interest. Flow cytometry analysis is a known method for measuring knockdown of protein expression. For secreted proteins, knockdown may be assessed in a fluid such as tissue culture media or blood, or serum or plasma derived therefrom. Serum protein levels can be measured by quantitative assay, e.g., ELISA, and used to detect knockdown. In some embodiments, “knockdown” may refer to some loss of expression of a particular gene product, for example a decrease in the amount of full-length, wild-type mRNA transcribed or translated into full-length protein, or a decrease in the amount of protein expressed by a population of cells. It is well understood what changes in an mRNA sequence would result in decreased expression of a wild-type or full-length protein. In some embodiments, “knockdown” may refer to some loss of expression of a particular gene product, e.g., TTR.

As used herein, “durable” in the context of a serum TTR or serum prealbumin knockdown (e.g., a durable knockdown) or “durably” reducing expression of the gene (e.g. the TTR gene) refers to a lasting effect, such as a lasting knockdown or a lasting reduction in gene expression. In some embodiments, a durable knockdown in serum TTR or serum prealbumin refers to a reduction (level relative to baseline) as measured at 14 days or 28 days after administration of the LNP composition that is maintained, e.g., for at least 6 months, 9 months, 1 year, 2 years, 3 years, 4 years, 5 years, or more. In some embodiments, the level maintained can vary. In some embodiments, the reduction correlates to a desired clinical efficacy for the disorder being treated. The level of reduction to achieve a desired clinical efficacy for a given disorder, e.g., ATTR, is known in the art. For example, a reduction by at least 60%, 65%, 70%, 75%, 80%, 85%, 90%, 95%, or more correlates to a desired clinical efficacy for a specific disorder. For example, a reduction by at least 60%, 65%, 70%, 75%, 80%, 85%, 90%, 95%, or more correlates to a desired clinical efficacy for ATTR.

As used herein, a “monogenic disorder” refers to a disorder that results from an aberrant expression or activity of a liver gene product. The monogenic disorder can be treated with an edit to the gene in the liver, or to a non-coding region that causes an aberrant expression or activity of a liver gene product. In some embodiments, the gene product is a protein. In some embodiments, the gene product is an RNA molecule. In some embodiments, an edit to the gene in the liver, or to a non-coding region that causes an aberrant expression or activity of a liver gene product, reduces the level (e.g., serum level) of the gene product. In some embodiments, the gene contains one or more modifications in the gene relative to wildtype. In some embodiments, the gene is wildtype. The monogenic disorder may be a genetic disorder that is amenable to treatment by a single gene edit.

As used herein, an “effective amount” refers to an amount of mRNA encoding a Cas nuclease and a guide RNA that reduces serum TTR level by at least 60% in a subject relative to a baseline serum TTR level and/or reduces serum TTR to less than about 50 μg/mL after administration of the mRNA encoding the Cas nuclease and the guide RNA. For instance, an LNP composition may comprise an effective amount of the mRNA encoding the Cas nuclease and the guide RNA, e.g., a guide RNA that targets TTR (the combined or total RNA). In some embodiments, an LNP composition delivers the mRNA encoding the Cas nuclease and the guide RNA, which can comprise an “effective amount” of RNA measured as total RNA. In some embodiments, an effective amount of the mRNA encoding the Cas nuclease and the guide RNA reduces serum TTR level by at least 60-70%, 70-80%, 80-90%, 90-95%, 95-98%, 98-99%, or 99-100% in a subject relative to a baseline serum TTR level. In some embodiments, an effective amount of the mRNA encoding the Cas nuclease and the guide RNA reduces serum prealbumin levels by at least 60% in a subject relative to a baseline serum prealbumin level. In some embodiments, an effective amount of the mRNA encoding the Cas nuclease and the guide RNA reduces serum prealbumin level by at least 60-70%, 70-80%, 80-90%, 90-95%, 95-98%, 98-99%, or 99-100% in a subject relative to a baseline serum prealbumin level. In some embodiments, an effective amount of the mRNA encoding the Cas nuclease and the guide RNA reduces serum TTR to less than about 50 μg/mL, less than about 40 μg/mL, less than about 30 μg/mL, less than about 20 μg/mL, or less than about 10 μg/mL after administration of the mRNA encoding the Cas nuclease and the guide RNA.

As used herein, a “biosafety metric” refers to a clinical metric used to monitor for safety events associated with administration of the LNP composition described herein to a human subject. A biosafety metric may allow for a determination of a safety event, including an adverse event (NCI-CTCAE Grade 3 or higher), a serious adverse event, an adverse event of special interest, and/or a treatment-emergent adverse event (CTCAE Grade 3 or higher), as described herein. Guidelines for defining the severity of a safety event (e.g., adverse event) are known in the art (e.g., Common Terminology Criteria for Adverse Events (CTCAE) including National Cancer Institute (NCI)-CTCAE, version 5.0). In some embodiments, a level of a biosafety metric is measured prior to administration of the LNP composition. In some embodiments, a level of a biosafety metric is measured following administration of the LNP composition. In some embodiments, a level of a biosafety metric is measured prior to and following the administration of the LNP composition, thereby allowing for a comparison of the levels of the biosafety metric before and after treatment with the LNP composition to determine a change, e.g., an acceptable change. As used herein, an “acceptable” change refers to a change in biosafety metric level, wherein the resulting change does not constitute a safety event (e.g., an adverse event (NCI-CTCAE Grade 3 or higher), a serious adverse event, an adverse event of special interest, a treatment-emergent adverse event (CTCAE Grade 3 or higher), and/or an event that otherwise requires discontinuation of the study drug as determined by a clinician. In some embodiments, a level of a biosafety metric measured prior to administration of the LNP composition can serve as a baseline for comparison against one or more levels of the biosafety metric measured following administration of the LNP composition (e.g., measurements taken at specific intervals following administration can be compared against baseline level). In some embodiments, a baseline is the last available measurement taken prior to administration of the LNP composition. In some embodiments, the biosafety metric is not compared against a baseline if the value alone can be used determine a safety event.

As used herein, “safe and well-tolerated” refers to the absence of a safety event as described herein, e.g., the absence of: an adverse event (NCI-CTCAE Grade 3 or higher), a serious adverse event, an adverse event of special interest, a treatment-emergent adverse event (CTCAE Grade 3 or higher), and/or an event that otherwise requires discontinuation of the study drug as determined by a clinician. In some embodiments, “safe and well-tolerated” includes patients who experience NCI-CTCAE Grade 3 or higher that is unrelated to administration of the composition described herein, e.g., LNP composition comprising an effective amount of an mRNA encoding Cas nuclease and a guide RNA targeting TTR, and/or resolves, e.g., with or without intervention, after an acceptable period of time for said event.

In some embodiments, an adverse event of special interest includes, e.g., infusion-related reaction (IRR) (e.g., requiring treatment or discontinuation of infusion, and/or Grade 3 or higher); incidence of cytokine release syndrome; abnormal coagulation findings defined by clinically relevant abnormal bleeding or thrombotic or hemorrhagic incidence or CTCAE≥Grade 2 abnormal blood test results; acute liver injury evidenced by CTCAE≥Grade 2 elevation in ALT, CTCAE≥Grade 2 elevation in AST, CTCAE≥Grade 2 elevation in total bilirubin, CTCAE≥Grade 2 elevation in GLDH; an event attributed to impacts on the spleen (splenic hemorrhage, splenic infarction, sometimes thrombocytopenia, sometimes anemia or lymphopenia with specific abnormal findings on study of the blood cells on microscopy); an event attributed to impacts on the adrenal glands; clinically relevant symptoms of hypothyroidism; decreased thyroxine (T4 levels) below normal range; and an ophthalmic event consistent with Vitamin A deficiency.

In some embodiments, an adverse event is any untoward medical occurrence in a subject administered a study drug or has undergone study procedures and which does not necessarily have a causal relationship with the treatment. In some embodiments, an adverse event is an unintended sign (including an abnormal laboratory finding), symptom, or disease temporally associated with the treatment, whether or not related to the medicinal (investigational) product. In some embodiments, an adverse event that induces clinical signs or symptoms. In some embodiments, an adverse event requires active intervention. In some embodiments, an adverse event requires interruption or discontinuation of the treatment. In some embodiments, an adverse event is an abnormality that is clinically significant in the opinion of the investigator. Grading criteria for adverse events are known in the art, such as, e.g., Common Terminology Criteria for Adverse Events (CTCAE), including National Cancer Institute (NCI)-CTCAE.

Biosafety metrics include known laboratory assessments relating generally to, e.g., coagulation, hematology, clinical chemistry, urinalysis, and other bioanalytical assessments (e.g., cytokines, complement). Particular biosafety metrics include, but are not limited to: liver enzyme levels (e.g., an elevation in alanine aminotransferase (ALT) or aspartate aminotransferase (AST)>5×ULN for more than 4 weeks after administration of a treatment, ALT or AST>3×ULN and total bilirubin>2×ULN (Hy's law) after administration of a treatment), levels of activated partial thromboplastin time (aPTT) (e.g., an elevation in aPTT)>5×ULN for more than 4 weeks after administration of a treatment), levels of prothrombin time (PT), levels of thrombin generation time (TGT) (e.g., peak height, lag time, and/or endogenous thrombin potential), levels of fibrinogen, prothrombin international normalized (INR) ratio, levels, levels of d-dimer, laboratory parameters consistent with disseminated intravascular coagulation, changes in hematology values (e.g. a CTCAE>Grade 2 abnormal blood test results after administration of a treatment), changes in chemistry values, changes in coagulation, changes in urinalysis, levels of Glutamate Dehydrogenase, levels of C-reactive protein, levels of complement (C3, C4, C3a, C5a, Bb), levels of cytokines (GM-CSF, INF-γ, IL-1β, IL-4, IL-5, IL-6, IL-8, IL-10, IL-13, IL-23, TNF-α, IL-17, MCP-1), thyroxine (T4 levels) (e.g. a decrease in levels below normal range or clinically relevant symptoms/signs of hypothyroidism after administration of a treatment), acute liver injury (e.g. a CTCAE>Grade 2 elevations in ALT, AST, total bilirubin or GLDH or clinically relevant symptoms/signs of liver injury after administration of a treatment), and changes in a 12-Lead Electrocardiogram, Additional biosafety metrics, including those associated with administration of an LNP composition, are known in the art. Similarly, acceptable levels and/or changes in the biosafety metrics are known in the art and may be assessed by routine methods, e.g., by a clinician or laboratory.

As used herein, a “clinical efficacy metric” refers to a metric used to assess amelioration of disease in a human subject treated with the LNP composition described herein. In some embodiments, a level of a clinical efficacy metric is measured following administration of the LNP composition. In some embodiments, a level of a clinical efficacy metric is measured prior to and following the administration of the LNP composition, thereby allowing for a comparison of the levels of the clinical efficacy metric before and after treatment with the LNP composition. In some embodiments, a level of a clinical metric measured prior to administration of the LNP composition can serve as a baseline or control for comparison against one or more levels of the clinical metric measured following administration of the LNP composition. In some embodiments, a baseline is the last available measurement taken prior to administration of the LNP composition.

For disorders characterized by transthyretin amyloid, clinical efficacy metrics include, but are not limited to: a decrease in serum TTR (e.g. a 60% decrease of serum TTR as measured by ELISA after administration of a treatment), a decrease in serum TTR (e.g. at least a 60% decrease of serum TTR as measured by mass spectrometry after administration of a treatment), a decrease in serum prealbumin, a reduction in Polyneuropathy Disability (PND) Score, a reduction in Familial Amyloid Polyneuropathy (FAP) stage, a decrease in Neuropathy Impairment Score (NIS), a decrease in Modified Neurological Impairment Score (mNIS+7), a decrease in Neuropathy Impairment Score (NIS)-Lower Limb (LL), an increase in Modified Body Mass Index (mBMI)≥25 kg/m2×g/L, an increase in 6-minute walk test (6-MWT)≥30 meters, and increase 10-Meter Walk Test (10-MWT)≥0.1 meters/second. Additional clinical efficacy metrics include an improvement in serum Neurofilament Light Chain (NfL) levels, an improvement in quality of life as assessed by Norfolk Quality of Life-Diabetic Neuropathy, an improvement in quality of life as assessed by EuroQOL (EQ)-5D-5L, an improvement in cardiac MRI, an improvement in N-terminal prohormone of brain natriuretic peptide (NT-proBNP) levels; an improvement in Troponin I levels, an improvement in New York Health Association (NYHA) classification, an improvement as scored by the Kansas City Cardiomyopathy Questionnaire (KCCQ). Additional clinical efficacy metrics, including TTR amyloidosis, are known in the art. Similarly, “clinically significant improvement” in a clinical efficacy metric, i.e., levels and/or changes in clinical efficacy metric(s) indicative of amelioration of disease, including TTR amyloidosis, are known in the art and may be assessed by routine methods, e.g., by a clinician or laboratory. For example, serum TTR level is a clinical efficacy metric for TTR amyloidosis. A “clinically significant improvement” in this clinical efficacy metric for the treatment of TTR amyloidosis includes at least 60%, 70%, 80%, 85%, 90%, or 95% reduction of serum TTR level after treatment as compared to baseline, e.g., prior to treatment, e.g., with the LNP composition described here. For example, serum prealbumin level is also a clinical efficacy metric for TTR amyloidosis. A “clinically significant improvement” in this clinical efficacy metric for the treatment of TTR amyloidosis includes at least 60%, 70%, 80%, 85%, 90%, or 95% reduction of serum prealbumin level after treatment as compared to baseline, e.g., prior to treatment, e.g., with the LNP composition described herein. While “TTR” is synonymous with “prealbumin”, “serum prealbumin level” indicates a different assay for measuring this protein level as compared to an assay for measuring “serum TTR level”; both assays measure the same protein.

As used herein, the term “lipid nanoparticle” (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”-lamellar phase lipid bilayers that, in some embodiments, are substantially spherical- and, in more particular embodiments, can comprise an aqueous core, e.g., comprising a substantial portion of RNA molecules), a dispersed phase in an emulsion, micelles, or an internal phase in a suspension. See also, e.g., WO2017173054A1 and WO2019067992A1, the contents of which are hereby incorporated by reference in their entirety.

As used herein, the phrase “pharmaceutically acceptable” means that which is useful in preparing a pharmaceutical composition that is generally non-toxic and is not biologically undesirable and that are not otherwise unacceptable for pharmaceutical use.

As used herein, systemic administration may be by intravenous infusion. “Infusion” refers to an active administration of one or more agents with an infusion time of, for example, approximately 2 hours. In some embodiments, an LNP, e.g., comprising an mRNA encoding a Cas nuclease (such as Cas9) described herein and a gRNA described herein is systemically administered to a human subject.

As used herein, “infusion prophylaxis” refers to a regimen administered to a subject before treatment (e.g., comprising administration of an LNP) comprising, for example, administering intravenous steroid (e.g., dexamethasone 10 mg); intravenous H1 blocker (e.g., diphenhydramine 50 mg) or oral H1 blocker (e.g., cetirizine 10 mg); and intravenous or oral H2 blocker (e.g., famotidine 20 mg).

### I. Compositions Targeting a Gene

Disclosed herein are methods for editing a gene of interest (e.g., TTR) in the liver of a human subject, modifying the gene in a hepatocyte of the subject, or treating a disease, as well as related compositions, including compositions for use in such methods. In general, disclosed herein are LNP compositions comprising an mRNA encoding a Cas nuclease, e.g., Cas9, and a guide RNA that targets a gene, e.g., a guide RNA that targets the TTR gene. The subjects treated with such methods and compositions may have wild-type or non-wild type gene of interest sequences, such as, for example, subjects with ATTR, which may be ATTR wt or a hereditary (or familial) form of ATTR.

In some embodiments, methods disclosed herein comprise systemic administration of a lipid nanoparticle system for in vivo liver-targeted delivery of a guide RNA and an mRNA encoding a Cas nuclease.

1. Guide RNA (gRNAs)

The guide RNA used in the disclosed methods and compositions comprises a guide sequence targeting a gene of interest (e.g., the TTR gene). Exemplary guide sequences targeting the TTR gene are shown in Sequence Table, as are exemplary generic sgRNA structures and conserved portions of guide RNAs. Guide sequences may further comprise additional nucleotides to form a crRNA, e.g., with the following exemplary nucleotide sequence following the Guide Sequence at its 3′ end: GUUUUAGAGCUAUGCUGUUUUG (SEQ ID NO: 33). In the case of a sgRNA, the above Guide Sequences may further comprise additional nucleotides to form a sgRNA, e.g., with the following exemplary nucleotide sequence following the 3′ end of the Guide Sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 32) in 5′ to 3′ orientation. In some embodiments, the gRNA may comprise a guide sequence within a generic sgRNA structure or it may comprise a guide sequence and a sgRNA conserved region, such as exemplary sequences shown in the Sequence Table. In some embodiments, the sgRNA is modified. In some embodiments, the sgRNA comprises the modification pattern shown below in SEQ ID NO: 19, where N is any natural or non-natural nucleotide, and where the totality of the N's comprise a guide sequence as described herein and the modified sgRNA comprises the following sequence: mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 19), where “N” may be any natural or non-natural nucleotide. For example, encompassed herein is SEQ ID NO: 19, where the N's are replaced with any of the guide sequences disclosed herein. The modifications may remain as shown in SEQ ID NO: 19 despite the substitution of N's for the nucleotides of a guide. That is, although the nucleotides of the guide replace the “N's”, the first three nucleotides are 2′OMe modified and there are phosphorothioate linkages between the first and second nucleotides, the second and third nucleotides and the third and fourth nucleotides.

In some embodiments, the gRNA comprises a guide sequence that directs a Cas nuclease, which can be a nuclease (e.g., a Cas9 nuclease such as SpyCas9), to a target DNA sequence. The gRNA may comprise a crRNA comprising 18, 19, or 20 contiguous nucleotides of a guide sequence. In some embodiments, the gRNA comprises a crRNA comprising a sequence with about 75%, 80%, 85%, 90%, 95%, 96%, 97%, 98%, 99%, or 100% identity to at least 18, 19, or 20 contiguous nucleotides of a guide sequence. In some embodiments, the gRNA comprises a crRNA comprising a sequence with about 75%, 80%, 85%, 90%, 95%, 96%, 97%, 98%, 99%, or 100% identity to a guide sequence. The gRNA may further comprise a trRNA. In each composition and method embodiment described herein, the crRNA and trRNA may be associated as a single RNA (sgRNA), or may be on separate RNAs (dgRNA). In the context of sgRNAs, the crRNA and trRNA components may be covalently linked, e.g., via a phosphodiester bond or other covalent bond.

In some embodiments, the guide sequences, including sgRNA sequences and modified sequences, in Sequence Table are encompassed.

In some embodiments, a guide RNA that targets the TTR gene comprises any one or more of SEQ ID NOs: 15, 16, 34, 35, and 38-54, or an 18-, 19-, or 20-nucleotide portion thereof. In some embodiments, a sgRNA that targets the TTR gene comprises any one or more of SEQ ID NOs: 15, 16, 34, 35, and 38-54, or an 18-, 19-, or 20-nucleotide portion thereof. In some embodiments, an LNP composition disclosed herein comprises a guide RNA that targets the TTR gene comprises any one or more of SEQ ID NOs: 15, 16, 34, 35, and 38-54, or an 18-, 19-, or 20-nucleotide portion thereof.

In some embodiments, a guide RNA that targets the TTR gene comprises SEQ ID NO: 15 or an 18-, 19-, or 20-nucleotide portion thereof. In some embodiments, a sgRNA that targets the TTR gene comprises SEQ ID NO: 15 or an 18-, 19-, or 20-nucleotide portion thereof. In some embodiments, an LNP composition disclosed herein comprises a guide RNA that targets the TTR gene comprises any one or more of SEQ ID NO: 15 or an 18-, 19-, or 20-nucleotide portion thereof.

In some embodiments, a guide RNA that targets the TTR gene comprises SEQ ID NO: 16 or an 18-, 19-, or 20-nucleotide portion thereof. In some embodiments, a sgRNA that targets the TTR gene comprises SEQ ID NO: 16 or an 18-, 19-, or 20-nucleotide portion thereof. In some embodiments, an LNP composition disclosed herein comprises a guide RNA that targets the TTR gene comprises any one or more of SEQ ID NO: 16 or an 18-, 19-, or 20-nucleotide portion thereof.

In some embodiments, a guide RNA that targets the TTR gene comprises SEQ ID NO: 34 or an 18-, 19-, or 20-nucleotide portion thereof. In some embodiments, a sgRNA that targets the TTR gene comprises SEQ ID NO: 34 or an 18-, 19-, or 20-nucleotide portion thereof. In some embodiments, an LNP composition disclosed herein comprises a guide RNA that targets the TTR gene comprises any one or more of SEQ ID NO: 34 or an 18-, 19-, or 20-nucleotide portion thereof.

In some embodiments, a guide RNA that targets the TTR gene comprises SEQ ID NO: 35 or an 18-, 19-, or 20-nucleotide portion thereof. In some embodiments, a sgRNA that targets the TTR gene comprises SEQ ID NO: 35 or an 18-, 19-, or 20-nucleotide portion thereof. In some embodiments, an LNP composition disclosed herein comprises a guide RNA that targets the TTR gene comprises any one or more of SEQ ID NO: 35 or an 18-, 19-, or 20-nucleotide portion thereof.

In some embodiments, a guide RNA that targets the TTR gene comprises SEQ ID NO: 38 or an 18-, 19-, or 20-nucleotide portion thereof. In some embodiments, a sgRNA that targets the TTR gene comprises SEQ ID NO: 38 or an 18-, 19-, or 20-nucleotide portion thereof. In some embodiments, an LNP composition disclosed herein comprises a guide RNA that targets the TTR gene comprises any one or more of SEQ ID NO: 38 or an 18-, 19-, or 20-nucleotide portion thereof.

In some embodiments, a guide RNA that targets the TTR gene comprises SEQ ID NO: 39 or an 18-, 19-, or 20-nucleotide portion thereof. In some embodiments, a sgRNA that targets the TTR gene comprises SEQ ID NO: 39 or an 18-, 19-, or 20-nucleotide portion thereof. In some embodiments, an LNP composition disclosed herein comprises a guide RNA that targets the TTR gene comprises any one or more of SEQ ID NO: 39 or an 18-, 19-, or 20-nucleotide portion thereof.

In some embodiments, a guide RNA that targets the TTR gene comprises SEQ ID NO: 40 or an 18-, 19-, or 20-nucleotide portion thereof. In some embodiments, a sgRNA that targets the TTR gene comprises SEQ ID NO: 40 or an 18-, 19-, or 20-nucleotide portion thereof. In some embodiments, an LNP composition disclosed herein comprises a guide RNA that targets the TTR gene comprises any one or more of SEQ ID NO: 40 or an 18-, 19-, or 20-nucleotide portion thereof.

In some embodiments, a guide RNA that targets the TTR gene comprises SEQ ID NO: 41 or an 18-, 19-, or 20-nucleotide portion thereof. In some embodiments, a sgRNA that targets the TTR gene comprises SEQ ID NO: 41 or an 18-, 19-, or 20-nucleotide portion thereof. In some embodiments, an LNP composition disclosed herein comprises a guide RNA that targets the TTR gene comprises any one or more of SEQ ID NO: 41 or an 18-, 19-, or 20-nucleotide portion thereof.

In some embodiments, a guide RNA that targets the TTR gene comprises SEQ ID NO: 42 or an 18-, 19-, or 20-nucleotide portion thereof. In some embodiments, a sgRNA that targets the TTR gene comprises SEQ ID NO: 42 or an 18-, 19-, or 20-nucleotide portion thereof. In some embodiments, an LNP composition disclosed herein comprises a guide RNA that targets the TTR gene comprises any one or more of SEQ ID NO: 42 or an 18-, 19-, or 20-nucleotide portion thereof.

In some embodiments, a guide RNA that targets the TTR gene comprises SEQ ID NO: 43 or an 18-, 19-, or 20-nucleotide portion thereof. In some embodiments, a sgRNA that targets the TTR gene comprises SEQ ID NO: 43 or an 18-, 19-, or 20-nucleotide portion thereof. In some embodiments, an LNP composition disclosed herein comprises a guide RNA that targets the TTR gene comprises any one or more of SEQ ID NO: 43 or an 18-, 19-, or 20-nucleotide portion thereof.

In some embodiments, a guide RNA that targets the TTR gene comprises SEQ ID NO: 44 or an 18-, 19-, or 20-nucleotide portion thereof. In some embodiments, a sgRNA that targets the TTR gene comprises SEQ ID NO: 44 or an 18-, 19-, or 20-nucleotide portion thereof. In some embodiments, an LNP composition disclosed herein comprises a guide RNA that targets the TTR gene comprises any one or more of SEQ ID NO: 44 or an 18-, 19-, or 20-nucleotide portion thereof.

In some embodiments, a guide RNA that targets the TTR gene comprises SEQ ID NO: 45 or an 18-, 19-, or 20-nucleotide portion thereof. In some embodiments, a sgRNA that targets the TTR gene comprises SEQ ID NO: 45 or an 18-, 19-, or 20-nucleotide portion thereof. In some embodiments, an LNP composition disclosed herein comprises a guide RNA that targets the TTR gene comprises any one or more of SEQ ID NO: 45 or an 18-, 19-, or 20-nucleotide portion thereof.

In some embodiments, a guide RNA that targets the TTR gene comprises SEQ ID NO: 46 or an 18-, 19-, or 20-nucleotide portion thereof. In some embodiments, a sgRNA that targets the TTR gene comprises SEQ ID NO: 46 or an 18-, 19-, or 20-nucleotide portion thereof. In some embodiments, an LNP composition disclosed herein comprises a guide RNA that targets the TTR gene comprises any one or more of SEQ ID NO: 46 or an 18-, 19-, or 20-nucleotide portion thereof.

In some embodiments, a guide RNA that targets the TTR gene comprises SEQ ID NO: 47 or an 18-, 19-, or 20-nucleotide portion thereof. In some embodiments, a sgRNA that targets the TTR gene comprises SEQ ID NO: 47 or an 18-, 19-, or 20-nucleotide portion thereof. In some embodiments, an LNP composition disclosed herein comprises a guide RNA that targets the TTR gene comprises any one or more of SEQ ID NO: 47 or an 18-, 19-, or 20-nucleotide portion thereof.

In some embodiments, a guide RNA that targets the TTR gene comprises SEQ ID NO: 48 or an 18-, 19-, or 20-nucleotide portion thereof. In some embodiments, a sgRNA that targets the TTR gene comprises SEQ ID NO: 48 or an 18-, 19-, or 20-nucleotide portion thereof. In some embodiments, an LNP composition disclosed herein comprises a guide RNA that targets the TTR gene comprises any one or more of SEQ ID NO: 48 or an 18-, 19-, or 20-nucleotide portion thereof.

In some embodiments, a guide RNA that targets the TTR gene comprises SEQ ID NO: 49 or an 18-, 19-, or 20-nucleotide portion thereof. In some embodiments, a sgRNA that targets the TTR gene comprises SEQ ID NO: 49 or an 18-, 19-, or 20-nucleotide portion thereof. In some embodiments, an LNP composition disclosed herein comprises a guide RNA that targets the TTR gene comprises any one or more of SEQ ID NO: 49 or an 18-, 19-, or 20-nucleotide portion thereof.

In some embodiments, a guide RNA that targets the TTR gene comprises SEQ ID NO: 50 or an 18-, 19-, or 20-nucleotide portion thereof. In some embodiments, a sgRNA that targets the TTR gene comprises SEQ ID NO: 50 or an 18-, 19-, or 20-nucleotide portion thereof. In some embodiments, an LNP composition disclosed herein comprises a guide RNA that targets the TTR gene comprises any one or more of SEQ ID NO: 50 or an 18-, 19-, or 20-nucleotide portion thereof.

In some embodiments, a guide RNA that targets the TTR gene comprises SEQ ID NO: 51 or an 18-, 19-, or 20-nucleotide portion thereof. In some embodiments, a sgRNA that targets the TTR gene comprises SEQ ID NO: 51 or an 18-, 19-, or 20-nucleotide portion thereof. In some embodiments, an LNP composition disclosed herein comprises a guide RNA that targets the TTR gene comprises any one or more of SEQ ID NO: 51 or an 18-, 19-, or 20-nucleotide portion thereof.

In some embodiments, a guide RNA that targets the TTR gene comprises SEQ ID NO: 52 or an 18-, 19-, or 20-nucleotide portion thereof. In some embodiments, a sgRNA that targets the TTR gene comprises SEQ ID NO: 52 or an 18-, 19-, or 20-nucleotide portion thereof. In some embodiments, an LNP composition disclosed herein comprises a guide RNA that targets the TTR gene comprises any one or more of SEQ ID NO: 52 or an 18-, 19-, or 20-nucleotide portion thereof.

In some embodiments, a guide RNA that targets the TTR gene comprises SEQ ID NO: 53 or an 18-, 19-, or 20-nucleotide portion thereof. In some embodiments, a sgRNA that targets the TTR gene comprises SEQ ID NO: 53 or an 18-, 19-, or 20-nucleotide portion thereof. In some embodiments, an LNP composition disclosed herein comprises a guide RNA that targets the TTR gene comprises any one or more of SEQ ID NO: 53 or an 18-, 19-, or 20-nucleotide portion thereof.

In some embodiments, a guide RNA that targets the TTR gene comprises SEQ ID NO: 54 or an 18-, 19-, or 20-nucleotide portion thereof. In some embodiments, a sgRNA that targets the TTR gene comprises SEQ ID NO: 54 or an 18-, 19-, or 20-nucleotide portion thereof. In some embodiments, an LNP composition disclosed herein comprises a guide RNA that targets the TTR gene comprises any one or more of SEQ ID NO: 54 or an 18-, 19-, or 20-nucleotide portion thereof.

Any of the above TTR guide RNAs may include a generic sgRNA structure of the Sequences Table, or, e.g. a guide RNA conserved region structure as shown in the Sequence Table.

In each of the composition, use, and method embodiments described herein, the guide RNA may comprise two RNA molecules as a “dual guide RNA” or “dgRNA”. The dgRNA comprises a first RNA molecule comprising a crRNA comprising, e.g., a guide sequence, and a second RNA molecule comprising, e.g., a trRNA. The first and second RNA molecules may not be covalently linked, but may form a RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) comprising a guide sequence covalently linked to a trRNA. The sgRNA may comprise 18, 19, or 20 or more contiguous nucleotides of a guide sequence. In some embodiments, the sgRNA may comprise 20 contiguous nucleotides of a guide sequence. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more nucleotides. In some embodiments, the trRNA may comprise certain secondary structures, such as, for example, one or more hairpin or stem-loop structures, or one or more bulge structures.

The guide RNAs provided herein can be useful for recognizing (e.g., hybridizing to) a target sequence in the gene of interest. For example, the gene of interest target sequence may be recognized and cleaved by a provided Cas cleavase comprising a guide RNA. Thus, a Cas nuclease, such as a Cas cleavase, may be directed by a guide RNA to a target sequence of the gene of interest, where the guide sequence of the guide RNA hybridizes with the target sequence and the Cas nuclease, such as a Cas cleavase, cleaves the target sequence.

In some embodiments, the selection of the one or more guide RNAs is determined based on target sequences within the gene of interest.

Without being bound by any particular theory, mutations (e.g., frameshift mutations resulting from indels occurring as a result of a nuclease-mediated DSB or editing a gene of interest) in certain regions of the gene may be less tolerable than mutations in other regions of the gene, thus the location of a DSB is an important factor in the amount or type of protein knockdown that may result. In some embodiments, a gRNA complementary or having complementarity to a target sequence within the gene of interest is used to direct the Cas nuclease to a particular location in the gene of interest.

2. Modification of gRNAs

In some embodiments, the gRNA is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. In some embodiments, a modified gRNA is synthesized with a non-canonical nucleoside or nucleotide, is here called “modified.”

A modified guide RNA may comprise nucleosides or nucleoside analogs which have nitrogenous heterocyclic bases or base analogs linked together along a backbone, including conventional RNA, DNA, mixed RNA-DNA, and polymers that are analogs thereof. A guide RNA “backbone” can be made up of a variety of linkages, including one or more of sugar-phosphodiester linkages, phosphorothioate linkages, or combinations thereof. Sugar moieties of a guide RNA can be ribose, deoxyribose, or similar compounds with substitutions, e.g., 2′ methoxy. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion of chemical modifications for a guide RNA, see The Biochemistry of the Nucleic Acids 5-36, Adams et al., ed., 11th ed., 1992). Guide RNAs can comprise only conventional RNA or DNA sugars, bases and linkages, or can include both conventional components and substitutions (e.g., conventional bases with 2′ methoxy linkages, or polymers containing both conventional bases and one or more base analogs). RNA and DNA have different sugar moieties and can differ by the presence of uracil or analogs thereof in RNA and thymine or analogs thereof in DNA.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases.

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C* U*, or G* may be used to denote a nucleotide that is linked to the next (e.g., 3′) nucleotide with a PS bond.

In this application, the terms “mA*,” “mC*,” “mU*,” or “mG*” may be used to denote a nucleotide that has been substituted with 2′-O-Me and that is linked to the next (e.g., 3′) nucleotide with a PS bond.

The diagram below shows the substitution of S-into a nonbridging phosphate oxygen, generating a PS bond in lieu of a phosphodiester bond:

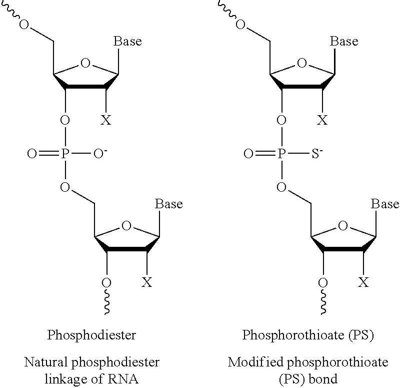

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds. In some embodiments, the first three nucleotides at the 5′ terminus, and the last three nucleotides at the 3′ terminus comprise a 2′-O-methyl (2′-O-Me) modified nucleotide, for example.

In some embodiments, the guide RNA comprises a modified sgRNA. In some embodiments, the sgRNA comprises the modification pattern shown in SEQ ID No: 19, where N is any natural or non-natural nucleotide, and where the totality of the N's comprise a guide sequence that directs a nuclease to a target sequence.

In some embodiments, the guide RNA comprises a sgRNA shown in any one of Table 2 of WO0201906787, the contents of which are hereby incorporated in their entirety. In some embodiments, the guide RNA comprises a sgRNA comprising any one of the guide sequences of Table 1 of WO02019067872, the contents of which are hereby incorporated in their entirety, and the nucleotides of SEQ ID No: 32 or 62, wherein the nucleotides of SEQ ID No: 32 or 62 are on the 3′ end of the guide sequence, and wherein the guide sequence may be modified as shown in SEQ ID No: 19.

### 3. RNA Comprising an Open Reading Frame Encoding a Cas Nuclease

Any RNA comprising an ORF encoding a Cas nuclease, e.g. a Cas9 nuclease such as an S. pyogenes Cas9, disclosed herein may be combined in a composition or method with any of the gRNAs disclosed herein. In any of the embodiments set forth herein, the nucleic acid comprising an open reading frame encoding a Cas nuclease may be an mRNA.

Codons that Increase Translation and/or that Correspond to Highly Expressed tRNAs; Exemplary Codon Sets

In some embodiments, the nucleic acid comprises an ORF having codons that increase translation in a mammal, such as a human. In further embodiments, the nucleic acid comprises an ORF having codons that increase translation in an organ, such as the liver, of a human. In further embodiments, the nucleic acid comprises an ORF having codons that increase translation in a cell type, such as a hepatocyte, of a human. An increase in translation in a hepatocyte, liver, or human, etc., can be determined relative to the extent of translation wild-type sequence of the ORF, or relative to an ORF having a codon distribution matching the codon distribution of the organism from which the ORF was derived or the organism that contains the most similar ORF at the amino acid level, such as S. pyogenes, S. aureus, or another prokaryote as the case may be for prokaryotically-derived Cas nucleases, such as the Cas nucleases from other prokaryotes described below. Alternatively, in some embodiments, an increase in translation for a Cas9 sequence in a mammal, cell type, organ of a mammal, human, organ of a human, etc., is determined relative to translation of an ORF with the sequence of SEQ ID NO: 36 with all else equal, including any applicable point mutations, heterologous domains, and the like. Codons useful for increasing expression in a human, including the human liver and human hepatocytes, can be codons corresponding to highly expressed tRNAs in the human liver/hepatocytes, which are discussed in Dittmar K A, PLos Genetics 2(12): e221 (2006). In some embodiments, at least about 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammal, such as a human. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian organ, such as a human organ. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian liver, such as a human liver. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian hepatocyte, such as a human hepatocyte.

Alternatively, codons corresponding to highly expressed tRNAs in an organism (e.g., human) in general may be used.

In some embodiments, at least 75%, 80%, 85%, 90%, 95%, 96%, 97%, 98%, 99%, or 100% of the codons in an ORF are codons from a codon set shown in Table 3 (e.g., the low U, low A, or low A/U codon set). The codons in the low A and low A/U sets use codons that minimize the indicated nucleotides while also using codons corresponding to highly expressed tRNAs where more than one option is available. In some embodiments, at least 75%, 80%, 85%, 90%, 95%, 96%, 97%, 98%, 99%, or 100% of the codons in an ORF are codons from the low U codon set shown in Table 3. In some embodiments, at least 75%, 80%, 85%, 90%, 95%, 96%, 97%, 98%, 99%, or 100% of the codons in an ORF are codons from the low A codon set shown in Table 3. In some embodiments, at least 75%, 80%, 85%, 90%, 95%, 96%, 97%, 98%, 99%, or 100% of the codons in an ORF are codons from the low A/U codon set shown in Table 3.

TABLE 3
Exemplary Codon Sets.

| Amino Acid | Low U | Low A | Low A/U |
|---|---|---|---|
| Gly | GGC | GGC | GGC |
| Glu | GAG | GAG | GAG |
| Asp | GAC | GAC | GAC |
| Val | GTG | GTG | GTG |
| Ala | GCC | GCC | GCC |
| Arg | AGA | CGG | CGG |
| Ser | AGC | TCC | AGC |
| Lys | AAG | AAG | AAG |
| Asn | AAC | AAC | AAC |
| Met | ATG | ATG | ATG |
| Ile | ATC | ATC | ATC |
| Thr | ACC | ACC | ACC |
| Trp | TGG | TGG | TGG |
| Cys | TGC | TGC | TGC |
| Tyr | TAC | TAC | TAC |
| Leu | CTG | CTG | CTG |
| Phe | TTC | TTC | TTC |
| Gln | CAG | CAG | CAG |
| His | CAC | CAC | CAC |

### Exemplary Sequences

In some embodiments, the ORF encoding the Cas nuclease comprises a sequence with at least 95%, 96%, 97%, 98%, 99%, 99.5%, or 100% identity to any of SEQ ID NOs: 1-12 and 36.

In some embodiments, the mRNA comprises an ORF encoding a Cas nuclease, wherein the Cas nuclease comprises an amino acid sequence with at least 95%, 96%, 97%, 98%, 99%, 99.5%, or 100% identity to any of SEQ ID NOs: 13-14.

In some embodiments, the ORF encoding the Cas nuclease comprises a sequence that is codon optimized according to the sequences provided in Table 3 from any of SEQ ID NOS: 1-12 and 36 or a sequence with at least 95%, 96%, 97%, 98%, 99%, 99.5%, or 100% identity to any of SEQ ID NOs: 1-12 and 36.

As used herein, a first sequence is considered to “comprise a sequence with at least X % identity to” a second sequence if an alignment of the first sequence to the second sequence shows that X % or more of the positions of the second sequence in its entirety are matched by the first sequence. Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

Additional Features of RNA, mRNAs, and ORFs

Any of the additional features described herein may be combined to the extent feasible with any of the embodiments described above.

### Encoded Cas Nuclease

In some embodiments, the Cas nuclease is a Class 2 Cas nuclease. In some embodiments, the Cas nuclease has cleavase activity, which can also be referred to as double-strand endonuclease activity or nickase activity. In some embodiments, the Cas nuclease is a Class 2 Cas nuclease (which may be, e.g., a Cas nuclease of Type II, V, or VI). Class 2 Cas nucleases include, for example, Cas9, Cpf1, C2c1, C2c2, and C2c3 proteins and modifications thereof. Examples of Cas9 nucleases include those of the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1. Other examples of Cas nucleases include a Csm or Cmr complex of a type III CRISPR system or the Cas10, Csm1, or Cmr2 subunit thereof; and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., Nat. Rev. Microbiol. 9:467-477 (2011); Makarova et al., Nat. Rev. Microbiol, 13:722-36 (2015); Shmakov et al., Molecular Cell, 60:385-397 (2015). In some embodiments, the Cas nuclease is a Cas cleavase, e.g. a Cas9 cleavase. In some embodiments, the Cas nuclease is a Cas nickase, e.g. a Cas9 nickase. In some embodiments, the Cas nuclease is an S. pyogenes Cas9 nuclease, e.g. a cleavase.

Non-limiting exemplary species that the Cas nuclease, e.g. Cas9 nuclease, can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is a Cas9 nuclease from Streptococcus pyogenes. In some embodiments, the Cas nuclease is a Cas9 nuclease from Streptococcus thermophilus. In some embodiments, the Cas nuclease is a Cas9 nuclease from Neisseria meningitidis. In some embodiments, the Cas nuclease is a Cas9 nuclease is from Staphylococcus aureus. In some embodiments, the Cas nuclease is a Cpf1 nuclease from Francisella novicida. In some embodiments, the Cas nuclease is a Cpf1 nuclease from Acidaminococcus sp. In some embodiments, the Cas nuclease is a Cpf1 nuclease from Lachnospiraceae bacterium ND2006. In further embodiments, the Cas nuclease is a Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

Wild type Cas9 has two nuclease domains: RuvC and HNH. The RuvC domain cleaves the non-target DNA strand, and the HINH domain cleaves the target strand of DNA. In some embodiments, the Cas9 nuclease comprises more than one RuvC domain and/or more than one HNH domain. In some embodiments, the Cas9 nuclease is a wild type Cas9. In some embodiments, the Cas9 nuclease is capable of inducing a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

### Poly-A Tail

In some embodiments, the RNA (e.g. mRNA) further comprises a poly-adenylated (poly-A) tail. In some instances, the poly-A tail is “interrupted” with one or more non-adenine nucleotide “anchors” at one or more locations within the poly-A tail, e.g. when it is encoded by a plasmid. The poly-A tails may comprise at least 8 consecutive adenine nucleotides, and in some embodiments, the poly-A tail also comprises one or more non-adenine nucleotide. As used herein, “non-adenine nucleotides” refer to any natural or non-natural nucleotides that do not comprise adenine. Guanine, thymine, and cytosine nucleotides are exemplary non-adenine nucleotides. Thus, the poly-A tails on the polynucleotide (e.g. mRNA) described herein may comprise consecutive adenine nucleotides located 3′ to nucleotides encoding a Cas nuclease or a sequence of interest. In some instances, the poly-A tails on mRNA comprise non-consecutive adenine nucleotides located 3′ to nucleotides encoding a Cas nuclease or a sequence of interest, wherein non-adenine nucleotides interrupt the adenine nucleotides at regular or irregularly spaced intervals.

In some embodiments, the poly-A tail is encoded in the plasmid used for in vitro transcription of mRNA and becomes part of the transcript. The poly-A sequence encoded in the plasmid, i.e., the number of consecutive adenine nucleotides in the poly-A sequence, may not be exact, e.g., a 100 poly-A sequence (SEQ ID NO: 58) in the plasmid may result in up to 100 poly-A sequence (SEQ ID NO: 58) in the transcribed mRNA. In some embodiments, the poly-A tail is not encoded in the plasmid, and is added by PCR tailing or enzymatic tailing, e.g., using E. coli poly (A) polymerase.

### UTRs; Kozak Sequences

In some embodiments, the RNA encoding a Cas nuclease (e.g. mRNA) comprises a 5′ UTR, a 3′ UTR, or 5′ and 3′ UTRs. In some embodiments, the RNA (e.g. mRNA) comprises at least one UTR from Hydroxysteroid 17-Beta Dehydrogenase 4 (HSD17B4 or HSD), e.g., a 5′ UTR from HSD. In some embodiments, the RNA (e.g. mRNA) comprises at least one UTR from a globin mRNA, for example, human alpha globin (HBA) mRNA, human beta globin (HBB) mRNA, or Xenopus laevis beta globin (XBG) mRNA. In some embodiments, the polynucleotide (e.g. mRNA) comprises a 5′ UTR, 3′ UTR, or 5′ and 3′ UTRs from a globin mRNA, such as HBA, HBB, or XBG. In some embodiments, the polynucleotide (e.g. mRNA) comprises a 5′ UTR from bovine growth hormone, cytomegalovirus (CMV), mouse Hba-al, HSD, an albumin gene, HBA, HBB, or XBG. In some embodiments, the polynucleotide (e.g. mRNA) comprises a 3′ UTR from bovine growth hormone, cytomegalovirus, mouse Hba-al, HSD, an albumin gene, HBA, HBB, or XBG. In some embodiments, the polynucleotide (e.g. mRNA) comprises 5′ and 3′ UTRs from bovine growth hormone, cytomegalovirus, mouse Hba-al, HSD, an albumin gene, HBA, HBB, XBG, heat shock protein 90 (Hsp90), glyceraldehyde 3-phosphate dehydrogenase (GAPDH), beta-actin, alpha-tubulin, tumor protein (p53), or epidermal growth factor receptor (EGFR).

In some embodiments, the polynucleotide (e.g. mRNA) comprises 5′ and 3′ UTRs that are from the same source, e.g., a constitutively expressed mRNA such as actin, albumin, or a globin such as HBA, HBB, or XBG.

In some embodiments, the polynucleotide (e.g. mRNA) comprises a Kozak sequence. Kozak sequences are known in the art. The Kozak sequence can affect translation initiation and the overall yield of a polypeptide translated from a nucleic acid. A Kozak sequence includes a methionine codon that can function as the start codon. A minimal Kozak sequence is NNNRUGN wherein at least one of the following is true: the first N is A or G and the second N is G. In the context of a nucleotide sequence, R means a purine (A or G). In some embodiments, the Kozak sequence is gccgccRccAUGG (SEQ ID NO: 37) with zero mismatches or with up to one, two, three, or four mismatches to positions in lowercase.

### Modified Nucleotides

In some embodiments, the mRNA comprising an ORF encoding a Cas nuclease comprises a modified uridine at some or all uridine positions. In some embodiments, the modified uridine is a uridine modified at the 5 position, e.g., with a halogen or C1-C3 alkoxy. In some embodiments, the modified uridine is a pseudouridine modified at the 1 position, e.g., with a C1-C3 alkyl. The modified uridine can be, for example, pseudouridine, N1-methyl-pseudouridine, 5-methoxyuridine, 5-iodouridine, or a combination thereof. In some embodiments the modified uridine is 5-methoxyuridine. In some embodiments the modified uridine is 5-iodouridine. In some embodiments the modified uridine is pseudouridine. In some embodiments the modified uridine is N1-methyl-pseudouridine. In some embodiments, the modified uridine is a combination of pseudouridine and N1-methyl-pseudouridine.

In some embodiments, at least 90%, 95%, 98%, 99%, or 100% of the uridine positions in the nucleic acid are modified uridines. In some embodiments, 85-95%, or 90-100% of the uridine positions in the nucleic acid are modified uridines, e.g., N1-methyl pseudouridine, pseudouridine, or a combination thereof. In some embodiments, 85-95%, or 90-100% of the uridine positions in the nucleic acid are pseudouridine. In some embodiments, 85-95%, or 90-100% of the uridine positions in the nucleic acid are N1-methyl pseudouridine.

### 5′ Cap

In some embodiments, mRNA comprising an ORF encoding a Cas nuclease (e.g., Cas9) comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the nucleic acid, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33): 12025-30; Abbas et al. (2017) Proc Natl Acad Sci USA 114(11): E2106-E2115.

A cap can be included in an RNA co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl (3′-O-methyl) GpppG and 7-methyl (3′deoxy) GpppG,” RNA 7:1486-1495. The ARCA structure is shown below.

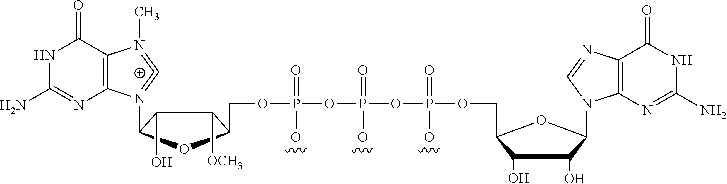

CleanCap™ AG (m7G (5′) ppp (5′) (2′OMeA) pG; TriLink Biotechnologies Cat. No. N-7113) or CleanCap™ GG (m7G (5′) ppp (5′) (2′OMeG) pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below. CleanCap™ structures are sometimes referred to herein using the last three digits of the catalog numbers listed above (e.g., “CleanCap™ 113” for TriLink Biotechnologies Cat. No. N-7113).

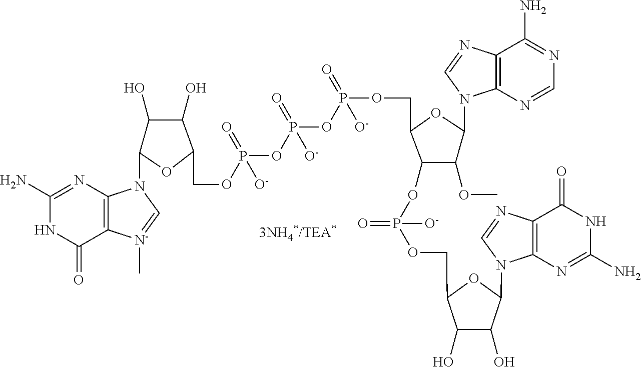

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its DI subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479. For additional discussion of caps and capping approaches, see, e.g., WO2017/053297 and Ishikawa et al., Nucl. Acids. Symp. Ser. (2009) No. 53, 129-130.

### 4. Delivery of Nucleic Acid Compositions

In some embodiments, a method of inducing a double-stranded break (DSB) or gene editing within the gene of interest (e.g., TTR) is provided comprising administering a composition comprising a guide RNA as described herein. In some embodiments, one or more of guide sequences are administered to induce a DSB in the gene of interest. The guide RNA is administered together with an RNA (e.g., mRNA) encoding a Cas nuclease (e.g., Cas9). The Cas nuclease may be an S. pyogenes Cas9. In particular embodiments, the guide RNA is chemically modified. In some embodiments, the guide RNA and the RNA encoding a Cas nuclease are administered in an LNP described herein, such as an LNP comprising a Lipid A. In further embodiments, the LNP comprises a lipid component that includes Lipid A, a helper lipid (e.g., cholesterol), a stealth lipid (e.g., a PEG lipid, such as PEG2k-DMG), and optionally a neutral lipid (e.g., DSPC).

In some embodiments, a method of inducing a double-stranded break (DSB) within the gene of interest (e.g., TTR) is provided comprising administering a LNP composition comprising a guide RNA, such as a chemically modified guide RNA. In some embodiments, one or more of sgRNAs are administered to induce a DSB in the gene of interest. The guide RNA is administered together with a RNA described herein encoding a Cas nuclease (e.g., Cas9). The Cas nuclease may be an S. pyogenes Cas9. In particular embodiments, the guide RNA is chemically modified. In some embodiments, the guide RNA and the RNA encoding a Cas nuclease are administered in an LNP described herein, such as an LNP comprising a Lipid A, a helper lipid (e.g., cholesterol), a stealth lipid (e.g., a PEG lipid, such as PEG2k-DMG), and optionally a neutral lipid (e.g., DSPC).

In some embodiments, a method of modifying the gene of interest (e.g., TTR) is provided comprising administering a composition comprising a guide RNA, such as a chemically modified guide RNA. In some embodiments, one or more of the sgRNAs are administered to modify the gene of interest. The guide RNA is administered together with a RNA described herein encoding a Cas nuclease (e.g., Cas9). The Cas nuclease may be an S. pyogenes Cas9. In particular embodiments, the guide RNA is chemically modified. In some embodiments, the guide RNA and the RNA encoding a Cas nuclease are administered in an LNP described herein, such as an LNP comprising a Lipid A, a helper lipid (e.g., cholesterol), a stealth lipid (e.g., a PEG lipid, such as PEG2k-DMG), and optionally a neutral lipid (e.g., DSPC).

In some embodiments, a method of treating a disease (e.g., ATTR) is provided comprising administering a composition comprising one or more of the guide RNAsThe guide RNA is administered together with a RNA described hereinencoding a Cas nuclease such as e.g., Cas9. The Cas nuclease may be an S. pyogenes Cas9. In particular embodiments, the guide RNA is chemically modified. In some embodiments, the guide RNA and the nucleic acid encoding a Cas nuclease are administered in an LNP described herein, such as an LNP comprising Lipid A, a helper lipid (e.g., cholesterol), a stealth lipid (e.g., a PEG lipid, such as PEG2k-DMG), and optionally a neutral lipid (e.g., DSPC).

In some embodiments, a method of reducing a gene product (e.g., TTR) is provided comprising administering one or more guide RNAs. In some embodiments, gRNAs comprising one or more of guide sequences are administered to reduce or prevent the accumulation of a gene product. The gRNA is administered together with a nucleic acid encoding a Cas nuclease e.g., Cas9. The Cas nuclease may be an S. pyogenes Cas9. In particular embodiments, the guide RNA is chemically modified. In some embodiments, the guide RNA and the RNA encoding a Cas nuclease are administered in an LNP described herein, such as an LNP comprising Lipid A, a helper lipid (e.g., cholesterol), a stealth lipid (e.g., a PEG lipid, such as PEG2k-DMG), and optionally a neutral lipid (e.g., DSPC).

In some embodiments, a method of reducing a gene product concentration is provided comprising administering one or more guide RNAs as described herein. In some embodiments, gRNAs comprising one or more of guide sequences are administered to reduce or prevent the accumulation of the gene product. The gRNA is administered together with a nucleic acid encoding a Cas nuclease e.g., Cas9. The Cas nuclease may be an S. pyogenes Cas9. In particular embodiments, the guide RNA is chemically modified. In some embodiments, the guide RNA and the RNA encoding a Cas nuclease are administered in an LNP described herein, such as an LNP comprising Lipid A), a helper lipid (e.g., cholesterol), a stealth lipid (e.g., a PEG lipid, such as PEG2k-DMG), and optionally a neutral lipid (e.g., DSPC).

In some embodiments, a method of reducing or preventing the accumulation of the gene product of a subject is provided comprising one or more guide RNAs as described herein. In some embodiments, a method of reducing or preventing the accumulation of the gene product of a subject is provided comprising administering a composition comprising one or more of sgRNAs. In some embodiments, gRNAs comprising one or more guide sequences are administered to reduce or prevent the accumulation of TTR in amyloids or amyloid fibrils. The gRNA is administered together with a RNA encoding a Cas nuclease e.g., Cas9. The Cas nuclease may be an S. pyogenes Cas9. In particular embodiments, the guide RNA is chemically modified. In some embodiments, the guide RNA and the nucleic acid encoding a Cas nuclease are administered in an LNP described herein, such as an LNP comprising Lipid A), a helper lipid (e.g., cholesterol), a stealth lipid (e.g., a PEG lipid, such as PEG2k-DMG), and optionally a neutral lipid (e.g., DSPC).

In some embodiments, the gRNA comprising a guide sequence together with a Cas nuclease translated from the nucleic acid induce DSBs, and non-homologous ending joining (NHEJ) during repair leads to a mutation in the TTR gene. In some embodiments, NHEJ leads to a deletion or insertion of a nucleotide(s), which induces a frame shift or nonsense mutation in the TTR gene.

### 5. Lipid Compositions

In some embodiments, the nucleic acid compositions described herein, comprising a gRNA and a nucleic acid described herein encoding a Cas nuclease e.g. Cas9, are formulated in or administered via a lipid nanoparticle; see e.g., WO2017173054A1 entitled “LIPID NANOPARTICLE FORMULATIONS FOR CRISPR/CAS COMPONENTS,” and WO2019067992A1 entitled “FORMULATIONS,” the contents of which, in particular the LNP compositions disclosed therein, are hereby incorporated by reference in their entirety. Lipid nanoparticles (LNPs) known to those of skill in the art to be capable of delivering therapeutic RNAs to subjects may be utilized with the guide RNAs described herein and the nucleic acid encoding a Casnuclease.

Compositions comprising LNPs may include two active substances, a guide RNA and an RNA encoding a Cas nuclease, together with a lipid component comprising an ionizable lipid. By lipid nanoparticle is meant a particle that comprises a plurality of (i.e. more than one) lipid molecules physically associated with each other by intermolecular forces.

### Ionizable Lipids

Lipid compositions for delivery of CRISPR/Cas mRNA and guide RNA components to a liver cell may comprise Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl(9Z,12Z)-octadeca-9,12-dienoate. Lipid A can be depicted as:

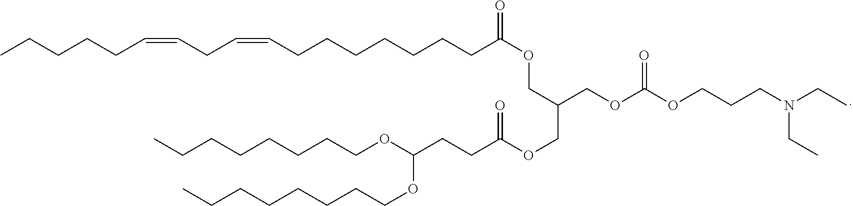 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86).

### Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In one embodiment, the helper lipid may be cholesterol.

“Stealth lipids” are lipids that alter the length of time the nanoparticles can exist in vivo (e.g., in the blood), and a stealth lipid may be a PEG lipid. Stealth lipids may assist in the formulation process by, for example, reducing particle aggregation and controlling particle size. Stealth lipids used herein may modulate pharmacokinetic properties of the LNP. Stealth lipids suitable for use in a lipid composition of the disclosure include, but are not Typically, the PEG lipid comprises a lipid moiety and a polymer moiety based on PEG. PEG lipids known in the art are contemplated, including lipids comprising a “PEG-2K,” also termed “PEG 2000,” which has an average molecular weight of about 2,000 daltons. PEG-2K is represented herein by the following formula (I), wherein n is 45, meaning that the number averaged degree of polymerization comprises about 45 subunits. However, other PEG embodiments known in the art may be used.

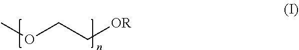

In any of the embodiments described herein, the PEG lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3 [beta]-oxy) carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol) ether), 1,2-dimyristoyl-sn-glycero-3-phosphoethanolamine-N-[methoxy (polyethylene glycol)-2000] (PEG2k-DMPE), or 1,2-dimyristoyl-rac-glycero-3-methoxypolyethylene glycol-2000 (PEG2k-DMG), 1,2-distearoyl-sn-glycero-3-phosphoethanolamine-N-[methoxy (polyethylene glycol)-2000] (PEG2k-DSPE) (cat. #880120C from Avanti Polar Lipids, Alabaster, Alabama, USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy (polyethylene glycol)-2000] (PEG2k-DSA). In one embodiment, the PEG lipid may be PEG2k-DMG.

In some embodiments, the PEG lipid includes a glycerol group. In some embodiments, the PEG lipid includes a dimyristoylglycerol (DMG) group. In some embodiments, the PEG lipid comprises PEG2k. In some embodiments, the PEG lipid is a PEG-DMG. In some embodiments, the PEG lipid is a PEG2k-DMG. In some embodiments, the PEG lipid is 1,2-dimyristoyl-rac-glycero-3-methoxypolyethylene glycol-2000. In some embodiments, the PEG2k-DMG is 1,2-dimyristoyl-rac-glycero-3-methoxypolyethylene glycol-2000.

### LNP Formulations

The LNP composition may comprise a lipid component and an RNA component that includes a Cas nuclease mRNA (e.g. a Class 2 Cas nuclease mRNA, such as a Cas9 mRNA), and a gRNA. In some embodiments, an LNP composition includes an mRNA encoding a Class 2 Cas nuclease and a gRNA as the RNA component. In certain embodiments, an LNP composition may comprise the RNA component, Lipid A, a helper lipid, a neutral lipid, and a stealth lipid. In certain LNP compositions, the helper lipid is cholesterol. In certain compositions, the neutral lipid is DSPC. In additional embodiments, the stealth lipid is PEG2k-DMG.

In certain embodiments, lipid compositions are described according to the respective molar ratios of the component lipids in the formulation. Embodiments of the present disclosure provide lipid compositions described according to the respective molar ratios of the component lipids in the formulation. In one embodiment, the mol-% of the ionizable lipid such as Lipid A may be from about 40 mol-% to 60 mol-%, optionally about 50 mol-%. In one embodiment, the mol-% of the ionizable lipid may be about 55 mol-%. In some embodiments, the ionizable lipid mol-% of the LNP batch will be ±30%, ±25%, ±20%, ±15%, ±10%, ±5%, or ±2.5% of the target mol-%. In some embodiments, the ionizable lipid mol-% of the LNP batch will be ±4 mol-%, ±3 mol-%, ±2 mol-%, ±1.5 mol-%, ±1 mol-%, ±0.5 mol-%, or ±0.25 mol-% of the target mol-%. All mol-% numbers are given as a fraction of the lipid components of the LNP composition.

In one embodiment, the mol-% of the neutral lipid, e.g., neutral phospholipid, may be from about 5 mol-% to 15 mol-%, optionally about 9 mol-%. In some embodiments, the neutral lipid mol-% of the LNP batch will be ±30%, ±25%, ±20%, ±15%, ±10%, ±5%, or ±2.5% of the target neutral lipid mol-%.

In one embodiment, the mol-% of the helper lipid may be from about 20 mol-% to 60 mol-%. In one embodiment, the mol-% of the helper lipid may be from about 25 mol-% to 55 mol-%, optionally, the mol-% of the helper lipid may be from about 30 mol-% to 40 mol-%. In one embodiment, the mol-% of the helper lipid is adjusted based on ionizable lipid, neutral lipid, and PEG lipid concentrations to bring the lipid component to 100 mol-%. In one embodiment, the mol-% of the helper lipid is adjusted based on ionizable lipid and PEG lipid concentrations to bring the lipid component to at least 99 mol-%. In some embodiments, the helper mol-% of the LNP batch will be ±30%, ±25%, ±20%, ±15%, ±10%, ±5%, or ±2.5% of the target mol-%.

In one embodiment, the mol-% of the PEG lipid may be from about 1 mol-% to 10 mol-%. In one embodiment, the mol-% of the PEG lipid may be from about 2 mol-% to 4 mol-%. In one embodiment, the mol-% of the PEG lipid may be from about 2.5 mol-% to 4 mol-%. In one embodiment, the mol-% of the PEG lipid may be about 3 mol-%. In some embodiments, the PEG lipid mol-% of the LNP batch will be ±30%, ±25%, ±20%, ±15%, ±10%, ±5%, or ±2.5% of the target PEG lipid mol-%.

In certain embodiments, the cargo includes a nucleic acid (e.g., mRNA) encoding a Cas nuclease (e.g. a Cas nuclease, a Class 2 Cas nuclease, or Cas9), and a gRNA. In one embodiment, the ionizable lipid is Lipid A. In various embodiments, an LNP composition comprises an ionizable lipid (e.g. Lipid A), a neutral lipid, a helper lipid, and a PEG lipid. In certain embodiments, the helper lipid is cholesterol. In certain embodiments, the neutral lipid is DSPC. In specific embodiments, PEG lipid is PEG2k-DMG. In some embodiments, an LNP composition may comprise a Lipid A, a helper lipid, a neutral lipid, and a PEG lipid. In additional embodiments, an LNP composition comprises Lipid A, cholesterol, DSPC, and PEG2k-DMG.

Embodiments of the present disclosure also provide lipid compositions described according to the molar ratio between the positively charged ionizable groups of the ionizable lipid (N) and the negatively charged phosphate groups (P) of the nucleic acid to be encapsulated. This may be mathematically represented by the ratio N/P. In some embodiments, an LNP composition may comprise a lipid component that comprises an ionizable lipid, a helper lipid, a neutral lipid, and a PEG lipid; and a nucleic acid component, wherein the N/P ratio is about 3 to 10. In one embodiment, the N/P ratio may be about 5 to 7, optionally the N/P ratio may be about 6. In one embodiment, the N/P ratio may be 6±1. In one embodiment, the N/P ratio may be 6±0.5. In some embodiments, the N/P ratio will be ±30%, ±25%, ±20%, ±15%, ±10%, ±5%, or ±2.5% of the target N/P ratio.

In some embodiments, the RNA component may comprise RNA, such as a nucleic acid disclosed herein, e.g., encoding a Cas nuclease described herein (such as a Cas9 mRNA described herein), and a gRNA described herein. In some embodiments, the RNA component comprises a Cas nuclease mRNA described herein and a gRNA described herein. In some embodiments, the RNA component comprises a Class 2 Cas nuclease mRNA described herein and a gRNA described herein. In any of the foregoing embodiments, the gRNA may be an sgRNA described herein, such as a chemically modified sgRNA described herein.

In certain embodiments, the LNP compositions include a Cas nuclease mRNA (such as a Class 2 Cas mRNA) described herein and at least one gRNA described herein. In certain embodiments, the LNP composition includes a ratio of gRNA to Cas nuclease mRNA, such as Class 2 Cas nuclease mRNA from about 10:1 to 1:10. In some embodiments the ratio of gRNA to Cas nuclease mRNA, such as Class 2 Cas nuclease is about 1:1. In some embodiments the ratio of gRNA to Cas nuclease mRNA, such as Class 2 Cas nuclease is about 1:2. In some embodiments the ratio of gRNA to Cas nuclease mRNA, such as Class 2 Cas nuclease is about 1:3.

In some embodiments, LNPs are formed by mixing an aqueous RNA solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer, e.g., for in vivo administration of LNPs, may be used.

In some embodiments, microfluidic mixing, T-mixing, or cross-mixing is used. In certain aspects, flow rates, junction size, junction geometry, junction shape, tube diameter, solutions, and/or RNA and lipid concentrations may be varied. LNPs or LNP compositions may be concentrated or purified, e.g., via dialysis, tangential flow filtration, or chromatography. The LNPs may be composed of 4 lipids including Lipid A; DSPC; cholesterol; and DMG-PEG2k. In some embodiments, the LNP is suspended and formulated in an aqueous buffer of 50 mM Tris, 45 mM NaCl, and 5% (w/v) sucrose, pH 7.4.

Dynamic Light Scattering (“DLS”) can be used to characterize the polydispersity index (“pdi”) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero.

In some embodiments, LNPs disclosed herein have a size of 50 to 100 nm. In some embodiments, the LNPs have a size of 85 to 90 nm. Unless indicated otherwise, all sizes referred to herein are the average sizes (diameters) of the fully formed nanoparticles, as measured by dynamic light scattering on a Malvern Zetasizer. The nanoparticle sample is diluted in phosphate buffered saline (PBS) so that the count rate is approximately 200-400 kcts. The data is presented as a weighted-average of the intensity measure.

In some embodiments, LNPs associated with the gRNAs disclosed herein and RNA (e.g., mRNA) encoding a Cas nuclease (e.g. Cas9, Spy Cas9) disclosed herein are for use in preparing a medicament for treating ATTR. In some embodiments, LNPs associated with the gRNAs disclosed herein and RNA (e.g., mRNA) encoding a Cas nuclease (e.g. Cas9, Spy Cas9) disclosed herein are for use in preparing a medicament for reducing or preventing accumulation and aggregation of TTR in amyloids or amyloid fibrils in subjects having ATTR. In some embodiments, LNPs associated with the gRNAs disclosed herein and RNA (e.g., mRNA) encoding a Cas nuclease (e.g. Cas9, Spy Cas9) disclosed herein are for use in preparing a medicament for reducing serum TTR concentration. In some embodiments, LNPs associated with the gRNAs disclosed herein and RNA (e.g., mRNA) encoding a Cas nuclease (e.g. Cas9, Spy Cas9) disclosed herein are for use in preparing a medicament for reducing serum prealbumin concentration. In some embodiments, LNPs associated with the gRNAs disclosed herein and RNA (e.g., mRNA) encoding an Cas nuclease (e.g. Cas9, Spy Cas9) disclosed herein are for use in treating ATTR in a subject, such as a mammal, e.g., a primate such as a human. In some embodiments, LNPs associated with the gRNAs disclosed herein and RNA (e.g., mRNA) encoding a Cas nuclease (e.g. Cas9, Spy Cas9) disclosed herein are for use in reducing or preventing accumulation and aggregation of TTR in amyloids or amyloid fibrils in subjects having ATTR, such as a mammal, e.g., a primate such as a human. In some embodiments, LNPs associated with the gRNAs disclosed herein and RNA (e.g., mRNA) encoding a Cas nuclease (e.g. Cas9, Spy Cas9) disclosed herein are for use in reducing serum TTR concentration in a subject, such as a mammal, e.g., a primate such as a human. In some embodiments, LNPs associated with the gRNAs disclosed herein and RNA (e.g., mRNA) encoding a Cas nuclease (e.g. Cas9, Spy Cas9) disclosed herein are for use in reducing serum prealbumin concentration in a subject, such as a mammal, e.g., a primate such as a human.

In some instances, the lipid component comprises: 48-53 mol-% Lipid A; about 8-10 mol-% DSPC; and 1.5-10 mol-% PEG lipid (PEG2k-DMG), wherein the remainder of the lipid component is cholesterol, and wherein the N/P ratio of the LNP composition is 3-8±0.2.

In some embodiments, the LNP comprises a lipid component and the lipid component comprises, consists essentially of, or consists of: about 50 mol-% ionizable lipid such as Lipid A; about 9 mol-% neutral lipid such as DSPC; about 3 mol-% of a stealth lipid such as a PEG lipid, such as PEG2k-DMG, and the remainder of the lipid component is helper lipid such as cholesterol, wherein the N/P ratio of the LNP composition is about 6. In some embodiments, the ionizable lipid is Lipid A. In some embodiments, the neutral lipid is DSPC. In some embodiments, the stealth lipid is a PEG lipid. In some embodiments, the stealth lipid is a PEG2k-DMG. In some embodiments, the helper lipid is cholesterol. In some embodiments, the LNP comprises a lipid component and the lipid component comprises: about 50 mol-% Lipid A; about 9 mol-% DSPC; about 3 mol-% of PEG2k-DMG, and the remainder of the lipid component is cholesterol wherein the N/P ratio of the LNP composition is about 6.

### II. Methods of Systemic Delivery

In some embodiments, the LNP composition described herein (e.g., comprising an mRNA encoding a Cas nuclease, e.g., Cas9; and a guide RNA that targets a gene, e.g., a guide RNA that targets the TTR gene) is administered systemically. As used herein, systemic administration refers to broad biodistribution within an organism, e.g., intravenous administration, intraperitoneal injection, etc.

In some embodiments, a single administration of the LNP composition described herein is sufficient to knockdown expression of the target protein. In some embodiments, a single administration of the LNP composition is sufficient to knockdown expression of the target protein in a population of cells. In other embodiments, more than one administration of the LNP composition may be beneficial to maximize editing via cumulative effects. For example, the LNP composition can be administered a second or third time (a “follow-on dose”), e.g., a second dose or a third dose. The dose of the second dose or third dose can be determined by a clinician to provide, e.g., about or greater than 60%, 70%, 80%, or 90% reduction in serum TTR and/or serum prealbumin as compared to baseline levels (e.g., the level prior to first LNP administration). The more than one administration (second dose or third dose) can be administered as a weight-based (e.g., 0.7 mg/kg or 1.0 mg/kg) or fixed dose (e.g., about 60 mg, 70 mg, 80 mg, or 90 mg), regardless of how the first dose was administered (weight-based or fixed dose).

In some embodiments, the LNP composition described herein is administered by infusion with an infusion time between about 2 hours and 4 hours. In some embodiments, the LNP composition described herein is administered by infusion with an infusion time between about 2 hours and 3 hours. In some embodiments, the LNP composition described herein is administered by infusion with an infusion time between about 3 hours and 4 hours. In some embodiments, the LNP composition described herein is administered by infusion with an infusion time between about 4 hours and 5 hours. In some embodiments, the LNP composition described herein is administered by infusion with an infusion time of about 4 hours. In some embodiments, the LNP composition described herein is administered by infusion with an infusion time of at least 2 hours. In some embodiments, the LNP composition described herein is administered by infusion with an infusion time of at least 3 hours. In some embodiments, the LNP composition described herein is administered by infusion with an infusion time of at least 4 hours.

### III. Dose

### 1. Weight-Based Dose

In some embodiments, the LNP composition described herein (e.g., comprising an effective amount of mRNA encoding a Cas nuclease, e.g., Cas9; and a guide RNA that targets a gene, e.g., a guide RNA that targets the TTR gene (the total or combined RNA)) is administered using a weight-based dose. In some embodiments, the effective amount of the mRNA encoding a Cas nuclease and the guide RNA that targets the TTR gene is a combined dose of about 0.1 mg/kg to 2 mg/kg. In some embodiments, the effective amount of the mRNA encoding a Cas nuclease and the guide RNA that targets the TTR gene is a combined dose of about 0.3 mg/kg to 1 mg/kg. In some embodiments, the effective amount of the mRNA encoding a Cas nuclease and the guide RNA that targets the TTR gene is a combined dose of about 0.1 mg/kg. In some embodiments, the effective amount of the mRNA encoding a Cas nuclease and the guide RNA that targets the TTR gene is a combined dose of about 0.3 mg/kg. In some embodiments, the effective amount of the mRNA encoding a Cas nuclease and the guide RNA that targets the TTR gene is a combined dose of about 0.7 mg/kg. In some embodiments, the effective amount of the mRNA encoding a Cas nuclease and the guide RNA that targets the TTR gene is a combined dose of about 1 mg/kg. The LNP composition may be administered in an effective amount, in that the LNP composition-when dosed based on total RNA-administers an effective amount of an mRNA encoding a Cas nuclease and a guide RNA that targets the TTR gene.

In other embodiments, subjects who receive the 0.1 mg/kg dose may receive more than one administration of the LNP composition to maximize editing via cumulative effects. For example, the LNP composition can be administered 2, 3, 4, 5, or more times, such as 2 times—e.g., a second administration, a third administration, a fourth administration, or a fifth administration. In some embodiments, the LNP composition is administered to a human subject that has previously been administered the LNP composition. In some embodiments, the LNP composition is administered to a human subject that has previously been administered the LNP composition and has not achieved a greater than 60%, greater than 70%, or greater than 80% reduction in serum TTR (e.g., a less than 60%, less than 70%, or less than 80% decrease of serum TTR as measured by ELISA after administration of the LNP composition) as determined at, e.g., 28 days after the first LNP administration. In some embodiments, the LNP composition is administered to a human subject that has previously been administered the LNP composition and has not achieved a greater than 60%, greater than 70%, or greater than 80% reduction in serum TTR (e.g., a less than 60%, less than 70%, or less than 80% decrease of serum TTR as measured by mass spectrometry or ELISA after administration of the LNP composition) as determined at, e.g., 28 days after the first LNP administration. In some embodiments, the LNP composition is administered to a human subject that has previously been administered the LNP composition and has not achieved a greater than 60%, greater than 70%, or greater than 80% reduction in serum prealbumin (e.g., a less than 60%, less than 70%, or less than 80% decrease of serum prealbumin as measured by, e.g., turbidity assay after administration of the LNP composition) as determined at, e.g., 28 days after the first LNP administration. In some embodiments, the LNP composition is administered to a human subject that has previously been administered the LNP composition and has not achieved a greater than 60%, greater than 70%, or greater than 80% reduction in serum prealbumin (e.g., a less than 60%, less than 70%, or less than 80% decrease in serum prealbumin after administration of the LNP composition) as determined at, e.g., 28 days after the first LNP administration.

### 2. Fixed Dose

In some embodiments, the LNP composition described herein (e.g., comprising an effective amount of mRNA encoding a Cas nuclease, e.g., Cas9, and a guide RNA that targets a gene, e.g., a guide RNA that targets the TTR gene (the total or combined dose)) is administered using a fixed dose. The fixed dose may be about 25-150 mg in a human subject. In some embodiments, the effective amount of the mRNA encoding a Cas nuclease and the guide RNA that targets the TTR gene is a combined dose of about 5 mg to 75 mg, optionally about 25 mg to 75 mg, about 25 mg to 60 mg, about 25 mg to 80 mg, or about 25 mg to 115 mg. In some embodiments, the effective amount of the mRNA encoding a Cas nuclease and the guide RNA that targets the TTR gene is a combined dose of about 50 mg to 150 mg, optionally about 50 mg to 100 mg or about 75 mg to 150 mg. In some embodiments, the effective amount of the mRNA encoding a Cas nuclease and the guide RNA that targets the TTR gene is a combined dose of about 5 mg to 9 mg. In some embodiments, the effective amount of the mRNA encoding a Cas nuclease and the guide RNA that targets the TTR gene is a combined dose of about 15 mg to 27 mg. In some embodiments, the effective amount of the mRNA encoding a Cas nuclease and the guide RNA that targets the TTR gene is a combined dose of about 50 mg to 90 mg. In some embodiments, the effective amount of the mRNA encoding a Cas nuclease and the guide RNA that targets the TTR gene is a combined dose of about 35 mg to 65 mg. In some embodiments, the effective amount of the mRNA encoding a Cas nuclease and the guide RNA that targets the TTR gene is a combined dose of about 5 mg to 180 mg. In some embodiments, the effective amount of the mRNA encoding a Cas nuclease and the guide RNA that targets the TTR gene is a combined dose of about 50 mg to 70 mg. In some embodiments, the effective amount of the mRNA encoding a Cas nuclease and the guide RNA that targets the TTR gene is a combined dose of about 60 mg to 80 mg. In some embodiments, the effective amount of the mRNA encoding a Cas nuclease and the guide RNA that targets the TTR gene is a combined dose of about 70 mg to 90 mg. In some embodiments, the effective amount of the mRNA encoding a Cas nuclease and the guide RNA that targets the TTR gene is a combined dose of about 80 mg to 100 mg. The LNP composition may be administered in an effective amount, in that the LNP composition-when dosed based on total RNA-administers an effective amount of an mRNA encoding a Cas nuclease and a guide RNA that targets the TTR gene.

In some embodiments, the LNP composition described herein (e.g., comprising an effective amount of mRNA encoding a Cas nuclease, e.g., Cas9, and a guide RNA that targets a gene, e.g., a guide RNA that targets the TTR gene (the total or combined dose)) is administered using a fixed dose. The fixed dose may be 25-150 mg in a human subject. In some embodiments, the effective amount of the mRNA encoding a Cas nuclease and the guide RNA that targets the TTR gene is a combined dose of 5 mg to 75 mg, optionally 25 mg to 75 mg, 25 mg to 60 mg, 25 mg to 80 mg, or 25 mg to 115 mg. In some embodiments, the effective amount of the mRNA encoding a Cas nuclease and the guide RNA that targets the TTR gene is a combined dose of 50 mg to 150 mg, optionally 50 mg to 100 mg or 75 mg to 150 mg. In some embodiments, the effective amount of the mRNA encoding a Cas nuclease and the guide RNA that targets the TTR gene is a combined dose of 5 mg to 9 mg. In some embodiments, the effective amount of the mRNA encoding a Cas nuclease and the guide RNA that targets the TTR gene is a combined dose of 15 mg to 27 mg. In some embodiments, the effective amount of the mRNA encoding a Cas nuclease and the guide RNA that targets the TTR gene is a combined dose of 50 mg to 90 mg. In some embodiments, the effective amount of the mRNA encoding a Cas nuclease and the guide RNA that targets the TTR gene is a combined dose of 35 mg to 65 mg. In some embodiments, the effective amount of the mRNA encoding a Cas nuclease and the guide RNA that targets the TTR gene is a combined dose of 5 mg to 180 mg. In some embodiments, the effective amount of the mRNA encoding a Cas nuclease and the guide RNA that targets the TTR gene is a combined dose of 50 mg to 70 mg. In some embodiments, the effective amount of the mRNA encoding a Cas nuclease and the guide RNA that targets the TTR gene is a combined dose of 60 mg to 80 mg. In some embodiments, the effective amount of the mRNA encoding a Cas nuclease and the guide RNA that targets the TTR gene is a combined dose of 70 mg to 90 mg. In some embodiments, the effective amount of the mRNA encoding a Cas nuclease and the guide RNA that targets the TTR gene is a combined dose of 80 mg to 100 mg.

In some embodiments, the effective amount of the mRNA encoding a Cas nuclease and the guide RNA that targets the TTR gene is a combined dose of about 7 mg to 9 mg. In some embodiments, the effective amount of the mRNA encoding a Cas nuclease and the guide RNA that targets the TTR gene is a combined dose of about 25 mg to 27 mg. In some embodiments, the effective amount of the mRNA encoding a Cas nuclease and the guide RNA that targets the TTR gene is a combined dose of about 44 mg to 68 mg. In some embodiments, the effective amount of the mRNA encoding a Cas nuclease and the guide RNA that targets the TTR gene is a combined dose of about 59 mg to 111 mg.

In some embodiments, the effective amount of the mRNA encoding a Cas nuclease and the guide RNA that targets the TTR gene is a combined dose of 7 mg to 9 mg. In some embodiments, the effective amount of the mRNA encoding a Cas nuclease and the guide RNA that targets the TTR gene is a combined dose of 25 mg to 27 mg. In some embodiments, the effective amount of the mRNA encoding a Cas nuclease and the guide RNA that targets the TTR gene is a combined dose of 44 mg to 68 mg. In some embodiments, the effective amount of the mRNA encoding a Cas nuclease and the guide RNA that targets the TTR gene is a combined dose of 59 mg to 111 mg.

In some embodiments, the effective amount of the mRNA encoding a Cas nuclease and the guide RNA that targets the TTR gene is a combined dose of about 25 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, or 150 mg. In some embodiments, the effective amount of the mRNA encoding a Cas nuclease and the guide RNA that targets the TTR gene is a combined dose of 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139, 140 mg, 141 mg, 142 mg, 143 mg, 144 mg, 145 mg, 146 mg, 147 mg 148 mg, or 150 mg. In some embodiments, the effective amount of the mRNA encoding a Cas nuclease and the guide RNA that targets the TTR gene is a combined dose of about 80 mg. In some embodiments, the effective amount of the mRNA encoding a Cas nuclease and the guide RNA that targets the TTR gene is a combined dose of 80 mg.

In other embodiments, subjects who receive a dose of about 5 mg to 9 mg may receive more than one administration of the LNP composition to maximize editing via cumulative effects. For example, the LNP composition can be administered 2, 3, 4, 5, or more times, such as 2 times—e.g., a second administration, a third administration, a fourth administration, or a fifth administration. In some embodiments, the LNP composition is administered to a human subject that has previously been administered the LNP composition. In some embodiments, the LNP composition is administered to a human subject that has previously been administered the LNP composition and has not achieved a greater than 60%, greater than 70%, or greater than 80% reduction in serum TTR (e.g. a less than 60%, less than 70%, or less than 80% decrease of serum TTR as measured by ELISA after administration of the LNP composition) as determined at, e.g., 28 days after the first LNP administration. In some embodiments, the LNP composition is administered to a human subject that has previously been administered the LNP composition and has not achieved a greater than 60%, greater than 70%, or greater than 80% reduction in serum TTR (e.g., a less than 60%, less than 70%, or less than 80% decrease of serum TTR as measured by ELISA or mass spectrometry after administration of the LNP composition) as determined at, e.g., 28 days after the first LNP administration. In some embodiments, the LNP composition is administered to a human subject that has previously been administered the LNP composition and has not achieved a greater than 60%, greater than 70%, or greater than 80% reduction in serum prealbumin (e.g., a less than 60%, less than 70%, or less than 80% decrease of serum prealbumin as measured by, e.g., turbidity assay after administration of the LNP composition) as determined at, e.g., 28 days after the first LNP administration. In some embodiments, the LNP composition is administered to a human subject that has previously been administered the LNP composition and has not achieved a greater than 60%, greater than 70%, or greater than 80% reduction in serum prealbumin (e.g., a less than 60%, less than 70%, or less than 80% decrease of serum prealbumin after administration of the LNP composition) as determined at, e.g., 28 days after the first LNP administration.

In some embodiments of the invention, “about” means within +5% of the stated value, e.g., a range of 76 mg-84 mg for a value that is about 80 mg. In some embodiments of the invention, “about” means within +10% of the stated value.

### IV. Methods of Use

### 1. Methods of In Vivo Editing

Methods of in vivo editing of a gene in the liver of a human subject having a monogenic disorder are provided herein. In some embodiments, the method of in vivo editing of the gene comprises systemically administering to the human subject the LNP composition described herein (e.g., comprising an mRNA encoding a Cas nuclease, e.g., Cas9; and a guide RNA that targets a gene, e.g., a guide RNA that targets the TTR gene). In some embodiments, the in vivo editing occurs at the site targeted by the guide RNA in a hepatocyte of the subject.

Methods of in vivo editing of a TTR gene in the liver a human subject are also provided herein. In some embodiments, the method of in vivo editing of the TTR gene comprises systemically administering to the human subject a LNP composition described herein (e.g., comprising an mRNA encoding a Cas nuclease, e.g., Cas9; and a guide RNA that targets the TTR gene). In some embodiments, the in vivo editing of the TTR gene occurs at the site targeted by the guide RNA in a hepatocyte of the subject.

In these embodiments, administration of the LNP composition to the subject may be associated with a change in a biosafety metric. In some embodiments, the subject is assessed to determine whether the change in the biosafety metric is an acceptable change. In some embodiments, an acceptable change can be determined by a clinician and/or laboratory. In some embodiments, an acceptable change can be one that does not qualify as a safety event, including an adverse event (NCI-CTCAE Grade not greater than or equal to 3), a serious adverse event, an adverse event of special interest, and/or a treatment-emergent adverse event (CTCAE Grade not greater than or equal to 3), as described herein. Biosafety metrics, including those associated with administration of an LNP composition, are known in the art. Acceptable levels and/or changes in the biosafety metrics are known in the art and may be assessed by routine methods.

In some embodiments, an acceptable biosafety metric level is one that falls within the subject inclusion criteria and/or does not fall within the subject exclusion criteria described herein.

In some embodiments, an acceptable change in a biosafety metric level is a change that is acceptable after a period of time, e.g., initially falls outside of acceptable levels but stabilizes to an acceptable level by, e.g., day 2, 3, 4, 5, 6, 7, 14 or 28 after administration. In some embodiments, an acceptable change in a biosafety metric level is a change in a level that falls within 150% of the upper limit of normal for said biosafety metric and/or within 50% of the lower limit of normal for said biosafety metric, e.g., within 150% of the prothrombin ULN and/or within 50% of the LLN of fibrinogen.

In some embodiments, an acceptable biosafety metric level (or an acceptable change in a biosafety metric level) is one that does not constitute an adverse event of Grade 3 or higher according to CTCAE guidelines, including National Cancer Institute (NCI)-CTCAE guidelines, version 5.0. In some embodiments, a change in a biosafety metric level (e.g., one or more levels associated with a laboratory parameter, vital sign, ECG data, physical exam, etc., as described herein) constitutes as an adverse event if the change, e.g., induces clinical signs or symptoms; requires active intervention; requires interruption or discontinuation of the LNP composition; and/or the change in the biosafety metric is clinically significant, as determined by a clinician.

In some embodiments, an adverse event is any untoward medical occurrence in a subject administered a study drug or has undergone study procedures and which does not necessarily have a causal relationship with the treatment. In some embodiments, an adverse event is an unintended sign (including an abnormal laboratory finding), symptom, or disease temporally associated with the treatment, whether or not related to the medicinal (investigational) product. In some embodiments, an adverse event that induces clinical signs or symptoms. In some embodiments, an adverse event requires active intervention. In some embodiments, an adverse event requires interruption or discontinuation of the treatment. In some embodiments, an adverse event is an abnormality that is clinically significant in the opinion of the investigator. Grading criteria for adverse events are known in the art, such as, e.g., Common Terminology Criteria for Adverse Events (CTCAE), including National Cancer Institute (NCI)-CTCAE.

In some embodiments, an acceptable biosafety metric level (or an acceptable change in a biosafety metric level) is one that does not constitute a serious adverse event. In some embodiments, a serious adverse event results in death. In some embodiments, a serious adverse event is life threatening (e.g., places the subject at immediate risk of death as determined by a clinician). In some embodiments, a serious adverse event results in persistent or significant disability. In some embodiments, a serious adverse event results in incapacity or substantial disruption of the ability to conduct normal life functions. In some embodiments, a serious adverse event results in congenital anomaly or birth defect. In some embodiments, a serious adverse event requires inpatient hospitalization or leads to prolongation of hospitalization.

In some embodiments, an acceptable biosafety metric level (or an acceptable change in a biosafety metric level) is one that does not constitute an adverse event of special interest. In some embodiments, an adverse event of special interest includes, e.g., infusion-related reaction (IRR) (e.g., requiring treatment or discontinuation of infusion, and/or Grade 3 or higher), incidence of thrombosis, incidence of hemorrhage, CTCAE≥Grade 2 abnormal blood test results, CTCAE≥Grade 2 elevation in ALT, CTCAE≥Grade 2 elevation in AST, CTCAE≥Grade 2 elevation in total bilirubin, CTCAE≥Grade 2 elevation in GLDH, incidence of cytokine release syndrome, an event attributed to impacts on the spleen (splenic hemorrhage, splenic infarction, sometimes thrombocytopenia, sometimes anemia or lymphopenia with specific abnormal findings on study of the blood cells on microscopy), an event attributed to impacts on the adrenal glands, clinically relevant symptoms or hypothyroidism, and an ophthalmic event consistent with Vitamin A deficiency.

In some embodiments, an acceptable biosafety metric level (or an acceptable change in a biosafety metric level) is one that does not constitute a Common Terminology Criteria for Adverse Events (CTCAE) grade equal to or greater than 3 for a treatment-emergent adverse event. In some embodiments, the treatment-emergent adverse event is a nervous system disorder (e.g., headache, peripheral sensory neuropathy). In some embodiments, the treatment-emergent adverse event is a gastrointestinal disorder (e.g., diarrhoea, nausea). In some embodiments, the treatment-emergent adverse event is an injury, poisoning and procedural complications (e.g., infusion related reaction, skin abrasion). In some embodiments, the treatment-emergent adverse event is an ear and labyrinth disorders (e.g., vertigo positional). In some embodiments, the treatment-emergent adverse event is an eye disorder (e.g. foreign body sensation in eyes). In some embodiments, the treatment-emergent adverse event is a general disorder or an administration site condition (e.g., catheter site swelling). In some embodiments, the treatment-emergent adverse event is an infection or infestation (e.g., acute sinusitis). In some embodiments, the treatment-emergent adverse event is a decrease in thyroxine. In some embodiments, the treatment-emergent adverse event is a respiratory, thoracic or mediastinal disorders (e.g., rhinorrhea). In some embodiments, the treatment-emergent adverse event is a skin and subcutaneous tissue disorder (e.g., pruritus, rash).

The method of in vivo editing may comprise measuring known laboratory assessments relating generally to, e.g., coagulation, hematology, clinical chemistry, urinalysis, and other bioanalytical assessments (e.g., cytokines, complement). Particular biosafety metrics include, but are not limited to one or more of the following non-limiting biosafety metrics: liver enzyme, levels of activated partial thromboplastin time (aPTT), levels of prothrombin time (PT), levels of thrombin generation time (TGT) (e.g., peak height, lag time, and/or endogenous thrombin potential), levels of fibrinogen, prothrombin international normalized (INR) ratio, level of d-dimer, vitamin A, vitamin B12, retinol binding protein (RBP), thyroid-stimulating hormone (TSH), free thyroxine, free triiodothyronine (T3), HBV, HBsAg, HCV Ab, laboratory parameters consistent with disseminated intravascular coagulation, changes in hematology values, changes in chemistry values, changes in coagulation, changes in urinalysis, levels of Glutamate Dehydrogenase, levels of C-reactive protein, levels of complement (C3, C4, C3a, C5a, Bb), levels of cytokines (GM-CSF, INF-γ, IL-1β, IL-4, IL-5, IL-6, IL-8, IL-10, IL-13, IL-23, TNF-α, IL-17, MCP-1), thyroxine (T4 levels) (e.g. a decrease in levels below normal range or clinically relevant symptoms/signs of hypothyroidism after administration of a treatment), acute liver injury (e.g. a CTCAE>Grade 2 elevations in ALT, AST, total bilirubin or GLDH or clinically relevant symptoms/signs of liver injury after administration of a treatment), and changes in a 12-Lead Electrocardiogram.

Other biosafety metrics relating to, e.g., hematology, coagulation, clinical chemistry, and urinalysis are known in the art. For example, biosafety metrics relating to hematology include, but are not limited to, platelet count, RBC count, hemoglobin, hematocrit, RBC indices (MCV, MCH, MCHC, RDW), % reticulocytes, WBC count with differential (neutrophils, lymphocytes, monocytes, eosinophils, basophils). For example, biosafety metrics relating to coagulation include, but are not limited to, aPTT, PT, INR, fibrinogen, d-dimer, and TGT. For example, biosafety metrics relating to clinical chemistry include, but are not limited to, albumin, blood urea nitrogen, creatinine, glucose non-fasting, potassium, sodium, chloride, carbon dioxide, calcium, AST, ALT, alkaline phosphatase, total and direct bilirubin, total protein, creatine kinase, lactose dehydrogenase, total cholesterol, and LDL cholesterol. For example, biosafety metrics relating to urinalysis include, but are not limited to, specific gravity, pH, glucose, protein, blood, ketones, bilirubin, urobilinogen, nitrite, and leukocyte esterase.

In some embodiments, a level of a biosafety metric is measured following administration of the LNP composition. In some embodiments, a level of a biosafety metric is measured prior to and following the administration of the LNP composition, thereby allowing for a comparison of the levels of the biosafety metric before and after treatment with the LNP composition. In some embodiments, a level of a biosafety metric measured prior to administration of the LNP composition can serve as a baseline for comparison against one or more levels of the biosafety metric measured following administration of the LNP composition. In some embodiments, a baseline is the last available measurement taken prior to administration of the LNP composition. In these embodiments, the administration of the LNP composition results in an acceptable change in liver enzyme levels (e.g., no more than an elevation in ALT or AST>5×ULN for more than 4 weeks after administration of a treatment, ALT or AST>3×ULN and total bilirubin>2×ULN (Hy's law) after administration of a treatment). In these embodiments, the administration of the composition results in an acceptable change in levels of activated partial thromboplastin time (aPTT) (e.g., an elevation in aPTT)>5×ULN for more than 4 weeks after administration of a treatment). In these embodiments, the administration of the composition results in an acceptable change in levels of prothrombin time (PT). In these embodiments, the administration of the composition results in an acceptable change in levels of thrombin generation time (TGT) (e.g., peak height, lag time, and/or endogenous thrombin potential). In these embodiments, the administration of the composition results in an acceptable change in levels of fibrinogen. In some embodiments, the administration of the composition results in an acceptable change in the prothrombin international normalized (INR) ratio. In these embodiments, the administration of the composition results in an acceptable change in level of d-dimer. In these embodiments, the administration of the composition results in an acceptable change in laboratory parameters consistent with disseminated intravascular coagulation. In these embodiments, the administration of the composition results in an acceptable change in hematology values (e.g. a CTCAE>Grade 2 abnormal blood test results after administration of a treatment). In these embodiments, the administration of the composition results in an acceptable change in chemistry values. In these embodiments, the administration of the composition results in an acceptable change in abnormal coagulation findings defined by clinically relevant abnormal bleeding. In these embodiments, the administration of the composition results in an acceptable change in urinalysis. In these embodiments, the administration of the composition results in an acceptable change in levels of glutamate dehydrogenase. In these embodiments, the administration of the composition results in an acceptable change in levels of C-reactive protein. In these embodiments, the administration of the composition results in an acceptable change in levels of complement. In these embodiments, the administration of the composition results in an acceptable change in levels of cytokines.

In these embodiments, the administration of the composition results in an acceptable change in a biosafety metric level that does not constitute a treatment-emergent adverse event of Grade 3 or higher according to CTCAE guidelines. In these embodiments, the administration of the composition results in an acceptable change in a biosafety metric level that does not constitute an incidence of thrombosis. In these embodiments, the administration of the composition results in an acceptable change in a biosafety metric level that does not constitute an incidence of hemorrhage. In these embodiments, the administration of the composition results in an acceptable change in a biosafety metric level that does not constitute an incidence of disseminated intravascular coagulation. In these embodiments, the administration of the composition results in an acceptable change in a biosafety metric level that does not constitute an incidence of cytokine release syndrome. In these embodiments, the administration of the composition results in an acceptable change in a biosafety metric level that does not constitute an incidence attributed to impacts on the spleen (splenic hemorrhage, splenic infarction, sometimes thrombocytopenia, sometimes anemia or lymphopenia with specific abnormal findings on study of the blood cells on microscopy). In these embodiments, the administration of the composition results in an acceptable change in a biosafety metric level that does not constitute in an incidence attributed to impacts on the adrenal glands. In these embodiments, the administration of the composition results in an acceptable change in a biosafety metric level that does not constitute an ophthalmic incidence consistent with Vitamin A deficiency. In these embodiments, the administration of the composition results in an acceptable change in levels of thyroxine (T4 levels) (e.g. does not a decrease in levels below normal range nor constitute clinically relevant symptoms/signs of hypothyroidism after administration of a treatment). In these embodiments, the administration of the composition results in an acceptable change in a biosafety metric level that does not constitute an acute liver injury (e.g. a CTCAE>Grade 2 elevations in ALT, AST, total bilirubin or GLDH or clinically relevant symptoms/signs of liver injury after administration of a treatment). In these embodiments, the administration of the composition results in an acceptable change in a 12-Lead Electrocardiogram as determined by a clinician.

In some embodiments, the method of in vivo editing of the gene comprises systemically administering to the human subject the LNP composition comprising an effective amount of an mRNA encoding Cas9, and the administration results in an acceptable change in levels of anti-Cas antibodies (e.g., anti-Cas9 antibodies).

In some embodiments, the administration of the LNP composition results in an acceptable change in the pharmacokinetics of Lipid A. In some embodiments, the administration of the LNP composition results in an acceptable change in the pharmacokinetics of DMG-PEG2k. In some embodiments, the administration of the LNP composition results in an acceptable change in the pharmacokinetics of Cas9 mRNA. In some embodiments, the administration of the LNP composition results in an acceptable change in the pharmacokinetics of sgRNA.

### 2. Methods of Treatment

Methods of treating a human subject by in vivo editing of a gene in the liver are provided herein. In some embodiments, the present method treats a monogenic disorder that results from an aberrant expression or activity of a liver gene product. The monogenic disorder can be treated with an edit (e.g., a single edit) to the gene in the liver, or to a non-coding region that causes an aberrant expression or activity of a liver gene product. In some embodiments, the gene product is a protein. In some embodiments, the gene product is an RNA molecule. In some embodiments, an edit to the gene in the liver, or to a non-coding region that causes an aberrant expression or activity of a liver gene product, reduces the level (e.g., serum level) of the gene product. In some embodiments, the present methods target and edit the TTR gene in the liver (e.g., in the hepatocyte). In some embodiments, the monogenic disorder is ATTR.

In some embodiments, the method of in vivo editing of the gene comprises systemically administering to the human subject a LNP composition described herein (e.g., comprising an effective amount of an mRNA encoding a Cas nuclease, e.g., Cas9; and a guide RNA that targets a gene, e.g., a guide RNA that targets the TTR gene) and determining a level of a biosafety metric. In some embodiments, the method of treatment comprises in vivo editing of the gene that occurs at the site targeted by the guide RNA in a hepatocyte of the subject.

In some embodiments, a method for treating a human subject having a monogenic disorder comprises systemically administering to the human subject a LNP composition described herein (e.g., comprising an effective amount of an mRNA encoding a Cas nuclease, e.g., Cas9; and a guide RNA that targets a gene, e.g., a guide RNA that targets the TTR gene), determining a first level of a biosafety metric in the subject prior to administration; determining a second level of the biosafety metric in the subject a period of time after administration; and assessing the change between the first and the second levels of the biosafety metric.

In some embodiments, the method of treatment comprises administering a guide RNA that targets a gene in a hepatocyte in the subject.

In some embodiments, the method for treating a human subject having a monogenic disorder comprises systemically administering to the human subject a LNP composition described herein (e.g., comprising an effective amount of an mRNA encoding a Cas nuclease, e.g., Cas9; and a guide RNA that targets a gene, e.g., a guide RNA that targets the TTR gene) to edit the gene in the liver. In some embodiments, the method for treating a human subject having a monogenic disorder comprises systemically administering to the human subject a LNP composition described herein (e.g., comprising an effective amount of an mRNA encoding a Cas nuclease, e.g., Cas9; and a guide RNA that targets a gene, e.g., a guide RNA that targets the TTR gene) to knockdown production of the aberrant liver gene product. In some embodiments, the method of treatment knocks down production of the liver gene product in a population of cells. In some embodiments, the method of treatment results in knockdown of the liver gene product long-term (e.g., a durable knockdown) after a single edit in the liver. In some embodiments, the method of treatment comprises administering the LNP composition described herein more than once to maximize editing, e.g., via cumulative effects, e.g., 1, 2, 3, 4, or 5 times. In some embodiments, the method of treatment comprises administering the LNP composition described herein more than once (e.g., twice) to achieve an effective reduction (e.g., achieve at least 60% reduction in the level of a liver gene product relative to a baseline level).

The method of treatment comprises administering the LNP composition described herein and further determining a level of a biosafety metric. In these embodiments, administration of the LNP composition to the subject may be associated with a change in a biosafety metric. In some embodiments, the subject is assessed to determine whether the change in the biosafety metric is an acceptable change. In some embodiments, an acceptable change can be determined by a clinician and/or laboratory. In some embodiments, an acceptable change can be one that does not qualify as a safety event, including an adverse event (NCI-CTCAE Grade not greater than or equal to 3), a serious adverse event, an adverse event of special interest, and/or a treatment-emergent adverse event (CTCAE Grade not greater than or equal to 3), as described herein. Biosafety metrics, including those associated with administration of an LNP composition, are known in the art. Acceptable levels and/or changes in the biosafety metrics are known in the art and may be assessed by routine methods.

In some embodiments, an acceptable biosafety metric level is one that falls within the subject inclusion criteria and/or does not fall within the subject exclusion criteria described herein.

In some embodiments, an acceptable change in a biosafety metric level is a change that is acceptable after a period of time, e.g., initially falls outside of acceptable levels but stabilizes to an acceptable level by, e.g., day 2, 3, 4, 5, 6, 7, 14 or 28 after administration. In some embodiments, an acceptable change in a biosafety metric level is a change in a level that falls within 150% of the upper limit of normal for said biosafety metric and/or within 50% of the lower limit of normal for said biosafety metric, e.g., within 150% of the prothrombin ULN and/or within 50% of the LLN of fibrinogen.

In some embodiments, an acceptable biosafety metric level (or an acceptable change in a biosafety metric level) is one that does not constitute an adverse event of Grade 3 or higher according to CTCAE guidelines, including National Cancer Institute (NCI)-CTCAE guidelines, version 5.0. In some embodiments, a change in a biosafety metric level (e.g., one or more levels associated with a laboratory parameter, vital sign, ECG data, physical exam, etc., as described herein) constitutes as an adverse event if the change, e.g., induces clinical signs or symptoms; requires active intervention; requires interruption or discontinuation of the LNP composition; and/or the change in the biosafety metric is clinically significant, as determined by a clinician.

In some embodiments, an adverse event is any untoward medical occurrence in a subject administered a study drug or has undergone study procedures and which does not necessarily have a causal relationship with the treatment. In some embodiments, an adverse event is an unintended sign (including an abnormal laboratory finding), symptom, or disease temporally associated with the treatment, whether or not related to the medicinal (investigational) product. In some embodiments, an adverse event induces clinical signs or symptoms. In some embodiments, an adverse event requires active intervention. In some embodiments, an adverse event requires interruption or discontinuation of the treatment. In some embodiments, an adverse event is an abnormality that is clinically significant in the opinion of the investigator. Grading criteria for adverse events are known in the art, such as, e.g., Common Terminology Criteria for Adverse Events (CTCAE), including National Cancer Institute (NCI)-CTCAE.

In some embodiments, an acceptable biosafety metric level (or an acceptable change in a biosafety metric level) is one that does not constitute a serious adverse event. In some embodiments, a serious adverse event results in death. In some embodiments, a serious adverse event is life threatening (e.g., places the subject at immediate risk of death as determined by a clinician). In some embodiments, a serious adverse event results in persistent or significant disability. In some embodiments, a serious adverse event results in incapacity or substantial disruption of the ability to conduct normal life functions. In some embodiments, a serious adverse event results in congenital anomaly or birth defect. In some embodiments, a serious adverse event requires inpatient hospitalization or leads to prolongation of hospitalization.

In some embodiments, an acceptable biosafety metric level (or an acceptable change in a biosafety metric level) is one that does not constitute an adverse event of special interest. In some embodiments, an adverse event of special interest includes, e.g., infusion-related reaction (IRR) (e.g., requiring treatment or discontinuation of infusion, and/or Grade 3 or higher), incidence of thrombosis, incidence of hemorrhage, CTCAE≥Grade 2 abnormal blood test results, CTCAE≥Grade 2 elevation in ALT, CTCAE≥Grade 2 elevation in AST, CTCAE≥Grade 2 elevation in total bilirubin, CTCAE≥Grade 2 elevation in GLDH, incidence of cytokine release syndrome, an event attributed to impacts on the spleen (splenic hemorrhage, splenic infarction, sometimes thrombocytopenia, sometimes anemia or lymphopenia with specific abnormal findings on study of the blood cells on microscopy), an event attributed to impacts on the adrenal glands, clinically relevant symptoms or hypothyroidism, and an ophthalmic event consistent with Vitamin A deficiency.

In some embodiments, an acceptable biosafety metric level (or an acceptable change in a biosafety metric level) is one that does not constitute a Common Terminology Criteria for Adverse Events (CTCAE) grade equal to or greater than 3 for a treatment-emergent adverse event. In some embodiments, the treatment-emergent adverse event is a nervous system disorder (e.g., headache, peripheral sensory neuropathy). In some embodiments, the treatment-emergent adverse event is a gastrointestinal disorder (e.g., diarrhoea, nausea). In some embodiments, the treatment-emergent adverse event is an injury, poisoning and procedural complications (e.g., infusion related reaction, skin abrasion). In some embodiments, the treatment-emergent adverse event is an ear and labyrinth disorders (e.g., vertigo positional). In some embodiments, the treatment-emergent adverse event is an eye disorder (e.g. foreign body sensation in eyes). In some embodiments, the treatment-emergent adverse event is a general disorder or an administration site condition (e.g., catheter site swelling). In some embodiments, the treatment-emergent adverse event is an infection or infestation (e.g., acute sinusitis). In some embodiments, the treatment-emergent adverse event is a decrease in thyroxine. In some embodiments, the treatment-emergent adverse event is a respiratory, thoracic or mediastinal disorders (e.g., rhinorrhoea). In some embodiments, the treatment-emergent adverse event is a skin and subcutaneous tissue disorder (e.g., pruritus, rash).

The method of in vivo editing may comprise measuring known laboratory assessments relating generally to, e.g., coagulation, hematology, clinical chemistry, urinalysis, and other bioanalytical assessments (e.g., cytokines, complement). Particular biosafety metrics include, but are not limited to one or more of the following non-limiting biosafety metrics: liver enzyme, levels of activated partial thromboplastin time (aPTT), levels of prothrombin time (PT), levels of thrombin generation time (TGT) (e.g., peak height, lag time, and/or endogenous thrombin potential), levels of fibrinogen, prothrombin international normalized (INR) ratio, level of d-dimer, vitamin A, vitamin B12, retinol binding protein (RBP), thyroid-stimulating hormone (TSH), free thyroxine, free triiodothyronine (T3), HBV, HBsAg, HCV Ab, laboratory parameters consistent with disseminated intravascular coagulation, changes in hematology values, changes in chemistry values, changes in coagulation, changes in urinalysis, levels of Glutamate Dehydrogenase, levels of C-reactive protein, levels of complement (C3, C4, C3a, C5a, Bb), levels of cytokines (GM-CSF, INF-g, IL-1b, IL-4, IL-5, IL-6, IL-8, IL-10, IL-13, IL-23, TNF-α, IL-17, MCP-1), thyroxine (T4 levels) (e.g. a decrease in levels below normal range or clinically relevant symptoms/signs of hypothyroidism after administration of a treatment), acute liver injury (e.g. a CTCAE>Grade 2 elevations in ALT, AST, total bilirubin or GLDH or clinically relevant symptoms/signs of liver injury after administration of a treatment), and changes in a 12-Lead Electrocardiogram.

Other biosafety metrics relating to, e.g., hematology, coagulation, clinical chemistry, and urinalysis are known in the art. For example, biosafety metrics relating to hematology include, but are not limited to, platelet count, RBC count, hemoglobin, hematocrit, RBC indices (MCV, MCH, MCHC, RDW), % reticulocytes, WBC count with differential (neutrophils, lymphocytes, monocytes, eosinophils, basophils). For example, biosafety metrics relating to coagulation include, but are not limited to, aPTT, PT, INR, fibrinogen, d-dimer, and TGT. For example, biosafety metrics relating to clinical chemistry include, but are not limited to, albumin, blood urea nitrogen, creatinine, glucose non-fasting, potassium, sodium, chloride, carbon dioxide, calcium, AST, ALT, alkaline phosphatase, total and direct bilirubin, total protein, creatine kinase, lactose dehydrogenase, total cholesterol, and LDL cholesterol. For example, biosafety metrics relating to urinalysis include, but are not limited to, specific gravity, pH, glucose, protein, blood, ketones, bilirubin, urobilinogen, nitrite, and leukocyte esterase.

In some embodiments, a level of a biosafety metric is measured following administration of the LNP composition. In some embodiments, a level of a biosafety metric is measured prior to and following the administration of the LNP composition, thereby allowing for a comparison of the levels of the biosafety metric before and after treatment with the LNP composition. In some embodiments, a level of a biosafety metric measured prior to administration of the LNP composition can serve as a baseline for comparison against one or more levels of the biosafety metric measured following administration of the LNP composition. In some embodiments, a baseline is the last available measurement taken prior to administration of the LNP composition. In these embodiments, the administration of the LNP composition results in an acceptable change in liver enzyme levels (e.g., no more than an elevation in ALT or AST>5×ULN for more than 4 weeks after administration of a treatment, ALT or AST>3×ULN and total bilirubin>2×ULN (Hy's law) after administration of a treatment). In these embodiments, the administration of the composition results in an acceptable change in levels of activated partial thromboplastin time (aPTT) (e.g., an elevation in aPTT)>5×ULN for more than 4 weeks after administration of a treatment). In these embodiments, the administration of the composition results in an acceptable change in levels of prothrombin time (PT). In these embodiments, the administration of the composition results in an acceptable change in levels of thrombin generation time (TGT) (e.g., peak height, lag time, and/or endogenous thrombin potential). In these embodiments, the administration of the composition results in an acceptable change in levels of fibrinogen. In these embodiments, the administration of the composition results in an acceptable change in the prothrombin international normalized (INR) ratio. In these embodiments, the administration of the composition results in an acceptable change in level of d-dimer. In these embodiments, the administration of the composition results in an acceptable change in laboratory parameters consistent with disseminated intravascular coagulation. In these embodiments, the administration of the composition results in an acceptable change in hematology values (e.g. a CTCAE>Grade 2 abnormal blood test results after administration of a treatment). In these embodiments, the administration of the composition results in an acceptable change in chemistry values. In these embodiments, the administration of the composition results in an acceptable change in abnormal coagulation findings defined by clinically relevant abnormal bleeding. In these embodiments, the administration of the composition results in an acceptable change in urinalysis. In these embodiments, the administration of the composition results in an acceptable change in levels of glutamate dehydrogenase. In these embodiments, the administration of the composition results in an acceptable change in levels of C-reactive protein. In these embodiments, the administration of the composition results in an acceptable change in levels of complement. In these embodiments, the administration of the composition results in an acceptable change in levels of cytokines.

In these embodiments, the administration of the composition results in an acceptable change in a biosafety metric level that does not constitute a treatment-emergent adverse event of Grade 3 or higher according to CTCAE guidelines. In these embodiments, the administration of the composition results in an acceptable change in a biosafety metric level that does not constitute an incidence of thrombosis. In these embodiments, the administration of the composition results in an acceptable change in a biosafety metric level that does not constitute an incidence of hemorrhage. In these embodiments, the administration of the composition results in an acceptable change in a biosafety metric level that does not constitute an incidence of disseminated intravascular coagulation. In these embodiments, the administration of the composition results in an acceptable change in a biosafety metric level that does not constitute an incidence of cytokine release syndrome. In these embodiments, the administration of the composition results in an acceptable change in a biosafety metric level that does not constitute an incidence attributed to impacts on the spleen (splenic hemorrhage, splenic infarction, sometimes thrombocytopenia, sometimes anemia or lymphopenia with specific abnormal findings on study of the blood cells on microscopy). In these embodiments, the administration of the composition results in an acceptable change in a biosafety metric level that does not constitute in an incidence attributed to impacts on the adrenal glands. In these embodiments, the administration of the composition results in an acceptable change in a biosafety metric level that does not constitute an ophthalmic incidence consistent with Vitamin A deficiency. In these embodiments, the administration of the composition results in an acceptable change in levels of thyroxine (T4 levels) (e.g. does not a decrease in levels below normal range nor constitute clinically relevant symptoms/signs of hypothyroidism after administration of a treatment). In these embodiments, the administration of the composition results in an acceptable change in a biosafety metric level that does not constitute an acute liver injury (e.g. a CTCAE>Grade 2 elevations in ALT, AST, total bilirubin or GLDH or clinically relevant symptoms/signs of liver injury after administration of a treatment). In these embodiments, the administration of the composition results in an acceptable change in a 12-Lead Electrocardiogram as determined by a clinician.

Methods of treating a human subject by in vivo editing of a TTR gene in the liver are provided herein. In some embodiments, the method of in vivo editing of the TTR gene comprises systemically administering to the human subject a LNP composition described herein (e.g., comprising an effective amount of an mRNA encoding a Cas nuclease, e.g., Cas9; and a guide RNA that targets the TTR gene) and determining a level of a biosafety metric as described above. In some embodiments, in vivo editing the TTR gene occurs at the site targeted by the guide RNA in a hepatocyte of the subject.

In some embodiments, provided herein are methods for treating a human subject suffering from amyloidosis associated with TTR (ATTR), as described herein. In some embodiments, the methods are for treating a human subject suffering from hereditary ATTR (ATTRv). In some embodiments, the methods are for treating a human subject suffering from non-hereditary (wildtype) ATTR (ATTRwt). In some embodiments, the methods are for treating a human subject suffering from ATTRv-PN. In some embodiments, the methods are for treating a human subject suffering from familial amyloid cardiomyopathy (FAC, also known as ATTRv-CM). In some embodiments, the methods are for treating a human subject suffering from wildtype ATTR (ATTRwt-CM). In some embodiments, the methods are for treating a human subject suffering from ATTR-CM, NYHA Class I, Class II, or Class III.

In some embodiments, provided herein is a method for treating amyloidosis associated with TTR (ATTR) in a human subject, comprising systemically administering to the human subject a LNP composition described herein (e.g., comprising an effective amount of an mRNA encoding a Cas nuclease, e.g., Cas9; and a guide RNA that targets a gene, e.g., a guide RNA that targets the TTR gene), thereby treating ATTR, wherein the administration of the composition results in a clinically significant improvement in a level of a clinical metric in the subject as compared to a baseline level of the clinical metric.

In some embodiments, provided herein is a method for treating amyloidosis associated with TTR (ATTR) in a human subject, comprising systemically administering to the human subject a LNP composition described herein (e.g., comprising an effective amount of an mRNA encoding a Cas nuclease, e.g., Cas9; and a guide RNA that targets a gene, e.g., a guide RNA that targets the TTR gene), thereby treating ATTR, wherein the mRNA encoding a Cas nuclease and the guide RNA that targets the TTR gene are administered at a combined dose of about 25 to about 100 mg.

In some embodiments, provided herein is a method for treating amyloidosis associated with TTR (ATTR) in a human subject, comprising systemically administering to the human subject a LNP composition described herein (e.g., comprising an effective amount of an mRNA encoding a Cas nuclease, e.g., Cas9; and a guide RNA that targets a gene, e.g., a guide RNA that targets the TTR gene), thereby treating ATTR, wherein the administration of the composition reduces serum TTR relative to baseline serum. In some embodiments, the ATTR is hereditary transthyretin amyloidosis. In some embodiments, the ATTR is wild-type transthyretin amyloidosis. In some embodiments, the ATTR is hereditary transthyretin amyloidosis with polyneuropathy. In some embodiments, the ATTR is hereditary transthyretin amyloidosis with cardiomyopathy. In embodiments where the ATTR is wildtype transthyretin amyloidosis with cardiomyopathy, the subject is classified under the New York Health Association (NYHA) classification as Class I, Class II, or Class III. In some embodiments, the ATTR is ATTRv-PN and/or ATTR-CM. In some embodiments, the LNP comprises (9Z, 12Z)-3-((4,4-bis(octyloxy)butanoyl)oxy)-2-((((3-(diethylamino)propoxy)carbonyl)oxy)methyl)propyl octadeca-9, 12-dienoate. In some embodiments, the LNP comprises a PEG lipid. In embodiments where the LNP comprises a PEG lipid, the PEG lipid comprises dimyristoylglycerol (DMG). In embodiments where the PEG lipid comprises dimyristoylglycerol (DMG), the PEG lipid comprises PEG-2k. In some embodiments, the LNP composition has an N/P ratio about 5-7. In some embodiments, the guide RNA and Cas nuclease are present in a ratio ranging from about 5:1 to about 1:5 by weight. In some embodiments, the mRNA encodes a Class 2 Cas nuclease. In some embodiments, the mRNA encodes a Cas9 nuclease. In some embodiments, the mRNA encodes S. pyogenes Cas9. In some embodiments, the Cas nuclease is codon-optimized. In some embodiments, the guide RNA comprises at least one modification. In embodiments where the guide RNA comprises at least one modification, the guide RNA includes a 2′-O-methyl modified nucleotide or a phosphorothioate bond between nucleotides. In some embodiments, the effective amount of mRNA encoding a Cas nuclease and the guide RNA that targets the TTR gene is a combined dose of about 0.3 mg/kg to about 2 mg/kg. In some embodiments, the effective amount of mRNA encoding a Cas nuclease and the guide RNA that targets the TTR gene is a combined dose of about 0.3 mg/kg to about 1 mg/kg. In some embodiments, the effective amount of mRNA encoding a Cas nuclease and the guide RNA that targets the TTR gene is a combined dose of about 0.3 mg/kg. In some embodiments, the effective amount of mRNA encoding a Cas nuclease and the guide RNA that targets the TTR gene is a combined dose of about 0.7 mg/kg. In some embodiments, the effective amount of mRNA encoding a Cas nuclease and the guide RNA that targets the TTR gene is a combined dose of about 1.0 mg/kg. In some embodiments, effective amount of the mRNA encoding a Cas nuclease and the guide RNA that targets the TTR gene is a combined dose of about 25 mg to about 150 mg of total RNA. In some embodiments, the effective amount of mRNA encoding a Cas nuclease and the guide RNA that targets the TTR gene is a combined dose of about 25 mg to about 100 mg of total RNA. In some embodiments, the effective amount of mRNA encoding a Cas nuclease and the guide RNA that targets the TTR gene is a combined dose of about 50 mg to about 90 mg of total RNA. In some embodiments, the effective amount of mRNA encoding a Cas nuclease and the guide RNA that targets the TTR gene is a combined dose of about 40 mg of total RNA. In some embodiments, the effective amount of mRNA encoding a Cas nuclease and the guide RNA that targets the TTR gene is a combined dose of about 50 mg of total RNA. In some embodiments, the effective amount of mRNA encoding a Cas nuclease and the guide RNA that targets the TTR gene is a combined dose of about 60 mg of total RNA. In some embodiments, the effective amount of mRNA encoding a Cas nuclease and the guide RNA that targets the TTR gene is a combined dose of about 70 mg of total RNA. In some embodiments, the effective amount of mRNA encoding a Cas nuclease and the guide RNA that targets the TTR gene is a combined dose of about 80 mg of total RNA. In some embodiments, the effective amount of mRNA encoding a Cas nuclease and the guide RNA that targets the TTR gene is a combined dose of about 90 mg of total RNA. In some embodiments, the effective amount of mRNA encoding a Cas nuclease and the guide RNA that targets the TTR gene is a combined dose of about 100 mg of total RNA. In some embodiments, administration of the composition reduces serum TTR by 60-70%, 70-80%, 80-90%, 90-95%, 95-98%, 98-99%, or 99-100% as compared to baseline serum TTR before administration of the composition. In some embodiments, the serum TTR levels are less than about 50 μg/mL after administration of the composition. In some embodiments, the serum TTR levels are less than about 40 μg/mL after administration of the composition. In some embodiments, the serum TTR levels are less than about 30 μg/mL after administration of the composition. In some embodiments, the serum TTR levels are less than about 20 μg/mL after administration of the composition. In some embodiments, the serum TTR levels are less than about 10 μg/mL after administration of the composition. In some embodiments, the method further comprises administering a second dose of the LNP composition, wherein administration of the second dose reduces serum TTR levels by at least 80% relative to the baseline serum TTR level prior to administration of the first dose. In some embodiments, the method further comprises administering a second dose of the LNP composition, wherein administration of the second dose reduces serum TTR levels by at least 80% relative to the baseline serum TTR level prior to administration of the second dose and after administration of the first dose. In some embodiments, the composition is administered with a second therapeutic. In some embodiments, the second therapeutic is diflunisal or tafamidis.

In some embodiments, the human subject has been diagnosed with ATTR prior to treatment or is diagnosed with ATTR concurrently with treatment. In some embodiments, the human subject is diagnosed with ATTR based on genetic testing (e.g., a documented TTR mutation). In some embodiments, the human subject is diagnosed with ATTR based on a clinical diagnosis of sensorimotor peripheral neuropathy. In some embodiments, the human subject is diagnosed with ATTR based on a Neuropathy Impairment Score (NIS)≥5 and ≤130 prior to treatment. In some embodiments, the human subject is diagnosed with ATTR based on a documented tissue deposition of TTR amyloid by biopsy or by validated noninvasive imaging. In some embodiments, a human subject is diagnosed with ATTR based on a Polyneuropathy Disability (PND) score≤3b.

In some embodiments, the human subject has progression of ATTRv-PN symptoms prior to treatment. In some embodiments, the human subject has an increase in Polyneuropathy Disability (PND) score≥1 point. In some embodiments, the human subject has an increase Familial Amyloid Polyneuropathy (FAP) stage≥1 point. In some embodiments, the human subject has an increase in Neuropathy Impairment Score (NIS)≥5 points. In some embodiments, the human subject has an increase in NIS-Lower Limb (LL)≥ 5 points. In some embodiments, the human subject has a decrease in Modified Body-Mass Index (mBMI)≥25 kg/m2×g/L. In some embodiments, the human subject has a decrease in 6-minute walk test≥30 meters. In some embodiments, the human subject has a decrease in 10-meter walk test≥0.1 m/s.

In some embodiments, provided herein are methods for treating a subject who has progression of ATTR while receiving a TTR-reducing therapy. In some embodiments, the method of treatment comprises systemically administering to the human subject a LNP composition described herein (e.g., comprising an effective amount of an mRNA encoding a Cas nuclease, e.g., Cas9; and a guide RNA that targets a gene, e.g., a guide RNA that targets the TTR gene), wherein the subject has been or is currently being treated by a different ATTR therapy. In some embodiments, the subject has progression of ATTR while on the different ATTR therapy, e.g., as measured by a clinical efficacy metric such as mNIS+7 score. In some embodiments, the subject to be administered the LNP composition described herein has been or is currently being treated with inotersen and exhibits progression of ATTR. In some embodiments, the subject to be administered the LNP composition described herein has been or is currently being treated with patisiran and has progression of ATTR. In some embodiments, the subject to be administered the LNP composition described herein has been or is currently being treated with diflunisal and has progression of ATTR. In some embodiments, the subject to be administered the LNP composition described herein has been or is currently being treated with tafamidis and has progression of ATTR.

In some embodiments, the method of in vivo editing of the TTR gene comprises systemically administering to the human subject a LNP composition described herein (e.g., comprising an effective amount of an mRNA encoding a Cas nuclease, e.g., Cas9; and a guide RNA that targets the TTR gene) and results in a clinically significant improvement in a level of a clinical metric.

In some embodiments, a method of treating amyloidosis associated with TTR comprises administering the LNP composition described herein and further determining one or more clinical efficacy metrics, which include, but are not limited to: a decrease in serum TTR (e.g. a 60% decrease of serum TTR as measured by ELISA after administration of a treatment), a decrease in serum TTR (e.g. a 60% decrease of serum TTR as measured by mass spectrometry after administration of a treatment), a decrease in serum prealbumin, a reduction in Polyneuropathy Disability (PND) Score, a reduction in Familial Amyloid Polyneuropathy (FAP) stage, a decrease in Neuropathy Impairment Score (NIS), a decrease in Modified Neurological Impairment Score (mNIS+7), a decrease in Neuropathy Impairment Score (NIS)-Lower Limb (LL), an increase in Modified Body Mass Index (mBMI)≥25 kg/m2× g/L, an increase in 6-minute walk test (6-MWT)≥30 meters, and increase 10-Meter Walk Test (10-MWT)≥0.1 meters/second. Additional clinical efficacy metrics include an improvement in serum Neurofilament Light Chain (NfL) levels, an improvement in quality of life as assessed by Norfolk Quality of Life-Diabetic Neuropathy, an improvement in quality of life as assessed by EuroQOL (EQ)-5D-5L, an improvement in cardiac MRI (e.g., a decrease in extracellular volume), an improvement in N-terminal prohormone of brain natriuretic peptide (NT-proBNP) levels; an improvement in Troponin I levels, an improvement in New York Health Association (NYHA) classification, and an improvement as scored by the Kansas City Cardiomyopathy Questionnaire (KCCQ). Additional clinical efficacy metrics, including metrics for assessing efficacy for TTR amyloidosis, are known in the art. Similarly, levels and/or changes in clinical efficacy metrics indicative of amelioration of disease, including TTR amyloidosis, are known in the art and may be assessed by routine methods, e.g., by a clinician or laboratory.

In some embodiments, a method of treating amyloidosis associated with TTR comprises administering the LNP composition described herein and measuring a clinical efficacy metric following administration of the LNP composition. In some embodiments, a method of treating amyloidosis associated with TTR comprises administering the LNP composition described herein and measuring a clinical efficacy metric prior to and following the administration of the LNP composition, thereby allowing for a comparison of the levels of the clinical efficacy metric before and after treatment with the LNP composition.

For example, serum TTR level is a clinical efficacy metric for TTR amyloidosis. In some embodiments, the method of treating amyloidosis associated with TTR comprises administering the LNP composition described herein and reducing TTR level, e.g., serum TTR level in the subject. In some embodiments, the method of treating amyloidosis associated with TTR comprises administering the LNP composition described herein and reducing TTR level, e.g., serum TTR level in the subject by at least 60%, 65%, 70%, 75%, 80%, 85%, 90%, 95%, or more after treatment (e.g., 14 days or 28 days after administration of the LNP composition) as compared to baseline, e.g., prior to treatment. In some embodiments, the method of treating amyloidosis associated with TTR described herein yields at least 60% reduction in TTR level, e.g., serum TTR level, after treatment (e.g., 14 days or 28 days after administration of the LNP composition) as compared to baseline. In some embodiments, the method of treating amyloidosis associated with TTR described herein yields at least 70% reduction in TTR level, e.g., serum TTR level, after treatment (e.g., 14 days or 28 days after administration of the LNP composition) as compared to baseline. In some embodiments, the method of treating amyloidosis associated with TTR described herein yields at least 80% reduction in TTR level, e.g., serum TTR level, after treatment (e.g., 14 days or 28 days after administration of the LNP composition) as compared to baseline. In some embodiments, the method of treating amyloidosis associated with TTR described herein yields at least 85% reduction in TTR level, e.g., serum TTR level, after treatment (e.g., 14 days or 28 days after administration of the LNP composition) as compared to baseline. In some embodiments, the method of treating amyloidosis associated with TTR described herein yields at least 90% reduction in TTR level, e.g., serum TTR level, after treatment (e.g., 14 days or 28 days after administration of the LNP composition) as compared to baseline. In some embodiments, the method of treating amyloidosis associated with TTR described herein yields at least 95% reduction in TTR level, e.g., serum TTR level, after treatment (e.g., 14 days or 28 days after administration of the LNP composition) as compared to baseline. In some embodiments, the method of treating amyloidosis associated with TTR comprises administering the LNP composition described herein and reducing TTR level, e.g., serum TTR level in the subject by at least 80%, 81%, 82%, 83%, 84%, 85%, 86%, 87%, 88%, 89%, 90%, 91%, 92%, 93%, 94%, 95%, 96%, 97%, 98%, or 99% after treatment (e.g., 14 days or 28 days after administration of the LNP composition) as compared to baseline.

Methods of measuring serum levels of TTR are known in the art, e.g., ELISA.

For example, serum prealbumin level is a clinical efficacy metric for TTR amyloidosis. In some embodiments, the method of treating amyloidosis associated with TTR comprises administering the LNP composition described herein and reducing TTR level, e.g., serum prealbumin level in the subject. In some embodiments, the method of treating amyloidosis associated with TTR comprises administering the LNP composition described herein and reducing TTR level, e.g., serum prealbumin level in the subject by at least 60%, 65%, 70%, 75%, 80%, 85%, 90%, 95%, or more after treatment (e.g., 14 days or 28 days after administration of the LNP composition) as compared to baseline, e.g., prior to treatment. In some embodiments, the method of treating amyloidosis associated with TTR described herein yields at least 60% reduction in TTR level, e.g., serum prealbumin level, after treatment (e.g., 14 days or 28 days after administration of the LNP composition) as compared to baseline. In some embodiments, the method of treating amyloidosis associated with TTR described herein yields at least 70% reduction in TTR level, e.g., serum prealbumin level, after treatment (e.g., 14 days or 28 days after administration of the LNP composition) as compared to baseline. In some embodiments, the method of treating amyloidosis associated with TTR described herein yields at least 80% reduction in TTR level, e.g., serum prealbumin level, after treatment (e.g., 14 days or 28 days after administration of the LNP composition) as compared to baseline. In some embodiments, the method of treating amyloidosis associated with TTR described herein yields at least 85% reduction in TTR level, e.g., serum prealbumin level, after treatment (e.g., 14 days or 28 days after administration of the LNP composition) as compared to baseline. In some embodiments, the method of treating amyloidosis associated with TTR described herein yields at least 90% reduction in TTR level, e.g., serum prealbumin level, after treatment (e.g., 14 days or 28 days after administration of the LNP composition) as compared to baseline. In some embodiments, the method of treating amyloidosis associated with TTR described herein yields at least 95% reduction in TTR level, e.g., serum prealbumin level, after treatment (e.g., 14 days or 28 days after administration of the LNP composition) as compared to baseline. Methods of measuring serum levels of prealbumin are known in the art, e.g., ELISA.

In other embodiments, administration of the LNP composition reduces serum TTR levels in a subject to less than about 50 μg/mL. In some embodiments, administration of the LNP composition reduces serum TTR levels to less than about 40 μg/mL. In some embodiments, administration of the LNP composition reduces serum TTR levels to less than about 30 μg/mL. In some embodiments, administration of the LNP composition reduces serum TTR levels to less than about 20 μg/mL. In some embodiments, administration of the LNP composition reduces serum TTR levels to less than about 10 μg/mL.

In some embodiments, the treatment results in a decrease in serum prealbumin as compared to baseline levels. In some embodiments, the treatment results in a decrease from baseline of at least 1 point in Polyneuropathy Disability (PND) Score. In some embodiments, the treatment results in a decrease from baseline of at least 1 point in Familial Amyloid Polyneuropathy (FAP) stage. In some embodiments, the treatment results in a decrease of at least 1 point in Neuropathy Impairment Score (NIS) as compared to baseline. In some embodiments, the treatment results in a decrease in Modified Neurological Impairment Score (mNIS+7) as compared to baseline. In some embodiments, the treatment results in a decrease in Neuropathy Impairment Score (NIS)-Lower Limb (LL) as compared to baseline. In some embodiments, the treatment results in an increase in Modified Body Mass Index (e.g., (mBMI)≥25 kg/m2×g/L) as compared to baseline. In some embodiments, the treatment results in an increase in 6-minute walk test (6-MWT)≥30 meters as compared to baseline. In some embodiments, the treatment results in an increase 10-Meter Walk Test (10-MWT)≥0.1 meters/second as compared to baseline. In some embodiments, the treatment results in an improvement in serum Neurofilament Light Chain (NfL) levels as compared to baseline. In some embodiments, the treatment results in an improvement in quality of life as assessed by Norfolk Quality of Life-Diabetic Neuropathy as compared to baseline. In some embodiments, the treatment results in an improvement in quality of life as assessed by EuroQOL (EQ)-5D-5L as compared to baseline. In some embodiments, the treatment results in an improvement in cardiac MRI (e.g., cardiac imaging of amyloid fibrils) as compared to baseline. In some embodiments, the treatment results in an improvement in N-terminal prohormone of brain natriuretic peptide (NT-proBNP) levels as compared to baseline, In some embodiments, the treatment results in an improvement in Troponin I levels as compared to baseline. In some embodiments, the treatment results in an improvement in New York Health Association (NYHA) classification as compared to baseline. In some embodiments, the treatment results in an improvement as scored by the Kansas City Cardiomyopathy Questionnaire (KCCQ) as compared to baseline.

In some embodiments, treatment slows or halts progression of FAP. In some embodiments, treatment results in improvement, stabilization, or slowing of change in symptoms of sensorimotor neuropathy or autonomic neuropathy.

In some embodiments, treatment results in improvement, stabilization, or slowing of change in symptoms of FAC. In some embodiments, treatment results in improvement, stabilization, or slowing of change symptoms of restrictive cardiomyopathy or congestive heart failure.

In some embodiments, efficacy of treatment is measured by improvement or slowing of progression in symptoms of sensorimotor or autonomic neuropathy. In some embodiments, efficacy of treatment is measured by an increase or a slowing of decrease in ability to move an area of the body or to feel in any area of the body. In some embodiments, efficacy of treatment is measured by improvement or a slowing of decrease in the ability to swallow; breath; use arms, hands, legs, or feet; or walk. In some embodiments, efficacy of treatment is measured by improvement or a slowing of progression of neuralgia. In some embodiments, the neuralgia is characterized by pain, burning, tingling, or abnormal feeling. In some embodiments, efficacy of treatment is measured by improvement or a slowing of increase in postural hypotension, dizziness, gastrointestinal dysmotility, bladder dysfunction, or sexual dysfunction. In some embodiments, efficacy of treatment is measured by improvement or a slowing of progression of weakness. In some embodiments, efficacy of treatment is measured using electromyogram, nerve conduction tests, or subject-reported outcomes.

In some embodiments, efficacy of treatment is measured by improvement or slowing of progression of symptoms of congestive heart failure or CHF. In some embodiments, efficacy of treatment is measured by a decrease or a slowing of increase in shortness of breath, trouble breathing, fatigue, or swelling in the ankles, feet, legs, abdomen, or veins in the neck. In some embodiments, efficacy of treatment is measured by improvement or a slowing of progression of fluid buildup in the body, which may be assessed by measures such as weight gain, frequent urination, or nighttime cough. In some embodiments, efficacy of treatment is measured using cardiac biomarker tests (such as B-type natriuretic peptide [BNP] or N-terminal pro b-type natriuretic peptide [NT-proBNP]), lung function tests, chest x-rays, or electrocardiogra

In some embodiments, the treatment results in an increased survival time of the subject. In some embodiments, the treatment slows or halts disease progression. In some embodiments, the efficacy of treatment with the compositions described herein is seen at 2 weeks, 4 weeks, 2 months, 4 months, 6 months, 9 months, 1 year, 2 years, 3 years, 4 years, 5 years, or 10 years after delivery.

In other embodiments, the LNP composition is also administered with a second therapeutic agent. In some embodiments, the second therapeutic agent is a stabilizer of the tetrameric form of TTR. In some embodiments, the second therapeutic is diflunisal or tafamidis.

a. Subject Inclusion Criteria

In some embodiments, a subject having TTR amyloidosis (ATTRv-PN and ATTR-CM) to whom the LNP composition described herein (e.g., comprising an mRNA encoding a Cas nuclease, e.g., Cas9; and a guide RNA that targets a gene, e.g., a guide RNA that targets the TTR gene) is administered is assessed for one or more of the following subject inclusion criteria.

i. ATTRv-PN Subject Inclusion Criteria

In some embodiments, a human subject has been or concurrently is diagnosed with ATTR prior to treatment. In some embodiments, a human subject is diagnosed with ATTR based on genetic testing (e.g., a documented TTR mutation). In some embodiments, a human subject is diagnosed with ATTR based on a clinical diagnosis of sensorimotor peripheral neuropathy. In some embodiments, a human subject is diagnosed with ATTR based on a Neuropathy Impairment Score (NIS)≥5 and ≤130 prior to treatment. In some embodiments, a human subject is diagnosed with ATTR based on a documented tissue deposition of TTR amyloid by biopsy or by validated noninvasive imaging. In some embodiments, a human subject is diagnosed with ATTR based on a Polyneuropathy Disability (PND) score≤3b.

In some embodiments, a human subject has progression of ATTRv-PN symptoms prior to treatment. In some embodiments, the human subject has an increase in Polyneuropathy Disability (PND) score≥1 point. In some embodiments, the human subject has an increase in Familial Amyloid Polyneuropathy (FAP) stage≥1 point. In some embodiments, the human subject has an increase in Neuropathy Impairment Score (NIS)≥5 points. In some embodiments, the human subject has an increase in NIS-Lower Limb (LL)≥5 points. In some embodiments, the human subject has a decrease in Modified Body-Mass Index (mBMI)≥25 kg/m2×g/L. In some embodiments, the human subject has a decrease in 6-minute walk test≥30 meters. In some embodiments, the human subject has a decrease in 10-meter walk test≥0.1 m/s. Assessment of these and other inclusion criteria are known in the art.

In some embodiments, the human subject is between 18 years of age and 80 years of age at the time of administration. In some embodiments, the human subject has a diagnosis of peripheral neuropathy (PN) due to TTR amyloidosis (ATTR) based on a documented TTR mutation (e.g. whole TTR gene sequencing information). In some embodiments, the human subject has a diagnosis of sensorimotor peripheral neuropathy. In some embodiments, the human subject has a Neuropathy Impairment Score (NIS)≥5 and ≤130. In some embodiments, the human subject has a documented tissue deposition of TTR amyloid by biopsy or by validated noninvasive imaging. In some embodiments, the human subject has a Polyneuropathy Disability (PND) score≤3b. In some embodiments, the human subject has a body weight between about 50 kg and 90 kg. In some embodiments, the human subject has a body weight between about 50 kg and 120 kg. In some embodiments, the human subject has an aspartate aminotransferase (AST) level≤upper limit of normal (ULN) range at screening. In some embodiments, the human subject has an alanine aminotransferase (ALT) level≤ upper limit of normal (ULN) range at screening. In some embodiments, the human subject has a total bilirubin level≤upper limit of normal (ULN) range at screening. In some embodiments, the human subject has an international normalized ratio (INR)≤upper limit of normal (ULN) range at screening. In some embodiments, the human subject has an estimated glomerular filtration rate (GFR)>45 mL/min/1.73m2, (e.g., as measured by the Modification of Diet in Renal Disease equation) at screening. In some embodiments, the human subject has a platelet count≥100,000 cells/mm3 at screening. In some embodiments, the human subject has an N-terminal prohormone of brain natriuretic peptide (NT-proBNP)<2,000 μg/mL at screening. In some embodiments, the human subject has a low density lipoprotein (LDL) cholesterol<200 mg/dL at screening. In some embodiments, the human subject has a vitamin A≥lower limit of normal (LLN) at screening. In some embodiments, the human subject has a thyroid-stimulating hormone (TSH) within normal range at screening. In some embodiments, the human subject has a vitamin B12 level≥LLN at screening. In some embodiments, the human subject has echocardiogram. In some embodiments, the human subject is male and must agree to not donate sperm for 84 days after administration.

ii. ATTR-CM Subject Inclusion Criteria

In some embodiments, a human subject has a documented diagnosis of transthyretin (ATTR) amyloidosis with cardiomyopathy, classified as hereditary ATTR (ATTRv) amyloidosis with cardiomyopathy or wild type cardiomyopathy (ATTRwt).

In some embodiments, a human subject has at least one prior hospitalization for heart failure and/or clinical evidence of heart failure. In some embodiments, a human subject has New York Heart Association (NYHA) Class I-III heart failure.

In some embodiments, a human subject receives oral diuretic therapy at least three times weekly at a dose that has been consistently maintained (or changed by no more than 50%) for at least 21 days prior to screening.

In some embodiments, a human subject is clinically stable with no cardiovascular related hospitalizations within 4 weeks prior to administration of the compositions described herein.

In some embodiments, a human subject's symptoms of heart failure are optimally managed and clinically stable as assessed by the investigator.

In some embodiments, a human subject is able to complete≥150 meters on the 6-minute walk test (6-MWT) during the screening period.

In some embodiments, a human subject has a body weight of at least 45 kg at screening.

In some embodiments, a human subject meets certain laboratory criteria during screening. In some embodiments, a human subject has aspartate aminotransferase (AST), alanine aminotransferase (ALT), and total bilirubin≤upper limit of normal (ULN) range (unless subject has Gilbert's Syndrome). In some embodiments, for a human subject with a history of Gilbert's Syndrome, the subject has total bilirubin≤2×ULN at screening. In some embodiments, a human subject has an estimated glomerular filtration rate (eGFR)>30 mL/min/1.73m2 as measured by the CKD-EPI. In some embodiments, a human subject has a platelet count≥100,000 cells/mm3. In some embodiments, a human subject has activated partial thromboplastin time (aPTT), prothrombin time (PT), fibrinogen and d-dimer levels within the normal range or deemed clinically non-significant by the investigator. In some embodiments, a human subject has NT-proBNP>600 μg/mL (or, if patient has known diagnosis of atrial fibrillation, NT-proBNP>1,000 μg/mL). In some embodiments, a human subject has low density lipoprotein (LDL) cholesterol<200 mg/dL at screening, with or without pharmacotherapy. In some embodiments, a human subject has vitamin A≥lower limit of normal (LLN). In some embodiments, a human subject has thyroid-stimulating hormone (TSH) measurement within the normal range. In some embodiments, the human subject meets all of the laboratory criteria described above at screening.

In some embodiments, a human subject limits alcohol consumption to 1 alcoholic drink per day during screening and through 28-days after treatment with the composition described herein.

In some embodiments, a human subject is a male and/or female subject who is 18 to 90 years of age (inclusive), e.g., at the time of signing informed consent. In some embodiments, a female subject is postmenopausal (e.g., no menses for 12 months without an alternative medical cause prior to screening. In some embodiments, a high follicle-stimulating hormone (FSH) level in the postmenopausal range may be used to confirm a post-menopausal state in women not using hormonal contraception or hormonal replacement therapy. In some embodiments, in the absence of 12 months of amenorrhea, a single FSH measurement is insufficient.). In some embodiments, a female subject is surgically sterile (e.g., hysterectomy, bilateral salpingectomy, and bilateral oophorectomy) at least 1 month prior to screening. In some embodiments, a male subject with partner(s) of child-bearing potential or who are pregnant agree to using a condom prior to screening and for 84 days after study drug administration. In some embodiments, a male subject agrees not to donate sperm for 84 days after study drug administration. The timeframe may be extended beyond the 84 days, if sperm donation is contraindicated based on country-specific guidelines.

In some embodiments, a human subject is assessed for risk of transmission or contraction of SARS-COV-2 determined acceptable to proceed with an elective procedure at the health care facility (e.g., document that vaccination series completed, recent PCR test negative, or such testing no longer required, etc.).

In some embodiments, a human subject agrees not to participate in another interventional study for a minimum of 28 days post dosing.

In some embodiments, during the screening period, a human subject has 3 blood pressure measurements with an appropriately sized cuff recorded, each less than 140/90 mmHg. If during screening, the blood pressure is ≥140/90 mmHg, the subject may receive new or modified anti-hypertensive therapy and continue with screening until subject has 3 blood pressure measurements <140/90 mmHg before proceeding with administration of the compositions described herein.

b. Subject Exclusion Criteria

In some embodiments, a subject having TTR amyloidosis (ATTRv-PN and ATTR-CM) to whom the LNP composition described herein (e.g., comprising an mRNA encoding a Cas nuclease, e.g., Cas9; and a guide RNA that targets a gene, e.g., a guide RNA that targets the TTR gene) is administered is assessed for one or more of the following subject exclusion criteria.

i. ATTRv-PN Subject Exclusion Criteria

In some embodiments, the human subject does not have amyloidosis attributable to non-TTR protein, e.g., amyloid light-chain (AL) amyloidosis. In some embodiments, the human subject does not have leptomeningeal transthyretin amyloidosis. In some embodiments, the human subject does not have a hypersensitivity to any lipid nanoparticles (LNP) component or has previously received LNP and experienced any treatment-related laboratory abnormalities or adverse events (e.g., an ALT or AST>3×ULN if baseline was normal or >3× baseline if baseline was above normal after receiving an LNP containing product, an INR, aPTT or d-dimer>1.5×ULN if baseline was normal or >1.5× Baseline if baseline was above normal after receiving an LNP containing product, a LNP treatment-related adverse event classified as CTCAE Grade 3 or higher, an infusion-related reaction (IRR) to an LNP containing product requiring treatment or discontinuation of infusion). In some embodiments, the human subject does not have other known causes of sensorimotor or autonomic neuropathy (e.g., diabetic neuropathy, autoimmune disease-associated neuropathy). In some embodiments, the human subject does not have Type 1 diabetes mellitus or diagnosis of Type 2 diabetes mellitus for >5 years. In some embodiments, the human subject does not have current or prior NYHA class III or IV symptoms due to heart failure or worsening of heart failure symptoms within 90 days prior to or during screening. In some embodiments, the human subject has not had cardiovascular hospitalization or invasive procedure within 90 days. In some embodiments, the human subject has not had an invasive cardiovascular procedure (e.g., coronary stent, pacemaker placement, etc.). In some embodiments, the human subject does not have vitamin A supplementation. In some embodiments, the human subject does not have a pre-treatment medication regimen. In some embodiments, the human subject does not have a history of use of antiplatelet (e.g., aspirin, clopidogrel) or antithrombotic therapy (e.g., warfarin, dabigatran, apixaban) within 14 days of administration. In some embodiments, the human subject does not have a history of thrombophilia, or positive genetic test for Factor V Leiden and/or prothrombin 20210. In some embodiments, the human subject does not have an anticipated survival of less than 2 years. In some embodiments, the human subject does not have ophthalmologic findings consistent with Vitamin A deficiency. In some embodiments, the human subject does not have a history of cirrhosis. In some embodiments, the human subject does not have known or suspected systemic viral, parasitic, or fungal infection or received antibiotics for bacterial infection. In some embodiments, the human subject does not have a history of Hepatitis B or C infection or positive Hepatitis B surface antigen (HBsAg) or Hepatitis C Virus antibody (HCV Ab) test. In some embodiments, the human subject does not have a history of positive human immunodeficiency virus (HIV) status. In some embodiments, the human subject has not had prior liver, heart or other solid organ transplant or bone marrow transplant or anticipated transplant within 1 year of administration. In some embodiments, the human subject does not have a history of active malignancy within 5 years prior to screening or during the screening period, except for basal cell carcinoma of skin, curatively resected squamous cell carcinoma of skin, cervical carcinoma in situ curatively treated, or low-grade prostate adenocarcinoma for which appropriate management is observation alone. In some embodiments, the human subject does not have a history of alcohol or drug abuse within 3 years prior to screening. In some embodiments, the human subject is not female and of child-bearing potential or is breastfeeding. In some embodiments, the human subject does not have a positive Severe Acute Respiratory Syndrome Coronavirus-2 (SARS-COV-2) polymerase change reaction (PCR) test within 7 days of administration.

Assessment of these and other exclusion criteria are known in the art.

ii. ATTR-CM Subject Exclusion Criteria

In some embodiments, a human subject does not have amyloidosis attributable to non-TTR protein, e.g., amyloid light-chain (AL) amyloidosis. In some embodiments, a human subject does not have known leptomeningeal transthyretin amyloidosis.

In some embodiments, a human subject does not have known hypersensitivity to any lipid nanoparticles (LNP) component. In some embodiments, a human subject did not previously receive LNP and experience any treatment related laboratory abnormalities or adverse event (AE), e.g., ALT or AST>3×ULN if baseline was normal or >3× baseline if baseline was above normal after receiving an LNP containing product; INR, aPTT or d-dimer>1.5×ULN if baseline was normal or >1.5× baseline if baseline was above normal after receiving an LNP containing product; any LNP treatment-related adverse event classified as CTCAE Grade 3 or higher; infusion-related reaction (IRR) to an LNP containing product requiring treatment or discontinuation of infusion (in some embodiments, slowing of the infusion rate to mitigate an infusion-related reaction is not considered exclusionary); and/or any LNP treatment-related adverse event which in the opinion of the investigator should be exclusionary.

In some embodiments, a human subject does not use of the following TTR-directed therapy for ATTR within the specified timeframe:
patisiran (small interfering ribonucleic acid (siRNA) therapeutic formulated LNP), e.g., prior history of use and/or last dose administered less than 90 days prior to study drug administration.
inotersen (antisense oligonucleotide (ASO)), e.g., prior history of use and/or last dose administered less than 160 days prior to study drug administration.
vutrisiran (investigational siRNA therapeutic GalNAc conjugate), e.g., prior history of use.
tafamidis (TTR stabilizer), e.g., subject is on stable dose for a minimum of 14 days prior to study drug administration.
diflunisal (TTR stabilizer), e.g., last dose administered less than 14 days prior to study drug administration.
doxycycline and/or tauroursodeoxycholic acid (TTR matrix solvent), e.g., last dose administered less than 14 days prior to study drug administration.
experimental TTR stabilizer (e.g., AG-10), e.g., last dose administered less than 6 months prior to study drug administration.
any other investigational agent for the treatment of ATTRv-CM, e.g., last dose administered less than 30 days or 5 half-lives, whichever is longer, prior to study drug administration.

In some embodiments, a human subject does not have heart failure that, in the opinion of the investigator, is caused by ischemic heart disease (e.g., prior myocardial infarction with documented history of cardiac enzymes and ECG changes), hypertension, or uncorrected valvular disease and not primarily due to transthyretin amyloid cardiomyopathy.

In some embodiments, a human subject does not have a history of sustained ventricular tachycardia or aborted ventricular fibrillation or with a history of atrioventricular (AV) nodal or sinoatrial (SA) nodal dysfunction for which a pacemaker is indicated but will not be placed. In some embodiments, a human subject does not have pacemaker or defibrillator placement, initiation of or change in anti-arrhythmic medication within 28 days prior to study drug administration.

In some embodiments, a human subject is not unable or unwilling to take Vitamin A supplementation.

In some embodiments, a human subject does not have a clinical assessment that indicates meaningful risk associated with ATTR-CM status administration of required pre-medications. In some embodiments, a human subject is not unable or unwilling to take the required pre-treatment medication regimen.

In some embodiments, a human subject does not have antithrombotic therapy with warfarin or heparin/heparin-derivatives within 14 days prior to study drug administration or anticipated need for warfarin anti-thrombotic therapy during the post study drug dosing period. In some embodiments, use of apixaban, dabigatran, edoxaban, or rivaroxaban is allowed if the dose is stable for 28 days prior to screening, stable through screening, and expected to remain stable for 90 days after study drug administration.

In some embodiments, a human subject does not have a history of thrombophilia, or a history of a positive genetic test for Factor V Leiden, prothrombin 20210, or any positive test for Protein S deficiency and/or Protein C deficiency.

In some embodiments, a human subject does not have an anticipated survival of less than 1 year, in the opinion of the investigator.

In some embodiments, a human subject does not have ophthalmologic findings consistent with Vitamin A deficiency on screening ophthalmologic examination.

In some embodiments, a human subject does not have a history of cirrhosis.

In some embodiments, a human subject does not have known or suspected systemic viral, parasitic, or fungal infection, or received antibiotics for bacterial infection within 14 days of screening.

In some embodiments, a human subject does not have a history of Hepatitis B or C infection or positive Hepatitis B surface antigen (HBsAg) or Hepatitis C Virus antibody (HCV Ab) test at screening. In some embodiments, a subject who has no evidence of cirrhosis, has completed a curative intent regimen for Hepatitis C, and is deemed by a gastroenterologist to have no active Hepatitis C and no increased risk for hepatotoxicity is not excluded.

In some embodiments, a human subject does not have a history of positive human immunodeficiency virus (HIV) status.

In some embodiments, a human subject does not have prior liver, heart or other solid organ transplant or bone marrow transplant or anticipated transplant within 1 year of screening. In some embodiments, prior history of or planned corneal transplant is not exclusionary.

In some embodiments, a human subject does not have a history of active malignancy within 3 years prior to screening or during the screening period, except for basal cell carcinoma of skin, curatively resected squamous cell carcinoma of skin, cervical carcinoma in situ curatively treated or low-grade prostate adenocarcinoma, for which appropriate management is observation.

In some embodiments, a human subject does not have a history of alcohol or drug abuse within 3 years prior to screening. In some embodiments, a female subject is not of child-bearing potential or breastfeeding. In some embodiments, a human subject does not have any condition, laboratory abnormality, or other reason that, in the investigator's opinion, could adversely affect the safety of the subject, impair the assessment of study results, or preclude compliance with the study.

### 3. Infusion Prophylaxis

In some embodiments, a method described herein, e.g., comprising administering to a subject the LNP composition described herein (e.g., comprising an mRNA encoding a Cas nuclease, e.g., Cas9; and a guide RNA that targets a gene, e.g., a guide RNA that targets the TTR gene), further comprises infusion prophylaxis. In some embodiments, an infusion prophylaxis is administered to a subject before administration of the gene editing composition. In some embodiments, the infusion prophylaxis regimen administered to a subject before administering the LNP composition comprises administering intravenous steroid; intravenous H1 blocker or oral H1 blocker; and intravenous or oral H2 blocker. The intravenous steroid may be dexamethasone, e.g., 10 mg. The intravenous H1 blocker may be diphenhydramine, e.g., 50 mg. The oral H1 blocker may be cetirizine, e.g., 10 mg. The intravenous or oral H2 blocker may be famotidine, e.g., 20 mg.

### EXAMPLES

### Example 1. LNP-Particle Based Composition for TTR Gene Editing

In Vitro Transcription (“IVT”) of Nuclease mRNA

Capped and polyadenylated mRNA containing N1-methyl pseudo-U was generated by in vitro transcription using routine methods. Briefly, a linearized plasmid DNA template and T7 RNA polymerase. Plasmid DNA containing a T7 promoter, a sequence for transcription, and a polyadenylation region was linearized with XbaI per manufacturer's protocol. The XbaI was inactivated by heating. The linearized plasmid was purified from enzyme and buffer salts. The IVT reaction to generate modified mRNA was performed by incubating at 37° C.: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase; 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated at 37° C. to remove the DNA template.

The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA was purified using LiCl precipitation, ammonium acetate precipitation, and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 el42). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame according to Sequence Table. When the sequences cited in this paragraph are referred to below with respect to RNAs, it is understood that Ts should be replaced with Us (which can be modified nucleosides as described above). Messenger RNAs used in the Examples include a 5′ cap and a 3′ polyadenylation sequence e.g., up to 100 nts and are identified in Table 3. Guide RNAs are chemically synthesized by methods known in the art.

Preparation of LNP Formulation Containing sgRNA and Cas9 mRNA

In general, the lipid nanoparticle components were dissolved in 100% ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL. The LNPs used contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), also called herein Lipid A, cholesterol, DSPC, and PEG2k-DMG in a 50:38:9:3 molar ratio, respectively. The LNPs were formulated with a lipid amine to RNA phosphate (N: P) molar ratio of about 6, and a ratio of gRNA to mRNA of 1:2 by weight. The LNPs used comprise a Cas9 mRNA and an sgRNA.

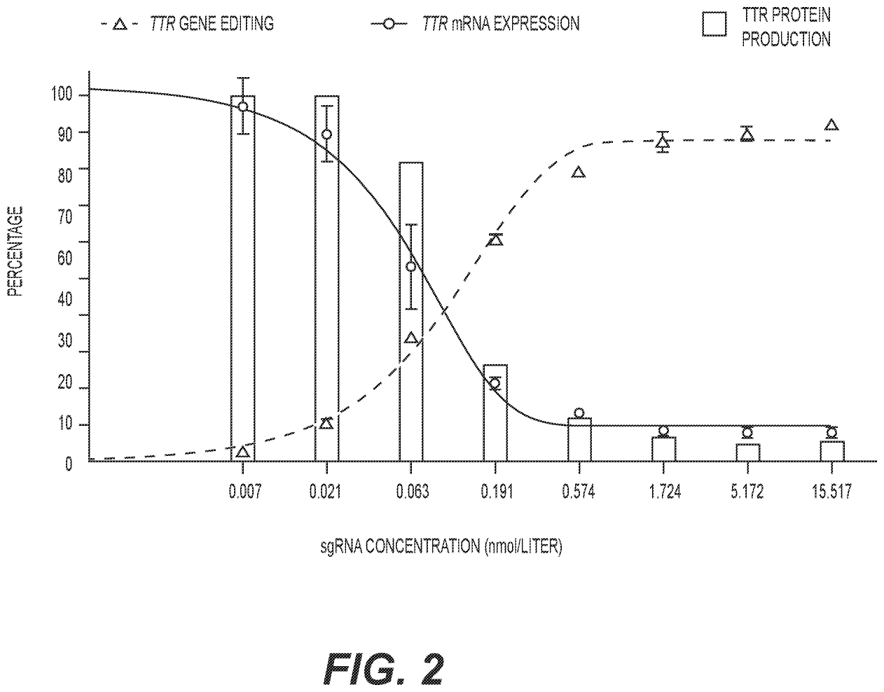

The LNPs were prepared using a cross-flow technique utilizing impinging jet mixing of the lipid in ethanol with two volumes of RNA solutions and one volume of water. The lipid in ethanol was mixed through a mixing cross with the two volumes of RNA solution. A fourth stream of water was mixed with the outlet stream of the cross through an inline tee (See WO2016010840 FIG. 2.). The LNPs were held for 1 hour at room temperature, and further diluted with water (approximately 1:1 v/v). Diluted LNPs were concentrated using tangential flow filtration on a flat sheet cartridge (Sartorius, 100 kD MWCO) and then buffer exchanged using PD-10 desalting columns (GE) into 50 mM Tris, 45 mM NaCl, 5% (w/v) sucrose, pH 7.5 (TSS). The resulting mixture was then filtered using a 0.2 μm sterile filter. The final LNPs were characterized to determine the encapsulation efficiency, polydispersity index, and average particle size. The final LNP was stored at 4° C. or −80° C. until further use.

### Next-Generation Sequencing (“NGS”) and Analysis for Editing Efficiency

Genomic DNA was extracted from cells or tissue according to methods known in the art, for example using QuickExtract DNA Extraction solution (Epicentre, Cat. QE09050) or Quick Extract (Lucigen, Cat. SS000035-D2). To quantitatively determine the efficiency of editing at the target location in the genome, sequencing was utilized to identify the presence of insertions and deletions introduced by gene editing. PCR primers were designed around the target site within the gene of interest (e.g. TTR), and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion or deletion (“indel”) was calculated.

The editing percentage (e.g., the “editing efficiency” or “percent editing”) is defined as the total number of sequence reads with insertions or deletions (“indels”) over the total number of sequence reads, including wild type.

### Example 2. Selection of sgRNA Targeting TTR Gene

A sgRNA targeting TTR gene sequence AAAGGCUGCUGAUGACACCU (SEQ ID No: 15; human genome build hg38 chromosome 18:31592987-31593007) was selected for efficient knockout and specificity after a comprehensive off-target characterization workflow that applied a combination of both in silico and empirical approaches. To select for a high therapeutic index (ratio of on-versus off-target editing), we performed genome-wide assays and targeted sequencing, to identify and verify candidate sgRNA off-target sites.

Genomic loci with the potential for off-target editing were discovered using complementary computational and laboratory-based approaches (Cas-OFFinder, GUIDE-seq and SITE-Seq). Subsequently, the mismatches between the potential off-target sites and the single-guide RNA (sgRNA) targeting sequence in NTLA-2001 were examined. Sites overlapping with protein coding exons were detected using an interval tree algorithm. Those having at least one nucleotide overlap with a protein coding exon were retained if they had four or fewer mismatches to the protospacer sequence. Three mismatches were allowed for potential off-target sites that did not overlap with an exon from the coding DNA sequence (CDS). Together both sets of sites were included in the curation of predicted potential off-target editing loci for NTLA-2001.

The CRISPR/Cas9 off-target discovery assay GUIDE-seq was performed in cells as previously described, with minor changes. Illumina next-generation sequencing (NGS) library preparation was performed according to the published protocol and sequencing was performed on both Illumina's MiSeq and HiSeq 2,500 with 150 base pair (bp) paired-end reads. GUIDE-seq was performed on NTLA-2001 in an HEK293 cell line (HEK293-Cas9) engineered to constitutively express Spy Cas9 green fluorescent protein (Spy Cas9-GFP) fusion protein.

The CRISPR/Cas9 off-target discovery assay SITE-Seq is a cell-free biochemical method that is among the most sensitive to potential off-target editing discovery because the assay is executed on deproteinated and purified genomic DNA (gDNA) to eliminate any substrate restrictions on CRISPR/Cas9 enzymatic activity. SITE-Seq was executed on human gDNA derived from peripheral blood mononuclear cells from two unique male blood donors. Each gDNA sample was digested with in vitro assembled ribonucleoprotein of Cas9 and the transthyretin [TTR] targeting sgRNA contained in NTLA-2001 to induce DNA cleavage at the on-target site and potential off-target sites with homology to the sgRNA sequence. After gDNA digestion, the free gDNA fragment ends were ligated with adapters to facilitate edited fragment enrichment and NGS library construction. The NGS libraries were sequenced as described in Example 1, and through bioinformatic analysis, the reads were analyzed to determine the genomic coordinates of the free DNA ends. Locations in the human genome with an accumulation of reads were then annotated as potential off-target sites.

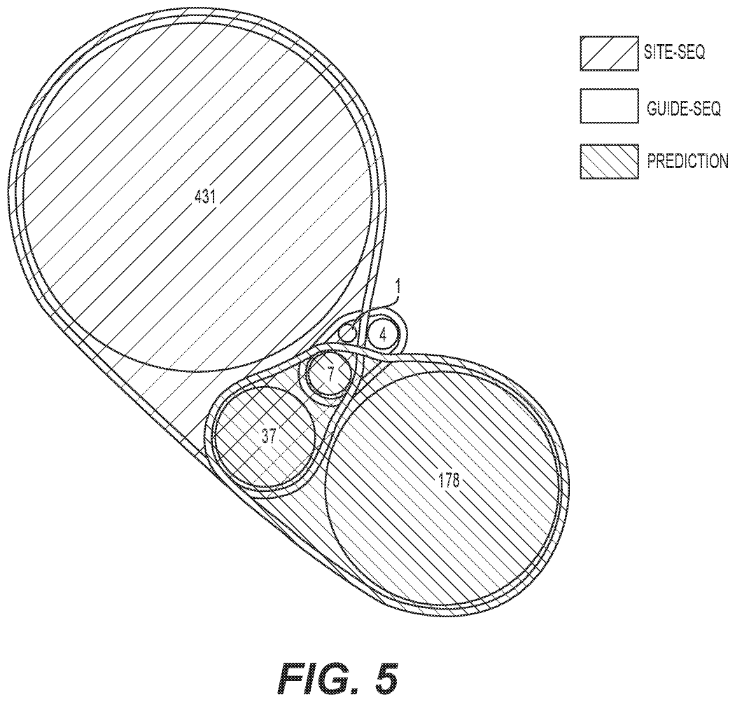

All potential off-target editing loci discovered by computational prediction with Cas-OFFinder, and empirical discovery assays GUIDE-seq and SITE-Seq were curated and annotated for validated off-target editing in NTLA-2001 genome edited cells. Potential off-target editing discovery for NTLA-2001 from Cas-OFFinder, GUIDE-seq and SITE-Seq resulted in a total of 658 sites including the NTLA-2001 on-target site. SITE-Seq discovered 476 (72.3%) sites, of which 431 (65.5%) were discovered exclusively by this method. Cas-OFFinder discovered 222 (33.7%) sites, of which 178 (27.1%) were discovered exclusively by this method. GUIDE-seq discovered 12 (1.8%) sites, of which 4 (0.6%) were discovered exclusively by this method (FIG. 5).

The false discovery rate was controlled for each discovery assay uniquely: (1) Cas-OFFinder was tuned to identify loci with up to three mismatches genome-wide and up to four mismatches in exonic DNA; (2) The cell-based assay GUIDE-Seq was optimized in a HEK293 cell-line engineered to constitutively express Cas9 and the maximum tolerated dose of double-stranded donor oligonucleotide was used; (3) The biochemical-based assay SITE-Seq was qualified for off-target discovery at 16 nM, 64 nM, and 256 nM Cas9 RNP concentrations, and the 64 nM Cas9 RNP digestion was selected as optimal to ensure capture of all potential off-target loci that might validate in edited cells while not reducing our validation sensitivity with the burden of a greater number of potential off-target loci.

Classification error rates were as follows.
Cas-OFFinder results allowing up to three mismatches genome-wide and up to four mismatches in exonic DNA identified 221 potential off-target loci. Based on validation data in edited cells:
   False positive rate=1−(3÷221)=98.6%
   False negative rate=1−(3÷7)=57.1%
GUIDE-Seq identified 11 potential off-target loci. Based on indel detection validation data in edited cells:
   False positive rate=1−(3÷11)=72.7%
   False negative rate=1−(3÷7)=57.1%
SITE-Seq identified 475 potential off-target loci. Based on indel detection validation data in edited cells:
   False positive rate=1−(7÷475)=98.5%
   False negative rate=1−(7÷7)=0%

### Example 3. Validation of Potential Off-Target Editing in Primary Human Hepatocytes

The maximum concentration of lipid nanoparticle [LNP] used to evaluate off target potential was chosen based on the maximum concentration of NTLA-2001 that does not induce cell toxicity in primary human hepatocytes (PHH). This concentration was 27-fold greater than the 90% effective concentration (EC₉₀; concentration that achieved >90% TTR protein knockdown in PHH).

Two complementary technologies were used to validate potential off-target editing in PHH. The first is a multiplex PCR technology called RNase H2-dependent PCR amplification and NGS (rhAMPSeq). This assay allows the simultaneous enrichment of on- and potential off-target loci in a single PCR reaction for amplicon-sequencing with NGS. The second technology was standard singleplex amplicon-sequencing (Amp-Seq) as described in Example 1, which was used to characterize those loci that failed inclusion criteria applied to rhAMPSeq.

Illumina Next Seq instrument was used to sequence the rhAMPSeq libraries with 150 base bp paired-end sequencing reads plus two 8 bp dual indexing reads. Sample-specific sequencing reads were then stitched and aligned to the human genome reference (build GRCh38) using bowtie2 (v2.2.6) followed by local re-alignment using the Smith-Waterman algorithm. Nucleotides within 10 base pairs of the potential Cas9 cut site were evaluated for indels to the human genome reference sequence. The site editing percentage was defined as the total number of sequencing reads with indels divided by the total number of sequencing reads.

There were seven validated off-target indels detected across two PHH donor lots after exposure to super-saturating concentrations of NTLA-2001 (Table 1). This approach was selected because off-target editing is directly proportional to on-target editing, therefore the detection of validated off-target editing was maximized by oversaturated genome editing with NTLA-2001.

TABLE 1
Validated off-target and on-target indel detection of genome editing in two donor
lots of primary human hepatocytes with super-saturating concentrations of NTLA-2001

| | | PHH lot 1 | | PHH lot 2 | |
|---|---|---|---|---|---|
| Site description | Annotation | Mean Δ indel (%) | p value | Mean Δ indel (%) | p value |
| On-target | On-target | 92.57 ± 7.85 | 0.007 | 93.50 ± 0.10 | 1.91E−07 |
| Off-target 1 | Intergenic | 7.37 ± 0.72 | 0.002 | 1.43 ± 0.12 | 1.99E−04 |
| Off-target 2 | Intronic | 3.70 ± 0.10 | 3.90E−06 | 1.07 ± 0.15 | 4.11E−03 |
| Off-target 3 | Intergenic | 0.87 ± 0.06 | 3.50E−05 | 0.50 ± 0.20 | 0.037 |
| Off-target 4 | Intergenic | 1.50 ± 0.69 | 0.03 | 0.33 ± 0.06 | 0.001 |
| Off-target 5 | Intergenic | 0.30 ± 0.10 | 0.02 | 0.13 ± 0.06 | 0.029 |
| Off-target 6 | Intronic | 0.27 ± 0.06 | 0.01 | 0.10 ± 0.00 | 0.211 |
| Off-target 7 | Intergenic | 0.27 ± 0.06 | 0.01 | 0.10 ± 0.00 | 0.091 |

Values in bold represent validated off-target indels. PHH denotes primary human hepatocytes.

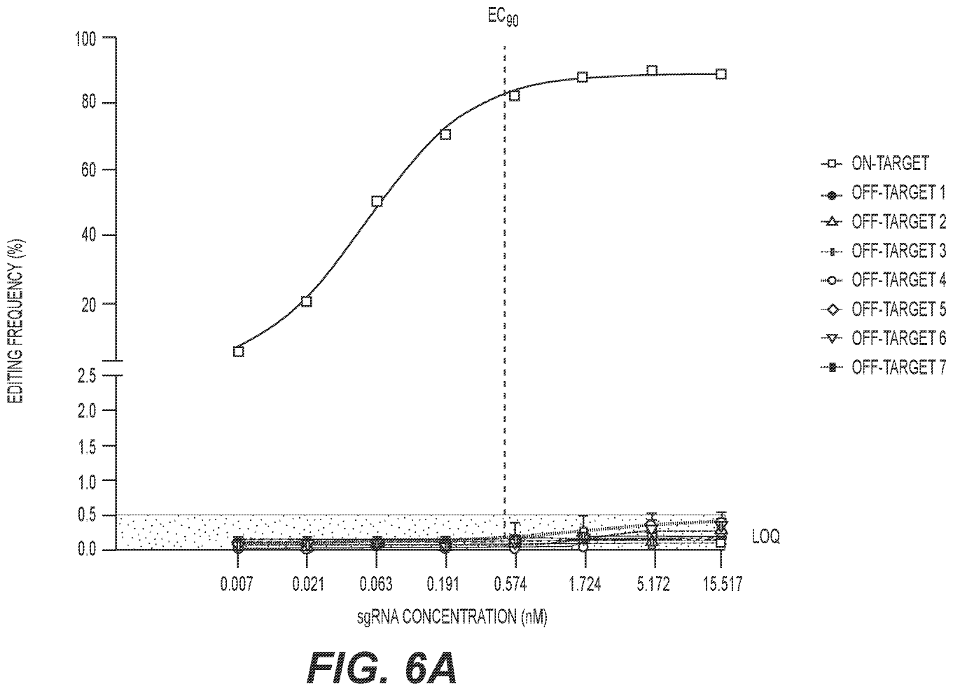

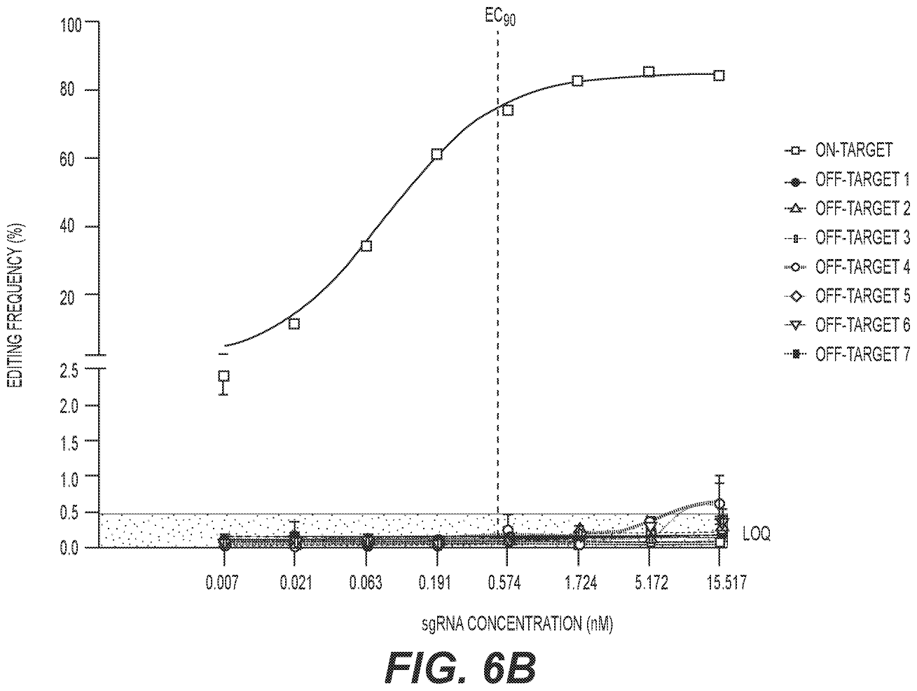

Five of the loci were located in intergenic regions of the human genome and two were located in introns of protein coding genes. These validated off-target editing loci were further characterized in a dose responsive manner of NTLA-2001 exposure (FIG. 6). This approach allowed us to more closely characterize the detection and frequency of off-target indel formation at therapeutically relevant TTR protein reduction.

Analysis of these specific off-target sequencing data with NGS revealed that there were zero validated off-target indels detected when treating PHH with NTLA-2001 up to 3-fold greater than the EC90 that achieved an average of 90% TTR protein reduction in PHH.

No truth-set exists to determine the false classification rates of potential off-target loci. Currently available whole genome sequencing technology is unsatisfactory in comparison to the sensitivity of off-target indels detected through targeted amplicon-sequencing. A total of 657 potential off-target were subjected to amplicon-sequencing validation that was qualified to detected >90% of indels down to a frequency of 0.2%.
Total failed validation sites=98.93%
Validated off-target loci=1.07%

### Example 4. In Vitro Evaluations of the Potency of NTLA-2001

In vitro dose-response and gene editing potency of NTLA-2001 were assessed in primary cell cultures of human hepatocytes.

In primary human hepatocytes, NTLA-2001 was highly potent (EC₅₀; 0.05 to 0.15 nM; EC₉₀; 0.17 to 0.67 nM) and demonstrated saturating levels of TTR gene editing (>93.7%), resulting in ≥91% reduction in TTR mRNA and ≥95% reduction in TTR protein (FIG. 2). NGS data demonstrated that NTLA-2001 induced knockout of the TTR gene.

FIG. 2 demonstrates the relationship between increasing concentration of guide RNA and consequent percentage of TTR gene editing, as well as TTR mRNA and protein reduction in a single lot of primary human hepatocytes. The primary indel patterns were a single nucleotide deletion or insertion at the cut site, inducing a frameshift mutation (data not shown).

### Example 5. Characterization of DNA Structural Variants after Genome Editing

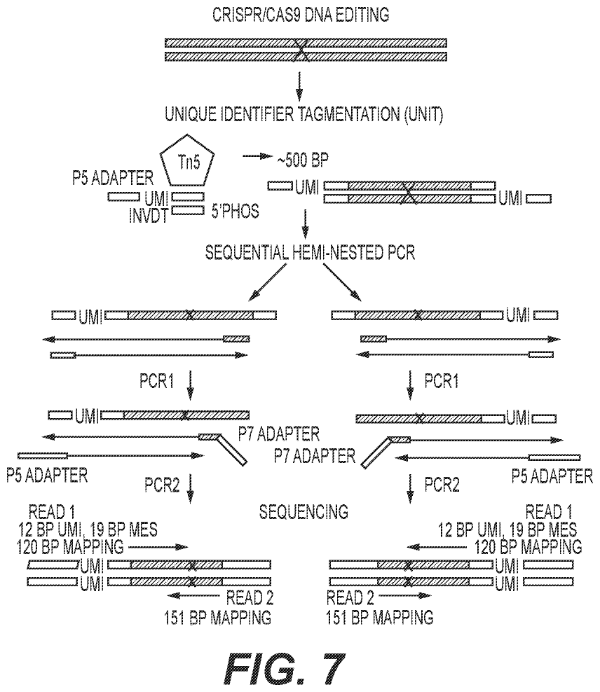

CRISPR/Cas9 genome editing has the potential to result in DNA structural variations (SVs) as a natural outcome of double-stranded DNA break repair. Potential DNA SVs include inter-chromosomal translocations, inversions, duplications, and deletions. To execute a comprehensive characterization of the potential DNA SVs that may occur after genome editing with NTLA-2001, Intellia developed and qualified the application of two complementary approaches: (1) short-read NGS with SV characterization assay; (2) long-read NGS with long-range PCR (FIG. 7). The results of these approaches revealed concordant and low (<1%) levels of DNA SVs that were in-line with published results of high efficiency CRISPR/Cas9 genome editing.

Analysis of SV characterization assay paired-end NGS data results in two possible outcomes. One is the concordant mapping of the paired-end sequencing reads. Concordant read mapping may potentially indicate balanced rearrangements. However, any balanced rearrangements would be indistinguishable from normal on-target editing, preserving the natural chromosomal structure. The alternative outcome would be discordant mapping of the paired-end sequencing reads. Discordant mapping can potentially indicate the presence of structural variations after DNA repair, such as inter-chromosomal translocation, inversion, or duplications.

The SV characterization assay was performed on two donor lots of PHH treated with NTLA-2001. High molecular weight gDNA was isolated and libraries were prepared as described above. NGS libraries were sequenced using Illumina MiSeq or NextSeq NGS technology using 150 bp paired-end sequencing reads and two 8 bp dual indexing reads. NGS reads were analyzed for DNA SVs using code developed in-house. Briefly, each read or read pair was aligned to the reference genome (GRCh38). Discordant reads, or split NGS alignments, were defined as read or read pair whose 5′ and 3′ ends were aligned to two different locations in the genome greater than the maximal size of DNA inserts anticipated from a wild-type genome (300 bp for single-read and 1,000 bp for read pair). When NGS aligned to more than one locus in the genome, the two fragments involved were used to classify the SV with the following criteria: (1) intra-chromosomal translocation; (2) inter-chromosomal; (3) inversion; and (4) duplication. If the alignment matched signatures of more than one class, then it was classified as ‘complex.’ Recurrent DNA SVs were defined as having greater than one unique molecular identifier representing the DNA SV detected.

NTLA-2001 genome edited PHH exhibited low (<1%) DNA SV repair outcomes at super saturating levels of on-target editing. The frequencies of DNA SV detected after genome editing with NTLA-2001 are concordant with previously reported results for high-efficiency editing gRNAs. None of the identified translocations were associated with any known risk, and the only recurrent translocations detected were acentric and dicentric fusions between the on-target sites of sister chromosomes.

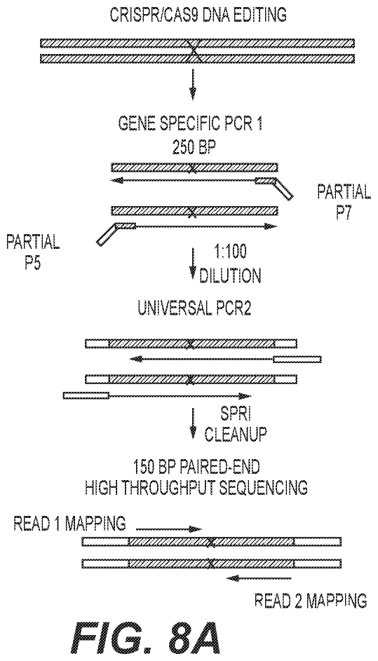

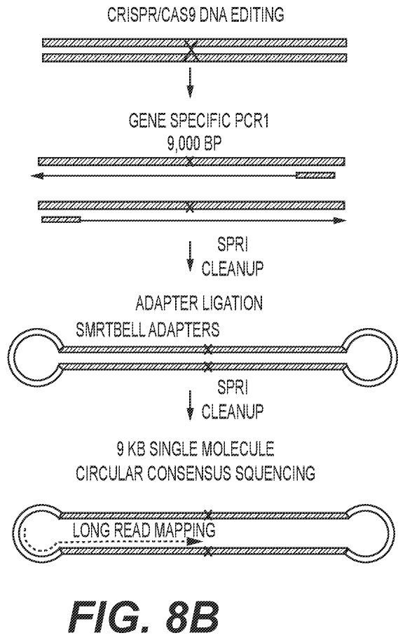

The potential for kilobase pair (Kb) deletions of DNA to result as a potential repair outcome after genome editing with CRISPR/Cas9 has been previously reported in mouse embryonic stem cells, mouse hematopoietic progenitors, and a human differentiated cell line. Targeted PCR-based amplicon sequencing with Illumina-based NGS is limited in its capacity to characterize and quantify large structural variants such as deletions >100 bp. Therefore, to characterize the potential for large deletions to occur as a result of DNA repair after genome editing with NTLA-2001, long-range PCR followed by long-read sequencing with Pacific Biosciences technology was conducted at the Icahn School of Medicine at Mt Sinai in New York (USA) and was qualified by determining the detection limit of a 966 bp deletion (FIG. 8).

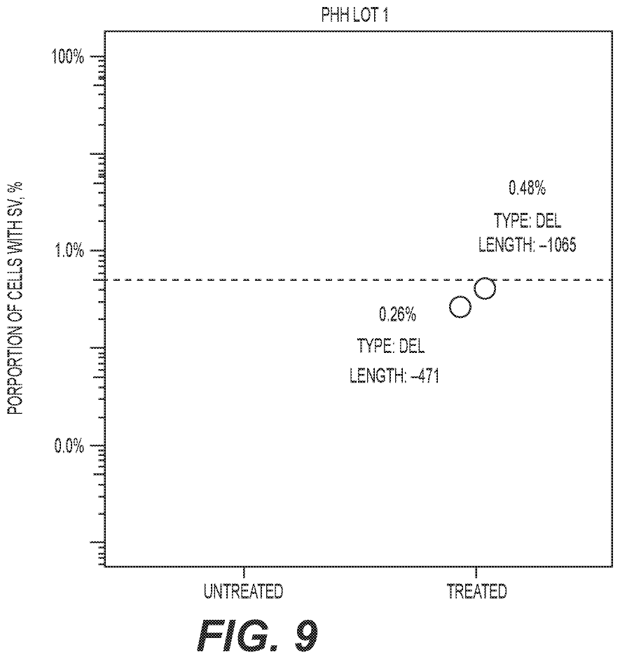

Two donor lots of PHH were treated with NTLA-2001 and gDNA was isolated for long-range PCR and sequenced using Pacific Biosciences Sequel II instrument at the Icahn School of Medicine at Mt Sinai, New York (USA). Analysis of on-target indel frequency with standard short-read Amp-Seq after genome editing with NTLA-2001 in PHH revealed on-target indel editing frequencies of 92.57±7.85% (lot 1) and 93.50±0.10% (lot 2). Analysis of long-range PCR followed by long-read sequencing with Pacific Biosciences technology of NTLA-2001 in PHH genome edited in vitro at super-saturating genome editing doses revealed a low frequency of two deletions, sized 471 bp and 1,065 bp, in one of two PHH donors with 0.26% and 0.48% of the reads, respectively (FIG. 9). The nearest gene to the on-target site of NTLA-2001 is ˜28 Kb away, therefore the DNA structural variants characterized in this report are considered productive genome editing outcomes of NTLA-2001 that resulted in a disruption of the TTR gene without additional unintended genomic alterations in coding DNA sequence.

### Example 6. Dose-Dependent and Durable Effects in Transgenic Mice

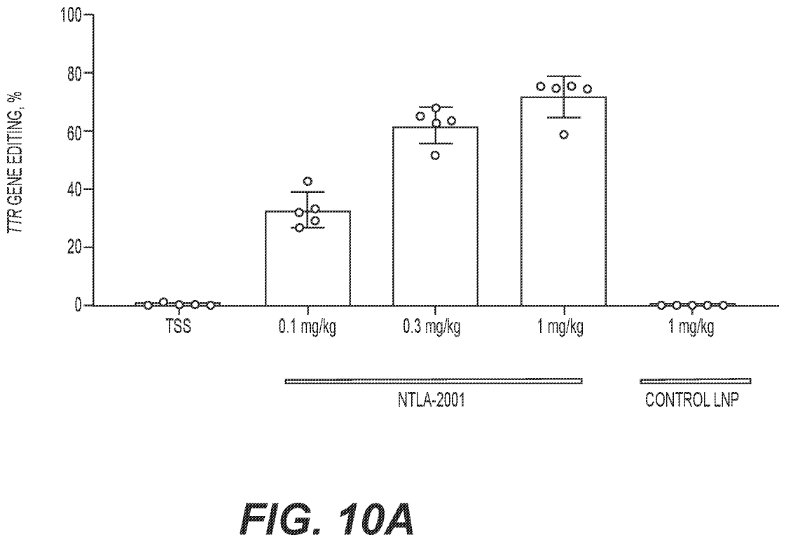

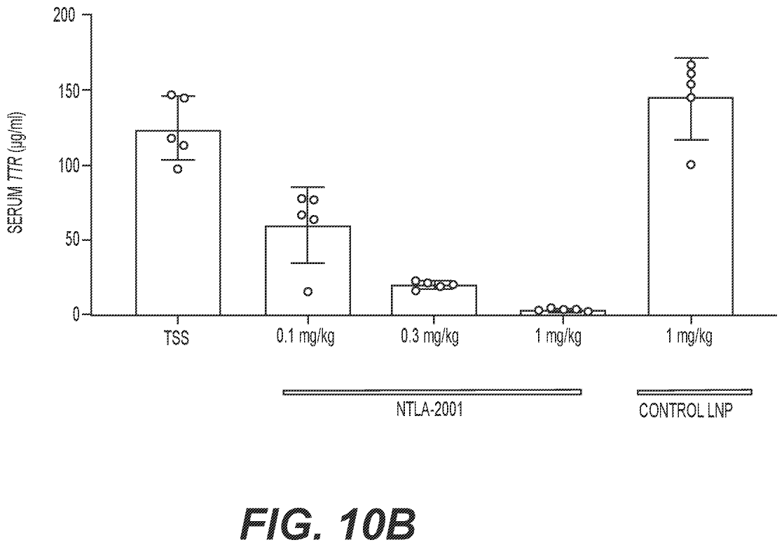

2001. Studies in transgenic mice revealed a dose-dependent and durable effect of NTLA-In a first experiment, huTTR transgenic mice treated with 0.1, 0.3, or 1 mg/kg NTLA-2001, 1 mg/kg non-targeting control lipid nanoparticle (LNP), or tris sucrose saline buffer control (n=5 mice per group). Liver TTR gene editing (panel A) and serum human TTR protein (panel B) were measured 7 days post-dose via next-generation sequencing and human transthyretin enzyme-linked immunosorbent assay respectively. Mean and standard deviation values are shown from the five mice treated in each group (FIG. 10).

Editing of the TTR gene reduced circulating serum TTR protein levels, which reached nadir by 4 weeks post dose and were still maximally suppressed at 12 months' observation.

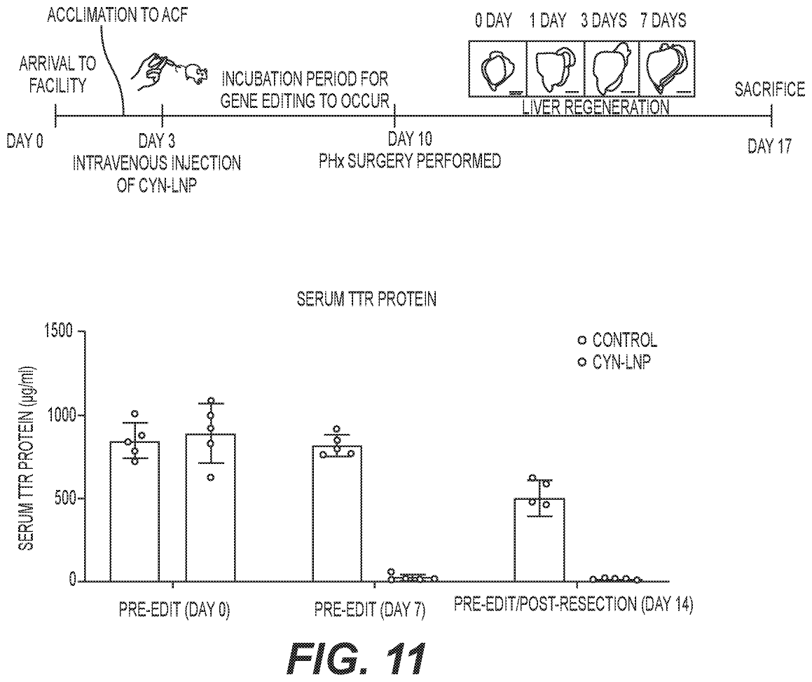

In a second experiment, after resection of two-thirds of the liver, and subsequent full-liver regeneration, gene editing percentage and corresponding protein levels were unchanged, supporting the permanent nature of the edit (FIG. 11). CD1 mice were treated with 1 mg/kg lipid nanoparticle (LNP) containing CRISPR/Cas9 mRNA and a single guide RNA targeting the TTR gene, or with tris sucrose saline (TSS) control (n=5 mice per group). At day 7, mice underwent partial hepatic resection (PHx) to remove approximately 70% of the liver. Serum TTR protein concentration was measured post dose at day 0, day 7 (pre-PHx), and day 17 (day 4 post-PHx) via TTR enzyme-linked immunosorbent assay. Mean and standard deviation values are shown from the five mice treated in each group.

Seven days following administration of the LNP formulation the animals demonstrated a 98% knockdown of serum TTR, which was maintained following the regeneration of the liver after PHx. These LNP-treated animals also demonstrated an identical TTR gene editing percentage (73%) both before and after the PHx, indicating that the genetic edits are maintained through the liver regeneration process.

### Example 7. LNP-Mediated Editing in Non-Human Primates

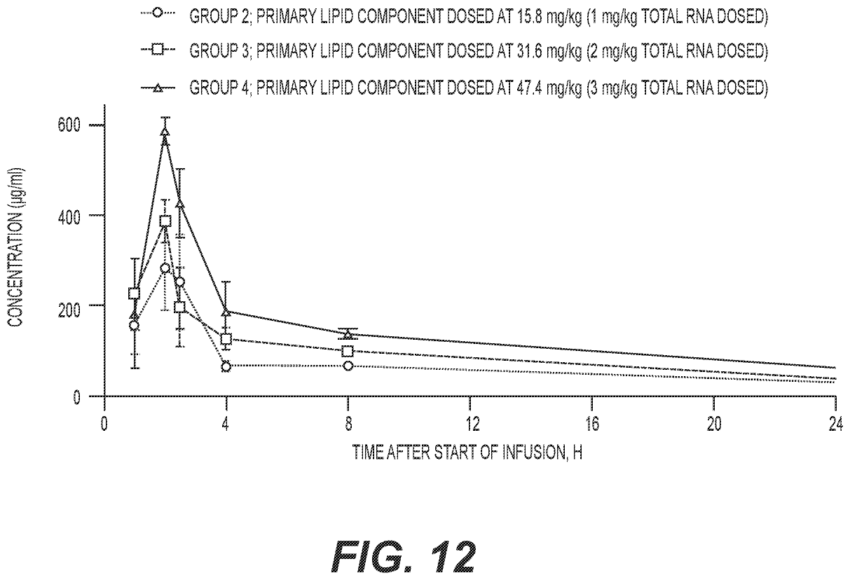

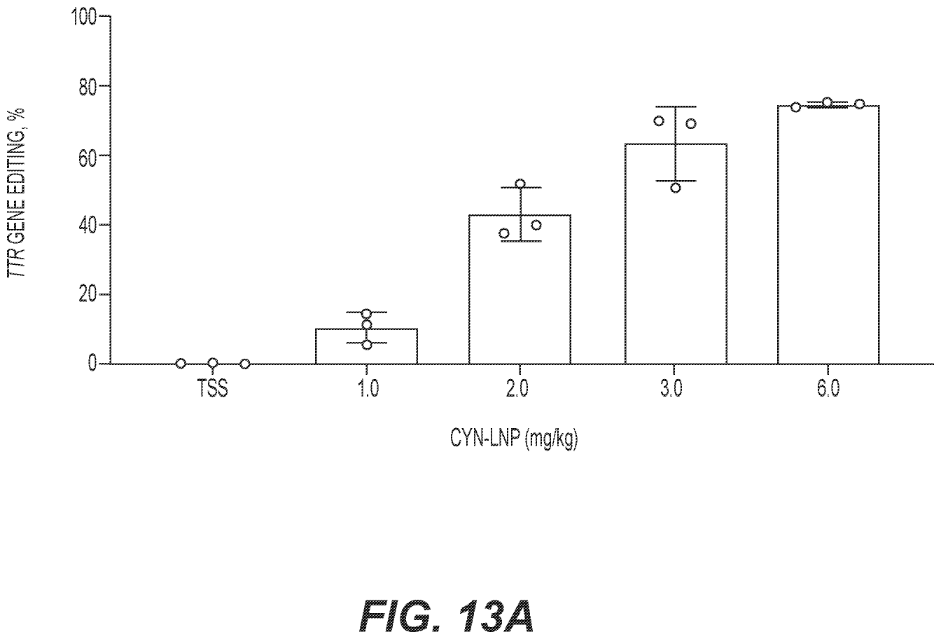

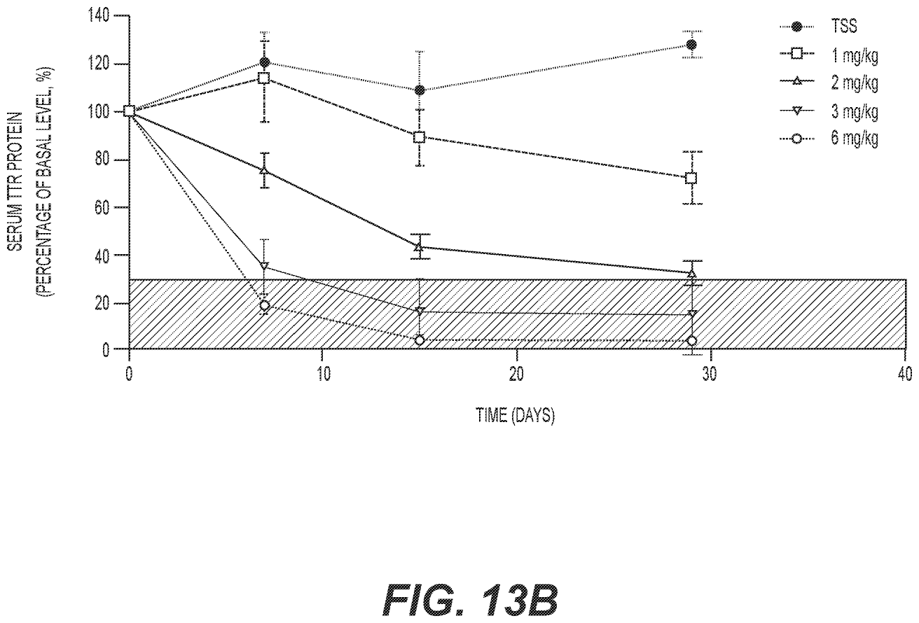

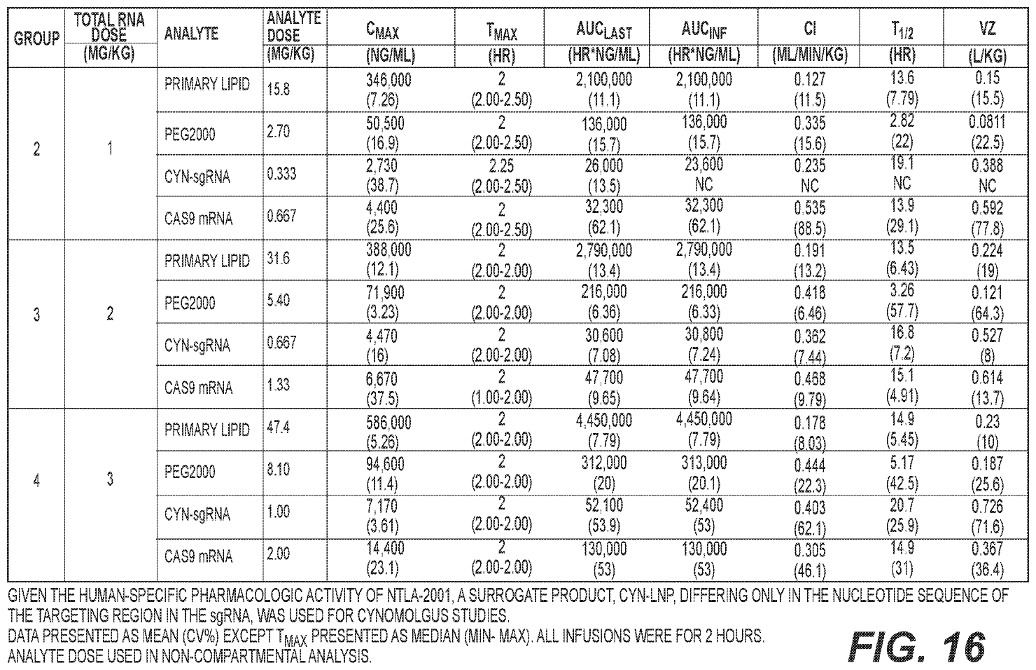

Three cynomolgus monkeys per dose group (1, 2, 3, and 6 mg/kg) were pretreated with dexamethasone at least 1 hour before Cyn-LNP infusion to mimic planned clinical pretreatment. Transthyretin (TTR) gene editing in liver was evaluated using next-generation sequencing on day 29. Serum TTR protein concentrations were assayed by liquid chromatography with tandem mass spectrometry and reported as a percentage of basal (day 0) value. TTR gene editing exhibited dose-responsiveness between 1 and 6 mg/kg (Panel A), which corresponded to diminished serum TTR protein levels relative to baseline (Panel B). Mean and standard deviation values are displayed for each treatment group. The shaded box in Panel B indicates the therapeutically relevant range of TTR protein reduction. (FIG. 13). Cynomolgus monkey studies demonstrated rapid initial distribution and clearance of the LNP components (FIG. 16 and FIG. 12).

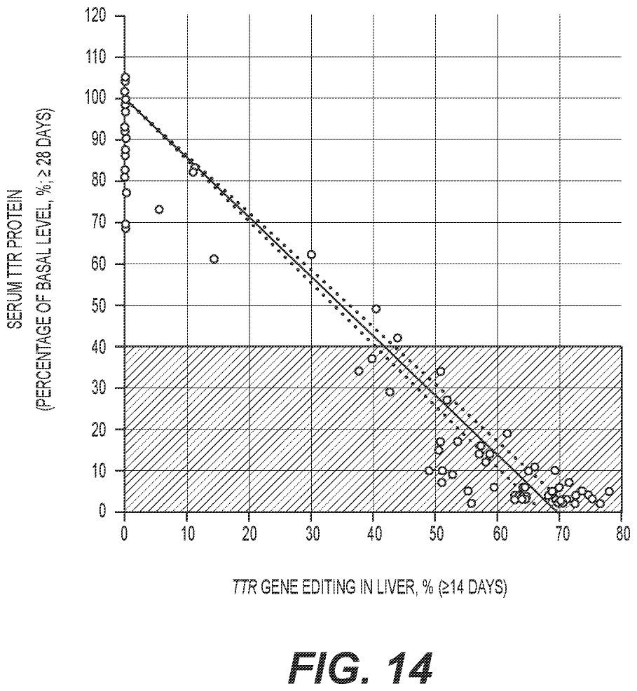

FIG. 14 is an integrated summary plot of single-dose pharmacokinetics of Cyn-LNP in cynomolgus monkeys.

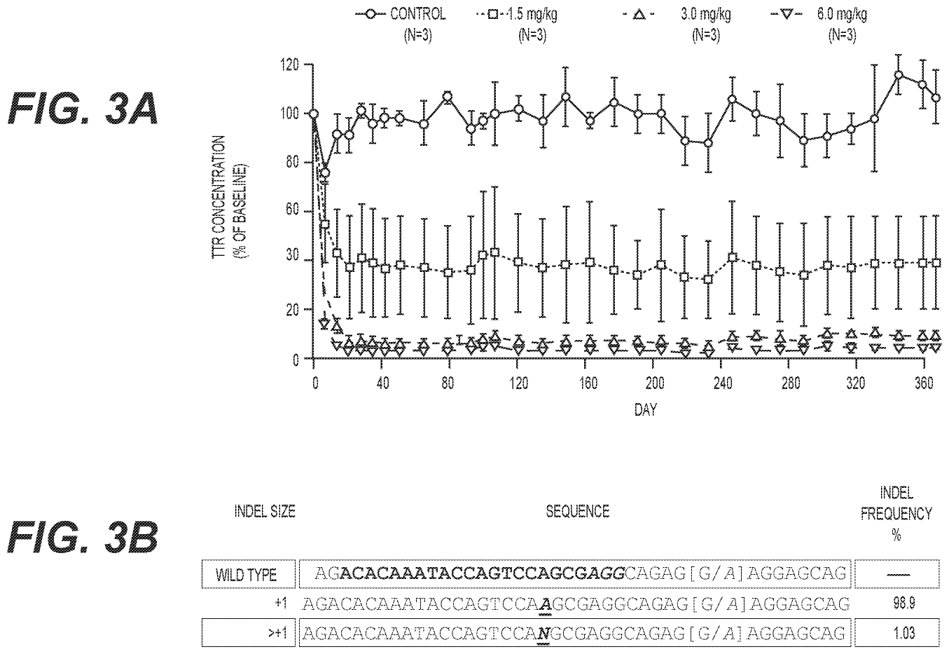

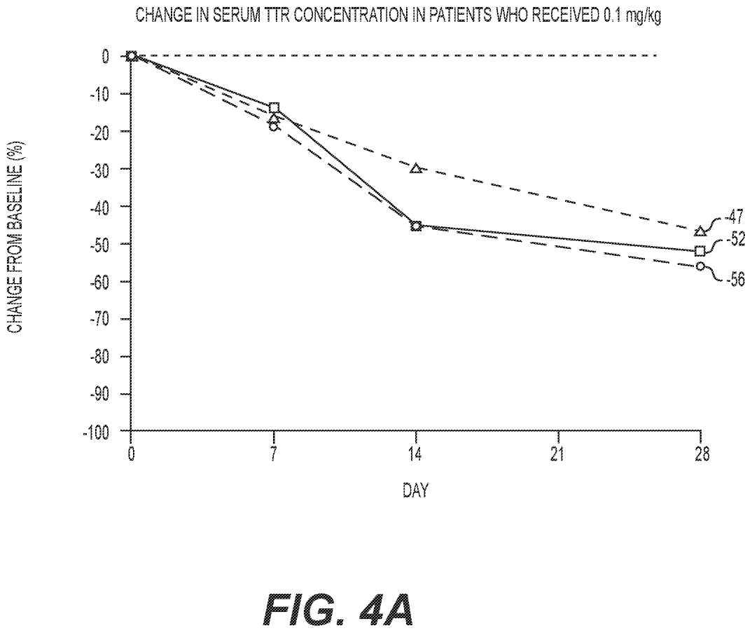

In addition, a single dose of Cyn-LNP at 3 or 6 mg/kg was associated with a gene-editing percentage (maximum) of 73% in whole liver and near-complete reduction of serum TTR (>94%) that was sustained over 12 months (FIG. 3A). Editing of the TTR gene was confirmed by NGS analysis of hepatic tissue (FIG. 3B).

FIG. 3A shows mean reduction in serum transthyretin (TTR) protein concentration as a proportion of baseline in cynomolgus monkeys (n=3 per cohort) receiving intravenous administration of the Cyn-LNP at doses of 1.5, 3.0, and 6.0 mg/kg (total RNA/body weight) on day 0 and followed up for 367 days. A control cohort receiving no treatment is provided for comparison. Vertical lines for each point indicate standard deviations across the three animals in each group. Panel B shows the results of next-generation sequencing data following Cyn-LNP administration to cynomolgus monkeys. The guide RNA target sequence is indicated in blue next to the required PAM sequence in red. The [G/A] represents a naturally occurring SNP among the cynomolgus monkeys used in the study. The nucleotide position of indels relative to the cynomolgus genome build mf5 chromosome 18 are as follows:
+1:50681549-50681550.
The primary indel pattern was a single nucleotide insertion at the cut site inducing a frameshift mutation. An “N” at the insertion site refers to multi-nucleotide insertions (AA, AGG, etc.) which in aggregate constituted 1.03% of all indels. The remaining fraction comprised deletions of varied length. sgRNA denotes single-guide RNA.

TABLE 2
Predicted transthyretin protein reduction in humans
based on modeling from non-human primate studies

Predicted reduction in TTR protein, %

| Cargo dose | | 4.6-fold more |
|---|---|---|
| (mg/kg) | Equipotent* | potent* in humans |
| 0.1 | 24 | 58 |
| 0.3 | 48 | 80 |

Predictions are based on a body weight of 70 kilograms for human subjects.

### Example 8. Clinical Trial

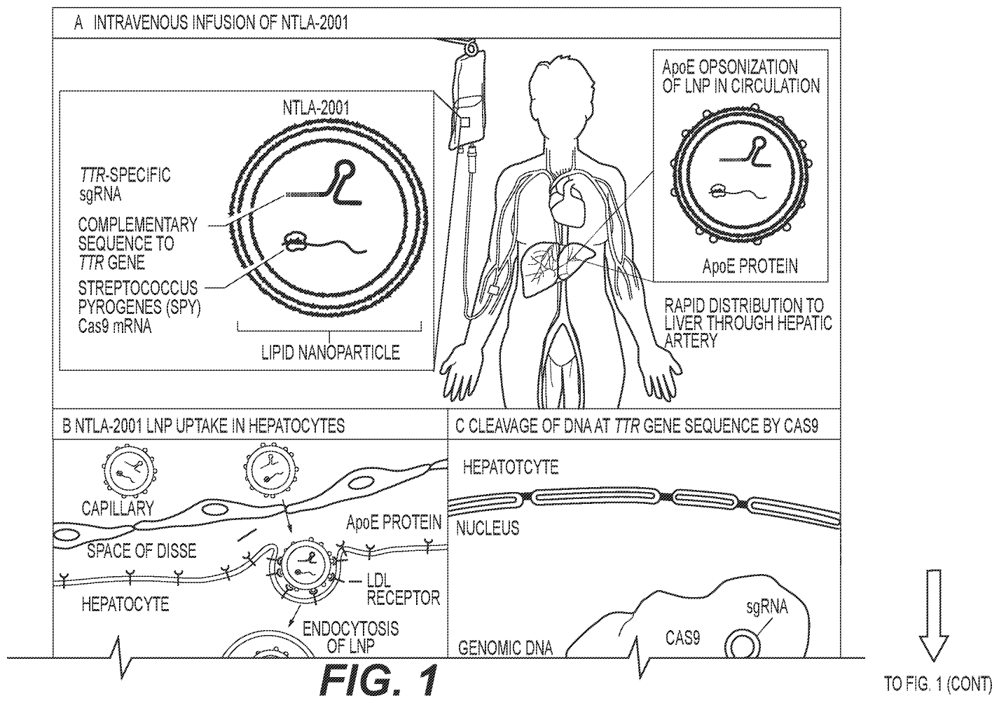

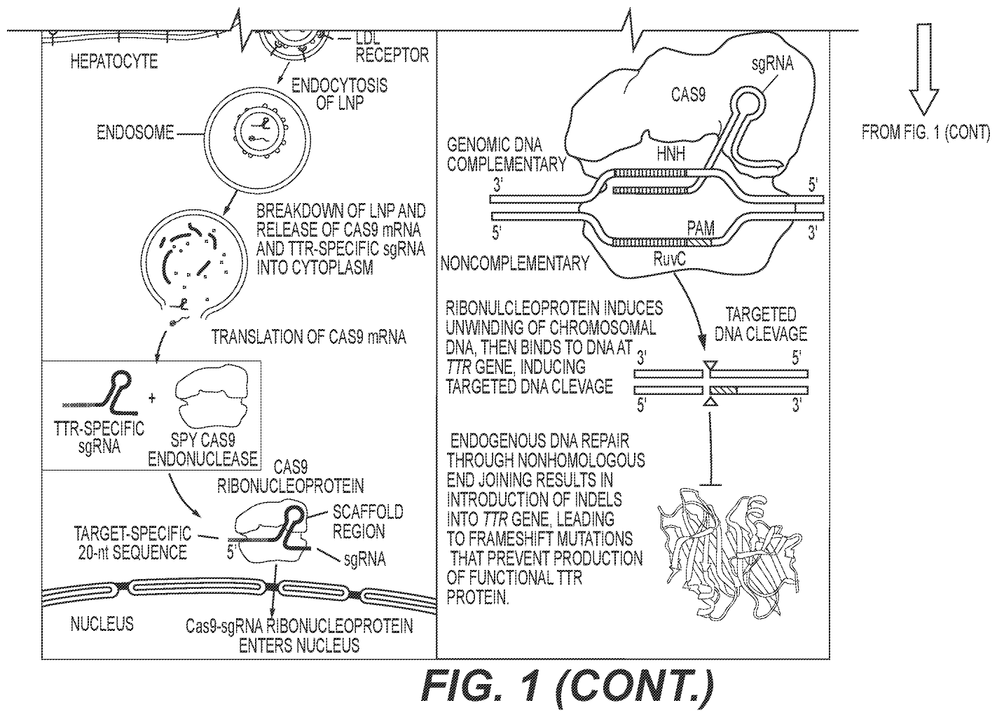

The overall treatment design is summarized in FIG. 1. Panel A shows the primary components of NTLA-2001. The carrier system for NTLA-2001 is a lipid nanoparticle (LNP). The LNP formulation is described herein. The active components of NTLA-2001 are a human-optimized messenger RNA (mRNA) molecule encoding Streptococcus pyogenes (Spy) Cas9 protein (an approximately 4400-nucleotide sequence with a molecular weight of approximately 1.5 MDa) and a single guide RNA (sgRNA) molecule (molecular weight of approximately 35 kDa) specific to the human gene encoding transthyretin (TTR). These components form the cargo of the LNP for drug administration. After intravenous administration of NTLA-2001 and entry into the circulation, the LNP is transported through the systemic circulation directly into the liver, where it is preferentially distributed. Panel B illustrates the transport of the NTLA-2001 LNP into the capillaries of the hepatic sinusoids inside the liver. Similar to other clinically approved LNPs, NTLA-2001 is opsonized by apolipoprotein E (ApoE) in the circulation and is then expected to undergo uptake by the low-density lipoprotein (LDL) receptor expressed on the surface of the hepatocytes, followed by endocytosis and endosome formation. After breakdown of the LNP and disruption of the endosomal membrane, the active components (the TTR-specific sgRNA and the mRNA encoding Cas9) are released into the cytoplasm. The Cas9 mRNA molecule is translated through the native ribosomal process, producing the Cas9 endonuclease enzyme. The TTR-specific sgRNA interacts with the Cas9 endonuclease, forming a clustered regularly interspaced short palindromic repeats (CRISPR)-Cas9 ribonucleoprotein (RNP) complex. Panel C shows that the Cas9 RNP complex is targeted for nuclear import and enters the nucleus, where it recognizes the protospacer-adjacent motif (PAM) on the noncomplementary DNA strand in TTR. A target-specific 20-nucleotide sequence at the 5′ end of the sgRNA binds to the DNA double helix at the target site, allowing the CRISPR-Cas9 complex to unwind the helix and access the target gene. Cas9 undergoes a series of conformational changes and nuclease domain activation (HNH and RuvC domains), resulting in DNA cleavage that is precisely targeted to the TTR sequence, as defined by the sgRNA complementary sequence. Endogenous DNA-repair mechanisms ligate the ends of the cut, potentially introducing insertions or deletions of bases (indels). The generation of an indel may result in the reduction of functional target-gene mRNA levels as a result of missense or nonsense mutations decreasing the amount of full-length mRNA, ultimately resulting in decreased levels of the target protein. Indels that result in abrogated production of the target protein, in this case TTR, are termed knockout mutations.

a. Polyneuropathy Dose Escalation Study

### Polyneuropathy Cohorts 1 and 2

### Enrollment

At one study site, three subjects were screened, of whom two were eligible and were recruited. One subject weighed above the upper limit allowed by study protocol at that time. At the other study site, four patients were screened, of whom four were eligible and were recruited. Patients were aged 46-64 years and 4/6 were male; body weight was 70-90 kg. Three patients had a p. T80A mutation, two a p.S97Y mutation, and one a p.H110D mutation. Three patients had received no prior therapy and three prior diflunisal. All six patients had a polyneuropathy disability score of 1 and a New York Heart Association Functional Classification of I. N-terminal pro-B-type natriuretic peptide (NT-proBNP) ranged between 50 and 596 ng/L.

### Clinical Trial Design and Eligibility

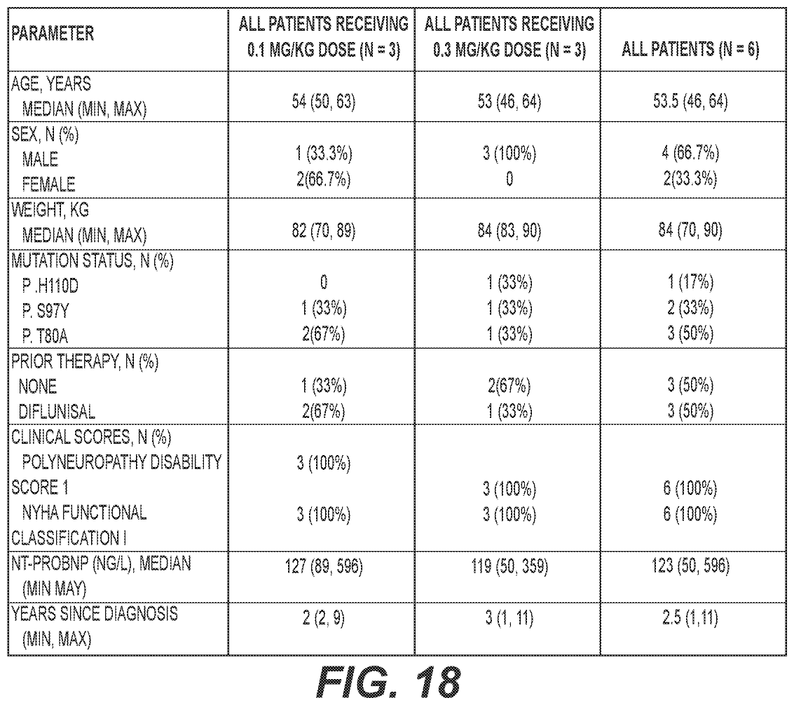

We report two initial cohorts (Cohorts 1 and 2) from Part 1 of a two-part, global, phase 1, open-label, multi-center study. Patients were treated with a single dose of NTLA-2001, total RNA/body mass 0.1 mg/kg or 0.3 mg/kg, administered intravenously, between November 2020 and April 2021. Also reported herein are data from these patients subsequently treated. Key eligibility criteria for Part 1 included age 18-80 years, diagnosis of polyneuropathy due to hATTR amyloidosis (with or without cardiomyopathy), body weight 50-90 kg at screening visit, and lack of access to approved treatments for ATTR amyloidosis. Patients with non-ATTR amyloidosis, known leptomeningeal ATTR amyloidosis, or prior history of RNA silencing therapy were excluded. Prior use of TTR stabilizers was permitted with a washout period (diflunisal: 3 days) (FIG. 18).

### Clinical Trial Safety

Safety studies in cynomolgus monkeys determined the no-observed-adverse-effect level (NOAEL) as a single administration of 3 mg/kg infused intravenously, equivalent to a dose of 1 mg/kg in humans. Following allometric scaling based on total body surface area and application of a safety factor of 10, the maximum recommended starting dose of NTLA-2001 for this study was 0.1 mg/kg. To mitigate against potential pro-inflammatory effects of intravenous LNP infusions, patients received glucocorticoid and histamine receptor type-1 and type-2 blockade before infusion.

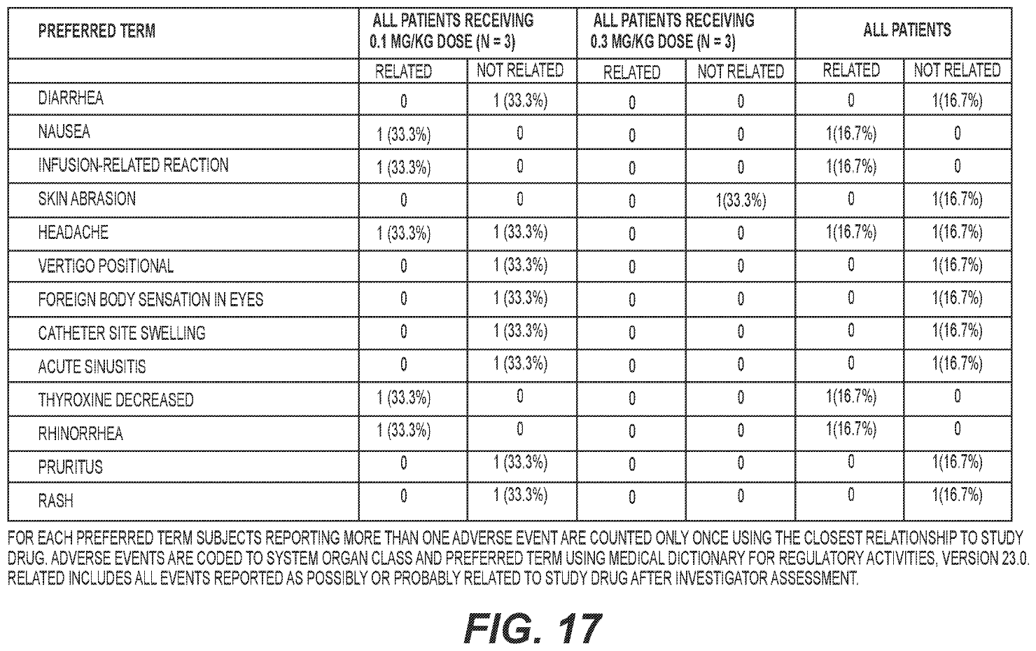

NTLA-2001 treatment was completed without interrupting the infusion. No protocol-specified stopping events were observed. Treatment-emergent adverse events were reported in 3 of 6 patients, all of which were mild (Grade 1) in severity. One patient experienced an adverse event of special interest (Grade 1 infusion-related reaction; see FIG. 17). No serious adverse events were observed.

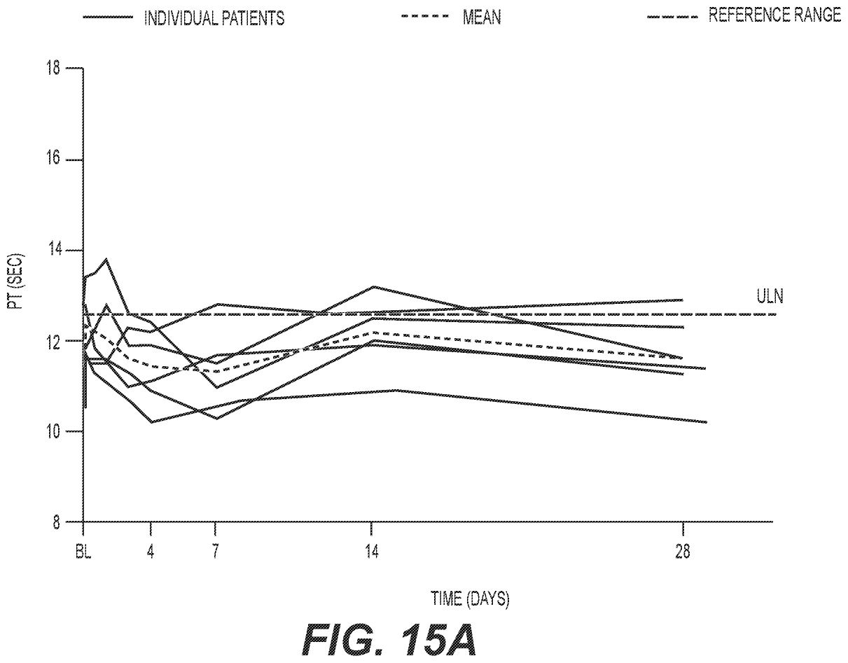

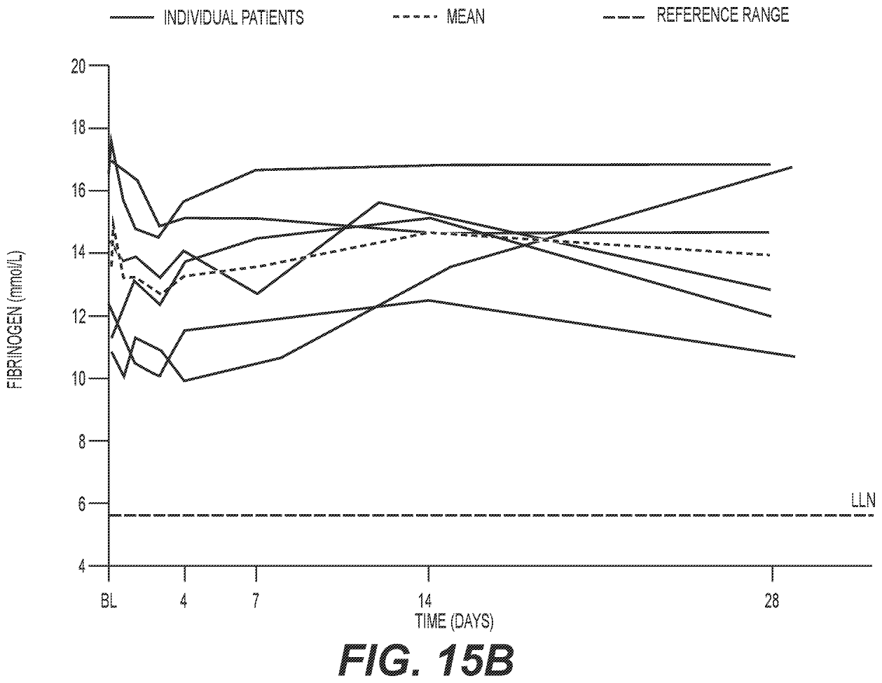

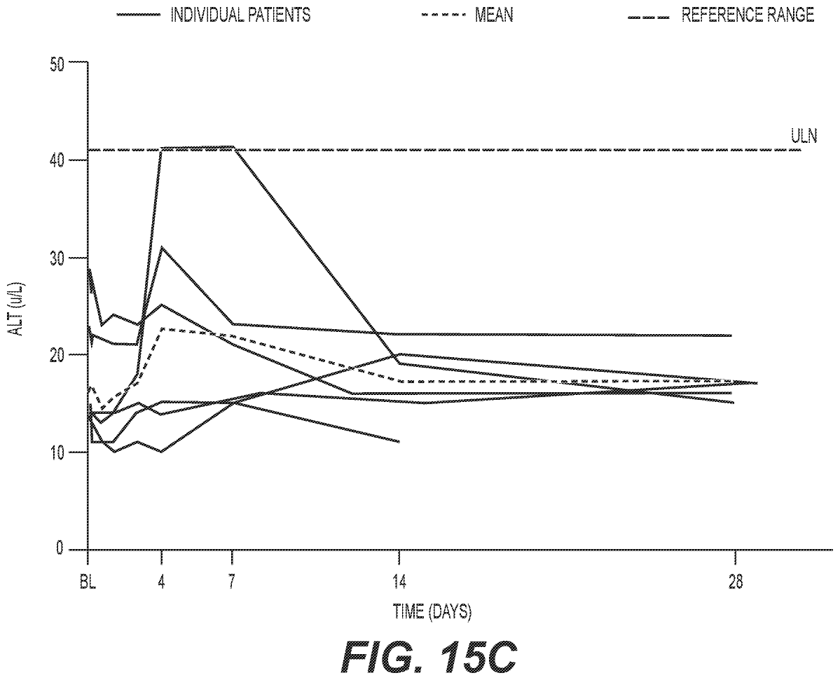

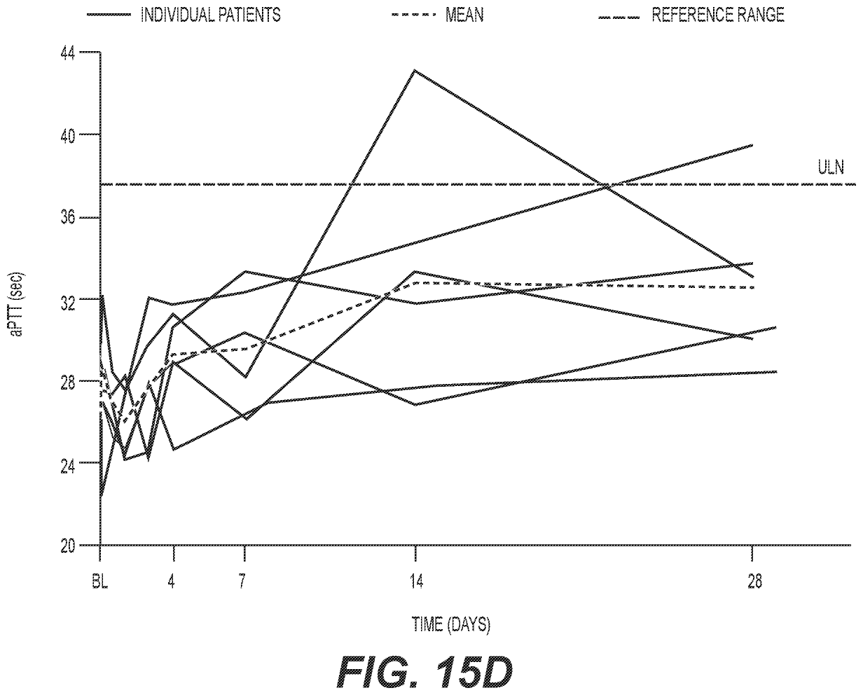

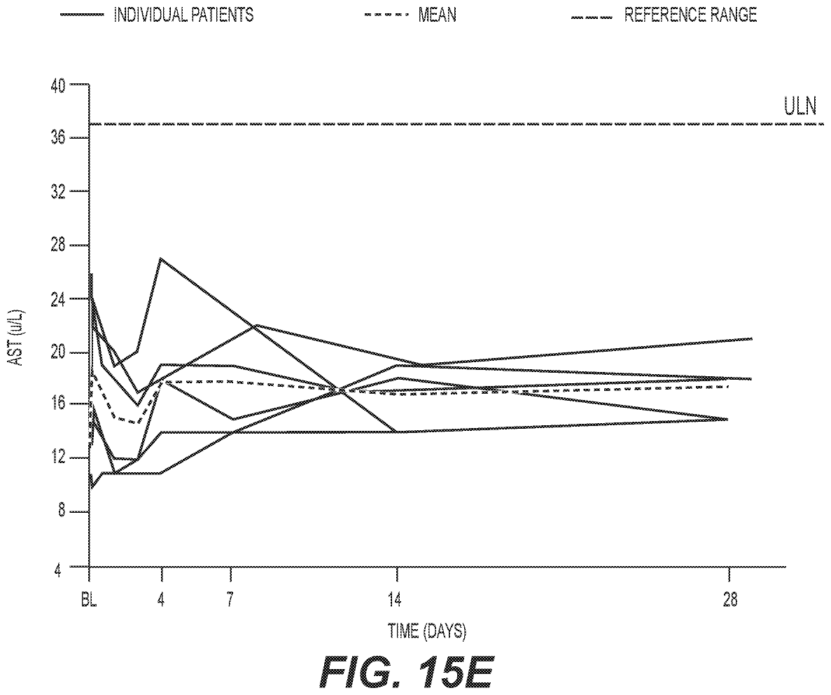

D-dimer levels were assessed by methods known in the art. Increased d-dimer levels were observed 4-24 hours after infusion in 5 of 6 patients; elevations were less than those observed at the NOAEL dose in nonhuman primates. The values returned to baseline in all 6 patients by day 7. Coagulation parameters activated partial thromboplastin time; and prothrombin time were assessed by methods known in the art and results remained within 1.2 times the upper limit of reference ranges. Fibrinogen and platelet counts were performed by methods known in the art and remained above the lower limit of the reference ranges. Liver function tests (aspartate aminotransferase and alanine aminotransferase) were by methods known in the art and results remained within normal limits (FIG. 15).

FIG. 15A displays prothrombin time; FIG. 15B, activated partial thromboplastin time; FIG. 15C, fibrinogen; FIG. 15D, alanine aminotransferase; and FIG. 15E, aspartate aminotransferase. Blue lines indicate individual subjects' results over time. The single red line indicates mean results over time. The horizontal dashed lines mark either the ULN or the LLN, as appropriate, for each parameter. Baseline is defined as the last available measurement taken prior to the start of infusion of study drug. Only results obtained from a central laboratory through day 28 are plotted.

ALT denotes alanine aminotransferase, aPTT activated partial thromboplastin time, AST aspartate aminotransferase, BL baseline, PT prothrombin time, LLN lower limit of normal and ULN upper limit of normal. Patients were monitored for assessment of treatment-emergent adverse events and laboratory findings. Serum samples were obtained at baseline and at weeks 1, 2 and 4 for analysis of TTR protein levels by an enzyme-linked immunosorbent assay (ELISA). Patients are evaluated for safety and therapeutic activity outcomes for 24 months from NTLA-2001 infusion.

### Pharmacokinetics

Interim pharmacokinetic data suggest that following intravenous (IV) infusion, NTLA-2001 ionizable lipid exhibited a rapid decline from peak levels followed by a secondary peak and then a log-linear phase.

### Clinical Trial Efficacy

To determine the pharmacodynamic effect of NTLA-2001, serum TTR levels were evaluated. A sandwich ELISA method was developed and validated as a quantitative assay using human plasma TTR (Sigma, P1742) from healthy subjects as reference standard.

Briefly, assay microplates (Nunc, 446612) were incubated overnight with 1 μg/ml polyclonal rabbit anti-human prealbumin antibody (Dako, A0002) in 0.05 M carbonate coating buffer pH 9.6. Plates were washed four times with TTR wash solution (TTRWS: 0.05% Tween-20, 1× Dulbecco's PBS), blocked with 1× Powerblock (Biogenix, HK085-5k) for 1 hour, and washed four times with TTRWS. Standards, controls and diluted study samples were incubated with the prepared plate for about 2 hours. Plate was washed four times with TTRWS then incubated for 1 hour with sheep anti-human prealbumin antibody (Bio-Rad, AHP1837) diluted 1:2, 500 in 1× Powerblock. The plate was washed four times with TTRWS then incubated for 1 hour with anti-sheep alkaline phosphatase conjugate antibody (Sigma, A5187) diluted 1:10,000 in 1× Powerblock. The plate was washed four times with TTRWS. The plate was developed using SIGMAFAST™ p-Nitrophenyl phosphate Tablets (Sigma-Aldrich, N1891) according to the manufacturer's instructions. After 30 minutes incubation with the development reagent, the reaction was stopped using 2 N Sodium Hydroxide solution. Absorbance was assessed by spectrophotometry. Standard curve, signal (OD) vs. Concentration was generated using human plasma TTR (Sigma, P1742) to quantify QC and unknown samples.

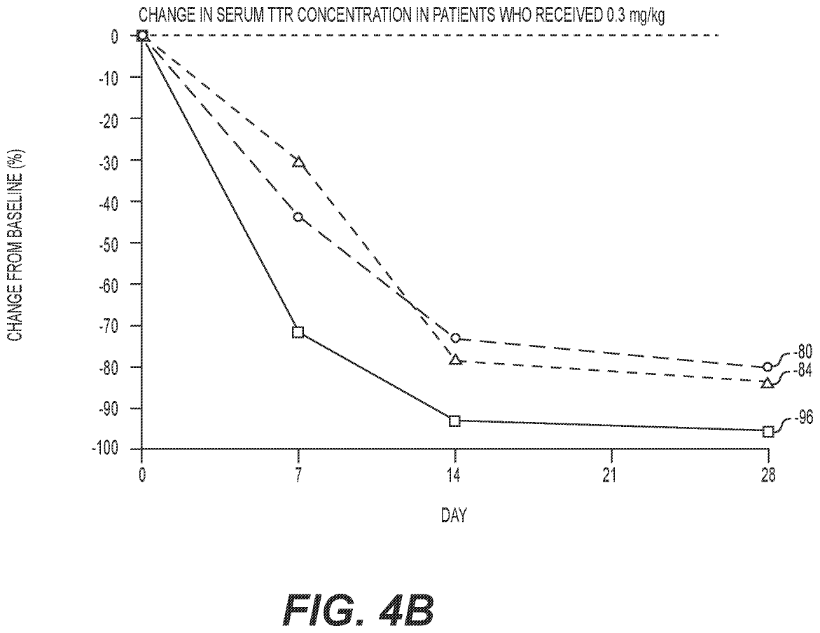

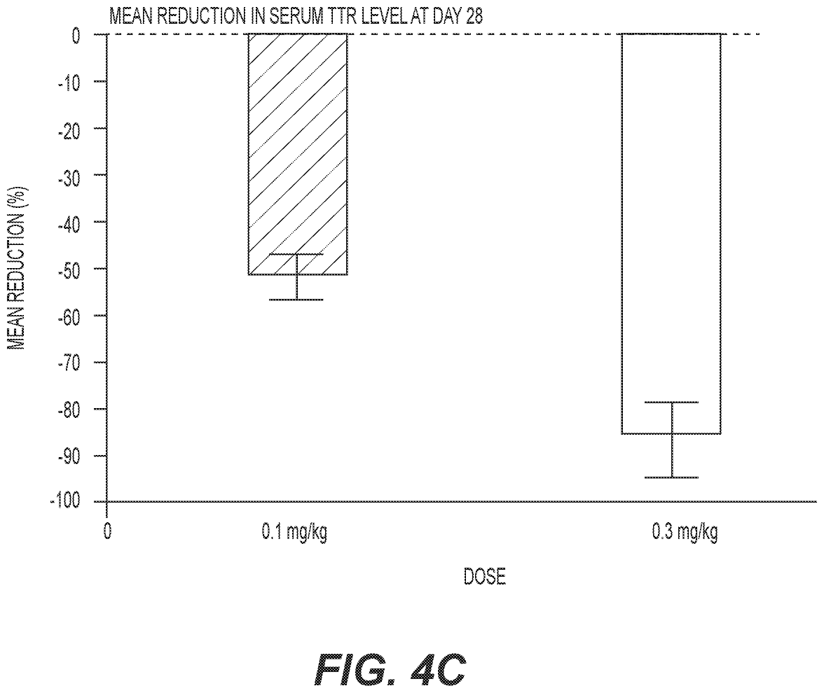

Reductions in serum TTR protein concentration from baseline were observed by day 14 and deepened by day 28 (FIG. 4A). At day 28, NTLA-2001 was associated with mean TTR reductions of 52% in Cohort 1 (dose level 0.1 mg/kg) and 87% in Cohort 2 (0.3 mg/kg; FIG. 4B). The effect was dose-dependent with greater reductions in TTR concentration in patients receiving a higher dose of NTLA-2001. Additionally, the effect of NTLA-2001 was reproducible across individuals at each dose level, with reductions at day 28 ranging from 47-56% (47%, 52%, and 56%) in Cohort 1 and from 80-96% (80%, 84%, and 96%) in Cohort 2 (FIG. 4C).

Panel A shows percentage change in total circulating serum transthyretin (TTR) protein from baseline for Cohort 1 (0.1 mg/kg). TTR protein was quantified by a validated enzyme-linked immunosorbent assay method following regulatory guidelines for biomarker method validation. Serum samples were measured once, with each sample tested in duplicate. As per good laboratory practice, no re-test was conducted for successful assay runs. For each patient in Cohort 1 (0.1 mg/kg), data are illustrated at post-dose day 7, 14, and 28 for percentage reductions in serum TTR protein over pre-dose baseline (mean concentration from three sampling time points). Panel B shows percentage change in total circulating serum TTR protein from baseline for Cohort 2 (0.3 m/kg). Methods and analysis are identical to those described in Panel A. Panel C illustrates mean (N=3 per cohort) percentage reduction in total circulating serum TTR protein from baseline at day 28 for both Cohort 1 and Cohort 2.

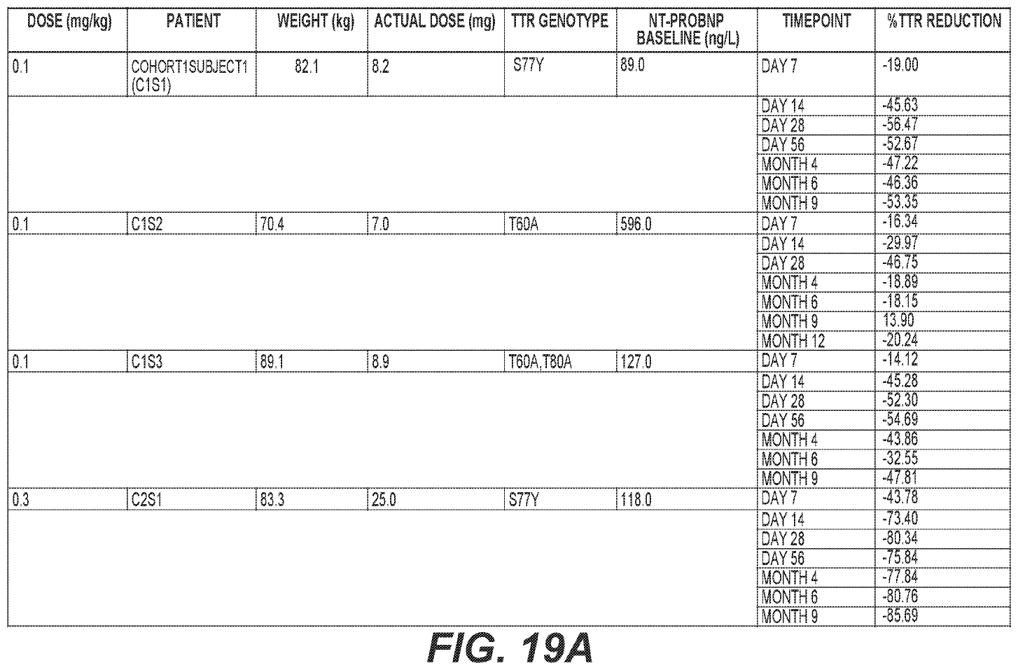

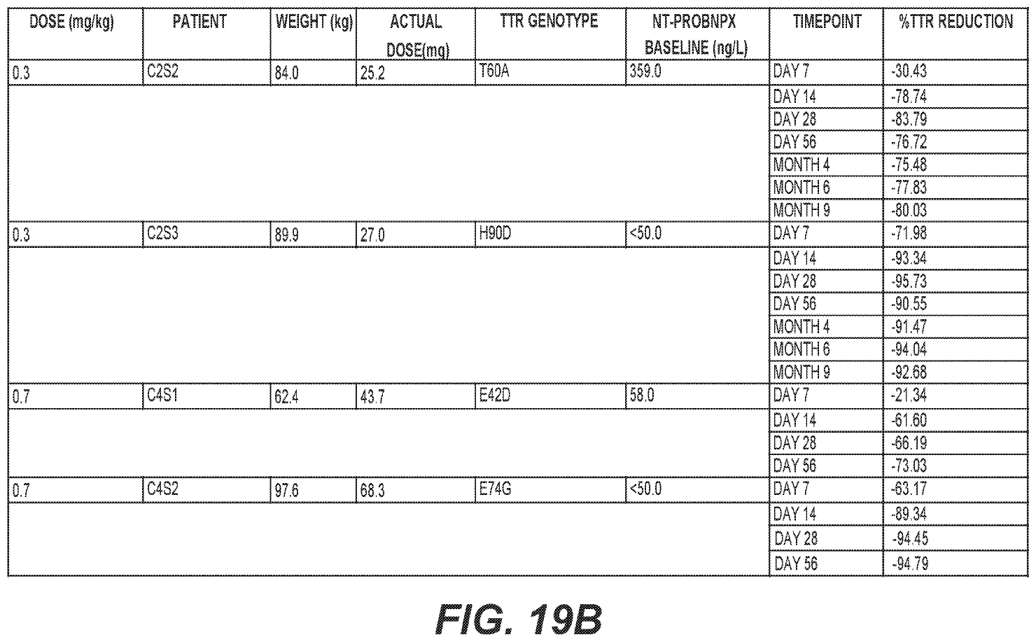

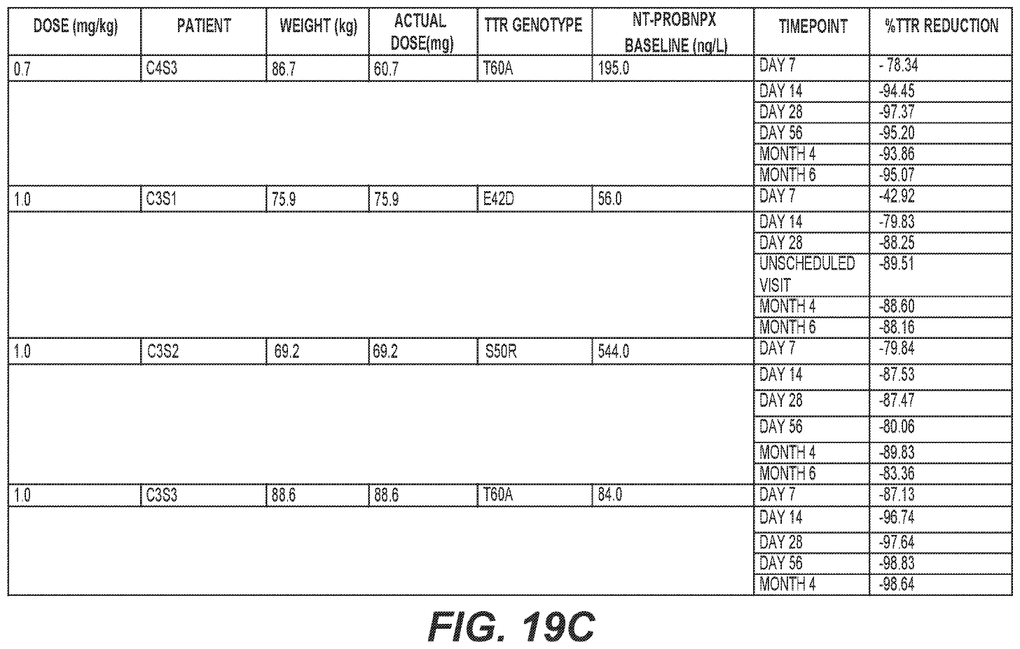

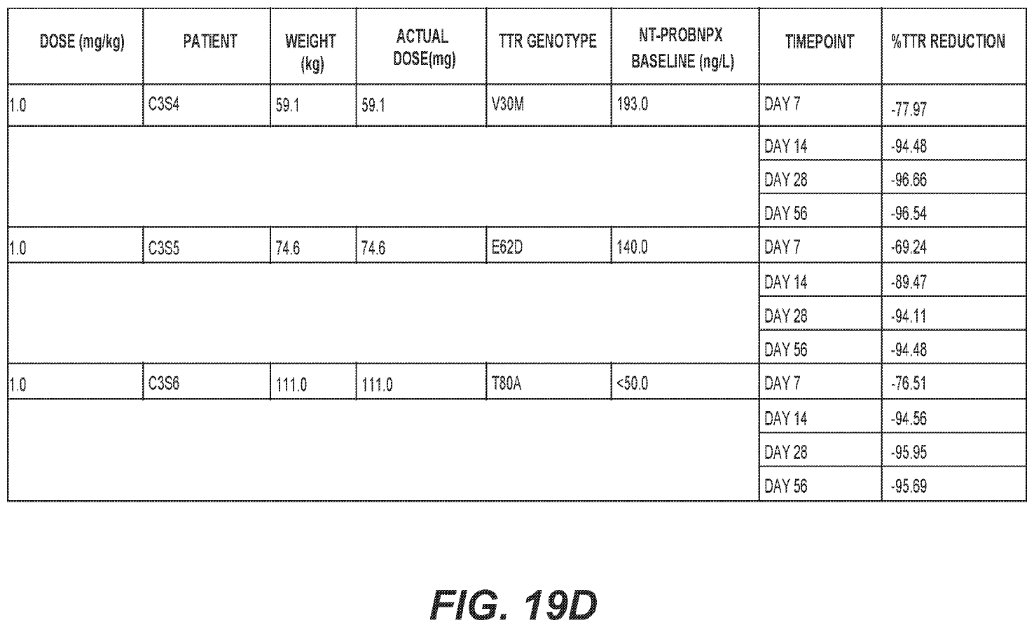

Reductions in serum TTR protein concentration from baseline were observed through day 28, as noted above. Reductions in serum TTR protein concentration from baseline were also observed through month 9 (cohort 1 subject 1 and cohort 1 subject 3, FIG. 19A) or through month 12 (cohort 1 subject 2, FIG. 19A) following treatment with NTLA-2001. Mean percent TTR reduction at day 28 was 52% in cohort 1 (dose level 0.1 mg/kg) and 87% in cohort 2 (dose level 0.3 mg/kg). Mean percent TTR reduction at month 2 was 54% in cohort 1 and 81% in cohort 2. At 9 months post-treatment, mean serum TTR reduction in cohort 2 was 86% (FIGS. 19A and 19B). Mean percent TTR reduction at month 12 was maintained at 89% in cohort 2 (Table 4).

### Polyneuropathy Cohorts 3 and 4

### Enrollment

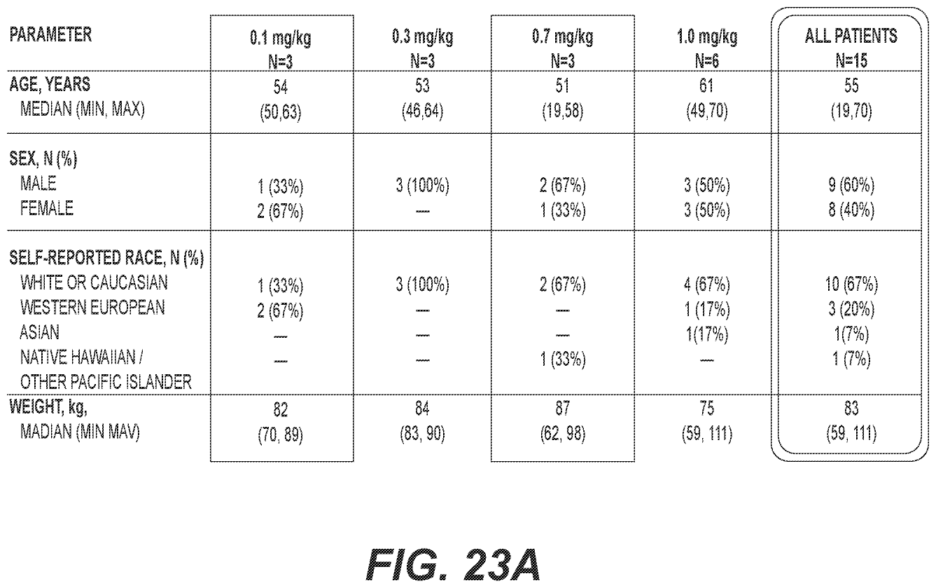

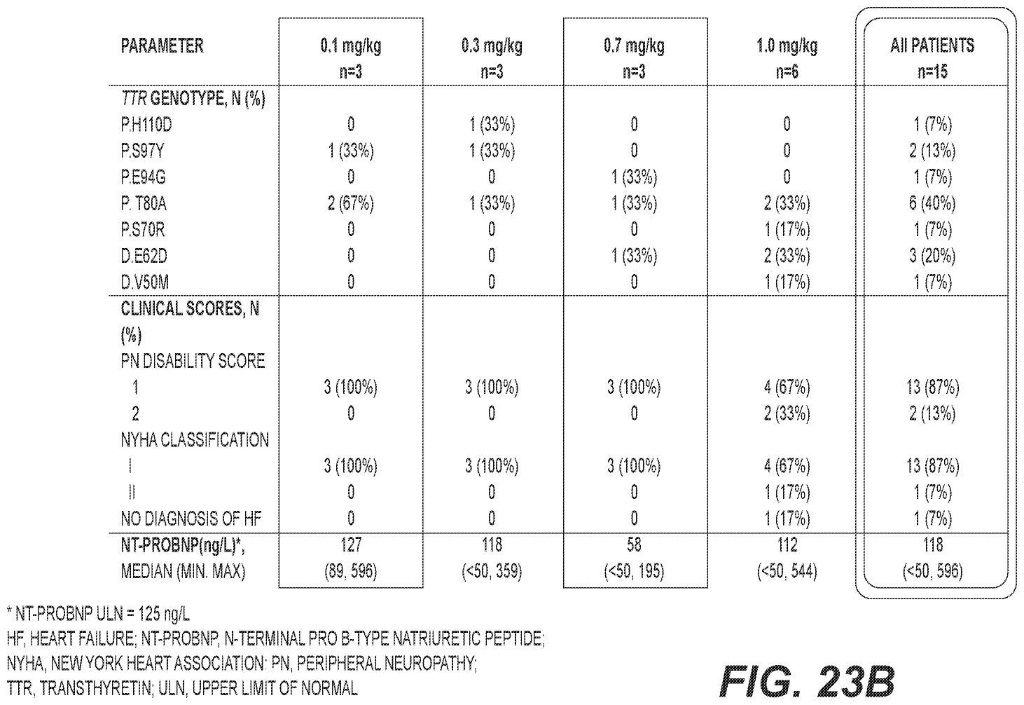

Six subjects were recruited for Cohort 3 and three subjects were recruited for Cohort 4. Subjects were aged 19-70 years; 5 of the 9 subjects were male; and body weight was 59-111 kg. Three subjects had a p. T80A mutation, two had a p.E62D mutation, one had a p.S70R mutation, one had a p. V50M mutation, and one had a p.E94G mutation. Seven subjects had a polyneuropathy disability score of 1, and two had a polyneuropathy disability score of 2. Seven subjects had a New York Heart Association Functional Classification of I, one had a classification of II, and one had no diagnosis of heart failure. N-terminal pro-B-type natriuretic peptide (NT-proBNP) ranged between 50 and 544 ng/L (FIGS. 23A and 23B).

### Clinical Trial Design and Eligibility

Included herein are interim results of Cohorts 3 and 4 from Part 1 of a two-part, global, phase 1, open-label, multi-center study. Patients were treated with a single dose of NTLA-2001, total RNA/body mass 1.0 mg/kg (6 patients in Cohort 3) or 0.7 mg/kg (3 patients in Cohort 4), administered intravenously. Enrollment and eligibility criteria are as described herein.

### Clinical Trial Safety

Safety studies in cynomolgus monkeys determined the no-observed-adverse-effect level (NOAEL) as a single administration of 3 mg/kg infused intravenously, equivalent to a dose of 1 mg/kg in humans. To mitigate against potential pro-inflammatory effects of intravenous LNP infusions, patients received glucocorticoid and histamine receptor type-1 and type-2 blockade before infusion.

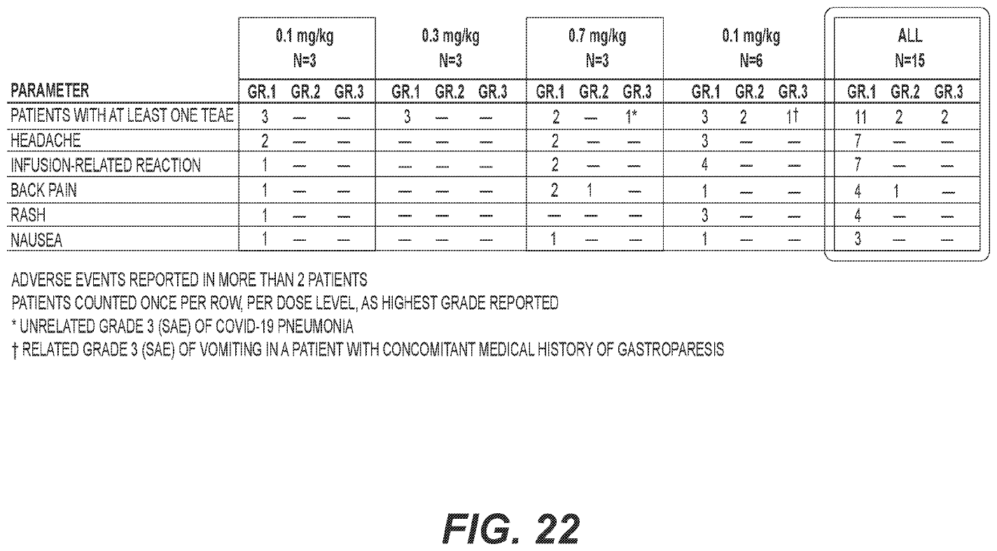

NTLA-2001 treatment was completed without interrupting the infusion. Treatment-emergent adverse events were reported in all 9 subjects. The majority of adverse events were mild in severity (FIG. 22). All infusion-related reactions were considered mild, resolving without clinical sequelae. A single related Grade 3 event (SAE) of vomiting was reported at the 1.0 mg/kg dose in a patient with underlying gastroparesis. No clinically significant laboratory findings observed, with transient Grade 1 liver enzyme elevations observed. No protocol-specified stopping events were observed.

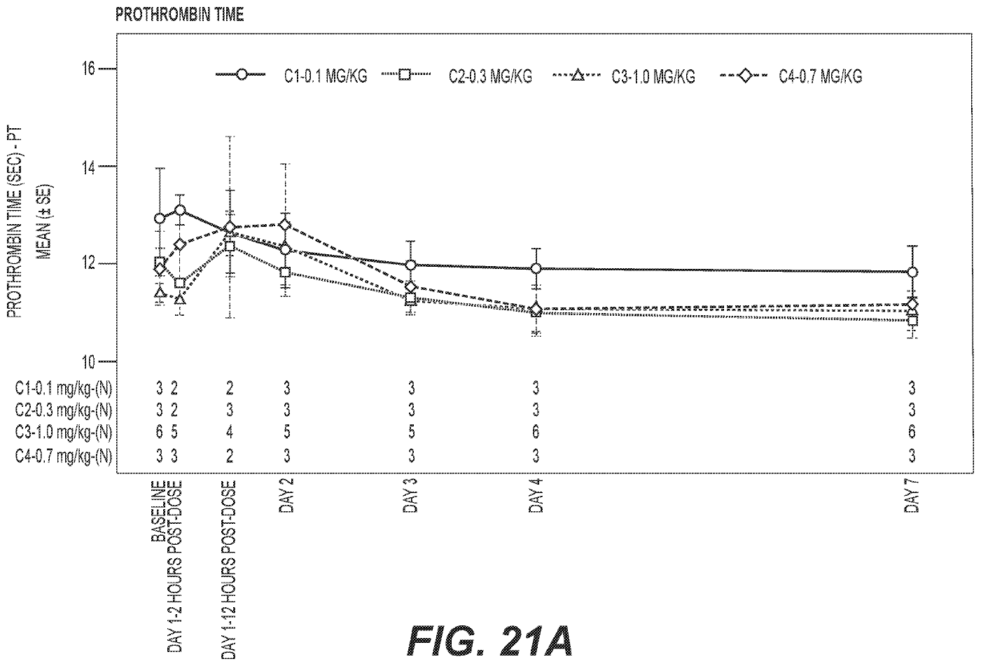

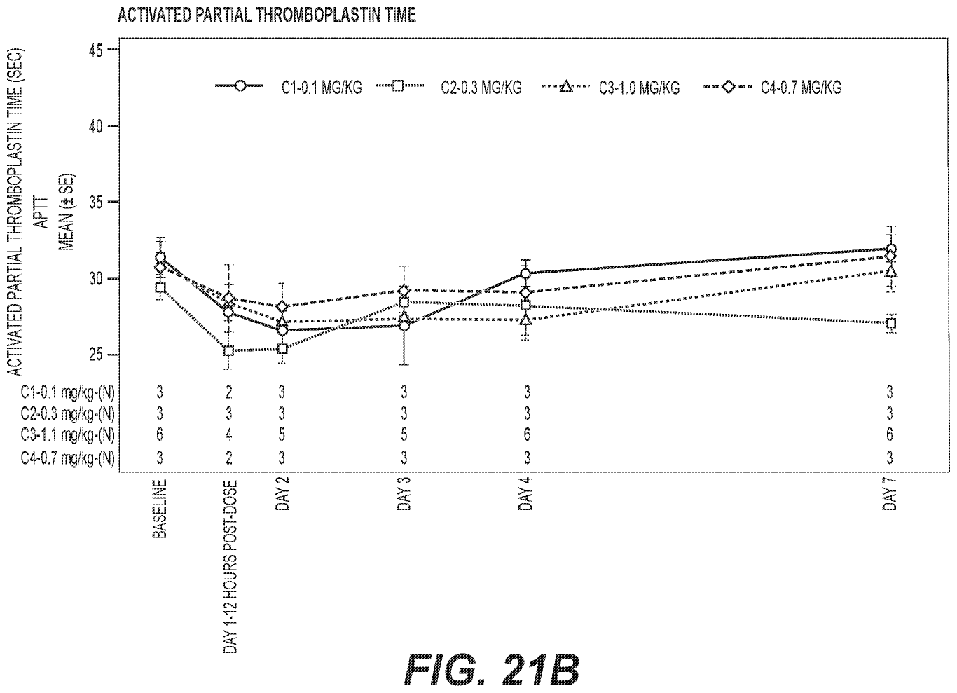

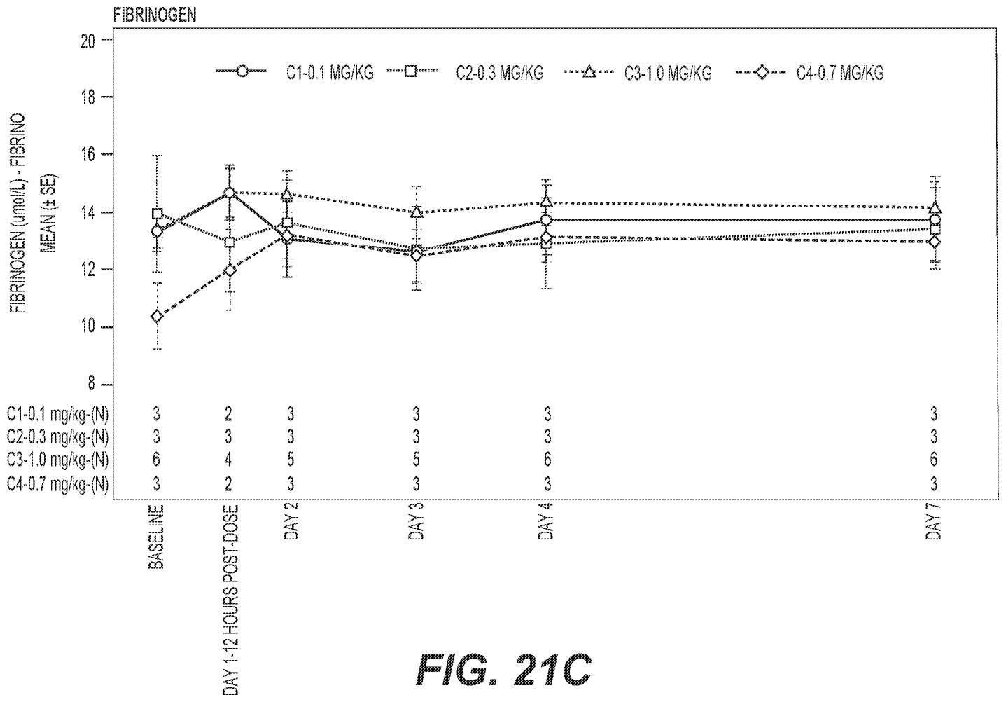

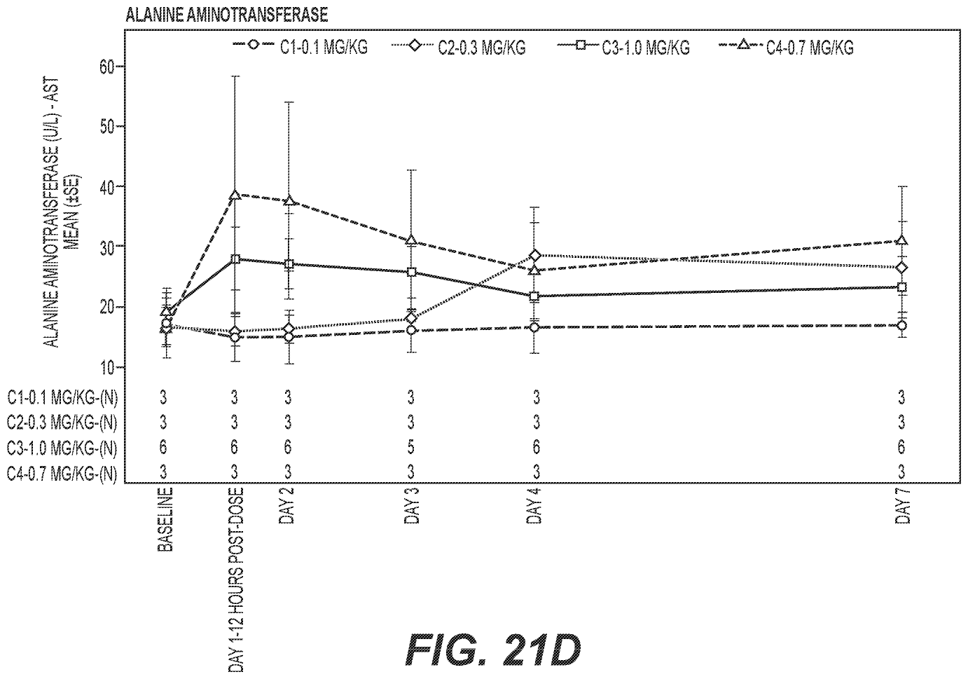

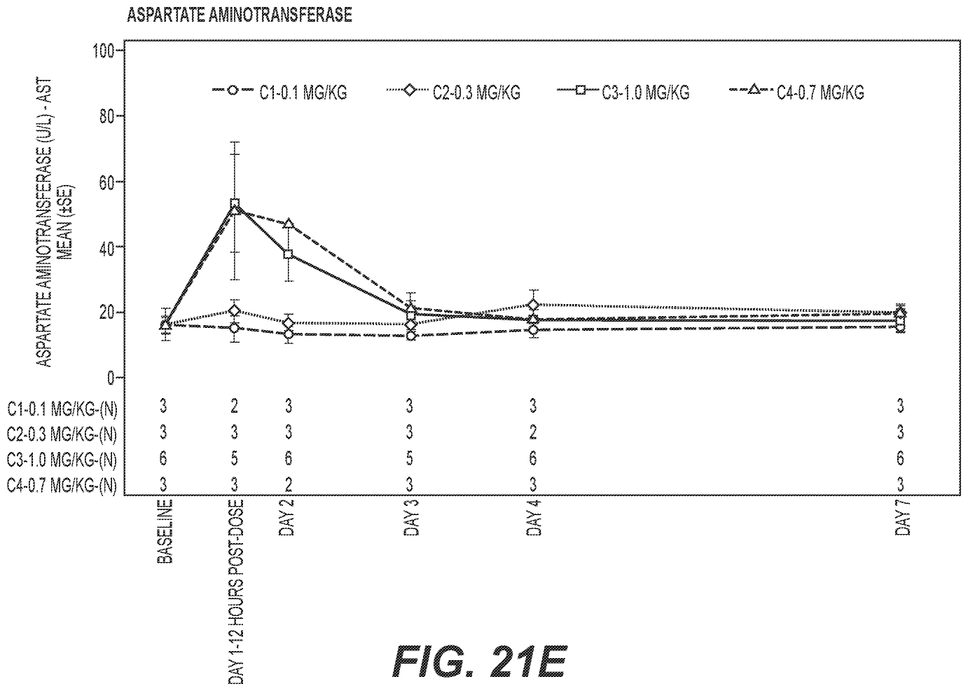

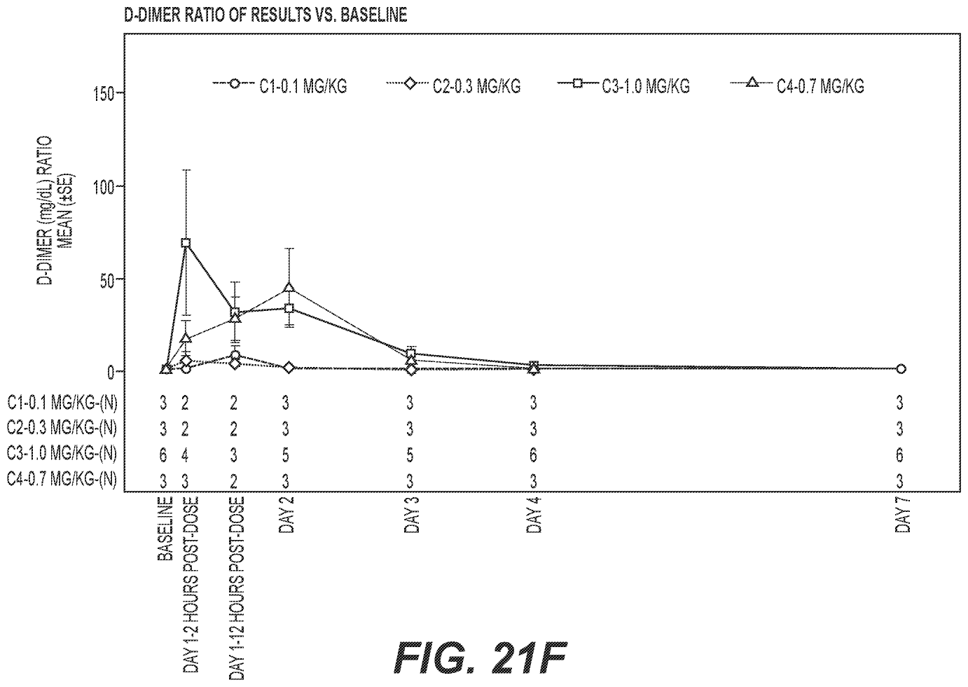

Liver function (aspartate aminotransferase and alanine aminotransferase), coagulation parameters (activated partial thromboplastin time, prothrombin time, fibrinogen), and d-dimer values were assessed by methods known in the art. Results remained within normal limits (FIG. 21).

FIG. 21A displays prothrombin time; FIG. 21B, activated partial prothrombin time; FIG. 21C, fibrinogen; FIG. 21D, alanine aminotransferase; FIG. 21E, aspartate aminotransferase; and FIG. 21F, d-dimer ratio. Data are shown as mean results over time for each cohort. Baseline is defined as the last available measurement taken prior to the start of infusion of study drug. Only results obtained from a central laboratory through day 7 are plotted.

ALT denotes alanine aminotransferase, aPTT activated partial thromboplastin time, AST aspartate aminotransferase, BL baseline, PT prothrombin time. Patients were monitored for assessment of treatment-emergent adverse events and laboratory findings. Serum samples were obtained at baseline and at weeks 1, 2 and 4, and month 2 for analysis of TTR protein levels by an enzyme-linked immunosorbent assay (ELISA). Patients are evaluated for safety and therapeutic activity outcomes for 24 months from NTLA-2001 infusion.

### Clinical Trial Efficacy

To determine the pharmacodynamic effect of NTLA-2001, serum TTR levels were evaluated. A sandwich ELISA method was developed and validated as a quantitative assay using human plasma TTR (Sigma, P1742) from healthy subjects as reference standard.

Briefly, assay microplates (Nunc, 446612) were incubated overnight with 1 μg/ml polyclonal rabbit anti-human prealbumin antibody (Dako, A0002) in 0.05 M carbonate coating buffer pH 9.6. Plates were washed four times with TTR wash solution (TTRWS: 0.05% Tween-20, 1× Dulbecco's PBS), blocked with 1× Powerblock (Biogenix, HK085-5k) for 1 hour, and washed four times with TTRWS. Standards, controls and diluted study samples were incubated with the prepared plate for about 2 hours. Plate was washed four times with TTRWS then incubated for 1 hour with sheep anti-human prealbumin antibody (Bio-Rad, AHP1837) diluted 1:2, 500 in 1× Powerblock. The plate was washed four times with TTRWS then incubated for 1 hour with anti-sheep alkaline phosphatase conjugate antibody (Sigma, A5187) diluted 1:10,000 in 1× Powerblock. The plate was washed four times with TTRWS. The plate was developed using SIGMAFAST™ p-Nitrophenyl phosphate Tablets (Sigma-Aldrich, N1891) according to the manufacturer's instructions. After 30 minutes incubation with the development reagent, the reaction was stopped using 2 N Sodium Hydroxide solution. Absorbance was assessed by spectrophotometry. Standard curve, signal (OD) vs. Concentration was generated using human plasma TTR (Sigma, P1742) to quantify QC and unknown samples.

### Interim Results Reporting 3 of 6 Subjects in Cohort 3 and 1 of 3 Subjects in Cohort 4

Reductions in serum TTR protein concentration from baseline were observed by day 14 and deepened by day 28. At day 7, one subject from Cohort 4 (0.7 mg/kg; subject 3 in FIG. 19C) had a reported 78% reduction in serum TTR. By day 14, the subject had a 94% reduction, and by day 28, a 97% reduction in serum TTR. At day 7, 3 subjects in Cohort 3 had a reduction that ranged from 43-88% (subject 1 43%, subject 2 80%, and subject 3 88%). At day 14, further reductions in serum TTR were observed (subject 1 80%, subject 2 88%, and subject 3 97%). At day 28, the reductions ranged from 88-98% (subject 1 88%, subject 2 88%, and subject 3 98%; FIG. 19).

### Interim Results Reporting 6 of 6 Subjects in Cohort 3 and 3 of 3 Subjects in Cohort 4

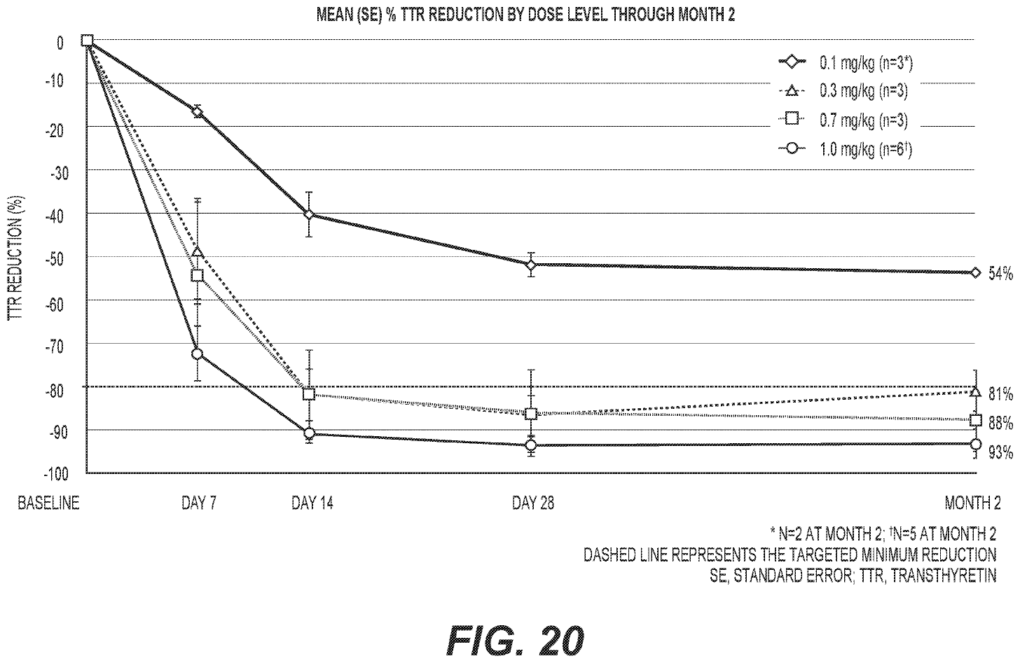

Reductions in serum TTR protein concentration from baseline were observed at day 7, day 14, day 28, day 56 (month 2), month 4 (some subjects), month 6 (some subjects), and month 6 (some subjects). All values for each subject available at the time of this interim result is shown in FIGS. 19B-19D. Mean percent TTR reduction at day 28 was 93% in cohort 3 (dose level 1 mg/kg) and 86% in cohort 4 (dose level 0.7 mg/kg). Mean percent TTR reduction at month 2 was 93% in cohort 3 and 88% in cohort 4. As depicted in FIG. 20, reductions were maintained at month 2. The reductions in percentage represent a change in total circulating serum transthyretin (TTR) protein for each subject from baseline for Cohort 3 (1 mg/kg) and Cohort 4 (0.7 mg/kg). Further updated serum TTR reduction information relative to results shown in FIGS. 19A-19D is shown in Table 4 below. Mean percent TTR reduction at month 6 was 93% for all subjects for cohort 3 (dose level 1 mg/kg) and 87% for all for subjects cohort 4 (dose level 0.7 mg/kg). As of this update, mean percent TTR reduction at month 9 for 3 out of 6 subjects in cohort 3 remained at 93%. ATTR protein was quantified by a validated enzyme-linked immunosorbent assay method following regulatory guidelines for biomarker method validation. Serum samples were measured once, with each sample tested in duplicate. As per good laboratory practice, no re-test was conducted for successful assay runs.

TABLE 4
Updated clinical trial subject data for polyneuropathy dose escalation study

| | | | | NT- | | | |
|---|---|---|---|---|---|---|---|
| | | | | proBNP | | | |
| Dose | | Weight | TTR | baseline | TTR | | % TTR |
| mpk | Patient | (kg) | Genotype | (ng/L) | (ug/mL) | Timepoint | Reduction |
| 0.1 | Cohort1Subject1 | 82.1 | S77Y | 89.0 | 149.94 | Day 7 | −19.00 |
| | (C1S1) | | | | | | |
| | | | | | 100.64 | Day 14 | −45.63 |
| | | | | | 80.58 | Day 28 | −56.47 |
| | | | | | 87.61 | Day 56 | −52.67 |
| | | | | | 97.7 | Month 4 | −47.22 |
| | | | | | 99.3 | Month 6 | −46.36 |
| | | | | | 86.36 | Month 9 | −53.35 |
| | | | | | 105.66 | Month 12 | −42.92 |
| 0.1 | C1S2 | 70.4 | T60A | 596.0 | 208.23 | Day 7 | −16.34 |
| | | | | | 174.31 | Day 14 | −29.97 |
| | | | | | 132.54 | Day 28 | −46.75 |
| | | | | | 201.88 | Month 4 | −18.89 |
| | | | | | 203.72 | Month 6 | −18.15 |
| | | | | | 283.50 | Month 9 | 13.90 |
| | | | | | 198.51 | Month 12 | −20.24 |
| | | | | | 236.53 | Month 18 | −4.97 |
| 0.1 | C1S3 | 89.1 | T60A, | 127.0 | 234.60 | Day 7 | −14.12 |
| | | | T80A | | | | |
| | | | | | 149.47 | Day 14 | −45.28 |
| | | | | | 130.28 | Day 28 | −52.30 |
| | | | | | 123.75 | Day 56 | −54.69 |
| | | | | | 153.35 | Month 4 | −43.86 |
| | | | | | 184.25 | Month 6 | −32.55 |
| | | | | | 142.57 | Month 9 | −47.81 |
| | | | | | 150.61 | Month 12 | −44.86 |
| 0.3 | C2S1 | 83.3 | S77Y | 118.0 | 130.94 | Day 7 | −43.78 |
| | | | | | 61.95 | Day 14 | −73.40 |
| | | | | | 45.78 | Day 28 | −80.34 |
| | | | | | 56.26 | Day 56 | −75.84 |
| | | | | | 51.62 | Month 4 | −77.84 |
| | | | | | 44.81 | Month 6 | −80.76 |
| | | | | | 33.34 | Month 9 | −85.69 |
| | | | | | 29.90 | Month 12 | −87.16 |
| 0.3 | C2S2 | 84.0 | T60A | 359.0 | 100.02 | Day 7 | −30.43 |
| | | | | | 30.57 | Day 14 | −78.74 |
| | | | | | 23.31 | Day 28 | −83.79 |
| | | | | | 33.47 | Day 56 | −76.72 |
| | | | | | 35.25 | Month 4 | −75.48 |
| | | | | | 31.88 | Month 6 | −77.83 |
| | | | | | 28.71 | Month 9 | −80.03 |
| | | | | | 20.03 | Month 12 | −86.07 |
| 0.3 | C2S3 | 89.9 | H90D | <50.0 | 84.08 | Day 7 | −71.98 |
| | | | | | 20.00 | Day 14 | −93.34 |
| | | | | | 12.81 | Day 28 | −95.73 |
| | | | | | 28.37 | Day 56 | −90.55 |
| | | | | | 25.61 | Month 4 | −91.47 |
| | | | | | 17.87 | Month 6 | −94.04 |
| | | | | | 21.97 | Month 9 | −92.68 |
| | | | | | 21.34 | Month 12 | −92.89 |
| 0.7 | C4S1 | 62.4 | E42D | 58.0 | 120.35 | Day 7 | −21.34 |
| | | | | | 58.76 | Day 14 | −61.60 |
| | | | | | 51.72 | Day 28 | −66.19 |
| | | | | | 41.26 | Day 56 | −73.03 |
| | | | | | 48.44 | Month 4 | −68.34 |
| | | | | | 46.19 | Month 6 | −69.81 |
| 0.7 | C4S2 | 97.6 | E74G | <50.0 | 77.36 | Day 7 | −63.17 |
| | | | | | 22.40 | Day 14 | −89.34 |
| | | | | | 11.66 | Day 28 | −94.45 |
| | | | | | 10.94 | Day 56 | −94.79 |
| | | | | | 8.33 | Month 4 | −96.03 |
| | | | | | 10.26 | Month 6 | −95.12 |
| 0.7 | C4S3 | 86.7 | T60A | 195.0 | 71.80 | Day 7 | −78.34 |
| | | | | | 18.39 | Day 14 | −94.45 |
| | | | | | 8.72 | Day 28 | −97.37 |
| | | | | | 15.93 | Day 56 | −95.20 |
| | | | | | 20.37 | Month 4 | −93.86 |
| | | | | | 16.36 | Month 6 | −95.07 |
| | | | | | 18.48 | Month 9 | −94.43 |
| 1.0 | C3S1 | 75.9 | E42D | 56.0 | 121.91 | Day 7 | −42.92 |
| | | | | | 43.08 | Day 14 | −79.83 |
| | | | | | 25.10 | Day 28 | −88.25 |
| | | | | | n/a | Unscheduled | −89.51 |
| | | | | | | Visit | |
| | | | | | 24.34 | Month 4 | −88.60 |
| | | | | | 25.30 | Month 6 | −88.16 |
| | | | | | 34.48 | Month 9 | −83.86 |
| 1.0 | C3S2 | 74.6 | E62D | 140.0 | 65.55 | Day 7 | −69.24 |
| | | | | | 22.43 | Day 14 | −89.47 |
| | | | | | 12.54 | Day 28 | −94.11 |
| | | | | | 11.75 | Day 56 | −94.48 |
| | | | | | 12.10 | Month 4 | −94.32 |
| | | | | | 10.43 | Month 6 | −95.11 |
| 1.0 | C3S3 | 111.0 | T80A | <50.0 | 36.70 | Day 7 | −76.51 |
| | | | | | 8.49 | Day 14 | −94.56 |
| | | | | | 6.33 | Day 28 | −95.95 |
| | | | | | 6.74 | Day 56 | −95.69 |
| | | | | | 5.98 | Month 4 | −96.17 |
| | | | | | 7.99 | Month 6 | −94.88 |
| 1.0 | C3S4 | 59.1 | V30M | 193.0 | 64.14 | Day 7 | −77.97 |
| | | | | | 16.07 | Day 14 | −94.48 |
| | | | | | 9.72 | Day 28 | −96.66 |
| | | | | | 10.07 | Day 56 | −96.54 |
| | | | | | 11.34 | Month 4 | −96.11 |
| | | | | | 9.97 | Month 6 | −96.58 |
| 1.0 | C3S5 | 69.2 | S50R | 544.0 | 43.01 | Day 7 | −79.84 |
| | | | | | 26.61 | Day 14 | −87.53 |
| | | | | | 26.74 | Day 28 | −87.47 |
| | | | | | 42.54 | Day 56 | −80.06 |
| | | | | | 21.70 | Month 4 | −89.83 |
| | | | | | 35.50 | Month 6 | −83.36 |
| | | | | | 8.94 | Month 9 | −95.81 |
| 1.0 | C3S6 | 88.6 | T60A | 84.0 | 44.95 | Day 7 | −87.13 |
| | | | | | 11.39 | Day 14 | −96.74 |
| | | | | | 8.26 | Day 28 | −97.64 |
| | | | | | 4.10 | Day 56 | −98.83 |
| | | | | | 4.75 | Month 4 | −98.64 |
| | | | | | 4.30 | Month 6 | −98.77 |
| | | | | | 4.65 | Month 9 | −98.67 |

### B. Polyneuropathy 80 mg Flat Dose Study

As of this update, one subject was recruited for the 80 mg flat dose study. The subject was aged 36 years, male. Clinical trial design and eligibility are as described herein for the polyneuropathy study, except that the subject was treated with a single dose of NTLA-2001 at a flat dose of 80 mg total RNA (guide RNA plus messenger RNA).

To determine the pharmacodynamic effect of NTLA-2001, serum TTR level was evaluated. A sandwich ELISA method was developed and validated as a quantitative assay using human plasma TTR (Sigma, P1742) from healthy subjects as reference standard, as described herein. Reduction in serum TTR protein concentration from baseline was observed by day 7. At day 7, the subject had a reported 58% reduction in serum TTR (absolute TTR concentration of 139 μg/ml).

### C. Cardiomyopathy Dose Escalation Study

### Enrollment Cohorts 1a and 2a

Recruitment for Cohort 1a and Cohort 2a is ongoing. Provided herein is recruitment information for subjects in Cohorts 1a and 2a where TTR reduction data are available. Three Cohort 1a subjects were recruited; the subjects were aged 71-75 years; all three subjects were male; and body weight range was 63-88 kg. For Cohort 1a, two of the three subjects had wildtype TTR; two subjects had a New York Heart Association (NYHA) Functional Classification of II, and one subject had a NYHA Functional Classification of I; NT-proBNP baseline levels ranged between 2103 pmol/L and 3637 pmol/L. One Cohort 2a subject was recruited; the patient is male, aged 75 years, with body weight of 71 kg. The subject in Cohort 2a had wildtype TTR; and a NYHA Functional Classification of III.

### Clinical Trial Design and Eligibility

Included herein are interim results of ongoing studies in Cohorts 1a and 2a from Part 1 of a two-part, global, phase 1, open-label, multi-center study. Subjects were treated with a single dose of NTLA-2001, total RNA/body mass 0.7 mg/kg (3 subjects in Cohort 1a) or 0.7 mg/kg (1 subject in Cohort 2a), administered intravenously. Enrollment and eligibility criteria are as described herein.

To determine the pharmacodynamic effect of NTLA-2001, serum TTR levels were evaluated. A sandwich ELISA method was developed and validated as a quantitative assay using human plasma TTR (Sigma, P1742) from healthy subjects as reference standard, as described herein. Reduction in serum TTR protein concentration from baseline was observed as summarized in Table 5 below.

TABLE 5
Clinical trial subject data for cardiomyopathy dose escalation study

| | | | | NT- | | | |
|---|---|---|---|---|---|---|---|
| | | | | proBNP | | | |
| Dose | | Weight | TTR | baseline | TTR | | % TTR |
| mpk | Patient | (kg) | Genotype | (pmol/L) | (ug/ml) | Timepoint | Reduction |
| 0.7 | Cohort1aSubject1 | 85 | n/a | 3637 | 115.37 | Day 7 | −57.93 |
| | (C1aS1) | | | | | | |
| | | | | | 37.94 | Day 14 | −86.16 |
| 0.7 | C1aS2 | 63 | WT | 2103 | 73.09 | Day 7 | −59.15 |
| | | | | | 18.83 | Day 14 | −89.48 |
| 0.7 | C1aS3 | 88 | WT | 2480 | 86.96 | Day 7 | −70.71 |
| | | | | | 23.76 | Day 14 | −92.00 |
| | | | | | 16.32 | Day 28 | −94.50 |
| 0.7 | C2aS1 | 71 | WT | 16690 | 127.89 | Day 7 | −50.04 |
| | | | | | 43.22 | Day 14 | −83.12 |
| | | | | | 17.90 | Day 28 | −93.01 |

### Example 9. Additional Clinical Assays

### Bioanalytical Methods and Clinical Pharmacology

Plasma pharmacokinetic methods were developed and validated to quantify 4 components of NTLA-2001: ionizable Lipid A (also referred to as LP01), DMG-PEG2k lipid, guide RNA, and mRNA. Lipid A and DMG-PEG2k are quantified by liquid chromatography with tandem mass spectrometry (LC-MS/MS) method. Assay signal [ratio of area under the curve of reference standard over internal standard (IS, using isotopic labelled reference standard)] vs. Concentration response standard curve is used. QCs and unknown sample signal are interpolated from standard curve to determine plasma concentrations. Both guide RNA and mRNA are quantified by qRT-PCR. Signal Cycle Threshold (Ct) vs. Concentration standard curve is used. QCs and unknown samples were interpolated from standard curve to quantify plasma concentrations. Urine PK methods were developed and validated for Lipid A and DMG-PEG2k to characterize excretion.

Immunogenicity methods were developed and validated to assess anti-drug antibody (ADA) to NTLA-2001 and anti-Cas9 protein (Cas9mRNA transgene product) antibodies. Both methods use Meso Scale Discovery Electrochemiluminescence (MSD-ECL) assays in sandwich format, where NTLA-2001 LNP or Cas9 protein was coated as capture antigens. Immobilized antibodies to drug or Cas9 protein was detected by anti-human IgM/IgG-sulfo tag detection antibodies. Sample analysis is planned in tier wise analysis using cut-points following regulatory guidelines to screen, confirm, and titer the antibody response. Confirmatory assay is based on drug or Cas9 protein competitive inhibition based on cut-point. Confirmed positive samples are tested for end-point titers. Additional pharmacodynamic methods included serum TTR by ELISA as primary PD, and LC-MS/MS as secondary PD, and prealbumin in vitro diagnostic method (IVD) for patient management and PD. A sandwich ELISA method was developed and validated as a quantitative assay using human plasma TTR from healthy subjects as a reference standard. Polyclonal antibodies are used as both capture and detection antibodies. The signal (OD) vs concentration standard curve is generated to quantify QC and unknown samples. The LC-MS/MS method was developed and validated using 3 surrogate peptides to quantify TTR including V30M mutant and corresponding wild type V30V, and a 3^{rd} peptide upstream similar to NHP LC-MS/MS peptide location for bridging NHP data. Signal (ratio of reference standard/isotope labelled IS for each peptide) vs. Concentration response is used as standard curve to interpolate QCs and unknown samples to determine serum concentration. Prealbumin method is based on turbidimetric principle as IVD methods where presence of TTR will form turbidity when polyclonal antisera to TTR is added to generate immune complexes. Additional PD biomarkers assays were developed and validated for exploratory purposes, including retinoid binding protein (RBP) by sandwich ELISA. Circulating neurofilament light chain (NfL) as an exploratory PD biomarker was quantified using a qualified method based on Quanterix Simoa platform; this biomarker is ATTR-PN specific.

Multiplex cytokines (GM-CSF, IFNg, IL-1b, IL-4, IL-5, IL-6, IL-8, IL-10, IL-12 (p70), IL-13, IL-17A, IL-23, TNFa) by Luminex and MCP-1 by ELISA were developed and validated to evaluate cytokine response after NTLA-2001 infusion. Complement components C3a, C5a, and Bb methods by ELISA were developed and validated to assess complement activation.

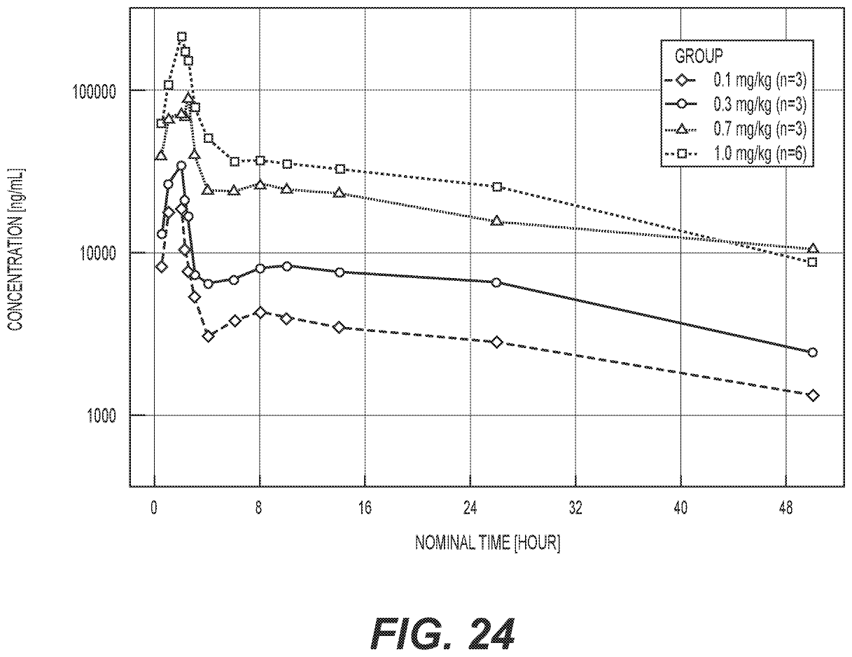

Preliminary plasma PK data are available for the four components of NTLA-2001 (ionizable Lipid A, DMG-PEG2k lipid, guide RNA, and mRNA). Following a single IV infusion of NTLA-2001 at doses from 0.1 to 1.0 mg/kg, LP01 exhibits a rapid decline from peak levels, followed by a secondary peak, and then a log-linear phase characterized by a mean (% CV) terminal t1/2 ranging from 19.74 (16.57) to 24.81 (23.55) h across this dose range. FIG. 24. Data for other components not shown.

### Example 10. Exposure-Response (ER) Analysis

An interim ER model was developed for NTLA-2001 using Day 28 TTR (% baseline) by assuming a sigmoidal relationship according to Equation 1 and using nonlinear least squares with R4.0.5 (The R Foundation for Statistical Computing, Vienna, Austria).

$$TTR ( \% baseline ) = 100 - {E}_{max} \cdot \frac{{EC}_{50}^{γ}}{{EC}_{50}^{γ} + {AUC}^{γ}} + ε \qquad Equation 1$$

where γ is the Hill coefficient, Emax is the maximum reduction in D28 TTR (% baseline), EC50 is the NTLA-2001 exposure corresponding to half-maximal effect on D28 TTR (% baseline), and ε is normally distributed with mean 0 and variance σ². Bootstrap (stratified by dose group, n=1000) confidence intervals (CI) on the mean prediction were generated; prediction intervals (PI) were generated via simulation from bootstrap estimates (n=1000) and ε.

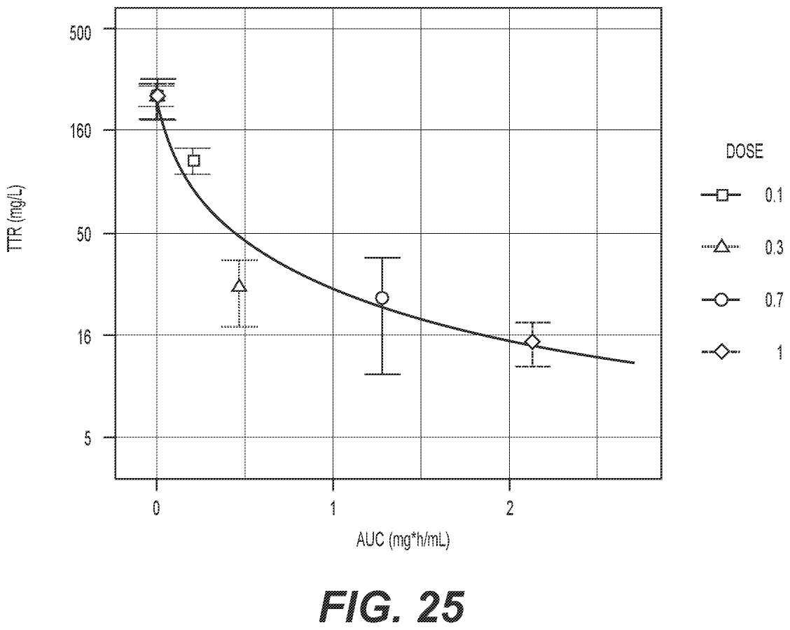

The model fit is shown in FIG. 25, which depicts the saturating ER relationship for NTLA-2001.

### Example 11. Population Pharmacokinetics (POPPK)

An interim POPPK model was developed for NTLA-2001 analyte LP01 based on a published model using NONMEM software (Version 7.5.0, ICON Clinical Research LLC, Blue Vell, PA). There were 290 observations in 15 ATTRv-PN subjects for this analysis. Parameter values for this model and goodness of fit plots were determined (not shown). The relationship between body weight and the estimated elimination clearance was determined, together with the modeled linear relationship. The relationship is less than proportional, i.e., a doubling of weight translates to a less than two-fold change in clearance.

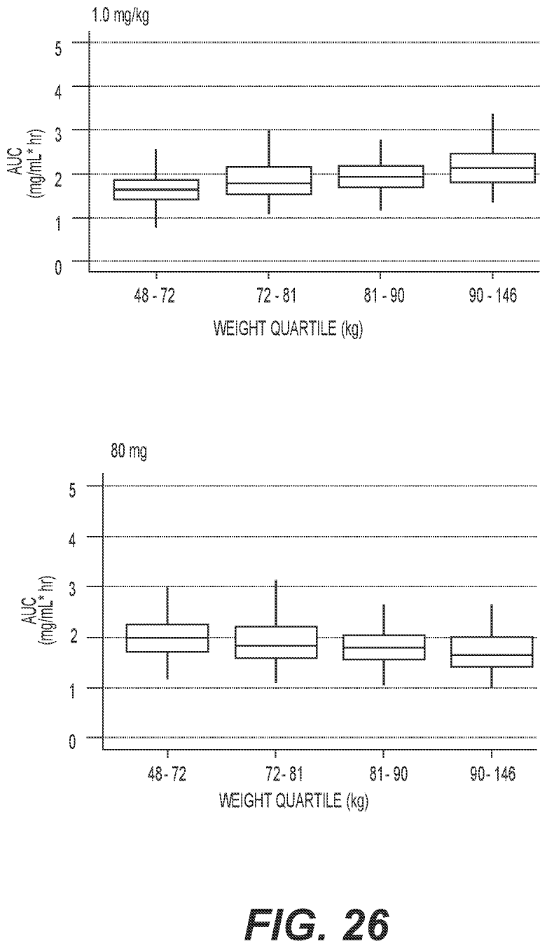

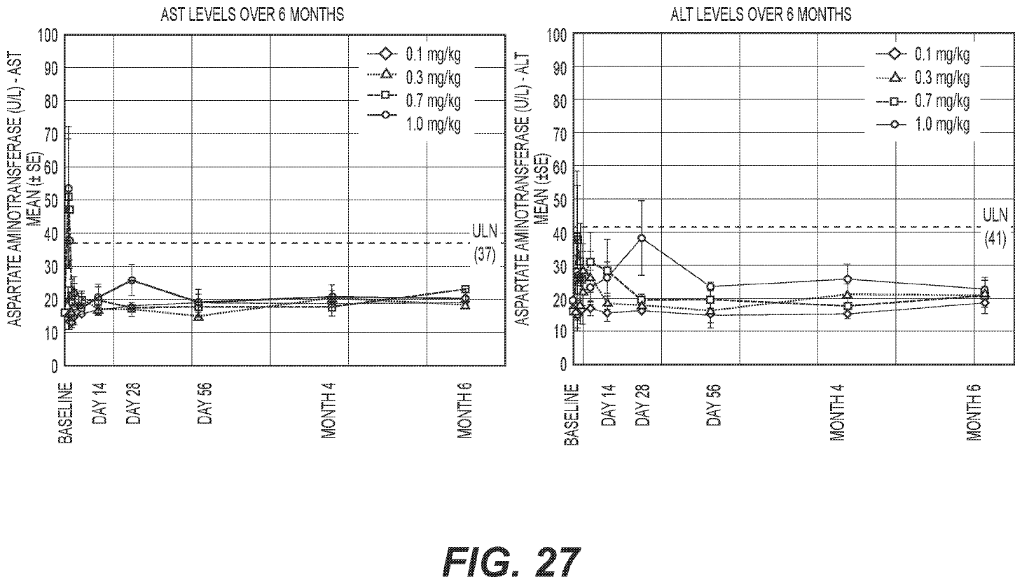

FIG. 26 provides the distribution of simulated AUC by weight quartile following administration of 1 mg/kg (left panel) and 80 mg (right panel) NTLA-2001. The POPPK simulations conducted in 10000 virtual subjects of median [5%, 95%] bodyweight of 81 [48, 146] kg suggest considerable overlap of LP01 AUC following 1 mg/kg NTLA-2001 across weight quartiles, but with a slight tendency toward increased median exposure with weight. Likewise, following 80 mg NTLA-2001, there is again overlap of simulated LP01 AUC across weight quartiles. The geometric mean and 5^{th} and 95^{th} percentile range of individual ratios (GMR) of NTLA-2001 AUC estimates following fixed to weight-based administration is 0.98 [0.74, 1.28]. The ratio of simulated mean exposures in the 4^{th} ([90.3-146] kg) to the 1^{st} weight quartile ([48-71.7] kg) is 1.25 for 1 mg/kg NTLA-2001 and 0.81 for 80 mg NTLA-2001. Simulations identified NTLA-2001 80 mg as the fixed dose equivalent to 1.0 mg/kg.

### Sequence Table

The following sequence table provides a listing of sequences disclosed herein. It is understood that if a DNA sequence (comprising Ts) is referenced with respect to an RNA, then Ts should be replaced with Us (which may be modified or unmodified depending on the context), and vice versa.

| SEQ | | |
|---|---|---|
| ID | | |
| No. | Description | Sequence |
| 1 | ORF | ATGGACAAGAAGTACAGCATCGGACTGGACATCGGAACAAACAGCGTCGGAT |
| | encoding | GGGCAGTCATCACAGACGAATACAAGGTCCCGAGCAAGAAGTTCAAGGTCCT |
| | Sp. Cas9 | GGGAAACACAGACAGACACAGCATCAAGAAGAACCTGATCGGAGCACTGCTG |
| | | TTCGACAGCGGAGAAACAGCAGAAGCAACAAGACTGAAGAGAACAGCAAGAA |
| | | GAAGATACACAAGAAGAAAGAACAGAATCTGCTACCTGCAGGAAATCTTCAG |
| | | CAACGAAATGGCAAAGGTCGACGACAGCTTCTTCCACAGACTGGAAGAAAGC |
| | | TTCCTGGTCGAAGAAGACAAGAAGCACGAAAGACACCCGATCTTCGGAAACA |
| | | TCGTCGACGAAGTCGCATACCACGAAAAGTACCCGACAATCTACCACCTGAG |
| | | AAAGAAGCTGGTCGACAGCACAGACAAGGCAGACCTGAGACTGATCTACCTG |
| | | GCACTGGCACACATGATCAAGTTCAGAGGACACTTCCTGATCGAAGGAGACC |
| | | TGAACCCGGACAACAGCGACGTCGACAAGCTGTTCATCCAGCTGGTCCAGAC |
| | | ATACAACCAGCTGTTCGAAGAAAACCCGATCAACGCAAGCGGAGTCGACGCA |
| | | AAGGCAATCCTGAGCGCAAGACTGAGCAAGAGCAGAAGACTGGAAAACCTGA |
| | | TCGCACAGCTGCCGGGAGAAAAGAAGAACGGACTGTTCGGAAACCTGATCGC |
| | | ACTGAGCCTGGGACTGACACCGAACTTCAAGAGCAACTTCGACCTGGCAGAA |
| | | GACGCAAAGCTGCAGCTGAGCAAGGACACATACGACGACGACCTGGACAACC |
| | | TGCTGGCACAGATCGGAGACCAGTACGCAGACCTGTTCCTGGCAGCAAAGAA |
| | | CCTGAGCGACGCAATCCTGCTGAGCGACATCCTGAGAGTCAACACAGAAATC |
| | | ACAAAGGCACCGCTGAGCGCAAGCATGATCAAGAGATACGACGAACACCACC |
| | | AGGACCTGACACTGCTGAAGGCACTGGTCAGACAGCAGCTGCCGGAAAAGTA |
| | | CAAGGAAATCTTCTTCGACCAGAGCAAGAACGGATACGCAGGATACATCGAC |
| | | GGAGGAGCAAGCCAGGAAGAATTCTACAAGTTCATCAAGCCGATCCTGGAAA |
| | | AGATGGACGGAACAGAAGAACTGCTGGTCAAGCTGAACAGAGAAGACCTGCT |
| | | GAGAAAGCAGAGAACATTCGACAACGGAAGCATCCCGCACCAGATCCACCTG |
| | | GGAGAACTGCACGCAATCCTGAGAAGACAGGAAGACTTCTACCCGTTCCTGA |
| | | AGGACAACAGAGAAAAGATCGAAAAGATCCTGACATTCAGAATCCCGTACTA |
| | | CGTCGGACCGCTGGCAAGAGGAAACAGCAGATTCGCATGGATGACAAGAAAG |
| | | AGCGAAGAAACAATCACACCGTGGAACTTCGAAGAAGTCGTCGACAAGGGAG |
| | | CAAGCGCACAGAGCTTCATCGAAAGAATGACAAACTTCGACAAGAACCTGCC |
| | | GAACGAAAAGGTCCTGCCGAAGCACAGCCTGCTGTACGAATACTTCACAGTC |
| | | TACAACGAACTGACAAAGGTCAAGTACGTCACAGAAGGAATGAGAAAGCCGG |
| | | CATTCCTGAGCGGAGAACAGAAGAAGGCAATCGTCGACCTGCTGTTCAAGAC |
| | | AAACAGAAAGGTCACAGTCAAGCAGCTGAAGGAAGACTACTTCAAGAAGATC |
| | | GAATGCTTCGACAGCGTCGAAATCAGCGGAGTCGAAGACAGATTCAACGCAA |
| | | GCCTGGGAACATACCACGACCTGCTGAAGATCATCAAGGACAAGGACTTCCT |
| | | GGACAACGAAGAAAACGAAGACATCCTGGAAGACATCGTCCTGACACTGACA |
| | | CTGTTCGAAGACAGAGAAATGATCGAAGAAAGACTGAAGACATACGCACACC |
| | | TGTTCGACGACAAGGTCATGAAGCAGCTGAAGAGAAGAAGATACACAGGATG |
| | | GGGAAGACTGAGCAGAAAGCTGATCAACGGAATCAGAGACAAGCAGAGCGGA |
| | | AAGACAATCCTGGACTTCCTGAAGAGCGACGGATTCGCAAACAGAAACTTCA |
| | | TGCAGCTGATCCACGACGACAGCCTGACATTCAAGGAAGACATCCAGAAGGC |
| | | ACAGGTCAGCGGACAGGGAGACAGCCTGCACGAACACATCGCAAACCTGGCA |
| | | GGAAGCCCGGCAATCAAGAAGGGAATCCTGCAGACAGTCAAGGTCGTCGACG |
| | | AACTGGTCAAGGTCATGGGAAGACACAAGCCGGAAAACATCGTCATCGAAAT |
| | | GGCAAGAGAAAACCAGACAACACAGAAGGGACAGAAGAACAGCAGAGAAAGA |
| | | ATGAAGAGAATCGAAGAAGGAATCAAGGAACTGGGAAGCCAGATCCTGAAGG |
| | | AACACCCGGTCGAAAACACACAGCTGCAGAACGAAAAGCTGTACCTGTACTA |
| | | CCTGCAGAACGGAAGAGACATGTACGTCGACCAGGAACTGGACATCAACAGA |
| | | CTGAGCGACTACGACGTCGACCACATCGTCCCGCAGAGCTTCCTGAAGGACG |
| | | ACAGCATCGACAACAAGGTCCTGACAAGAAGCGACAAGAACAGAGGAAAGAG |
| | | CGACAACGTCCCGAGCGAAGAAGTCGTCAAGAAGATGAAGAACTACTGGAGA |
| | | CAGCTGCTGAACGCAAAGCTGATCACACAGAGAAAGTTCGACAACCTGACAA |
| | | AGGCAGAGAGAGGAGGACTGAGCGAACTGGACAAGGCAGGATTCATCAAGAG |
| | | ACAGCTGGTCGAAACAAGACAGATCACAAAGCACGTCGCACAGATCCTGGAC |
| | | AGCAGAATGAACACAAAGTACGACGAAAACGACAAGCTGATCAGAGAAGTCA |
| | | AGGTCATCACACTGAAGAGCAAGCTGGTCAGCGACTTCAGAAAGGACTTCCA |
| | | GTTCTACAAGGTCAGAGAAATCAACAACTACCACCACGCACACGACGCATAC |
| | | CTGAACGCAGTCGTCGGAACAGCACTGATCAAGAAGTACCCGAAGCTGGAAA |
| | | GCGAATTCGTCTACGGAGACTACAAGGTCTACGACGTCAGAAAGATGATCGC |
| | | AAAGAGCGAACAGGAAATCGGAAAGGCAACAGCAAAGTACTTCTTCTACAGC |
| | | AACATCATGAACTTCTTCAAGACAGAAATCACACTGGCAAACGGAGAAATCA |
| | | GAAAGAGACCGCTGATCGAAACAAACGGAGAAACAGGAGAAATCGTCTGGGA |
| | | CAAGGGAAGAGACTTCGCAACAGTCAGAAAGGTCCTGAGCATGCCGCAGGTC |
| | | AACATCGTCAAGAAGACAGAAGTCCAGACAGGAGGATTCAGCAAGGAAAGCA |
| | | TCCTGCCGAAGAGAAACAGCGACAAGCTGATCGCAAGAAAGAAGGACTGGGA |
| | | CCCGAAGAAGTACGGAGGATTCGACAGCCCGACAGTCGCATACAGCGTCCTG |
| | | GTCGTCGCAAAGGTCGAAAAGGGAAAGAGCAAGAAGCTGAAGAGCGTCAAGG |
| | | AACTGCTGGGAATCACAATCATGGAAAGAAGCAGCTTCGAAAAGAACCCGAT |
| | | CGACTTCCTGGAAGCAAAGGGATACAAGGAAGTCAAGAAGGACCTGATCATC |
| | | AAGCTGCCGAAGTACAGCCTGTTCGAACTGGAAAACGGAAGAAAGAGAATGC |
| | | TGGCAAGCGCAGGAGAACTGCAGAAGGGAAACGAACTGGCACTGCCGAGCAA |
| | | GTACGTCAACTTCCTGTACCTGGCAAGCCACTACGAAAAGCTGAAGGGAAGC |
| | | CCGGAAGACAACGAACAGAAGCAGCTGTTCGTCGAACAGCACAAGCACTACC |
| | | TGGACGAAATCATCGAACAGATCAGCGAATTCAGCAAGAGAGTCATCCTGGC |
| | | AGACGCAAACCTGGACAAGGTCCTGAGCGCATACAACAAGCACAGAGACAAG |
| | | CCGATCAGAGAACAGGCAGAAAACATCATCCACCTGTTCACACTGACAAACC |
| | | TGGGAGCACCGGCAGCATTCAAGTACTTCGACACAACAATCGACAGAAAGAG |
| | | ATACACAAGCACAAAGGAAGTCCTGGACGCAACACTGATCCACCAGAGCATC |
| | | ACAGGACTGTACGAAACAAGAATCGACCTGAGCCAGCTGGGAGGAGACGGAG |
| | | GAGGAAGCCCGAAGAAGAAGAGAAAGGTCTAG |
| 2 | ORF | ATGGACAAGAAGTACTCCATCGGCCTGGACATCGGCACCAACTCCGTGGGCT |
| | encoding | GGGCCGTGATCACCGACGAGTACAAGGTGCCCTCCAAGAAGTTCAAGGTGCT |
| | Sp. Cas9 | GGGCAACACCGACCGGCACTCCATCAAGAAGAACCTGATCGGCGCCCTGCTG |
| | | TTCGACTCCGGCGAGACCGCCGAGGCCACCCGGCTGAAGCGGACCGCCCGGC |
| | | GGCGGTACACCCGGCGGAAGAACCGGATCTGCTACCTGCAGGAGATCTTCTC |
| | | CAACGAGATGGCCAAGGTGGACGACTCCTTCTTCCACCGGCTGGAGGAGTCC |
| | | TTCCTGGTGGAGGAGGACAAGAAGCACGAGCGGCACCCCATCTTCGGCAACA |
| | | TCGTGGACGAGGTGGCCTACCACGAGAAGTACCCCACCATCTACCACCTGCG |
| | | GAAGAAGCTGGTGGACTCCACCGACAAGGCCGACCTGCGGCTGATCTACCTG |
| | | GCCCTGGCCCACATGATCAAGTTCCGGGGCCACTTCCTGATCGAGGGCGACC |
| | | TGAACCCCGACAACTCCGACGTGGACAAGCTGTTCATCCAGCTGGTGCAGAC |
| | | CTACAACCAGCTGTTCGAGGAGAACCCCATCAACGCCTCCGGCGTGGACGCC |
| | | AAGGCCATCCTGTCCGCCCGGCTGTCCAAGTCCCGGCGGCTGGAGAACCTGA |
| | | TCGCCCAGCTGCCCGGCGAGAAGAAGAACGGCCTGTTCGGCAACCTGATCGC |
| | | CCTGTCCCTGGGCCTGACCCCCAACTTCAAGTCCAACTTCGACCTGGCCGAG |
| | | GACGCCAAGCTGCAGCTGTCCAAGGACACCTACGACGACGACCTGGACAACC |
| | | TGCTGGCCCAGATCGGCGACCAGTACGCCGACCTGTTCCTGGCCGCCAAGAA |
| | | CCTGTCCGACGCCATCCTGCTGTCCGACATCCTGCGGGTGAACACCGAGATC |
| | | ACCAAGGCCCCCCTGTCCGCCTCCATGATCAAGCGGTACGACGAGCACCACC |
| | | AGGACCTGACCCTGCTGAAGGCCCTGGTGCGGCAGCAGCTGCCCGAGAAGTA |
| | | CAAGGAGATCTTCTTCGACCAGTCCAAGAACGGCTACGCCGGCTACATCGAC |
| | | GGCGGCGCCTCCCAGGAGGAGTTCTACAAGTTCATCAAGCCCATCCTGGAGA |
| | | AGATGGACGGCACCGAGGAGCTGCTGGTGAAGCTGAACCGGGAGGACCTGCT |
| | | GCGGAAGCAGCGGACCTTCGACAACGGCTCCATCCCCCACCAGATCCACCTG |
| | | GGCGAGCTGCACGCCATCCTGCGGCGGCAGGAGGACTTCTACCCCTTCCTGA |
| | | AGGACAACCGGGAGAAGATCGAGAAGATCCTGACCTTCCGGATCCCCTACTA |
| | | CGTGGGCCCCCTGGCCCGGGGCAACTCCCGGTTCGCCTGGATGACCCGGAAG |
| | | TCCGAGGAGACCATCACCCCCTGGAACTTCGAGGAGGTGGTGGACAAGGGCG |
| | | CCTCCGCCCAGTCCTTCATCGAGCGGATGACCAACTTCGACAAGAACCTGCC |
| | | CAACGAGAAGGTGCTGCCCAAGCACTCCCTGCTGTACGAGTACTTCACCGTG |
| | | TACAACGAGCTGACCAAGGTGAAGTACGTGACCGAGGGCATGCGGAAGCCCG |
| | | CCTTCCTGTCCGGCGAGCAGAAGAAGGCCATCGTGGACCTGCTGTTCAAGAC |
| | | CAACCGGAAGGTGACCGTGAAGCAGCTGAAGGAGGACTACTTCAAGAAGATC |
| | | GAGTGCTTCGACTCCGTGGAGATCTCCGGCGTGGAGGACCGGTTCAACGCCT |
| | | CCCTGGGCACCTACCACGACCTGCTGAAGATCATCAAGGACAAGGACTTCCT |
| | | GGACAACGAGGAGAACGAGGACATCCTGGAGGACATCGTGCTGACCCTGACC |
| | | CTGTTCGAGGACCGGGAGATGATCGAGGAGCGGCTGAAGACCTACGCCCACC |
| | | TGTTCGACGACAAGGTGATGAAGCAGCTGAAGCGGCGGCGGTACACCGGCTG |
| | | GGGCCGGCTGTCCCGGAAGCTGATCAACGGCATCCGGGACAAGCAGTCCGGC |
| | | AAGACCATCCTGGACTTCCTGAAGTCCGACGGCTTCGCCAACCGGAACTTCA |
| | | TGCAGCTGATCCACGACGACTCCCTGACCTTCAAGGAGGACATCCAGAAGGC |
| | | CCAGGTGTCCGGCCAGGGCGACTCCCTGCACGAGCACATCGCCAACCTGGCC |
| | | GGCTCCCCCGCCATCAAGAAGGGCATCCTGCAGACCGTGAAGGTGGTGGACG |
| | | AGCTGGTGAAGGTGATGGGCCGGCACAAGCCCGAGAACATCGTGATCGAGAT |
| | | GGCCCGGGAGAACCAGACCACCCAGAAGGGCCAGAAGAACTCCCGGGAGCGG |
| | | ATGAAGCGGATCGAGGAGGGCATCAAGGAGCTGGGCTCCCAGATCCTGAAGG |
| | | AGCACCCCGTGGAGAACACCCAGCTGCAGAACGAGAAGCTGTACCTGTACTA |
| | | CCTGCAGAACGGCCGGGACATGTACGTGGACCAGGAGCTGGACATCAACCGG |
| | | CTGTCCGACTACGACGTGGACCACATCGTGCCCCAGTCCTTCCTGAAGGACG |
| | | ACTCCATCGACAACAAGGTGCTGACCCGGTCCGACAAGAACCGGGGCAAGTC |
| | | CGACAACGTGCCCTCCGAGGAGGTGGTGAAGAAGATGAAGAACTACTGGCGG |
| | | CAGCTGCTGAACGCCAAGCTGATCACCCAGCGGAAGTTCGACAACCTGACCA |
| | | AGGCCGAGCGGGGCGGCCTGTCCGAGCTGGACAAGGCCGGCTTCATCAAGCG |
| | | GCAGCTGGTGGAGACCCGGCAGATCACCAAGCACGTGGCCCAGATCCTGGAC |
| | | TCCCGGATGAACACCAAGTACGACGAGAACGACAAGCTGATCCGGGAGGTGA |
| | | AGGTGATCACCCTGAAGTCCAAGCTGGTGTCCGACTTCCGGAAGGACTTCCA |
| | | GTTCTACAAGGTGCGGGAGATCAACAACTACCACCACGCCCACGACGCCTAC |
| | | CTGAACGCCGTGGTGGGCACCGCCCTGATCAAGAAGTACCCCAAGCTGGAGT |
| | | CCGAGTTCGTGTACGGCGACTACAAGGTGTACGACGTGCGGAAGATGATCGC |
| | | CAAGTCCGAGCAGGAGATCGGCAAGGCCACCGCCAAGTACTTCTTCTACTCC |
| | | AACATCATGAACTTCTTCAAGACCGAGATCACCCTGGCCAACGGCGAGATCC |
| | | GGAAGCGGCCCCTGATCGAGACCAACGGCGAGACCGGCGAGATCGTGTGGGA |
| | | CAAGGGCCGGGACTTCGCCACCGTGCGGAAGGTGCTGTCCATGCCCCAGGTG |
| | | AACATCGTGAAGAAGACCGAGGTGCAGACCGGCGGCTTCTCCAAGGAGTCCA |
| | | TCCTGCCCAAGCGGAACTCCGACAAGCTGATCGCCCGGAAGAAGGACTGGGA |
| | | CCCCAAGAAGTACGGCGGCTTCGACTCCCCCACCGTGGCCTACTCCGTGCTG |
| | | GTGGTGGCCAAGGTGGAGAAGGGCAAGTCCAAGAAGCTGAAGTCCGTGAAGG |
| | | AGCTGCTGGGCATCACCATCATGGAGCGGTCCTCCTTCGAGAAGAACCCCAT |
| | | CGACTTCCTGGAGGCCAAGGGCTACAAGGAGGTGAAGAAGGACCTGATCATC |
| | | AAGCTGCCCAAGTACTCCCTGTTCGAGCTGGAGAACGGCCGGAAGCGGATGC |
| | | TGGCCTCCGCCGGCGAGCTGCAGAAGGGCAACGAGCTGGCCCTGCCCTCCAA |
| | | GTACGTGAACTTCCTGTACCTGGCCTCCCACTACGAGAAGCTGAAGGGCTCC |
| | | CCCGAGGACAACGAGCAGAAGCAGCTGTTCGTGGAGCAGCACAAGCACTACC |
| | | TGGACGAGATCATCGAGCAGATCTCCGAGTTCTCCAAGCGGGTGATCCTGGC |
| | | CGACGCCAACCTGGACAAGGTGCTGTCCGCCTACAACAAGCACCGGGACAAG |
| | | CCCATCCGGGAGCAGGCCGAGAACATCATCCACCTGTTCACCCTGACCAACC |
| | | TGGGCGCCCCCGCCGCCTTCAAGTACTTCGACACCACCATCGACCGGAAGCG |
| | | GTACACCTCCACCAAGGAGGTGCTGGACGCCACCCTGATCCACCAGTCCATC |
| | | ACCGGCCTGTACGAGACCCGGATCGACCTGTCCCAGCTGGGCGGCGACGGCG |
| | | GCGGCTCCCCCAAGAAGAAGCGGAAGGTGTGA |
| 3 | ORF | AUGGACAAGAAGUACAGCAUCGGCCUGGACAUCGGCACGAACAGCGUUGGCU |
| | encoding | GGGCUGUGAUCACGGACGAGUACAAGGUUCCCUCAAAGAAGUUCAAGGUGCU |
| | Sp. Cas9 | GGGCAACACGGACCGGCACAGCAUCAAGAAGAAUCUCAUCGGUGCACUGCUG |
| | | UUCGACAGCGGUGAGACGGCCGAAGCCACGCGGCUGAAGCGGACGGCCCGCC |
| | | GGCGGUACACGCGGCGGAAGAACCGGAUCUGCUACCUGCAGGAGAUCUUCAG |
| | | CAACGAGAUGGCCAAGGUGGACGACAGCUUCUUCCACCGGCUGGAGGAGAGC |
| | | UUCCUGGUGGAGGAGGACAAGAAGCACGAGCGGCACCCCAUCUUCGGCAACA |
| | | UCGUGGACGAAGUCGCCUACCACGAGAAGUACCCCACCAUCUACCACCUGCG |
| | | GAAGAAGCUGGUGGACUCGACUGACAAGGCCGACCUGCGGCUGAUCUACCUG |
| | | GCACUGGCCCACAUGAUAAAGUUCCGGGGCCACUUCCUGAUCGAGGGCGACC |
| | | UGAACCCUGACAACAGCGACGUGGACAAGCUGUUCAUCCAGCUGGUGCAGAC |
| | | CUACAACCAGCUGUUCGAGGAGAACCCCAUCAACGCCAGCGGCGUGGACGCC |
| | | AAGGCCAUCCUCAGCGCCCGCCUCAGCAAGAGCCGGCGGCUGGAGAAUCUCA |
| | | UCGCCCAGCUUCCAGGUGAGAAGAAGAAUGGGCUGUUCGGCAAUCUCAUCGC |
| | | ACUCAGCCUGGGCCUGACUCCCAACUUCAAGAGCAACUUCGACCUGGCCGAG |
| | | GACGCCAAGCUGCAGCUCAGCAAGGACACCUACGACGACGACCUGGACAAUC |
| | | UCCUGGCCCAGAUCGGCGACCAGUACGCCGACCUGUUCCUGGCUGCCAAGAA |
| | | UCUCAGCGACGCCAUCCUGCUCAGCGACAUCCUGCGGGUGAACACAGAGAUC |
| | | ACGAAGGCCCCCCUCAGCGCCAGCAUGAUAAAGCGGUACGACGAGCACCACC |
| | | AGGACCUGACGCUGCUGAAGGCACUGGUGCGGCAGCAGCUUCCAGAGAAGUA |
| | | CAAGGAGAUCUUCUUCGACCAGAGCAAGAAUGGGUACGCCGGGUACAUCGAC |
| | | GGUGGUGCCAGCCAGGAGGAGUUCUACAAGUUCAUCAAGCCCAUCCUGGAGA |
| | | AGAUGGACGGCACAGAGGAGCUGCUGGUGAAGCUGAACAGGGAGGACCUGCU |
| | | GCGGAAGCAGCGGACGUUCGACAAUGGGAGCAUCCCCCACCAGAUCCACCUG |
| | | GGUGAGCUGCACGCCAUCCUGCGGCGGCAGGAGGACUUCUACCCCUUCCUGA |
| | | AGGACAACAGGGAGAAGAUCGAGAAGAUCCUGACGUUCCGGAUCCCCUACUA |
| | | CGUUGGCCCCCUGGCCCGCGGCAACAGCCGGUUCGCCUGGAUGACGCGGAAG |
| | | AGCGAGGAGACGAUCACUCCCUGGAACUUCGAGGAAGUCGUGGACAAGGGUG |
| | | CCAGCGCCCAGAGCUUCAUCGAGCGGAUGACGAACUUCGACAAGAAUCUUCC |
| | | AAACGAGAAGGUGCUUCCAAAGCACAGCCUGCUGUACGAGUACUUCACGGUG |
| | | UACAACGAGCUGACGAAGGUGAAGUACGUGACAGAGGGCAUGCGGAAGCCCG |
| | | CCUUCCUCAGCGGUGAGCAGAAGAAGGCCAUCGUGGACCUGCUGUUCAAGAC |
| | | GAACCGGAAGGUGACGGUGAAGCAGCUGAAGGAGGACUACUUCAAGAAGAUC |
| | | GAGUGCUUCGACAGCGUGGAGAUCAGCGGCGUGGAGGACCGGUUCAACGCCA |
| | | GCCUGGGCACCUACCACGACCUGCUGAAGAUCAUCAAGGACAAGGACUUCCU |
| | | GGACAACGAGGAGAACGAGGACAUCCUGGAGGACAUCGUGCUGACGCUGACG |
| | | CUGUUCGAGGACAGGGAGAUGAUAGAGGAGCGGCUGAAGACCUACGCCCACC |
| | | UGUUCGACGACAAGGUGAUGAAGCAGCUGAAGCGGCGGCGGUACACGGGCUG |
| | | GGGCCGGCUCAGCCGGAAGCUGAUCAAUGGGAUCCGAGACAAGCAGAGCGGC |
| | | AAGACGAUCCUGGACUUCCUGAAGAGCGACGGCUUCGCCAACCGGAACUUCA |
| | | UGCAGCUGAUCCACGACGACAGCCUGACGUUCAAGGAGGACAUCCAGAAGGC |
| | | CCAGGUCAGCGGCCAGGGCGACAGCCUGCACGAGCACAUCGCCAAUCUCGCC |
| | | GGGAGCCCCGCCAUCAAGAAGGGGAUCCUGCAGACGGUGAAGGUGGUGGACG |
| | | AGCUGGUGAAGGUGAUGGGCCGGCACAAGCCAGAGAACAUCGUGAUCGAGAU |
| | | GGCCAGGGAGAACCAGACGACUCAAAAGGGGCAGAAGAACAGCAGGGAGCGG |
| | | AUGAAGCGGAUCGAGGAGGGCAUCAAGGAGCUGGGCAGCCAGAUCCUGAAGG |
| | | AGCACCCCGUGGAGAACACUCAACUGCAGAACGAGAAGCUGUACCUGUACUA |
| | | CCUGCAGAAUGGGCGAGACAUGUACGUGGACCAGGAGCUGGACAUCAACCGG |
| | | CUCAGCGACUACGACGUGGACCACAUCGUUCCCCAGAGCUUCCUGAAGGACG |
| | | ACAGCAUCGACAACAAGGUGCUGACGCGGAGCGACAAGAACCGGGGCAAGAG |
| | | CGACAACGUUCCCUCAGAGGAAGUCGUGAAGAAGAUGAAGAACUACUGGCGG |
| | | CAGCUGCUGAACGCCAAGCUGAUCACUCAACGGAAGUUCGACAAUCUCACGA |
| | | AGGCCGAGCGGGGUGGCCUCAGCGAGCUGGACAAGGCCGGGUUCAUCAAGCG |
| | | GCAGCUGGUGGAGACGCGGCAGAUCACGAAGCACGUGGCCCAGAUCCUGGAC |
| | | AGCCGGAUGAACACGAAGUACGACGAGAACGACAAGCUGAUCAGGGAAGUCA |
| | | AGGUGAUCACGCUGAAGAGCAAGCUGGUCAGCGACUUCCGGAAGGACUUCCA |
| | | GUUCUACAAGGUGAGGGAGAUCAACAACUACCACCACGCCCACGACGCCUAC |
| | | CUGAACGCUGUGGUUGGCACGGCACUGAUCAAGAAGUACCCCAAGCUGGAGA |
| | | GCGAGUUCGUGUACGGCGACUACAAGGUGUACGACGUGCGGAAGAUGAUAGC |
| | | CAAGAGCGAGCAGGAGAUCGGCAAGGCCACGGCCAAGUACUUCUUCUACAGC |
| | | AACAUCAUGAACUUCUUCAAGACAGAGAUCACGCUGGCCAAUGGUGAGAUCC |
| | | GGAAGCGGCCCCUGAUCGAGACGAAUGGUGAGACGGGUGAGAUCGUGUGGGA |
| | | CAAGGGGCGAGACUUCGCCACGGUGCGGAAGGUGCUCAGCAUGCCCCAGGUG |
| | | AACAUCGUGAAGAAGACAGAAGUCCAGACGGGUGGCUUCAGCAAGGAGAGCA |
| | | UCCUUCCAAAGCGGAACAGCGACAAGCUGAUCGCCCGCAAGAAGGACUGGGA |
| | | CCCCAAGAAGUACGGUGGCUUCGACAGCCCCACCGUGGCCUACAGCGUGCUG |
| | | GUGGUGGCCAAGGUGGAGAAGGGGAAGAGCAAGAAGCUGAAGAGCGUGAAGG |
| | | AGCUGCUGGGCAUCACGAUCAUGGAGCGGAGCAGCUUCGAGAAGAACCCCAU |
| | | CGACUUCCUGGAAGCCAAGGGGUACAAGGAAGUCAAGAAGGACCUGAUCAUC |
| | | AAGCUUCCAAAGUACAGCCUGUUCGAGCUGGAGAAUGGGCGGAAGCGGAUGC |
| | | UGGCCAGCGCCGGUGAGCUGCAGAAGGGGAACGAGCUGGCACUUCCCUCAAA |
| | | GUACGUGAACUUCCUGUACCUGGCCAGCCACUACGAGAAGCUGAAGGGGAGC |
| | | CCAGAGGACAACGAGCAGAAGCAGCUGUUCGUGGAGCAGCACAAGCACUACC |
| | | UGGACGAGAUCAUCGAGCAGAUCAGCGAGUUCAGCAAGCGGGUGAUCCUGGC |
| | | CGACGCCAAUCUCGACAAGGUGCUCAGCGCCUACAACAAGCACCGAGACAAG |
| | | CCCAUCAGGGAGCAGGCCGAGAACAUCAUCCACCUGUUCACGCUGACGAAUC |
| | | UCGGUGCCCCCGCUGCCUUCAAGUACUUCGACACGACGAUCGACCGGAAGCG |
| | | GUACACGUCGACUAAGGAAGUCCUGGACGCCACGCUGAUCCACCAGAGCAUC |
| | | ACGGGCCUGUACGAGACGCGGAUCGACCUCAGCCAGCUGGGUGGCGACGGUG |
| | | GUGGCAGCCCCAAGAAGAAGCGGAAGGUGUAG |
| 4 | ORF | AUGGACAAGAAGUACAGCAUCGGCCUCGACAUCGGCACCAACAGCGUCGGCU |
| | encoding | GGGCCGUCAUCACCGACGAGUACAAGGUCCCCAGCAAGAAGUUCAAGGUCCU |
| | Sp. Cas9 | CGGCAACACCGACCGCCACAGCAUCAAGAAGAACCUCAUCGGCGCCCUCCUC |
| | | UUCGACAGCGGCGAGACCGCCGAGGCCACCCGCCUCAAGCGCACCGCCCGCC |
| | | GCCGCUACACCCGCCGCAAGAACCGCAUCUGCUACCUCCAGGAGAUCUUCAG |
| | | CAACGAGAUGGCCAAGGUCGACGACAGCUUCUUCCACCGCCUCGAGGAGAGC |
| | | UUCCUCGUCGAGGAGGACAAGAAGCACGAGCGCCACCCCAUCUUCGGCAACA |
| | | UCGUCGACGAGGUCGCCUACCACGAGAAGUACCCCACCAUCUACCACCUCCG |
| | | CAAGAAGCUCGUCGACAGCACCGACAAGGCCGACCUCCGCCUCAUCUACCUC |
| | | GCCCUCGCCCACAUGAUCAAGUUCCGCGGCCACUUCCUCAUCGAGGGCGACC |
| | | UCAACCCCGACAACAGCGACGUCGACAAGCUCUUCAUCCAGCUCGUCCAGAC |
| | | CUACAACCAGCUCUUCGAGGAGAACCCCAUCAACGCCAGCGGCGUCGACGCC |
| | | AAGGCCAUCCUCAGCGCCCGCCUCAGCAAGAGCCGCCGCCUCGAGAACCUCA |
| | | UCGCCCAGCUCCCCGGCGAGAAGAAGAACGGCCUCUUCGGCAACCUCAUCGC |
| | | CCUCAGCCUCGGCCUCACCCCCAACUUCAAGAGCAACUUCGACCUCGCCGAG |
| | | GACGCCAAGCUCCAGCUCAGCAAGGACACCUACGACGACGACCUCGACAACC |
| | | UCCUCGCCCAGAUCGGCGACCAGUACGCCGACCUCUUCCUCGCCGCCAAGAA |
| | | CCUCAGCGACGCCAUCCUCCUCAGCGACAUCCUCCGCGUCAACACCGAGAUC |
| | | ACCAAGGCCCCCCUCAGCGCCAGCAUGAUCAAGCGCUACGACGAGCACCACC |
| | | AGGACCUCACCCUCCUCAAGGCCCUCGUCCGCCAGCAGCUCCCCGAGAAGUA |
| | | CAAGGAGAUCUUCUUCGACCAGAGCAAGAACGGCUACGCCGGCUACAUCGAC |
| | | GGCGGCGCCAGCCAGGAGGAGUUCUACAAGUUCAUCAAGCCCAUCCUCGAGA |
| | | AGAUGGACGGCACCGAGGAGCUCCUCGUCAAGCUCAACCGCGAGGACCUCCU |
| | | CCGCAAGCAGCGCACCUUCGACAACGGCAGCAUCCCCCACCAGAUCCACCUC |
| | | GGCGAGCUCCACGCCAUCCUCCGCCGCCAGGAGGACUUCUACCCCUUCCUCA |
| | | AGGACAACCGCGAGAAGAUCGAGAAGAUCCUCACCUUCCGCAUCCCCUACUA |
| | | CGUCGGCCCCCUCGCCCGCGGCAACAGCCGCUUCGCCUGGAUGACCCGCAAG |
| | | AGCGAGGAGACCAUCACCCCCUGGAACUUCGAGGAGGUCGUCGACAAGGGCG |
| | | CCAGCGCCCAGAGCUUCAUCGAGCGCAUGACCAACUUCGACAAGAACCUCCC |
| | | CAACGAGAAGGUCCUCCCCAAGCACAGCCUCCUCUACGAGUACUUCACCGUC |
| | | UACAACGAGCUCACCAAGGUCAAGUACGUCACCGAGGGCAUGCGCAAGCCCG |
| | | CCUUCCUCAGCGGCGAGCAGAAGAAGGCCAUCGUCGACCUCCUCUUCAAGAC |
| | | CAACCGCAAGGUCACCGUCAAGCAGCUCAAGGAGGACUACUUCAAGAAGAUC |
| | | GAGUGCUUCGACAGCGUCGAGAUCAGCGGCGUCGAGGACCGCUUCAACGCCA |
| | | GCCUCGGCACCUACCACGACCUCCUCAAGAUCAUCAAGGACAAGGACUUCCU |
| | | CGACAACGAGGAGAACGAGGACAUCCUCGAGGACAUCGUCCUCACCCUCACC |
| | | CUCUUCGAGGACCGCGAGAUGAUCGAGGAGCGCCUCAAGACCUACGCCCACC |
| | | UCUUCGACGACAAGGUCAUGAAGCAGCUCAAGCGCCGCCGCUACACCGGCUG |
| | | GGGCCGCCUCAGCCGCAAGCUCAUCAACGGCAUCCGCGACAAGCAGAGCGGC |
| | | AAGACCAUCCUCGACUUCCUCAAGAGCGACGGCUUCGCCAACCGCAACUUCA |
| | | UGCAGCUCAUCCACGACGACAGCCUCACCUUCAAGGAGGACAUCCAGAAGGC |
| | | CCAGGUCAGCGGCCAGGGCGACAGCCUCCACGAGCACAUCGCCAACCUCGCC |
| | | GGCAGCCCCGCCAUCAAGAAGGGCAUCCUCCAGACCGUCAAGGUCGUCGACG |
| | | AGCUCGUCAAGGUCAUGGGCCGCCACAAGCCCGAGAACAUCGUCAUCGAGAU |
| | | GGCCCGCGAGAACCAGACCACCCAGAAGGGCCAGAAGAACAGCCGCGAGCGC |
| | | AUGAAGCGCAUCGAGGAGGGCAUCAAGGAGCUCGGCAGCCAGAUCCUCAAGG |
| | | AGCACCCCGUCGAGAACACCCAGCUCCAGAACGAGAAGCUCUACCUCUACUA |
| | | CCUCCAGAACGGCCGCGACAUGUACGUCGACCAGGAGCUCGACAUCAACCGC |
| | | CUCAGCGACUACGACGUCGACCACAUCGUCCCCCAGAGCUUCCUCAAGGACG |
| | | ACAGCAUCGACAACAAGGUCCUCACCCGCAGCGACAAGAACCGCGGCAAGAG |
| | | CGACAACGUCCCCAGCGAGGAGGUCGUCAAGAAGAUGAAGAACUACUGGCGC |
| | | CAGCUCCUCAACGCCAAGCUCAUCACCCAGCGCAAGUUCGACAACCUCACCA |
| | | AGGCCGAGCGCGGCGGCCUCAGCGAGCUCGACAAGGCCGGCUUCAUCAAGCG |
| | | CCAGCUCGUCGAGACCCGCCAGAUCACCAAGCACGUCGCCCAGAUCCUCGAC |
| | | AGCCGCAUGAACACCAAGUACGACGAGAACGACAAGCUCAUCCGCGAGGUCA |
| | | AGGUCAUCACCCUCAAGAGCAAGCUCGUCAGCGACUUCCGCAAGGACUUCCA |
| | | GUUCUACAAGGUCCGCGAGAUCAACAACUACCACCACGCCCACGACGCCUAC |
| | | CUCAACGCCGUCGUCGGCACCGCCCUCAUCAAGAAGUACCCCAAGCUCGAGA |
| | | GCGAGUUCGUCUACGGCGACUACAAGGUCUACGACGUCCGCAAGAUGAUCGC |
| | | CAAGAGCGAGCAGGAGAUCGGCAAGGCCACCGCCAAGUACUUCUUCUACAGC |
| | | AACAUCAUGAACUUCUUCAAGACCGAGAUCACCCUCGCCAACGGCGAGAUCC |
| | | GCAAGCGCCCCCUCAUCGAGACCAACGGCGAGACCGGCGAGAUCGUCUGGGA |
| | | CAAGGGCCGCGACUUCGCCACCGUCCGCAAGGUCCUCAGCAUGCCCCAGGUC |
| | | AACAUCGUCAAGAAGACCGAGGUCCAGACCGGCGGCUUCAGCAAGGAGAGCA |
| | | UCCUCCCCAAGCGCAACAGCGACAAGCUCAUCGCCCGCAAGAAGGACUGGGA |
| | | CCCCAAGAAGUACGGCGGCUUCGACAGCCCCACCGUCGCCUACAGCGUCCUC |
| | | GUCGUCGCCAAGGUCGAGAAGGGCAAGAGCAAGAAGCUCAAGAGCGUCAAGG |
| | | AGCUCCUCGGCAUCACCAUCAUGGAGCGCAGCAGCUUCGAGAAGAACCCCAU |
| | | CGACUUCCUCGAGGCCAAGGGCUACAAGGAGGUCAAGAAGGACCUCAUCAUC |
| | | AAGCUCCCCAAGUACAGCCUCUUCGAGCUCGAGAACGGCCGCAAGCGCAUGC |
| | | UCGCCAGCGCCGGCGAGCUCCAGAAGGGCAACGAGCUCGCCCUCCCCAGCAA |
| | | GUACGUCAACUUCCUCUACCUCGCCAGCCACUACGAGAAGCUCAAGGGCAGC |
| | | CCCGAGGACAACGAGCAGAAGCAGCUCUUCGUCGAGCAGCACAAGCACUACC |
| | | UCGACGAGAUCAUCGAGCAGAUCAGCGAGUUCAGCAAGCGCGUCAUCCUCGC |
| | | CGACGCCAACCUCGACAAGGUCCUCAGCGCCUACAACAAGCACCGCGACAAG |
| | | CCCAUCCGCGAGCAGGCCGAGAACAUCAUCCACCUCUUCACCCUCACCAACC |
| | | UCGGCGCCCCCGCCGCCUUCAAGUACUUCGACACCACCAUCGACCGCAAGCG |
| | | CUACACCAGCACCAAGGAGGUCCUCGACGCCACCCUCAUCCACCAGAGCAUC |
| | | ACCGGCCUCUACGAGACCCGCAUCGACCUCAGCCAGCUCGGCGGCGACGGCG |
| | | GCGGCAGCCCCAAGAAGAAGCGCAAGGUCUAG |
| 5 | ORF | ATGGACAAGAAGTACAGCATCGGCCTGGACATCGGCACCAACAGCGTGGGCT |
| | encoding | GGGCCGTGATCACCGACGAGTACAAGGTGCCCAGCAAGAAGTTCAAGGTGCT |
| | Sp. Cas9 | GGGCAACACCGACCGGCACAGCATCAAGAAGAACCTGATCGGCGCCCTGCTG |
| | | TTCGACAGCGGCGAGACCGCCGAGGCCACCCGGCTGAAGCGGACCGCCCGGC |
| | | GGCGGTACACCCGGCGGAAGAACCGGATCTGCTACCTGCAGGAGATCTTCAG |
| | | CAACGAGATGGCCAAGGTGGACGACAGCTTCTTCCACCGGCTGGAGGAGAGC |
| | | TTCCTGGTGGAGGAGGACAAGAAGCACGAGCGGCACCCCATCTTCGGCAACA |
| | | TCGTGGACGAGGTGGCCTACCACGAGAAGTACCCCACCATCTACCACCTGCG |
| | | GAAGAAGCTGGTGGACAGCACCGACAAGGCCGACCTGCGGCTGATCTACCTG |
| | | GCCCTGGCCCACATGATCAAGTTCCGGGGCCACTTCCTGATCGAGGGCGACC |
| | | TGAACCCCGACAACAGCGACGTGGACAAGCTGTTCATCCAGCTGGTGCAGAC |
| | | CTACAACCAGCTGTTCGAGGAGAACCCCATCAACGCCAGCGGCGTGGACGCC |
| | | AAGGCCATCCTGAGCGCCCGGCTGAGCAAGAGCCGGCGGCTGGAGAACCTGA |
| | | TCGCCCAGCTGCCCGGCGAGAAGAAGAACGGCCTGTTCGGCAACCTGATCGC |
| | | CCTGAGCCTGGGCCTGACCCCCAACTTCAAGAGCAACTTCGACCTGGCCGAG |
| | | GACGCCAAGCTGCAGCTGAGCAAGGACACCTACGACGACGACCTGGACAACC |
| | | TGCTGGCCCAGATCGGCGACCAGTACGCCGACCTGTTCCTGGCCGCCAAGAA |
| | | CCTGAGCGACGCCATCCTGCTGAGCGACATCCTGCGGGTGAACACCGAGATC |
| | | ACCAAGGCCCCCCTGAGCGCCAGCATGATCAAGCGGTACGACGAGCACCACC |
| | | AGGACCTGACCCTGCTGAAGGCCCTGGTGCGGCAGCAGCTGCCCGAGAAGTA |
| | | CAAGGAGATCTTCTTCGACCAGAGCAAGAACGGCTACGCCGGCTACATCGAC |
| | | GGCGGCGCCAGCCAGGAGGAGTTCTACAAGTTCATCAAGCCCATCCTGGAGA |
| | | AGATGGACGGCACCGAGGAGCTGCTGGTGAAGCTGAACCGGGAGGACCTGCT |
| | | GCGGAAGCAGCGGACCTTCGACAACGGCAGCATCCCCCACCAGATCCACCTG |
| | | GGCGAGCTGCACGCCATCCTGCGGCGGCAGGAGGACTTCTACCCCTTCCTGA |
| | | AGGACAACCGGGAGAAGATCGAGAAGATCCTGACCTTCCGGATCCCCTACTA |
| | | CGTGGGCCCCCTGGCCCGGGGCAACAGCCGGTTCGCCTGGATGACCCGGAAG |
| | | AGCGAGGAGACCATCACCCCCTGGAACTTCGAGGAGGTGGTGGACAAGGGCG |
| | | CCAGCGCCCAGAGCTTCATCGAGCGGATGACCAACTTCGACAAGAACCTGCC |
| | | CAACGAGAAGGTGCTGCCCAAGCACAGCCTGCTGTACGAGTACTTCACCGTG |
| | | TACAACGAGCTGACCAAGGTGAAGTACGTGACCGAGGGCATGCGGAAGCCCG |
| | | CCTTCCTGAGCGGCGAGCAGAAGAAGGCCATCGTGGACCTGCTGTTCAAGAC |
| | | CAACCGGAAGGTGACCGTGAAGCAGCTGAAGGAGGACTACTTCAAGAAGATC |
| | | GAGTGCTTCGACAGCGTGGAGATCAGCGGCGTGGAGGACCGGTTCAACGCCA |
| | | GCCTGGGCACCTACCACGACCTGCTGAAGATCATCAAGGACAAGGACTTCCT |
| | | GGACAACGAGGAGAACGAGGACATCCTGGAGGACATCGTGCTGACCCTGACC |
| | | CTGTTCGAGGACCGGGAGATGATCGAGGAGCGGCTGAAGACCTACGCCCACC |
| | | TGTTCGACGACAAGGTGATGAAGCAGCTGAAGCGGCGGCGGTACACCGGCTG |
| | | GGGCCGGCTGAGCCGGAAGCTGATCAACGGCATCCGGGACAAGCAGAGCGGC |
| | | AAGACCATCCTGGACTTCCTGAAGAGCGACGGCTTCGCCAACCGGAACTTCA |
| | | TGCAGCTGATCCACGACGACAGCCTGACCTTCAAGGAGGACATCCAGAAGGC |
| | | CCAGGTGAGCGGCCAGGGCGACAGCCTGCACGAGCACATCGCCAACCTGGCC |
| | | GGCAGCCCCGCCATCAAGAAGGGCATCCTGCAGACCGTGAAGGTGGTGGACG |
| | | AGCTGGTGAAGGTGATGGGCCGGCACAAGCCCGAGAACATCGTGATCGAGAT |
| | | GGCCCGGGAGAACCAGACCACCCAGAAGGGCCAGAAGAACAGCCGGGAGCGG |
| | | ATGAAGCGGATCGAGGAGGGCATCAAGGAGCTGGGCAGCCAGATCCTGAAGG |
| | | AGCACCCCGTGGAGAACACCCAGCTGCAGAACGAGAAGCTGTACCTGTACTA |
| | | CCTGCAGAACGGCCGGGACATGTACGTGGACCAGGAGCTGGACATCAACCGG |
| | | CTGAGCGACTACGACGTGGACCACATCGTGCCCCAGAGCTTCCTGAAGGACG |
| | | ACAGCATCGACAACAAGGTGCTGACCCGGAGCGACAAGAACCGGGGCAAGAG |
| | | CGACAACGTGCCCAGCGAGGAGGTGGTGAAGAAGATGAAGAACTACTGGCGG |
| | | CAGCTGCTGAACGCCAAGCTGATCACCCAGCGGAAGTTCGACAACCTGACCA |
| | | AGGCCGAGCGGGGCGGCCTGAGCGAGCTGGACAAGGCCGGCTTCATCAAGCG |
| | | GCAGCTGGTGGAGACCCGGCAGATCACCAAGCACGTGGCCCAGATCCTGGAC |
| | | AGCCGGATGAACACCAAGTACGACGAGAACGACAAGCTGATCCGGGAGGTGA |
| | | AGGTGATCACCCTGAAGAGCAAGCTGGTGAGCGACTTCCGGAAGGACTTCCA |
| | | GTTCTACAAGGTGCGGGAGATCAACAACTACCACCACGCCCACGACGCCTAC |
| | | CTGAACGCCGTGGTGGGCACCGCCCTGATCAAGAAGTACCCCAAGCTGGAGA |
| | | GCGAGTTCGTGTACGGCGACTACAAGGTGTACGACGTGCGGAAGATGATCGC |
| | | CAAGAGCGAGCAGGAGATCGGCAAGGCCACCGCCAAGTACTTCTTCTACAGC |
| | | AACATCATGAACTTCTTCAAGACCGAGATCACCCTGGCCAACGGCGAGATCC |
| | | GGAAGCGGCCCCTGATCGAGACCAACGGCGAGACCGGCGAGATCGTGTGGGA |
| | | CAAGGGCCGGGACTTCGCCACCGTGCGGAAGGTGCTGAGCATGCCCCAGGTG |
| | | AACATCGTGAAGAAGACCGAGGTGCAGACCGGCGGCTTCAGCAAGGAGAGCA |
| | | TCCTGCCCAAGCGGAACAGCGACAAGCTGATCGCCCGGAAGAAGGACTGGGA |
| | | CCCCAAGAAGTACGGCGGCTTCGACAGCCCCACCGTGGCCTACAGCGTGCTG |
| | | GTGGTGGCCAAGGTGGAGAAGGGCAAGAGCAAGAAGCTGAAGAGCGTGAAGG |
| | | AGCTGCTGGGCATCACCATCATGGAGCGGAGCAGCTTCGAGAAGAACCCCAT |
| | | CGACTTCCTGGAGGCCAAGGGCTACAAGGAGGTGAAGAAGGACCTGATCATC |
| | | AAGCTGCCCAAGTACAGCCTGTTCGAGCTGGAGAACGGCCGGAAGCGGATGC |
| | | TGGCCAGCGCCGGCGAGCTGCAGAAGGGCAACGAGCTGGCCCTGCCCAGCAA |
| | | GTACGTGAACTTCCTGTACCTGGCCAGCCACTACGAGAAGCTGAAGGGCAGC |
| | | CCCGAGGACAACGAGCAGAAGCAGCTGTTCGTGGAGCAGCACAAGCACTACC |
| | | TGGACGAGATCATCGAGCAGATCAGCGAGTTCAGCAAGCGGGTGATCCTGGC |
| | | CGACGCCAACCTGGACAAGGTGCTGAGCGCCTACAACAAGCACCGGGACAAG |
| | | CCCATCCGGGAGCAGGCCGAGAACATCATCCACCTGTTCACCCTGACCAACC |
| | | TGGGCGCCCCCGCCGCCTTCAAGTACTTCGACACCACCATCGACCGGAAGCG |
| | | GTACACCAGCACCAAGGAGGTGCTGGACGCCACCCTGATCCACCAGAGCATC |
| | | ACCGGCCTGTACGAGACCCGGATCGACCTGAGCCAGCTGGGCGGCGACGGCG |
| | | GCGGCAGCCCCAAGAAGAAGCGGAAGGTGTGA |
| 6 | ORF | ATGGACAAGAAGTACTCCATCGGCCTGGCCATCGGCACCAACTCCGTGGGCT |
| | encoding | GGGCCGTGATCACCGACGAGTACAAGGTGCCCTCCAAGAAGTTCAAGGTGCT |
| | Sp. Cas9 | GGGCAACACCGACCGGCACTCCATCAAGAAGAACCTGATCGGCGCCCTGCTG |
| | nickase | TTCGACTCCGGCGAGACCGCCGAGGCCACCCGGCTGAAGCGGACCGCCCGGC |
| | | GGCGGTACACCCGGCGGAAGAACCGGATCTGCTACCTGCAGGAGATCTTCTC |
| | | CAACGAGATGGCCAAGGTGGACGACTCCTTCTTCCACCGGCTGGAGGAGTCC |
| | | TTCCTGGTGGAGGAGGACAAGAAGCACGAGCGGCACCCCATCTTCGGCAACA |
| | | TCGTGGACGAGGTGGCCTACCACGAGAAGTACCCCACCATCTACCACCTGCG |
| | | GAAGAAGCTGGTGGACTCCACCGACAAGGCCGACCTGCGGCTGATCTACCTG |
| | | GCCCTGGCCCACATGATCAAGTTCCGGGGCCACTTCCTGATCGAGGGCGACC |
| | | TGAACCCCGACAACTCCGACGTGGACAAGCTGTTCATCCAGCTGGTGCAGAC |
| | | CTACAACCAGCTGTTCGAGGAGAACCCCATCAACGCCTCCGGCGTGGACGCC |
| | | AAGGCCATCCTGTCCGCCCGGCTGTCCAAGTCCCGGCGGCTGGAGAACCTGA |
| | | TCGCCCAGCTGCCCGGCGAGAAGAAGAACGGCCTGTTCGGCAACCTGATCGC |
| | | CCTGTCCCTGGGCCTGACCCCCAACTTCAAGTCCAACTTCGACCTGGCCGAG |
| | | GACGCCAAGCTGCAGCTGTCCAAGGACACCTACGACGACGACCTGGACAACC |
| | | TGCTGGCCCAGATCGGCGACCAGTACGCCGACCTGTTCCTGGCCGCCAAGAA |
| | | CCTGTCCGACGCCATCCTGCTGTCCGACATCCTGCGGGTGAACACCGAGATC |
| | | ACCAAGGCCCCCCTGTCCGCCTCCATGATCAAGCGGTACGACGAGCACCACC |
| | | AGGACCTGACCCTGCTGAAGGCCCTGGTGCGGCAGCAGCTGCCCGAGAAGTA |
| | | CAAGGAGATCTTCTTCGACCAGTCCAAGAACGGCTACGCCGGCTACATCGAC |
| | | GGCGGCGCCTCCCAGGAGGAGTTCTACAAGTTCATCAAGCCCATCCTGGAGA |
| | | AGATGGACGGCACCGAGGAGCTGCTGGTGAAGCTGAACCGGGAGGACCTGCT |
| | | GCGGAAGCAGCGGACCTTCGACAACGGCTCCATCCCCCACCAGATCCACCTG |
| | | GGCGAGCTGCACGCCATCCTGCGGCGGCAGGAGGACTTCTACCCCTTCCTGA |
| | | AGGACAACCGGGAGAAGATCGAGAAGATCCTGACCTTCCGGATCCCCTACTA |
| | | CGTGGGCCCCCTGGCCCGGGGCAACTCCCGGTTCGCCTGGATGACCCGGAAG |
| | | TCCGAGGAGACCATCACCCCCTGGAACTTCGAGGAGGTGGTGGACAAGGGCG |
| | | CCTCCGCCCAGTCCTTCATCGAGCGGATGACCAACTTCGACAAGAACCTGCC |
| | | CAACGAGAAGGTGCTGCCCAAGCACTCCCTGCTGTACGAGTACTTCACCGTG |
| | | TACAACGAGCTGACCAAGGTGAAGTACGTGACCGAGGGCATGCGGAAGCCCG |
| | | CCTTCCTGTCCGGCGAGCAGAAGAAGGCCATCGTGGACCTGCTGTTCAAGAC |
| | | CAACCGGAAGGTGACCGTGAAGCAGCTGAAGGAGGACTACTTCAAGAAGATC |
| | | GAGTGCTTCGACTCCGTGGAGATCTCCGGCGTGGAGGACCGGTTCAACGCCT |
| | | CCCTGGGCACCTACCACGACCTGCTGAAGATCATCAAGGACAAGGACTTCCT |
| | | GGACAACGAGGAGAACGAGGACATCCTGGAGGACATCGTGCTGACCCTGACC |
| | | CTGTTCGAGGACCGGGAGATGATCGAGGAGCGGCTGAAGACCTACGCCCACC |
| | | TGTTCGACGACAAGGTGATGAAGCAGCTGAAGCGGCGGCGGTACACCGGCTG |
| | | GGGCCGGCTGTCCCGGAAGCTGATCAACGGCATCCGGGACAAGCAGTCCGGC |
| | | AAGACCATCCTGGACTTCCTGAAGTCCGACGGCTTCGCCAACCGGAACTTCA |
| | | TGCAGCTGATCCACGACGACTCCCTGACCTTCAAGGAGGACATCCAGAAGGC |
| | | CCAGGTGTCCGGCCAGGGCGACTCCCTGCACGAGCACATCGCCAACCTGGCC |
| | | GGCTCCCCCGCCATCAAGAAGGGCATCCTGCAGACCGTGAAGGTGGTGGACG |
| | | AGCTGGTGAAGGTGATGGGCCGGCACAAGCCCGAGAACATCGTGATCGAGAT |
| | | GGCCCGGGAGAACCAGACCACCCAGAAGGGCCAGAAGAACTCCCGGGAGCGG |
| | | ATGAAGCGGATCGAGGAGGGCATCAAGGAGCTGGGCTCCCAGATCCTGAAGG |
| | | AGCACCCCGTGGAGAACACCCAGCTGCAGAACGAGAAGCTGTACCTGTACTA |
| | | CCTGCAGAACGGCCGGGACATGTACGTGGACCAGGAGCTGGACATCAACCGG |
| | | CTGTCCGACTACGACGTGGACCACATCGTGCCCCAGTCCTTCCTGAAGGACG |
| | | ACTCCATCGACAACAAGGTGCTGACCCGGTCCGACAAGAACCGGGGCAAGTC |
| | | CGACAACGTGCCCTCCGAGGAGGTGGTGAAGAAGATGAAGAACTACTGGCGG |
| | | CAGCTGCTGAACGCCAAGCTGATCACCCAGCGGAAGTTCGACAACCTGACCA |
| | | AGGCCGAGCGGGGCGGCCTGTCCGAGCTGGACAAGGCCGGCTTCATCAAGCG |
| | | GCAGCTGGTGGAGACCCGGCAGATCACCAAGCACGTGGCCCAGATCCTGGAC |
| | | TCCCGGATGAACACCAAGTACGACGAGAACGACAAGCTGATCCGGGAGGTGA |
| | | AGGTGATCACCCTGAAGTCCAAGCTGGTGTCCGACTTCCGGAAGGACTTCCA |
| | | GTTCTACAAGGTGCGGGAGATCAACAACTACCACCACGCCCACGACGCCTAC |
| | | CTGAACGCCGTGGTGGGCACCGCCCTGATCAAGAAGTACCCCAAGCTGGAGT |
| | | CCGAGTTCGTGTACGGCGACTACAAGGTGTACGACGTGCGGAAGATGATCGC |
| | | CAAGTCCGAGCAGGAGATCGGCAAGGCCACCGCCAAGTACTTCTTCTACTCC |
| | | AACATCATGAACTTCTTCAAGACCGAGATCACCCTGGCCAACGGCGAGATCC |
| | | GGAAGCGGCCCCTGATCGAGACCAACGGCGAGACCGGCGAGATCGTGTGGGA |
| | | CAAGGGCCGGGACTTCGCCACCGTGCGGAAGGTGCTGTCCATGCCCCAGGTG |
| | | AACATCGTGAAGAAGACCGAGGTGCAGACCGGCGGCTTCTCCAAGGAGTCCA |
| | | TCCTGCCCAAGCGGAACTCCGACAAGCTGATCGCCCGGAAGAAGGACTGGGA |
| | | CCCCAAGAAGTACGGCGGCTTCGACTCCCCCACCGTGGCCTACTCCGTGCTG |
| | | GTGGTGGCCAAGGTGGAGAAGGGCAAGTCCAAGAAGCTGAAGTCCGTGAAGG |
| | | AGCTGCTGGGCATCACCATCATGGAGCGGTCCTCCTTCGAGAAGAACCCCAT |
| | | CGACTTCCTGGAGGCCAAGGGCTACAAGGAGGTGAAGAAGGACCTGATCATC |
| | | AAGCTGCCCAAGTACTCCCTGTTCGAGCTGGAGAACGGCCGGAAGCGGATGC |
| | | TGGCCTCCGCCGGCGAGCTGCAGAAGGGCAACGAGCTGGCCCTGCCCTCCAA |
| | | GTACGTGAACTTCCTGTACCTGGCCTCCCACTACGAGAAGCTGAAGGGCTCC |
| | | CCCGAGGACAACGAGCAGAAGCAGCTGTTCGTGGAGCAGCACAAGCACTACC |
| | | TGGACGAGATCATCGAGCAGATCTCCGAGTTCTCCAAGCGGGTGATCCTGGC |
| | | CGACGCCAACCTGGACAAGGTGCTGTCCGCCTACAACAAGCACCGGGACAAG |
| | | CCCATCCGGGAGCAGGCCGAGAACATCATCCACCTGTTCACCCTGACCAACC |
| | | TGGGCGCCCCCGCCGCCTTCAAGTACTTCGACACCACCATCGACCGGAAGCG |
| | | GTACACCTCCACCAAGGAGGTGCTGGACGCCACCCTGATCCACCAGTCCATC |
| | | ACCGGCCTGTACGAGACCCGGATCGACCTGTCCCAGCTGGGCGGCGACGGCG |
| | | GCGGCTCCCCCAAGAAGAAGCGGAAGGTGTGA |
| 7 | ORF | ATGGACAAGAAGTACAGCATCGGCCTGGCCATCGGCACCAACAGCGTGGGCT |
| | encoding | GGGCCGTGATCACCGACGAGTACAAGGTGCCCAGCAAGAAGTTCAAGGTGCT |
| | Sp. Cas9 | GGGCAACACCGACCGGCACAGCATCAAGAAGAACCTGATCGGCGCCCTGCTG |
| | nickase | TTCGACAGCGGCGAGACCGCCGAGGCCACCCGGCTGAAGCGGACCGCCCGGC |
| | | GGCGGTACACCCGGCGGAAGAACCGGATCTGCTACCTGCAGGAGATCTTCAG |
| | | CAACGAGATGGCCAAGGTGGACGACAGCTTCTTCCACCGGCTGGAGGAGAGC |
| | | TTCCTGGTGGAGGAGGACAAGAAGCACGAGCGGCACCCCATCTTCGGCAACA |
| | | TCGTGGACGAGGTGGCCTACCACGAGAAGTACCCCACCATCTACCACCTGCG |
| | | GAAGAAGCTGGTGGACAGCACCGACAAGGCCGACCTGCGGCTGATCTACCTG |
| | | GCCCTGGCCCACATGATCAAGTTCCGGGGCCACTTCCTGATCGAGGGCGACC |
| | | TGAACCCCGACAACAGCGACGTGGACAAGCTGTTCATCCAGCTGGTGCAGAC |
| | | CTACAACCAGCTGTTCGAGGAGAACCCCATCAACGCCAGCGGCGTGGACGCC |
| | | AAGGCCATCCTGAGCGCCCGGCTGAGCAAGAGCCGGCGGCTGGAGAACCTGA |
| | | TCGCCCAGCTGCCCGGCGAGAAGAAGAACGGCCTGTTCGGCAACCTGATCGC |
| | | CCTGAGCCTGGGCCTGACCCCCAACTTCAAGAGCAACTTCGACCTGGCCGAG |
| | | GACGCCAAGCTGCAGCTGAGCAAGGACACCTACGACGACGACCTGGACAACC |
| | | TGCTGGCCCAGATCGGCGACCAGTACGCCGACCTGTTCCTGGCCGCCAAGAA |
| | | CCTGAGCGACGCCATCCTGCTGAGCGACATCCTGCGGGTGAACACCGAGATC |
| | | ACCAAGGCCCCCCTGAGCGCCAGCATGATCAAGCGGTACGACGAGCACCACC |
| | | AGGACCTGACCCTGCTGAAGGCCCTGGTGCGGCAGCAGCTGCCCGAGAAGTA |
| | | CAAGGAGATCTTCTTCGACCAGAGCAAGAACGGCTACGCCGGCTACATCGAC |
| | | GGCGGCGCCAGCCAGGAGGAGTTCTACAAGTTCATCAAGCCCATCCTGGAGA |
| | | AGATGGACGGCACCGAGGAGCTGCTGGTGAAGCTGAACCGGGAGGACCTGCT |
| | | GCGGAAGCAGCGGACCTTCGACAACGGCAGCATCCCCCACCAGATCCACCTG |
| | | GGCGAGCTGCACGCCATCCTGCGGCGGCAGGAGGACTTCTACCCCTTCCTGA |
| | | AGGACAACCGGGAGAAGATCGAGAAGATCCTGACCTTCCGGATCCCCTACTA |
| | | CGTGGGCCCCCTGGCCCGGGGCAACAGCCGGTTCGCCTGGATGACCCGGAAG |
| | | AGCGAGGAGACCATCACCCCCTGGAACTTCGAGGAGGTGGTGGACAAGGGCG |
| | | CCAGCGCCCAGAGCTTCATCGAGCGGATGACCAACTTCGACAAGAACCTGCC |
| | | CAACGAGAAGGTGCTGCCCAAGCACAGCCTGCTGTACGAGTACTTCACCGTG |
| | | TACAACGAGCTGACCAAGGTGAAGTACGTGACCGAGGGCATGCGGAAGCCCG |
| | | CCTTCCTGAGCGGCGAGCAGAAGAAGGCCATCGTGGACCTGCTGTTCAAGAC |
| | | CAACCGGAAGGTGACCGTGAAGCAGCTGAAGGAGGACTACTTCAAGAAGATC |
| | | GAGTGCTTCGACAGCGTGGAGATCAGCGGCGTGGAGGACCGGTTCAACGCCA |
| | | GCCTGGGCACCTACCACGACCTGCTGAAGATCATCAAGGACAAGGACTTCCT |
| | | GGACAACGAGGAGAACGAGGACATCCTGGAGGACATCGTGCTGACCCTGACC |
| | | CTGTTCGAGGACCGGGAGATGATCGAGGAGCGGCTGAAGACCTACGCCCACC |
| | | TGTTCGACGACAAGGTGATGAAGCAGCTGAAGCGGCGGCGGTACACCGGCTG |
| | | GGGCCGGCTGAGCCGGAAGCTGATCAACGGCATCCGGGACAAGCAGAGCGGC |
| | | AAGACCATCCTGGACTTCCTGAAGAGCGACGGCTTCGCCAACCGGAACTTCA |
| | | TGCAGCTGATCCACGACGACAGCCTGACCTTCAAGGAGGACATCCAGAAGGC |
| | | CCAGGTGAGCGGCCAGGGCGACAGCCTGCACGAGCACATCGCCAACCTGGCC |
| | | GGCAGCCCCGCCATCAAGAAGGGCATCCTGCAGACCGTGAAGGTGGTGGACG |
| | | AGCTGGTGAAGGTGATGGGCCGGCACAAGCCCGAGAACATCGTGATCGAGAT |
| | | GGCCCGGGAGAACCAGACCACCCAGAAGGGCCAGAAGAACAGCCGGGAGCGG |
| | | ATGAAGCGGATCGAGGAGGGCATCAAGGAGCTGGGCAGCCAGATCCTGAAGG |
| | | AGCACCCCGTGGAGAACACCCAGCTGCAGAACGAGAAGCTGTACCTGTACTA |
| | | CCTGCAGAACGGCCGGGACATGTACGTGGACCAGGAGCTGGACATCAACCGG |
| | | CTGAGCGACTACGACGTGGACCACATCGTGCCCCAGAGCTTCCTGAAGGACG |
| | | ACAGCATCGACAACAAGGTGCTGACCCGGAGCGACAAGAACCGGGGCAAGAG |
| | | CGACAACGTGCCCAGCGAGGAGGTGGTGAAGAAGATGAAGAACTACTGGCGG |
| | | CAGCTGCTGAACGCCAAGCTGATCACCCAGCGGAAGTTCGACAACCTGACCA |
| | | AGGCCGAGCGGGGCGGCCTGAGCGAGCTGGACAAGGCCGGCTTCATCAAGCG |
| | | GCAGCTGGTGGAGACCCGGCAGATCACCAAGCACGTGGCCCAGATCCTGGAC |
| | | AGCCGGATGAACACCAAGTACGACGAGAACGACAAGCTGATCCGGGAGGTGA |
| | | AGGTGATCACCCTGAAGAGCAAGCTGGTGAGCGACTTCCGGAAGGACTTCCA |
| | | GTTCTACAAGGTGCGGGAGATCAACAACTACCACCACGCCCACGACGCCTAC |
| | | CTGAACGCCGTGGTGGGCACCGCCCTGATCAAGAAGTACCCCAAGCTGGAGA |
| | | GCGAGTTCGTGTACGGCGACTACAAGGTGTACGACGTGCGGAAGATGATCGC |
| | | CAAGAGCGAGCAGGAGATCGGCAAGGCCACCGCCAAGTACTTCTTCTACAGC |
| | | AACATCATGAACTTCTTCAAGACCGAGATCACCCTGGCCAACGGCGAGATCC |
| | | GGAAGCGGCCCCTGATCGAGACCAACGGCGAGACCGGCGAGATCGTGTGGGA |
| | | CAAGGGCCGGGACTTCGCCACCGTGCGGAAGGTGCTGAGCATGCCCCAGGTG |
| | | AACATCGTGAAGAAGACCGAGGTGCAGACCGGCGGCTTCAGCAAGGAGAGCA |
| | | TCCTGCCCAAGCGGAACAGCGACAAGCTGATCGCCCGGAAGAAGGACTGGGA |
| | | CCCCAAGAAGTACGGCGGCTTCGACAGCCCCACCGTGGCCTACAGCGTGCTG |
| | | GTGGTGGCCAAGGTGGAGAAGGGCAAGAGCAAGAAGCTGAAGAGCGTGAAGG |
| | | AGCTGCTGGGCATCACCATCATGGAGCGGAGCAGCTTCGAGAAGAACCCCAT |
| | | CGACTTCCTGGAGGCCAAGGGCTACAAGGAGGTGAAGAAGGACCTGATCATC |
| | | AAGCTGCCCAAGTACAGCCTGTTCGAGCTGGAGAACGGCCGGAAGCGGATGC |
| | | TGGCCAGCGCCGGCGAGCTGCAGAAGGGCAACGAGCTGGCCCTGCCCAGCAA |
| | | GTACGTGAACTTCCTGTACCTGGCCAGCCACTACGAGAAGCTGAAGGGCAGC |
| | | CCCGAGGACAACGAGCAGAAGCAGCTGTTCGTGGAGCAGCACAAGCACTACC |
| | | TGGACGAGATCATCGAGCAGATCAGCGAGTTCAGCAAGCGGGTGATCCTGGC |
| | | CGACGCCAACCTGGACAAGGTGCTGAGCGCCTACAACAAGCACCGGGACAAG |
| | | CCCATCCGGGAGCAGGCCGAGAACATCATCCACCTGTTCACCCTGACCAACC |
| | | TGGGCGCCCCCGCCGCCTTCAAGTACTTCGACACCACCATCGACCGGAAGCG |
| | | GTACACCAGCACCAAGGAGGTGCTGGACGCCACCCTGATCCACCAGAGCATC |
| | | ACCGGCCTGTACGAGACCCGGATCGACCTGAGCCAGCTGGGCGGCGACGGCG |
| | | GCGGCAGCCCCAAGAAGAAGCGGAAGGTGTGA |
| 8 | mRNA | GGGUCCCGCAGUCGGCGUCCAGCGGCUCUGCUUGUUCGUGUGUGUGUCGUUG |
| | encoding | CAGGCCUUAUUCGGAUCCGCCACCAUGGACAAGAAGUACAGCAUCGGACUGG |
| | Sp. Cas9 | ACAUCGGAACAAACAGCGUCGGAUGGGCAGUCAUCACAGACGAAUACAAGGU |
| | | CCCGAGCAAGAAGUUCAAGGUCCUGGGAAACACAGACAGACACAGCAUCAAG |
| | | AAGAACCUGAUCGGAGCACUGCUGUUCGACAGCGGAGAAACAGCAGAAGCAA |
| | | CAAGACUGAAGAGAACAGCAAGAAGAAGAUACACAAGAAGAAAGAACAGAAU |
| | | CUGCUACCUGCAGGAAAUCUUCAGCAACGAAAUGGCAAAGGUCGACGACAGC |
| | | UUCUUCCACAGACUGGAAGAAAGCUUCCUGGUCGAAGAAGACAAGAAGCACG |
| | | AAAGACACCCGAUCUUCGGAAACAUCGUCGACGAAGUCGCAUACCACGAAAA |
| | | GUACCCGACAAUCUACCACCUGAGAAAGAAGCUGGUCGACAGCACAGACAAG |
| | | GCAGACCUGAGACUGAUCUACCUGGCACUGGCACACAUGAUCAAGUUCAGAG |
| | | GACACUUCCUGAUCGAAGGAGACCUGAACCCGGACAACAGCGACGUCGACAA |
| | | GCUGUUCAUCCAGCUGGUCCAGACAUACAACCAGCUGUUCGAAGAAAACCCG |
| | | AUCAACGCAAGCGGAGUCGACGCAAAGGCAAUCCUGAGCGCAAGACUGAGCA |
| | | AGAGCAGAAGACUGGAAAACCUGAUCGCACAGCUGCCGGGAGAAAAGAAGAA |
| | | CGGACUGUUCGGAAACCUGAUCGCACUGAGCCUGGGACUGACACCGAACUUC |
| | | AAGAGCAACUUCGACCUGGCAGAAGACGCAAAGCUGCAGCUGAGCAAGGACA |
| | | CAUACGACGACGACCUGGACAACCUGCUGGCACAGAUCGGAGACCAGUACGC |
| | | AGACCUGUUCCUGGCAGCAAAGAACCUGAGCGACGCAAUCCUGCUGAGCGAC |
| | | AUCCUGAGAGUCAACACAGAAAUCACAAAGGCACCGCUGAGCGCAAGCAUGA |
| | | UCAAGAGAUACGACGAACACCACCAGGACCUGACACUGCUGAAGGCACUGGU |
| | | CAGACAGCAGCUGCCGGAAAAGUACAAGGAAAUCUUCUUCGACCAGAGCAAG |
| | | AACGGAUACGCAGGAUACAUCGACGGAGGAGCAAGCCAGGAAGAAUUCUACA |
| | | AGUUCAUCAAGCCGAUCCUGGAAAAGAUGGACGGAACAGAAGAACUGCUGGU |
| | | CAAGCUGAACAGAGAAGACCUGCUGAGAAAGCAGAGAACAUUCGACAACGGA |
| | | AGCAUCCCGCACCAGAUCCACCUGGGAGAACUGCACGCAAUCCUGAGAAGAC |
| | | AGGAAGACUUCUACCCGUUCCUGAAGGACAACAGAGAAAAGAUCGAAAAGAU |
| | | CCUGACAUUCAGAAUCCCGUACUACGUCGGACCGCUGGCAAGAGGAAACAGC |
| | | AGAUUCGCAUGGAUGACAAGAAAGAGCGAAGAAACAAUCACACCGUGGAACU |
| | | UCGAAGAAGUCGUCGACAAGGGAGCAAGCGCACAGAGCUUCAUCGAAAGAAU |
| | | GACAAACUUCGACAAGAACCUGCCGAACGAAAAGGUCCUGCCGAAGCACAGC |
| | | CUGCUGUACGAAUACUUCACAGUCUACAACGAACUGACAAAGGUCAAGUACG |
| | | UCACAGAAGGAAUGAGAAAGCCGGCAUUCCUGAGCGGAGAACAGAAGAAGGC |
| | | AAUCGUCGACCUGCUGUUCAAGACAAACAGAAAGGUCACAGUCAAGCAGCUG |
| | | AAGGAAGACUACUUCAAGAAGAUCGAAUGCUUCGACAGCGUCGAAAUCAGCG |
| | | GAGUCGAAGACAGAUUCAACGCAAGCCUGGGAACAUACCACGACCUGCUGAA |
| | | GAUCAUCAAGGACAAGGACUUCCUGGACAACGAAGAAAACGAAGACAUCCUG |
| | | GAAGACAUCGUCCUGACACUGACACUGUUCGAAGACAGAGAAAUGAUCGAAG |
| | | AAAGACUGAAGACAUACGCACACCUGUUCGACGACAAGGUCAUGAAGCAGCU |
| | | GAAGAGAAGAAGAUACACAGGAUGGGGAAGACUGAGCAGAAAGCUGAUCAAC |
| | | GGAAUCAGAGACAAGCAGAGCGGAAAGACAAUCCUGGACUUCCUGAAGAGCG |
| | | ACGGAUUCGCAAACAGAAACUUCAUGCAGCUGAUCCACGACGACAGCCUGAC |
| | | AUUCAAGGAAGACAUCCAGAAGGCACAGGUCAGCGGACAGGGAGACAGCCUG |
| | | CACGAACACAUCGCAAACCUGGCAGGAAGCCCGGCAAUCAAGAAGGGAAUCC |
| | | UGCAGACAGUCAAGGUCGUCGACGAACUGGUCAAGGUCAUGGGAAGACACAA |
| | | GCCGGAAAACAUCGUCAUCGAAAUGGCAAGAGAAAACCAGACAACACAGAAG |
| | | GGACAGAAGAACAGCAGAGAAAGAAUGAAGAGAAUCGAAGAAGGAAUCAAGG |
| | | AACUGGGAAGCCAGAUCCUGAAGGAACACCCGGUCGAAAACACACAGCUGCA |
| | | GAACGAAAAGCUGUACCUGUACUACCUGCAGAACGGAAGAGACAUGUACGUC |
| | | GACCAGGAACUGGACAUCAACAGACUGAGCGACUACGACGUCGACCACAUCG |
| | | UCCCGCAGAGCUUCCUGAAGGACGACAGCAUCGACAACAAGGUCCUGACAAG |
| | | AAGCGACAAGAACAGAGGAAAGAGCGACAACGUCCCGAGCGAAGAAGUCGUC |
| | | AAGAAGAUGAAGAACUACUGGAGACAGCUGCUGAACGCAAAGCUGAUCACAC |
| | | AGAGAAAGUUCGACAACCUGACAAAGGCAGAGAGAGGAGGACUGAGCGAACU |
| | | GGACAAGGCAGGAUUCAUCAAGAGACAGCUGGUCGAAACAAGACAGAUCACA |
| | | AAGCACGUCGCACAGAUCCUGGACAGCAGAAUGAACACAAAGUACGACGAAA |
| | | ACGACAAGCUGAUCAGAGAAGUCAAGGUCAUCACACUGAAGAGCAAGCUGGU |
| | | CAGCGACUUCAGAAAGGACUUCCAGUUCUACAAGGUCAGAGAAAUCAACAAC |
| | | UACCACCACGCACACGACGCAUACCUGAACGCAGUCGUCGGAACAGCACUGA |
| | | UCAAGAAGUACCCGAAGCUGGAAAGCGAAUUCGUCUACGGAGACUACAAGGU |
| | | CUACGACGUCAGAAAGAUGAUCGCAAAGAGCGAACAGGAAAUCGGAAAGGCA |
| | | ACAGCAAAGUACUUCUUCUACAGCAACAUCAUGAACUUCUUCAAGACAGAAA |
| | | UCACACUGGCAAACGGAGAAAUCAGAAAGAGACCGCUGAUCGAAACAAACGG |
| | | AGAAACAGGAGAAAUCGUCUGGGACAAGGGAAGAGACUUCGCAACAGUCAGA |
| | | AAGGUCCUGAGCAUGCCGCAGGUCAACAUCGUCAAGAAGACAGAAGUCCAGA |
| | | CAGGAGGAUUCAGCAAGGAAAGCAUCCUGCCGAAGAGAAACAGCGACAAGCU |
| | | GAUCGCAAGAAAGAAGGACUGGGACCCGAAGAAGUACGGAGGAUUCGACAGC |
| | | CCGACAGUCGCAUACAGCGUCCUGGUCGUCGCAAAGGUCGAAAAGGGAAAGA |
| | | GCAAGAAGCUGAAGAGCGUCAAGGAACUGCUGGGAAUCACAAUCAUGGAAAG |
| | | AAGCAGCUUCGAAAAGAACCCGAUCGACUUCCUGGAAGCAAAGGGAUACAAG |
| | | GAAGUCAAGAAGGACCUGAUCAUCAAGCUGCCGAAGUACAGCCUGUUCGAAC |
| | | UGGAAAACGGAAGAAAGAGAAUGCUGGCAAGCGCAGGAGAACUGCAGAAGGG |
| | | AAACGAACUGGCACUGCCGAGCAAGUACGUCAACUUCCUGUACCUGGCAAGC |
| | | CACUACGAAAAGCUGAAGGGAAGCCCGGAAGACAACGAACAGAAGCAGCUGU |
| | | UCGUCGAACAGCACAAGCACUACCUGGACGAAAUCAUCGAACAGAUCAGCGA |
| | | AUUCAGCAAGAGAGUCAUCCUGGCAGACGCAAACCUGGACAAGGUCCUGAGC |
| | | GCAUACAACAAGCACAGAGACAAGCCGAUCAGAGAACAGGCAGAAAACAUCA |
| | | UCCACCUGUUCACACUGACAAACCUGGGAGCACCGGCAGCAUUCAAGUACUU |
| | | CGACACAACAAUCGACAGAAAGAGAUACACAAGCACAAAGGAAGUCCUGGAC |
| | | GCAACACUGAUCCACCAGAGCAUCACAGGACUGUACGAAACAAGAAUCGACC |
| | | UGAGCCAGCUGGGAGGAGACGGAGGAGGAAGCCCGAAGAAGAAGAGAAAGGU |
| | | CUAGCUAGCCAUCACAUUUAAAAGCAUCUCAGCCUACCAUGAGAAUAAGAGA |
| | | AAGAAAAUGAAGAUCAAUAGCUUAUUCAUCUCUUUUUCUUUUUCGUUGGUGU |
| | | AAAGCCAACACCCUGUCUAAAAAACAUAAAUUUCUUUAAUCAUUUUGCCUCU |
| | | UUUCUCUGUGCUUCAAUUAAUAAAAAAUGGAAAGAACCUCGAGAAAAAAAAA |
| | | AAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAA |
| 9 | mRNA | GGGAAGCUCAGAAUAAACGCUCAACUUUGGCCGGAUCUGCCACCAUGGACAA |
| | encoding | GAAGUACUCCAUCGGCCUGGACAUCGGCACCAACUCCGUGGGCUGGGCCGUG |
| | Sp. Cas9 | AUCACCGACGAGUACAAGGUGCCCUCCAAGAAGUUCAAGGUGCUGGGCAACA |
| | | CCGACCGGCACUCCAUCAAGAAGAACCUGAUCGGCGCCCUGCUGUUCGACUC |
| | | CGGCGAGACCGCCGAGGCCACCCGGCUGAAGCGGACCGCCCGGCGGCGGUAC |
| | | ACCCGGCGGAAGAACCGGAUCUGCUACCUGCAGGAGAUCUUCUCCAACGAGA |
| | | UGGCCAAGGUGGACGACUCCUUCUUCCACCGGCUGGAGGAGUCCUUCCUGGU |
| | | GGAGGAGGACAAGAAGCACGAGCGGCACCCCAUCUUCGGCAACAUCGUGGAC |
| | | GAGGUGGCCUACCACGAGAAGUACCCCACCAUCUACCACCUGCGGAAGAAGC |
| | | UGGUGGACUCCACCGACAAGGCCGACCUGCGGCUGAUCUACCUGGCCCUGGC |
| | | CCACAUGAUCAAGUUCCGGGGCCACUUCCUGAUCGAGGGCGACCUGAACCCC |
| | | GACAACUCCGACGUGGACAAGCUGUUCAUCCAGCUGGUGCAGACCUACAACC |
| | | AGCUGUUCGAGGAGAACCCCAUCAACGCCUCCGGCGUGGACGCCAAGGCCAU |
| | | CCUGUCCGCCCGGCUGUCCAAGUCCCGGCGGCUGGAGAACCUGAUCGCCCAG |
| | | CUGCCCGGCGAGAAGAAGAACGGCCUGUUCGGCAACCUGAUCGCCCUGUCCC |
| | | UGGGCCUGACCCCCAACUUCAAGUCCAACUUCGACCUGGCCGAGGACGCCAA |
| | | GCUGCAGCUGUCCAAGGACACCUACGACGACGACCUGGACAACCUGCUGGCC |
| | | CAGAUCGGCGACCAGUACGCCGACCUGUUCCUGGCCGCCAAGAACCUGUCCG |
| | | ACGCCAUCCUGCUGUCCGACAUCCUGCGGGUGAACACCGAGAUCACCAAGGC |
| | | CCCCCUGUCCGCCUCCAUGAUCAAGCGGUACGACGAGCACCACCAGGACCUG |
| | | ACCCUGCUGAAGGCCCUGGUGCGGCAGCAGCUGCCCGAGAAGUACAAGGAGA |
| | | UCUUCUUCGACCAGUCCAAGAACGGCUACGCCGGCUACAUCGACGGCGGCGC |
| | | CUCCCAGGAGGAGUUCUACAAGUUCAUCAAGCCCAUCCUGGAGAAGAUGGAC |
| | | GGCACCGAGGAGCUGCUGGUGAAGCUGAACCGGGAGGACCUGCUGCGGAAGC |
| | | AGCGGACCUUCGACAACGGCUCCAUCCCCCACCAGAUCCACCUGGGCGAGCU |
| | | GCACGCCAUCCUGCGGCGGCAGGAGGACUUCUACCCCUUCCUGAAGGACAAC |
| | | CGGGAGAAGAUCGAGAAGAUCCUGACCUUCCGGAUCCCCUACUACGUGGGCC |
| | | CCCUGGCCCGGGGCAACUCCCGGUUCGCCUGGAUGACCCGGAAGUCCGAGGA |
| | | GACCAUCACCCCCUGGAACUUCGAGGAGGUGGUGGACAAGGGCGCCUCCGCC |
| | | CAGUCCUUCAUCGAGCGGAUGACCAACUUCGACAAGAACCUGCCCAACGAGA |
| | | AGGUGCUGCCCAAGCACUCCCUGCUGUACGAGUACUUCACCGUGUACAACGA |
| | | GCUGACCAAGGUGAAGUACGUGACCGAGGGCAUGCGGAAGCCCGCCUUCCUG |
| | | UCCGGCGAGCAGAAGAAGGCCAUCGUGGACCUGCUGUUCAAGACCAACCGGA |
| | | AGGUGACCGUGAAGCAGCUGAAGGAGGACUACUUCAAGAAGAUCGAGUGCUU |
| | | CGACUCCGUGGAGAUCUCCGGCGUGGAGGACCGGUUCAACGCCUCCCUGGGC |
| | | ACCUACCACGACCUGCUGAAGAUCAUCAAGGACAAGGACUUCCUGGACAACG |
| | | AGGAGAACGAGGACAUCCUGGAGGACAUCGUGCUGACCCUGACCCUGUUCGA |
| | | GGACCGGGAGAUGAUCGAGGAGCGGCUGAAGACCUACGCCCACCUGUUCGAC |
| | | GACAAGGUGAUGAAGCAGCUGAAGCGGCGGCGGUACACCGGCUGGGGCCGGC |
| | | UGUCCCGGAAGCUGAUCAACGGCAUCCGGGACAAGCAGUCCGGCAAGACCAU |
| | | CCUGGACUUCCUGAAGUCCGACGGCUUCGCCAACCGGAACUUCAUGCAGCUG |
| | | AUCCACGACGACUCCCUGACCUUCAAGGAGGACAUCCAGAAGGCCCAGGUGU |
| | | CCGGCCAGGGCGACUCCCUGCACGAGCACAUCGCCAACCUGGCCGGCUCCCC |
| | | CGCCAUCAAGAAGGGCAUCCUGCAGACCGUGAAGGUGGUGGACGAGCUGGUG |
| | | AAGGUGAUGGGCCGGCACAAGCCCGAGAACAUCGUGAUCGAGAUGGCCCGGG |
| | | AGAACCAGACCACCCAGAAGGGCCAGAAGAACUCCCGGGAGCGGAUGAAGCG |
| | | GAUCGAGGAGGGCAUCAAGGAGCUGGGCUCCCAGAUCCUGAAGGAGCACCCC |
| | | GUGGAGAACACCCAGCUGCAGAACGAGAAGCUGUACCUGUACUACCUGCAGA |
| | | ACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUC |
| | | GACAACAAGGUGCUGACCCGGUCCGACAAGAACCGGGGCAAGUCCGACAACG |
| | | UGCCCUCCGAGGAGGUGGUGAAGAAGAUGAAGAACUACUGGCGGCAGCUGCU |
| | | GAACGCCAAGCUGAUCACCCAGCGGAAGUUCGACAACCUGACCAAGGCCGAG |
| | | CGGGGCGGCCUGUCCGAGCUGGACAAGGCCGGCUUCAUCAAGCGGCAGCUGG |
| | | UGGAGACCCGGCAGAUCACCAAGCACGUGGCCCAGAUCCUGGACUCCCGGAU |
| | | GAACACCAAGUACGACGAGAACGACAAGCUGAUCCGGGAGGUGAAGGUGAUC |
| | | ACCCUGAAGUCCAAGCUGGUGUCCGACUUCCGGAAGGACUUCCAGUUCUACA |
| | | AGGUGCGGGAGAUCAACAACUACCACCACGCCCACGACGCCUACCUGAACGC |
| | | CGUGGUGGGCACCGCCCUGAUCAAGAAGUACCCCAAGCUGGAGUCCGAGUUC |
| | | GUGUACGGCGACUACAAGGUGUACGACGUGCGGAAGAUGAUCGCCAAGUCCG |
| | | AGCAGGAGAUCGGCAAGGCCACCGCCAAGUACUUCUUCUACUCCAACAUCAU |
| | | GAACUUCUUCAAGACCGAGAUCACCCUGGCCAACGGCGAGAUCCGGAAGCGG |
| | | CCCCUGAUCGAGACCAACGGCGAGACCGGCGAGAUCGUGUGGGACAAGGGCC |
| | | GGGACUUCGCCACCGUGCGGAAGGUGCUGUCCAUGCCCCAGGUGAACAUCGU |
| | | GAAGAAGACCGAGGUGCAGACCGGCGGCUUCUCCAAGGAGUCCAUCCUGCCC |
| | | AAGCGGAACUCCGACAAGCUGAUCGCCCGGAAGAAGGACUGGGACCCCAAGA |
| | | AGUACGGCGGCUUCGACUCCCCCACCGUGGCCUACUCCGUGCUGGUGGUGGC |
| | | CAAGGUGGAGAAGGGCAAGUCCAAGAAGCUGAAGUCCGUGAAGGAGCUGCUG |
| | | GGCAUCACCAUCAUGGAGCGGUCCUCCUUCGAGAAGAACCCCAUCGACUUCC |
| | | UGGAGGCCAAGGGCUACAAGGAGGUGAAGAAGGACCUGAUCAUCAAGCUGCC |
| | | CAAGUACUCCCUGUUCGAGCUGGAGAACGGCCGGAAGCGGAUGCUGGCCUCC |
| | | GCCGGCGAGCUGCAGAAGGGCAACGAGCUGGCCCUGCCCUCCAAGUACGUGA |
| | | ACUUCCUGUACCUGGCCUCCCACUACGAGAAGCUGAAGGGCUCCCCCGAGGA |
| | | CAACGAGCAGAAGCAGCUGUUCGUGGAGCAGCACAAGCACUACCUGGACGAG |
| | | AUCAUCGAGCAGAUCUCCGAGUUCUCCAAGCGGGUGAUCCUGGCCGACGCCA |
| | | ACCUGGACAAGGUGCUGUCCGCCUACAACAAGCACCGGGACAAGCCCAUCCG |
| | | GGAGCAGGCCGAGAACAUCAUCCACCUGUUCACCCUGACCAACCUGGGCGCC |
| | | CCCGCCGCCUUCAAGUACUUCGACACCACCAUCGACCGGAAGCGGUACACCU |
| | | CCACCAAGGAGGUGCUGGACGCCACCCUGAUCCACCAGUCCAUCACCGGCCU |
| | | GUACGAGACCCGGAUCGACCUGUCCCAGCUGGGCGGCGACGGCGGCGGCUCC |
| | | CCCAAGAAGAAGCGGAAGGUGUGACUAGCACCAGCCUCAAGAACACCCGAAU |
| | | GGAGUCUCUAAGCUACAUAAUACCAACUUACACUUUACAAAAUGUUGUCCCC |
| | | CAAAAUGUAGCCAUUCGUAUCUGCUCCUAAUAAAAAGAAAGUUUCUUCACAU |
| | | UCUCUCGAGAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAA |
| 10 | mRNA | GGGAAGCUCAGAAUAAACGCUCAACUUUGGCCGGAUCUGCCACCAUGGACAA |
| | encoding | GAAGUACAGCAUCGGCCUGGACAUCGGCACCAACAGCGUGGGCUGGGCCGUG |
| | Sp. Cas9 | AUCACCGACGAGUACAAGGUGCCCAGCAAGAAGUUCAAGGUGCUGGGCAACA |
| | | CCGACCGGCACAGCAUCAAGAAGAACCUGAUCGGCGCCCUGCUGUUCGACAG |
| | | CGGCGAGACCGCCGAGGCCACCCGGCUGAAGCGGACCGCCCGGCGGCGGUAC |
| | | ACCCGGCGGAAGAACCGGAUCUGCUACCUGCAGGAGAUCUUCAGCAACGAGA |
| | | UGGCCAAGGUGGACGACAGCUUCUUCCACCGGCUGGAGGAGAGCUUCCUGGU |
| | | GGAGGAGGACAAGAAGCACGAGCGGCACCCCAUCUUCGGCAACAUCGUGGAC |
| | | GAGGUGGCCUACCACGAGAAGUACCCCACCAUCUACCACCUGCGGAAGAAGC |
| | | UGGUGGACAGCACCGACAAGGCCGACCUGCGGCUGAUCUACCUGGCCCUGGC |
| | | CCACAUGAUCAAGUUCCGGGGCCACUUCCUGAUCGAGGGCGACCUGAACCCC |
| | | GACAACAGCGACGUGGACAAGCUGUUCAUCCAGCUGGUGCAGACCUACAACC |
| | | AGCUGUUCGAGGAGAACCCCAUCAACGCCAGCGGCGUGGACGCCAAGGCCAU |
| | | CCUGAGCGCCCGGCUGAGCAAGAGCCGGCGGCUGGAGAACCUGAUCGCCCAG |
| | | CUGCCCGGCGAGAAGAAGAACGGCCUGUUCGGCAACCUGAUCGCCCUGAGCC |
| | | UGGGCCUGACCCCCAACUUCAAGAGCAACUUCGACCUGGCCGAGGACGCCAA |
| | | GCUGCAGCUGAGCAAGGACACCUACGACGACGACCUGGACAACCUGCUGGCC |
| | | CAGAUCGGCGACCAGUACGCCGACCUGUUCCUGGCCGCCAAGAACCUGAGCG |
| | | ACGCCAUCCUGCUGAGCGACAUCCUGCGGGUGAACACCGAGAUCACCAAGGC |
| | | CCCCCUGAGCGCCAGCAUGAUCAAGCGGUACGACGAGCACCACCAGGACCUG |
| | | ACCCUGCUGAAGGCCCUGGUGCGGCAGCAGCUGCCCGAGAAGUACAAGGAGA |
| | | UCUUCUUCGACCAGAGCAAGAACGGCUACGCCGGCUACAUCGACGGCGGCGC |
| | | CAGCCAGGAGGAGUUCUACAAGUUCAUCAAGCCCAUCCUGGAGAAGAUGGAC |
| | | GGCACCGAGGAGCUGCUGGUGAAGCUGAACCGGGAGGACCUGCUGCGGAAGC |
| | | AGCGGACCUUCGACAACGGCAGCAUCCCCCACCAGAUCCACCUGGGCGAGCU |
| | | GCACGCCAUCCUGCGGCGGCAGGAGGACUUCUACCCCUUCCUGAAGGACAAC |
| | | CGGGAGAAGAUCGAGAAGAUCCUGACCUUCCGGAUCCCCUACUACGUGGGCC |
| | | CCCUGGCCCGGGGCAACAGCCGGUUCGCCUGGAUGACCCGGAAGAGCGAGGA |
| | | GACCAUCACCCCCUGGAACUUCGAGGAGGUGGUGGACAAGGGCGCCAGCGCC |
| | | CAGAGCUUCAUCGAGCGGAUGACCAACUUCGACAAGAACCUGCCCAACGAGA |
| | | AGGUGCUGCCCAAGCACAGCCUGCUGUACGAGUACUUCACCGUGUACAACGA |
| | | GCUGACCAAGGUGAAGUACGUGACCGAGGGCAUGCGGAAGCCCGCCUUCCUG |
| | | AGCGGCGAGCAGAAGAAGGCCAUCGUGGACCUGCUGUUCAAGACCAACCGGA |
| | | AGGUGACCGUGAAGCAGCUGAAGGAGGACUACUUCAAGAAGAUCGAGUGCUU |
| | | CGACAGCGUGGAGAUCAGCGGCGUGGAGGACCGGUUCAACGCCAGCCUGGGC |
| | | ACCUACCACGACCUGCUGAAGAUCAUCAAGGACAAGGACUUCCUGGACAACG |
| | | AGGAGAACGAGGACAUCCUGGAGGACAUCGUGCUGACCCUGACCCUGUUCGA |
| | | GGACCGGGAGAUGAUCGAGGAGCGGCUGAAGACCUACGCCCACCUGUUCGAC |
| | | GACAAGGUGAUGAAGCAGCUGAAGCGGCGGCGGUACACCGGCUGGGGCCGGC |
| | | UGAGCCGGAAGCUGAUCAACGGCAUCCGGGACAAGCAGAGCGGCAAGACCAU |
| | | CCUGGACUUCCUGAAGAGCGACGGCUUCGCCAACCGGAACUUCAUGCAGCUG |
| | | AUCCACGACGACAGCCUGACCUUCAAGGAGGACAUCCAGAAGGCCCAGGUGA |
| | | GCGGCCAGGGCGACAGCCUGCACGAGCACAUCGCCAACCUGGCCGGCAGCCC |
| | | CGCCAUCAAGAAGGGCAUCCUGCAGACCGUGAAGGUGGUGGACGAGCUGGUG |
| | | AAGGUGAUGGGCCGGCACAAGCCCGAGAACAUCGUGAUCGAGAUGGCCCGGG |
| | | AGAACCAGACCACCCAGAAGGGCCAGAAGAACAGCCGGGAGCGGAUGAAGCG |
| | | GAUCGAGGAGGGCAUCAAGGAGCUGGGCAGCCAGAUCCUGAAGGAGCACCCC |
| | | GUGGAGAACACCCAGCUGCAGAACGAGAAGCUGUACCUGUACUACCUGCAGA |
| | | ACGGCCGGGACAUGUACGUGGACCAGGAGCUGGACAUCAACCGGCUGAGCGA |
| | | CUACGACGUGGACCACAUCGUGCCCCAGAGCUUCCUGAAGGACGACAGCAUC |
| | | GACAACAAGGUGCUGACCCGGAGCGACAAGAACCGGGGCAAGAGCGACAACG |
| | | UGCCCAGCGAGGAGGUGGUGAAGAAGAUGAAGAACUACUGGCGGCAGCUGCU |
| | | GAACGCCAAGCUGAUCACCCAGCGGAAGUUCGACAACCUGACCAAGGCCGAG |
| | | CGGGGCGGCCUGAGCGAGCUGGACAAGGCCGGCUUCAUCAAGCGGCAGCUGG |
| | | UGGAGACCCGGCAGAUCACCAAGCACGUGGCCCAGAUCCUGGACAGCCGGAU |
| | | GAACACCAAGUACGACGAGAACGACAAGCUGAUCCGGGAGGUGAAGGUGAUC |
| | | ACCCUGAAGAGCAAGCUGGUGAGCGACUUCCGGAAGGACUUCCAGUUCUACA |
| | | AGGUGCGGGAGAUCAACAACUACCACCACGCCCACGACGCCUACCUGAACGC |
| | | CGUGGUGGGCACCGCCCUGAUCAAGAAGUACCCCAAGCUGGAGAGCGAGUUC |
| | | GUGUACGGCGACUACAAGGUGUACGACGUGCGGAAGAUGAUCGCCAAGAGCG |
| | | AGCAGGAGAUCGGCAAGGCCACCGCCAAGUACUUCUUCUACAGCAACAUCAU |
| | | GAACUUCUUCAAGACCGAGAUCACCCUGGCCAACGGCGAGAUCCGGAAGCGG |
| | | CCCCUGAUCGAGACCAACGGCGAGACCGGCGAGAUCGUGUGGGACAAGGGCC |
| | | GGGACUUCGCCACCGUGCGGAAGGUGCUGAGCAUGCCCCAGGUGAACAUCGU |
| | | GAAGAAGACCGAGGUGCAGACCGGCGGCUUCAGCAAGGAGAGCAUCCUGCCC |
| | | AAGCGGAACAGCGACAAGCUGAUCGCCCGGAAGAAGGACUGGGACCCCAAGA |
| | | AGUACGGCGGCUUCGACAGCCCCACCGUGGCCUACAGCGUGCUGGUGGUGGC |
| | | CAAGGUGGAGAAGGGCAAGAGCAAGAAGCUGAAGAGCGUGAAGGAGCUGCUG |
| | | GGCAUCACCAUCAUGGAGCGGAGCAGCUUCGAGAAGAACCCCAUCGACUUCC |
| | | UGGAGGCCAAGGGCUACAAGGAGGUGAAGAAGGACCUGAUCAUCAAGCUGCC |
| | | CAAGUACAGCCUGUUCGAGCUGGAGAACGGCCGGAAGCGGAUGCUGGCCAGC |
| | | GCCGGCGAGCUGCAGAAGGGCAACGAGCUGGCCCUGCCCAGCAAGUACGUGA |
| | | ACUUCCUGUACCUGGCCAGCCACUACGAGAAGCUGAAGGGCAGCCCCGAGGA |
| | | CAACGAGCAGAAGCAGCUGUUCGUGGAGCAGCACAAGCACUACCUGGACGAG |
| | | AUCAUCGAGCAGAUCAGCGAGUUCAGCAAGCGGGUGAUCCUGGCCGACGCCA |
| | | ACCUGGACAAGGUGCUGAGCGCCUACAACAAGCACCGGGACAAGCCCAUCCG |
| | | GGAGCAGGCCGAGAACAUCAUCCACCUGUUCACCCUGACCAACCUGGGCGCC |
| | | CCCGCCGCCUUCAAGUACUUCGACACCACCAUCGACCGGAAGCGGUACACCA |
| | | GCACCAAGGAGGUGCUGGACGCCACCCUGAUCCACCAGAGCAUCACCGGCCU |
| | | GUACGAGACCCGGAUCGACCUGAGCCAGCUGGGCGGCGACGGCGGCGGCAGC |
| | | CCCAAGAAGAAGCGGAAGGUGUGACUAGCACCAGCCUCAAGAACACCCGAAU |
| | | GGAGUCUCUAAGCUACAUAAUACCAACUUACACUUUACAAAAUGUUGUCCCC |
| | | CAAAAUGUAGCCAUUCGUAUCUGCUCCUAAUAAAAAGAAAGUUUCUUCACAU |
| | | UCUCUCGAGAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAA |
| 11 | mRNA | GGGAAGCUCAGAAUAAACGCUCAACUUUGGCCGGAUCUGCCACCAUGGACAA |
| | encoding | GAAGUACAGCAUCGGCCUGGACAUCGGCACGAACAGCGUUGGCUGGGCUGUG |
| | Sp. Cas9 | AUCACGGACGAGUACAAGGUUCCCUCAAAGAAGUUCAAGGUGCUGGGCAACA |
| | | CGGACCGGCACAGCAUCAAGAAGAAUCUCAUCGGUGCACUGCUGUUCGACAG |
| | | CGGUGAGACGGCCGAAGCCACGCGGCUGAAGCGGACGGCCCGCCGGCGGUAC |
| | | ACGCGGCGGAAGAACCGGAUCUGCUACCUGCAGGAGAUCUUCAGCAACGAGA |
| | | UGGCCAAGGUGGACGACAGCUUCUUCCACCGGCUGGAGGAGAGCUUCCUGGU |
| | | GGAGGAGGACAAGAAGCACGAGCGGCACCCCAUCUUCGGCAACAUCGUGGAC |
| | | GAAGUCGCCUACCACGAGAAGUACCCCACCAUCUACCACCUGCGGAAGAAGC |
| | | UGGUGGACUCGACUGACAAGGCCGACCUGCGGCUGAUCUACCUGGCACUGGC |
| | | CCACAUGAUAAAGUUCCGGGGCCACUUCCUGAUCGAGGGCGACCUGAACCCU |
| | | GACAACAGCGACGUGGACAAGCUGUUCAUCCAGCUGGUGCAGACCUACAACC |
| | | AGCUGUUCGAGGAGAACCCCAUCAACGCCAGCGGCGUGGACGCCAAGGCCAU |
| | | CCUCAGCGCCCGCCUCAGCAAGAGCCGGCGGCUGGAGAAUCUCAUCGCCCAG |
| | | CUUCCAGGUGAGAAGAAGAAUGGGCUGUUCGGCAAUCUCAUCGCACUCAGCC |
| | | UGGGCCUGACUCCCAACUUCAAGAGCAACUUCGACCUGGCCGAGGACGCCAA |
| | | GCUGCAGCUCAGCAAGGACACCUACGACGACGACCUGGACAAUCUCCUGGCC |
| | | CAGAUCGGCGACCAGUACGCCGACCUGUUCCUGGCUGCCAAGAAUCUCAGCG |
| | | ACGCCAUCCUGCUCAGCGACAUCCUGCGGGUGAACACAGAGAUCACGAAGGC |
| | | CCCCCUCAGCGCCAGCAUGAUAAAGCGGUACGACGAGCACCACCAGGACCUG |
| | | ACGCUGCUGAAGGCACUGGUGCGGCAGCAGCUUCCAGAGAAGUACAAGGAGA |
| | | UCUUCUUCGACCAGAGCAAGAAUGGGUACGCCGGGUACAUCGACGGUGGUGC |
| | | CAGCCAGGAGGAGUUCUACAAGUUCAUCAAGCCCAUCCUGGAGAAGAUGGAC |
| | | GGCACAGAGGAGCUGCUGGUGAAGCUGAACAGGGAGGACCUGCUGCGGAAGC |
| | | AGCGGACGUUCGACAAUGGGAGCAUCCCCCACCAGAUCCACCUGGGUGAGCU |
| | | GCACGCCAUCCUGCGGCGGCAGGAGGACUUCUACCCCUUCCUGAAGGACAAC |
| | | AGGGAGAAGAUCGAGAAGAUCCUGACGUUCCGGAUCCCCUACUACGUUGGCC |
| | | CCCUGGCCCGCGGCAACAGCCGGUUCGCCUGGAUGACGCGGAAGAGCGAGGA |
| | | GACGAUCACUCCCUGGAACUUCGAGGAAGUCGUGGACAAGGGUGCCAGCGCC |
| | | CAGAGCUUCAUCGAGCGGAUGACGAACUUCGACAAGAAUCUUCCAAACGAGA |
| | | AGGUGCUUCCAAAGCACAGCCUGCUGUACGAGUACUUCACGGUGUACAACGA |
| | | GCUGACGAAGGUGAAGUACGUGACAGAGGGCAUGCGGAAGCCCGCCUUCCUC |
| | | AGCGGUGAGCAGAAGAAGGCCAUCGUGGACCUGCUGUUCAAGACGAACCGGA |
| | | AGGUGACGGUGAAGCAGCUGAAGGAGGACUACUUCAAGAAGAUCGAGUGCUU |
| | | CGACAGCGUGGAGAUCAGCGGCGUGGAGGACCGGUUCAACGCCAGCCUGGGC |
| | | ACCUACCACGACCUGCUGAAGAUCAUCAAGGACAAGGACUUCCUGGACAACG |
| | | AGGAGAACGAGGACAUCCUGGAGGACAUCGUGCUGACGCUGACGCUGUUCGA |
| | | GGACAGGGAGAUGAUAGAGGAGCGGCUGAAGACCUACGCCCACCUGUUCGAC |
| | | GACAAGGUGAUGAAGCAGCUGAAGCGGCGGCGGUACACGGGCUGGGGCCGGC |
| | | UCAGCCGGAAGCUGAUCAAUGGGAUCCGAGACAAGCAGAGCGGCAAGACGAU |
| | | CCUGGACUUCCUGAAGAGCGACGGCUUCGCCAACCGGAACUUCAUGCAGCUG |
| | | AUCCACGACGACAGCCUGACGUUCAAGGAGGACAUCCAGAAGGCCCAGGUCA |
| | | GCGGCCAGGGCGACAGCCUGCACGAGCACAUCGCCAAUCUCGCCGGGAGCCC |
| | | CGCCAUCAAGAAGGGGAUCCUGCAGACGGUGAAGGUGGUGGACGAGCUGGUG |
| | | AAGGUGAUGGGCCGGCACAAGCCAGAGAACAUCGUGAUCGAGAUGGCCAGGG |
| | | AGAACCAGACGACUCAAAAGGGGCAGAAGAACAGCAGGGAGCGGAUGAAGCG |
| | | GAUCGAGGAGGGCAUCAAGGAGCUGGGCAGCCAGAUCCUGAAGGAGCACCCC |
| | | GUGGAGAACACUCAACUGCAGAACGAGAAGCUGUACCUGUACUACCUGCAGA |
| | | AUGGGCGAGACAUGUACGUGGACCAGGAGCUGGACAUCAACCGGCUCAGCGA |
| | | CUACGACGUGGACCACAUCGUUCCCCAGAGCUUCCUGAAGGACGACAGCAUC |
| | | GACAACAAGGUGCUGACGCGGAGCGACAAGAACCGGGGCAAGAGCGACAACG |
| | | UUCCCUCAGAGGAAGUCGUGAAGAAGAUGAAGAACUACUGGCGGCAGCUGCU |
| | | GAACGCCAAGCUGAUCACUCAACGGAAGUUCGACAAUCUCACGAAGGCCGAG |
| | | CGGGGUGGCCUCAGCGAGCUGGACAAGGCCGGGUUCAUCAAGCGGCAGCUGG |
| | | UGGAGACGCGGCAGAUCACGAAGCACGUGGCCCAGAUCCUGGACAGCCGGAU |
| | | GAACACGAAGUACGACGAGAACGACAAGCUGAUCAGGGAAGUCAAGGUGAUC |
| | | ACGCUGAAGAGCAAGCUGGUCAGCGACUUCCGGAAGGACUUCCAGUUCUACA |
| | | AGGUGAGGGAGAUCAACAACUACCACCACGCCCACGACGCCUACCUGAACGC |
| | | UGUGGUUGGCACGGCACUGAUCAAGAAGUACCCCAAGCUGGAGAGCGAGUUC |
| | | GUGUACGGCGACUACAAGGUGUACGACGUGCGGAAGAUGAUAGCCAAGAGCG |
| | | AGCAGGAGAUCGGCAAGGCCACGGCCAAGUACUUCUUCUACAGCAACAUCAU |
| | | GAACUUCUUCAAGACAGAGAUCACGCUGGCCAAUGGUGAGAUCCGGAAGCGG |
| | | CCCCUGAUCGAGACGAAUGGUGAGACGGGUGAGAUCGUGUGGGACAAGGGGC |
| | | GAGACUUCGCCACGGUGCGGAAGGUGCUCAGCAUGCCCCAGGUGAACAUCGU |
| | | GAAGAAGACAGAAGUCCAGACGGGUGGCUUCAGCAAGGAGAGCAUCCUUCCA |
| | | AAGCGGAACAGCGACAAGCUGAUCGCCCGCAAGAAGGACUGGGACCCCAAGA |
| | | AGUACGGUGGCUUCGACAGCCCCACCGUGGCCUACAGCGUGCUGGUGGUGGC |
| | | CAAGGUGGAGAAGGGGAAGAGCAAGAAGCUGAAGAGCGUGAAGGAGCUGCUG |
| | | GGCAUCACGAUCAUGGAGCGGAGCAGCUUCGAGAAGAACCCCAUCGACUUCC |
| | | UGGAAGCCAAGGGGUACAAGGAAGUCAAGAAGGACCUGAUCAUCAAGCUUCC |
| | | AAAGUACAGCCUGUUCGAGCUGGAGAAUGGGCGGAAGCGGAUGCUGGCCAGC |
| | | GCCGGUGAGCUGCAGAAGGGGAACGAGCUGGCACUUCCCUCAAAGUACGUGA |
| | | ACUUCCUGUACCUGGCCAGCCACUACGAGAAGCUGAAGGGGAGCCCAGAGGA |
| | | CAACGAGCAGAAGCAGCUGUUCGUGGAGCAGCACAAGCACUACCUGGACGAG |
| | | AUCAUCGAGCAGAUCAGCGAGUUCAGCAAGCGGGUGAUCCUGGCCGACGCCA |
| | | AUCUCGACAAGGUGCUCAGCGCCUACAACAAGCACCGAGACAAGCCCAUCAG |
| | | GGAGCAGGCCGAGAACAUCAUCCACCUGUUCACGCUGACGAAUCUCGGUGCC |
| | | CCCGCUGCCUUCAAGUACUUCGACACGACGAUCGACCGGAAGCGGUACACGU |
| | | CGACUAAGGAAGUCCUGGACGCCACGCUGAUCCACCAGAGCAUCACGGGCCU |
| | | GUACGAGACGCGGAUCGACCUCAGCCAGCUGGGUGGCGACGGUGGUGGCAGC |
| | | CCCAAGAAGAAGCGGAAGGUGUAGCUAGCACCAGCCUCAAGAACACCCGAAU |
| | | GGAGUCUCUAAGCUACAUAAUACCAACUUACACUUUACAAAAUGUUGUCCCC |
| | | CAAAAUGUAGCCAUUCGUAUCUGCUCCUAAUAAAAAGAAAGUUUCUUCACAU |
| | | UCUCUCGAGAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAA |
| 12 | mRNA | GGGAAGCUCAGAAUAAACGCUCAACUUUGGCCGGAUCUGCCACCAUGGACAA |
| | encoding | GAAGUACAGCAUCGGCCUCGACAUCGGCACCAACAGCGUCGGCUGGGCCGUC |
| | Sp. Cas9 | AUCACCGACGAGUACAAGGUCCCCAGCAAGAAGUUCAAGGUCCUCGGCAACA |
| | | CCGACCGCCACAGCAUCAAGAAGAACCUCAUCGGCGCCCUCCUCUUCGACAG |
| | | CGGCGAGACCGCCGAGGCCACCCGCCUCAAGCGCACCGCCCGCCGCCGCUAC |
| | | ACCCGCCGCAAGAACCGCAUCUGCUACCUCCAGGAGAUCUUCAGCAACGAGA |
| | | UGGCCAAGGUCGACGACAGCUUCUUCCACCGCCUCGAGGAGAGCUUCCUCGU |
| | | CGAGGAGGACAAGAAGCACGAGCGCCACCCCAUCUUCGGCAACAUCGUCGAC |
| | | GAGGUCGCCUACCACGAGAAGUACCCCACCAUCUACCACCUCCGCAAGAAGC |
| | | UCGUCGACAGCACCGACAAGGCCGACCUCCGCCUCAUCUACCUCGCCCUCGC |
| | | CCACAUGAUCAAGUUCCGCGGCCACUUCCUCAUCGAGGGCGACCUCAACCCC |
| | | GACAACAGCGACGUCGACAAGCUCUUCAUCCAGCUCGUCCAGACCUACAACC |
| | | AGCUCUUCGAGGAGAACCCCAUCAACGCCAGCGGCGUCGACGCCAAGGCCAU |
| | | CCUCAGCGCCCGCCUCAGCAAGAGCCGCCGCCUCGAGAACCUCAUCGCCCAG |
| | | CUCCCCGGCGAGAAGAAGAACGGCCUCUUCGGCAACCUCAUCGCCCUCAGCC |
| | | UCGGCCUCACCCCCAACUUCAAGAGCAACUUCGACCUCGCCGAGGACGCCAA |
| | | GCUCCAGCUCAGCAAGGACACCUACGACGACGACCUCGACAACCUCCUCGCC |
| | | CAGAUCGGCGACCAGUACGCCGACCUCUUCCUCGCCGCCAAGAACCUCAGCG |
| | | ACGCCAUCCUCCUCAGCGACAUCCUCCGCGUCAACACCGAGAUCACCAAGGC |
| | | CCCCCUCAGCGCCAGCAUGAUCAAGCGCUACGACGAGCACCACCAGGACCUC |
| | | ACCCUCCUCAAGGCCCUCGUCCGCCAGCAGCUCCCCGAGAAGUACAAGGAGA |
| | | UCUUCUUCGACCAGAGCAAGAACGGCUACGCCGGCUACAUCGACGGCGGCGC |
| | | CAGCCAGGAGGAGUUCUACAAGUUCAUCAAGCCCAUCCUCGAGAAGAUGGAC |
| | | GGCACCGAGGAGCUCCUCGUCAAGCUCAACCGCGAGGACCUCCUCCGCAAGC |
| | | AGCGCACCUUCGACAACGGCAGCAUCCCCCACCAGAUCCACCUCGGCGAGCU |
| | | CCACGCCAUCCUCCGCCGCCAGGAGGACUUCUACCCCUUCCUCAAGGACAAC |
| | | CGCGAGAAGAUCGAGAAGAUCCUCACCUUCCGCAUCCCCUACUACGUCGGCC |
| | | CCCUCGCCCGCGGCAACAGCCGCUUCGCCUGGAUGACCCGCAAGAGCGAGGA |
| | | GACCAUCACCCCCUGGAACUUCGAGGAGGUCGUCGACAAGGGCGCCAGCGCC |
| | | CAGAGCUUCAUCGAGCGCAUGACCAACUUCGACAAGAACCUCCCCAACGAGA |
| | | AGGUCCUCCCCAAGCACAGCCUCCUCUACGAGUACUUCACCGUCUACAACGA |
| | | GCUCACCAAGGUCAAGUACGUCACCGAGGGCAUGCGCAAGCCCGCCUUCCUC |
| | | AGCGGCGAGCAGAAGAAGGCCAUCGUCGACCUCCUCUUCAAGACCAACCGCA |
| | | AGGUCACCGUCAAGCAGCUCAAGGAGGACUACUUCAAGAAGAUCGAGUGCUU |
| | | CGACAGCGUCGAGAUCAGCGGCGUCGAGGACCGCUUCAACGCCAGCCUCGGC |
| | | ACCUACCACGACCUCCUCAAGAUCAUCAAGGACAAGGACUUCCUCGACAACG |
| | | AGGAGAACGAGGACAUCCUCGAGGACAUCGUCCUCACCCUCACCCUCUUCGA |
| | | GGACCGCGAGAUGAUCGAGGAGCGCCUCAAGACCUACGCCCACCUCUUCGAC |
| | | GACAAGGUCAUGAAGCAGCUCAAGCGCCGCCGCUACACCGGCUGGGGCCGCC |
| | | UCAGCCGCAAGCUCAUCAACGGCAUCCGCGACAAGCAGAGCGGCAAGACCAU |
| | | CCUCGACUUCCUCAAGAGCGACGGCUUCGCCAACCGCAACUUCAUGCAGCUC |
| | | AUCCACGACGACAGCCUCACCUUCAAGGAGGACAUCCAGAAGGCCCAGGUCA |
| | | GCGGCCAGGGCGACAGCCUCCACGAGCACAUCGCCAACCUCGCCGGCAGCCC |
| | | CGCCAUCAAGAAGGGCAUCCUCCAGACCGUCAAGGUCGUCGACGAGCUCGUC |
| | | AAGGUCAUGGGCCGCCACAAGCCCGAGAACAUCGUCAUCGAGAUGGCCCGCG |
| | | AGAACCAGACCACCCAGAAGGGCCAGAAGAACAGCCGCGAGCGCAUGAAGCG |
| | | CAUCGAGGAGGGCAUCAAGGAGCUCGGCAGCCAGAUCCUCAAGGAGCACCCC |
| | | GUCGAGAACACCCAGCUCCAGAACGAGAAGCUCUACCUCUACUACCUCCAGA |
| | | ACGGCCGCGACAUGUACGUCGACCAGGAGCUCGACAUCAACCGCCUCAGCGA |
| | | CUACGACGUCGACCACAUCGUCCCCCAGAGCUUCCUCAAGGACGACAGCAUC |
| | | GACAACAAGGUCCUCACCCGCAGCGACAAGAACCGCGGCAAGAGCGACAACG |
| | | UCCCCAGCGAGGAGGUCGUCAAGAAGAUGAAGAACUACUGGCGCCAGCUCCU |
| | | CAACGCCAAGCUCAUCACCCAGCGCAAGUUCGACAACCUCACCAAGGCCGAG |
| | | CGCGGCGGCCUCAGCGAGCUCGACAAGGCCGGCUUCAUCAAGCGCCAGCUCG |
| | | UCGAGACCCGCCAGAUCACCAAGCACGUCGCCCAGAUCCUCGACAGCCGCAU |
| | | GAACACCAAGUACGACGAGAACGACAAGCUCAUCCGCGAGGUCAAGGUCAUC |
| | | ACCCUCAAGAGCAAGCUCGUCAGCGACUUCCGCAAGGACUUCCAGUUCUACA |
| | | AGGUCCGCGAGAUCAACAACUACCACCACGCCCACGACGCCUACCUCAACGC |
| | | CGUCGUCGGCACCGCCCUCAUCAAGAAGUACCCCAAGCUCGAGAGCGAGUUC |
| | | GUCUACGGCGACUACAAGGUCUACGACGUCCGCAAGAUGAUCGCCAAGAGCG |
| | | AGCAGGAGAUCGGCAAGGCCACCGCCAAGUACUUCUUCUACAGCAACAUCAU |
| | | GAACUUCUUCAAGACCGAGAUCACCCUCGCCAACGGCGAGAUCCGCAAGCGC |
| | | CCCCUCAUCGAGACCAACGGCGAGACCGGCGAGAUCGUCUGGGACAAGGGCC |
| | | GCGACUUCGCCACCGUCCGCAAGGUCCUCAGCAUGCCCCAGGUCAACAUCGU |
| | | CAAGAAGACCGAGGUCCAGACCGGCGGCUUCAGCAAGGAGAGCAUCCUCCCC |
| | | AAGCGCAACAGCGACAAGCUCAUCGCCCGCAAGAAGGACUGGGACCCCAAGA |
| | | AGUACGGCGGCUUCGACAGCCCCACCGUCGCCUACAGCGUCCUCGUCGUCGC |
| | | CAAGGUCGAGAAGGGCAAGAGCAAGAAGCUCAAGAGCGUCAAGGAGCUCCUC |
| | | GGCAUCACCAUCAUGGAGCGCAGCAGCUUCGAGAAGAACCCCAUCGACUUCC |
| | | UCGAGGCCAAGGGCUACAAGGAGGUCAAGAAGGACCUCAUCAUCAAGCUCCC |
| | | CAAGUACAGCCUCUUCGAGCUCGAGAACGGCCGCAAGCGCAUGCUCGCCAGC |
| | | GCCGGCGAGCUCCAGAAGGGCAACGAGCUCGCCCUCCCCAGCAAGUACGUCA |
| | | ACUUCCUCUACCUCGCCAGCCACUACGAGAAGCUCAAGGGCAGCCCCGAGGA |
| | | CAACGAGCAGAAGCAGCUCUUCGUCGAGCAGCACAAGCACUACCUCGACGAG |
| | | AUCAUCGAGCAGAUCAGCGAGUUCAGCAAGCGCGUCAUCCUCGCCGACGCCA |
| | | ACCUCGACAAGGUCCUCAGCGCCUACAACAAGCACCGCGACAAGCCCAUCCG |
| | | CGAGCAGGCCGAGAACAUCAUCCACCUCUUCACCCUCACCAACCUCGGCGCC |
| | | CCCGCCGCCUUCAAGUACUUCGACACCACCAUCGACCGCAAGCGCUACACCA |
| | | GCACCAAGGAGGUCCUCGACGCCACCCUCAUCCACCAGAGCAUCACCGGCCU |
| | | CUACGAGACCCGCAUCGACCUCAGCCAGCUCGGCGGCGACGGCGGCGGCAGC |
| | | CCCAAGAAGAAGCGCAAGGUCUAGCUAGCACCAGCCUCAAGAACACCCGAAU |
| | | GGAGUCUCUAAGCUACAUAAUACCAACUUACACUUUACAAAAUGUUGUCCCC |
| | | CAAAAUGUAGCCAUUCGUAUCUGCUCCUAAUAAAAAGAAAGUUUCUUCACAU |
| | | UCUCUCGAGAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAA |
| | | AAAAAAAAA |
| 13 | amino acid | MDKKYSIGLDIGTNSVGWAVITDEYKVPSKKFKVLGNTDRHSIKKNLIGALL |
| | sequence for | FDSGETAEATRLKRTARRRYTRRKNRICYLQEIFSNEMAKVDDSFFHRLEES |
| | Sp. Cas9 | FLVEEDKKHERHPIFGNIVDEVAYHEKYPTIYHLRKKLVDSTDKADLRLIYL |
| | | ALAHMIKFRGHFLIEGDLNPDNSDVDKLFIQLVQTYNQLFEENPINASGVDA |
| | | KAILSARLSKSRRLENLIAQLPGEKKNGLFGNLIALSLGLTPNFKSNFDLAE |
| | | DAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYID |
| | | GGASQEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAWMTRK |
| | | SEETITPWNFEEVVDKGASAQSFIERMTNFDKNLPNEKVLPKHSLLYEYFTV |
| | | YNELTKVKYVTEGMRKPAFLSGEQKKAIVDLLFKTNRKVTVKQLKEDYFKKI |
| | | ECFDSVEISGVEDRENASLGTYHDLLKIIKDKDELDNEENEDILEDIVLTLT |
| | | LFEDREMIEERLKTYAHLFDDKVMKQLKRRRYTGWGRLSRKLINGIRDKQSG |
| | | KTILDFLKSDGFANRNFMQLIHDDSLTFKEDIQKAQVSGQGDSLHEHIANLA |
| | | GSPAIKKGILQTVKVVDELVKVMGRHKPENIVIEMARENQTTQKGQKNSRER |
| | | MKRIEEGIKELGSQILKEHPVENTQLQNEKLYLYYLQNGRDMYVDQELDINR |
| | | LSDYDVDHIVPQSFLKDDSIDNKVLTRSDKNRGKSDNVPSEEVVKKMKNYWR |
| | | QLLNAKLITQRKFDNLTKAERGGLSELDKAGFIKRQLVETRQITKHVAQILD |
| | | SRMNTKYDENDKLIREVKVITLKSKLVSDFRKDFQFYKVREINNYHHAHDAY |
| | | LNAVVGTALIKKYPKLESEFVYGDYKVYDVRKMIAKSEQEIGKATAKYFFYS |
| | | NIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVL |
| | | VVAKVEKGKSKKLKSVKELLGITIMERSSFEKNPIDFLEAKGYKEVKKDLII |
| | | KLPKYSLFELENGRKRMLASAGELQKGNELALPSKYVNFLYLASHYEKLKGS |
| | | PEDNEQKQLFVEQHKHYLDEIIEQISEFSKRVILADANLDKVLSAYNKHRDK |
| | | PIREQAENIIHLFTLTNLGAPAAFKYEDTTIDRKRYTSTKEVLDATLIHQSI |
| | | TGLYETRIDLSQLGGDGGGSPKKKRKV |
| 14 | amino acid | MDKKYSIGLAIGTNSVGWAVITDEYKVPSKKFKVLGNTDRHSIKKNLIGALL |
| | sequence | FDSGETAEATRLKRTARRRYTRRKNRICYLQEIFSNEMAKVDDSFFHRLEES |
| | Sp. Cas9 | FLVEEDKKHERHPIFGNIVDEVAYHEKYPTIYHLRKKLVDSTDKADLRLIYL |
| | nickase | ALAHMIKFRGHFLIEGDLNPDNSDVDKLFIQLVQTYNQLFEENPINASGVDA |
| | | KAILSARLSKSRRLENLIAQLPGEKKNGLFGNLIALSLGLTPNFKSNFDLAE |
| | | DAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYID |
| | | GGASQEEFYKFIKPILEKMDGTEELLVKLNREDLLRKQRTEDNGSIPHQIHL |
| | | GELHAILRRQEDFYPFLKDNREKIEKILTFRIPYYVGPLARGNSRFAWMTRK |
| | | SEETITPWNFEEVVDKGASAQSFIERMTNFDKNLPNEKVLPKHSLLYEYFTV |
| | | YNELTKVKYVTEGMRKPAFLSGEQKKAIVDLLFKTNRKVTVKQLKEDYFKKI |
| | | ECFDSVEISGVEDRFNASLGTYHDLLKIIKDKDELDNEENEDILEDIVLTLT |
| | | LFEDREMIEERLKTYAHLFDDKVMKQLKRRRYTGWGRLSRKLINGIRDKQSG |
| | | KTILDFLKSDGFANRNFMQLIHDDSLTFKEDIQKAQVSGQGDSLHEHIANLA |
| | | GSPAIKKGILQTVKVVDELVKVMGRHKPENIVIEMARENQTTQKGQKNSRER |
| | | MKRIEEGIKELGSQILKEHPVENTQLQNEKLYLYYLQNGRDMYVDQELDINR |
| | | LSDYDVDHIVPQSFLKDDSIDNKVLTRSDKNRGKSDNVPSEEVVKKMKNYWR |
| | | QLLNAKLITQRKFDNLTKAERGGLSELDKAGFIKRQLVETRQITKHVAQILD |
| | | SRMNTKYDENDKLIREVKVITLKSKLVSDFRKDFQFYKVREINNYHHAHDAY |
| | | LNAVVGTALIKKYPKLESEFVYGDYKVYDVRKMIAKSEQEIGKATAKYFFYS |
| | | NIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVL |
| | | VVAKVEKGKSKKLKSVKELLGITIMERSSFEKNPIDFLEAKGYKEVKKDLII |
| | | KLPKYSLFELENGRKRMLASAGELQKGNELALPSKYVNFLYLASHYEKLKGS |
| | | PEDNEQKQLFVEQHKHYLDEIIEQISEFSKRVILADANLDKVLSAYNKHRDK |
| | | PIREQAENIIHLFTLTNLGAPAAFKYFDTTIDRKRYTSTKEVLDATLIHQSI |
| | | TGLYETRIDLSQLGGDGGGSPKKKRKV |
| 15 | TTR guide | AAAGGCUGCUGAUGACACCU |
| | sequence | |
| 16 | TTR | AAAGGCUGCUGAUGACACCUGUUUUAGAGCUAGAAAUAGCAAGUUAAAAUAA |
| | sgRNA | GGCUAGUCCGUUAUCAACUUGAAAAAGUGGCACCGAGUCGGUGCUUUU |
| 17 | Not used | Not used |
| 18 | Generic | NNNNNNNNNNNNNNNNNNNNGUUUUAGAGCUAGAAAUAGCAAGUUAAAAUAA |
| | sgRNA | GGCUAGUCCGUUAUCAACUUGAAAAAGUGGCACCGAGUCGGUGCUUUU |
| 19 | Generic | mN*mN*mN*NNNNNNNNNNNNNNNNNGUUUUAGAmGmCmUmAmGmAmAmAmU |
| | sgRNA | mAmGmCAAGUUAAAAUAAGGCUAGUCCGUUAUCAmAmCmUmUmGmAmAmAmA |
| | modified | mAmGmUmGmGmCmAmCmCmGmAmGmUmCmGmGmUmGmCmU*mU*mU*mU |
| 20 | Generic | NNNNNNNNNNNNNNNNNNNNGUUUUAGAGCUAGAAAUAGCAAGUUAAAAUAA |
| | sgRNA | GGCUAGUCCGUUAUCAACUUGGCACCGAGUCGGUGC |
| 21 | Generic | mN*mN*mN*NNNNNNNNNNNNNNNNNGUUUUAGAGCUAGAAAUAGCAAGUUA |
| | sgRNA | AAAUAAGGCUAGUCCGUUAUCAACUUGGCACCGAGUCGG*mU*mG*mC |
| | modified | |
| 22 | Generic | NNNNNNNNNNNNNNNNNNNNGUUUUAGAGCUAGAAAUAGCAAGUUAAAAUAA |
| | sgRNA | GGCUAGUCCGUUAUCACGAAAGGGCACCGAGUCGGUGC |
| 23 | Generic | mN*mN*mN*NNNNNNNNNNNNNNNNNGUUUUAGAmGmCmUmAmGmAmAmAmU |
| | sgRNA | mAmGmCAAGUUAAAAUAAGGCUAGUCCGUUAUCACGAAAGGGCACCGAGUCG |
| | modified | G*mU*mG*mC |
| 24 | Generic | NNNNNNNNNNNNNNNNNNNNGUUUUAGAGCUAGAAAUAGCAAGUUAAAAUAA |
| | sgRNA | GGCUAGUCCGUUAUCAAAAAUGGCACCGAGUCGGUGC |
| 25 | Generic | mN*mN*mN*NNNNNNNNNNNNNNNNNGUUUUAGAmGmCmUmAmGmAmAmAmU |
| | sgRNA | mAmGmCAAGUUAAAAUAAGGCUAGUCCGUUAUCAAAAAUGGCACCGAGUCGG |
| | modified | *mU*mG*mC |
| 26 | Generic | NNNNNNNNNNNNNNNNNNNNGUUUUAGAGCUAGAAAUAGCAAGUUAAAAUAA |
| | sgRNA | GGCUAGUCCGUUAUCACAAGGGCACCGAGUCGGUGC |
| 27 | Generic | mN*mN*mN*NNNNNNNNNNNNNNNNNGUUUUAGAmGmCmUmAmGmAmAmAmU |
| | sgRNA | mAmGmCAAGUUAAAAUAAGGCUAGUCCGUUAUCACAAGGGCACCGAGUCGG* |
| | modified | mU*mG*mC |
| 28 | Generic | NNNNNNNNNNNNNNNNNNNNGUUUUAGAGCUAGAAAUAGCAAGUUAAAAUAA |
| | sgRNA | GGCUAGUCCGUUAUCAAAAUGGCACCGAGUCGGUGC |
| 29 | Generic | mN*mN*mN*NNNNNNNNNNNNNNNNNGUUUUAGAmGmCmUmAmGmAmAmAmU |
| | sgRNA | mAmGmCAAGUUAAAAUAAGGCUAGUCCGUUAUCAAAAUGGCACCGAGUCGG* |
| | modified | mU*mG*mC |
| 30 | Generic | NNNNNNNNNNNNNNNNNNNNGUUUUAGAGCGCGAAGCGCAAGUUAAAAUAAG |
| | sgRNA | GCUAGUCCGUUAUCAAAAUGGCACCGAGUCGGUGC |
| 31 | Generic | mN*mN*mN*NNNNNNNNNNNNNNNNNGUUUUAGAGCGCGAAGCGCAAGUUAA |
| | sgRNA | AAUAAGGCUAGUCCGUUAUCAAAAUGGCACCGAGUCGG*mU*mG*mC |
| | modified | |
| 62 | sgRNA | UUUUAGAGCUAGAAAUAGCAAGUUAAAAUAAGGCUAGUCCGUUAUCAACUUG |
| | conserved | AAAAAGUGGCACCGAGUCGGUGCUUUU |
| | region | |
| 33 | crRNA | GUUUUAGAGCUAUGCUGUUUUG |
| | conserved | |
| | region | |
| 34 | TTR | mA*mC*mA*CAAAUACCAGUCCAGCAGUUUUAGAmGmCmUmAmGmAmAmAmU |
| | targeting | mAmGmCAAGUUAAAAUAAGGCUAGUCCGUUAUCAmAmCmUmUmGmAmAmAmA |
| | guide | mAmGmUmGmGmCmAmCmCmGmAmGmUmCmGmGmUmGmCmU*mU*mU*mU |
| 35 | TTR | mA*mC*mA*CAAAUACCAGUCCAGCGGUUUUAGAmGmCmUmAmGmAmAmAmU |
| | targeting | mAmGmCAAGUUAAAAUAAGGCUAGUCCGUUAUCAmAmCmUmUmGmAmAmAmA |
| | guide | mAmGmUmGmGmCmAmCmCmGmAmGmUmCmGmGmUmGmCmU*mU*mU*mU |
| 36 | ORF | AUGGAUAAGAAGUACUCAAUCGGGCUGGAUAUCGGAACUAAUUCCGUGGGUU |
| | encoding | GGGCAGUGAUCACGGAUGAAUACAAAGUGCCGUCCAAGAAGUUCAAGGUCCU |
| | Sp. Cas9 | GGGGAACACCGAUAGACACAGCAUCAAGAAAAAUCUCAUCGGAGCCCUGCUG |
| | | UUUGACUCCGGCGAAACCGCAGAAGCGACCCGGCUCAAACGUACCGCGAGGC |
| | | GACGCUACACCCGGCGGAAGAAUCGCAUCUGCUAUCUGCAAGAGAUCUUUUC |
| | | GAACGAAAUGGCAAAGGUCGACGACAGCUUCUUCCACCGCCUGGAAGAAUCU |
| | | UUCCUGGUGGAGGAGGACAAGAAGCAUGAACGGCAUCCUAUCUUUGGAAACA |
| | | UCGUCGACGAAGUGGCGUACCACGAAAAGUACCCGACCAUCUACCAUCUGCG |
| | | GAAGAAGUUGGUUGACUCAACUGACAAGGCCGACCUCAGAUUGAUCUACUUG |
| | | GCCCUCGCCCAUAUGAUCAAAUUCCGCGGACACUUCCUGAUCGAAGGCGAUC |
| | | UGAACCCUGAUAACUCCGACGUGGAUAAGCUUUUCAUUCAACUGGUGCAGAC |
| | | CUACAACCAACUGUUCGAAGAAAACCCAAUCAAUGCUAGCGGCGUCGAUGCC |
| | | AAGGCCAUCCUGUCCGCCCGGCUGUCGAAGUCGCGGCGCCUCGAAAACCUGA |
| | | UCGCACAGCUGCCGGGAGAGAAAAAGAACGGACUUUUCGGCAACUUGAUCGC |
| | | UCUCUCACUGGGACUCACUCCCAAUUUCAAGUCCAAUUUUGACCUGGCCGAG |
| | | GACGCGAAGCUGCAACUCUCAAAGGACACCUACGACGACGACUUGGACAAUU |
| | | UGCUGGCACAAAUUGGCGAUCAGUACGCGGAUCUGUUCCUUGCCGCUAAGAA |
| | | CCUUUCGGACGCAAUCUUGCUGUCCGAUAUCCUGCGCGUGAACACCGAAAUA |
| | | ACCAAAGCGCCGCUUAGCGCCUCGAUGAUUAAGCGGUACGACGAGCAUCACC |
| | | AGGAUCUCACGCUGCUCAAAGCGCUCGUGAGACAGCAACUGCCUGAAAAGUA |
| | | CAAGGAGAUCUUCUUCGACCAGUCCAAGAAUGGGUACGCAGGGUACAUCGAU |
| | | GGAGGCGCUAGCCAGGAAGAGUUCUAUAAGUUCAUCAAGCCAAUCCUGGAAA |
| | | AGAUGGACGGAACCGAAGAACUGCUGGUCAAGCUGAACAGGGAGGAUCUGCU |
| | | CCGGAAACAGAGAACCUUUGACAACGGAUCCAUUCCCCACCAGAUCCAUCUG |
| | | GGUGAGCUGCACGCCAUCUUGCGGCGCCAGGAGGACUUUUACCCAUUCCUCA |
| | | AGGACAACCGGGAAAAGAUCGAGAAAAUUCUGACGUUCCGCAUCCCGUAUUA |
| | | CGUGGGCCCACUGGCGCGCGGCAAUUCGCGCUUCGCGUGGAUGACUAGAAAA |
| | | UCAGAGGAAACCAUCACUCCUUGGAAUUUCGAGGAAGUUGUGGAUAAGGGAG |
| | | CUUCGGCACAAAGCUUCAUCGAACGAAUGACCAACUUCGACAAGAAUCUCCC |
| | | AAACGAGAAGGUGCUUCCUAAGCACAGCCUCCUUUACGAAUACUUCACUGUC |
| | | UACAACGAACUGACUAAAGUGAAAUACGUUACUGAAGGAAUGAGGAAGCCGG |
| | | CCUUUCUGUCCGGAGAACAGAAGAAAGCAAUUGUCGAUCUGCUGUUCAAGAC |
| | | CAACCGCAAGGUGACCGUCAAGCAGCUUAAAGAGGACUACUUCAAGAAGAUC |
| | | GAGUGUUUCGACUCAGUGGAAAUCAGCGGGGUGGAGGACAGAUUCAACGCUU |
| | | CGCUGGGAACCUAUCAUGAUCUCCUGAAGAUCAUCAAGGACAAGGACUUCCU |
| | | UGACAACGAGGAGAACGAGGACAUCCUGGAAGAUAUCGUCCUGACCUUGACC |
| | | CUUUUCGAGGAUCGCGAGAUGAUCGAGGAGAGGCUUAAGACCUACGCUCAUC |
| | | UCUUCGACGAUAAGGUCAUGAAACAACUCAAGCGCCGCCGGUACACUGGUUG |
| | | GGGCCGCCUCUCCCGCAAGCUGAUCAACGGUAUUCGCGAUAAACAGAGCGGU |
| | | AAAACUAUCCUGGAUUUCCUCAAAUCGGAUGGCUUCGCUAAUCGUAACUUCA |
| | | UGCAAUUGAUCCACGACGACAGCCUGACCUUUAAGGAGGACAUCCAAAAAGC |
| | | ACAAGUGUCCGGACAGGGAGACUCACUCCAUGAACACAUCGCGAAUCUGGCC |
| | | GGUUCGCCGGCGAUUAAGAAGGGAAUUCUGCAAACUGUGAAGGUGGUCGACG |
| | | AGCUGGUGAAGGUCAUGGGACGGCACAAACCGGAGAAUAUCGUGAUUGAAAU |
| | | GGCCCGAGAAAACCAGACUACCCAGAAGGGCCAGAAAAACUCCCGCGAAAGG |
| | | AUGAAGCGGAUCGAAGAAGGAAUCAAGGAGCUGGGCAGCCAGAUCCUGAAAG |
| | | AGCACCCGGUGGAAAACACGCAGCUGCAGAACGAGAAGCUCUACCUGUACUA |
| | | UUUGCAAAAUGGACGGGACAUGUACGUGGACCAAGAGCUGGACAUCAAUCGG |
| | | UUGUCUGAUUACGACGUGGACCACAUCGUUCCACAGUCCUUUCUGAAGGAUG |
| | | ACUCGAUCGAUAACAAGGUGUUGACUCGCAGCGACAAGAACAGAGGGAAGUC |
| | | AGAUAAUGUGCCAUCGGAGGAGGUCGUGAAGAAGAUGAAGAAUUACUGGCGG |
| | | CAGCUCCUGAAUGCGAAGCUGAUUACCCAGAGAAAGUUUGACAAUCUCACUA |
| | | AAGCCGAGCGCGGCGGACUCUCAGAGCUGGAUAAGGCUGGAUUCAUCAAACG |
| | | GCAGCUGGUCGAGACUCGGCAGAUUACCAAGCACGUGGCGCAGAUCUUGGAC |
| | | UCCCGCAUGAACACUAAAUACGACGAGAACGAUAAGCUCAUCCGGGAAGUGA |
| | | AGGUGAUUACCCUGAAAAGCAAACUUGUGUCGGACUUUCGGAAGGACUUUCA |
| | | GUUUUACAAAGUGAGAGAAAUCAACAACUACCAUCACGCGCAUGACGCAUAC |
| | | CUCAACGCUGUGGUCGGUACCGCCCUGAUCAAAAAGUACCCUAAACUUGAAU |
| | | CGGAGUUUGUGUACGGAGACUACAAGGUCUACGACGUGAGGAAGAUGAUAGC |
| | | CAAGUCCGAACAGGAAAUCGGGAAAGCAACUGCGAAAUACUUCUUUUACUCA |
| | | AACAUCAUGAACUUUUUCAAGACUGAAAUUACGCUGGCCAAUGGAGAAAUCA |
| | | GGAAGAGGCCACUGAUCGAAACUAACGGAGAAACGGGCGAAAUCGUGUGGGA |
| | | CAAGGGCAGGGACUUCGCAACUGUUCGCAAAGUGCUCUCUAUGCCGCAAGUC |
| | | AAUAUUGUGAAGAAAACCGAAGUGCAAACCGGCGGAUUUUCAAAGGAAUCGA |
| | | UCCUCCCAAAGAGAAAUAGCGACAAGCUCAUUGCACGCAAGAAAGACUGGGA |
| | | CCCGAAGAAGUACGGAGGAUUCGAUUCGCCGACUGUCGCAUACUCCGUCCUC |
| | | GUGGUGGCCAAGGUGGAGAAGGGAAAGAGCAAAAAGCUCAAAUCCGUCAAAG |
| | | AGCUGCUGGGGAUUACCAUCAUGGAACGAUCCUCGUUCGAGAAGAACCCGAU |
| | | UGAUUUCCUCGAGGCGAAGGGUUACAAGGAGGUGAAGAAGGAUCUGAUCAUC |
| | | AAACUCCCCAAGUACUCACUGUUCGAACUGGAAAAUGGUCGGAAGCGCAUGC |
| | | UGGCUUCGGCCGGAGAACUCCAAAAAGGAAAUGAGCUGGCCUUGCCUAGCAA |
| | | GUACGUCAACUUCCUCUAUCUUGCUUCGCACUACGAAAAACUCAAAGGGUCA |
| | | CCGGAAGAUAACGAACAGAAGCAGCUUUUCGUGGAGCAGCACAAGCAUUAUC |
| | | UGGAUGAAAUCAUCGAACAAAUCUCCGAGUUUUCAAAGCGCGUGAUCCUCGC |
| | | CGACGCCAACCUCGACAAAGUCCUGUCGGCCUACAAUAAGCAUAGAGAUAAG |
| | | CCGAUCAGAGAACAGGCCGAGAACAUUAUCCACUUGUUCACCCUGACUAACC |
| | | UGGGAGCCCCAGCCGCCUUCAAGUACUUCGAUACUACUAUCGAUCGCAAAAG |
| | | AUACACGUCCACCAAGGAAGUUCUGGACGCGACCCUGAUCCACCAAAGCAUC |
| | | ACUGGACUCUACGAAACUAGGAUCGAUCUGUCGCAGCUGGGUGGCGAUGGCG |
| | | GUGGAUCUCCGAAAAAGAAGAGAAAGGUGUAAUGA |
| 37 | exemplary | gccgccRccAUGG |
| | Kozak | |
| | sequence | |
| 38 | TTR | mA*mA*mA*GGCUGCUGAUGACACCUGUUUUAGAGCUAGAAAUAGCAAGUUA |
| | sgRNA | AAAUAAGGCUAGUCCGUUAUCAACUUGAAAAAGUGGCACCGAGUCGGUGCU* |
| | | mU*mU*mU |
| 39 | TTR | mA*mA*mA*GGCUGCUGAUGACACCUGUUUUAGAmGmCmUmAmGmAmAmAmU |
| | sgRNA | mAmGmCAAGUUAAAAUAAGGCUAGUCCGUUAUCAmAmCmUmUmGmAmAmAmA |
| | | mAmGmUmGmGmCmAmCmCmGmAmGmUmCmGmGmUmGmCmU*mU*mU*mU |
| 40 | TTR | mA*mA*mA*mGGC*U*fG*fC*fU*fGAfUfGAC*fAfCCUmGUUUfUAGmA |
| | sgRNA | mGmCmUmAmGmAmAmAmUmAmGmCmAmAGUfUmAfAmAfAmUAmAmGmGmCm |
| | | UmAGUmCmCGUfUAmUmCAmAmCmUmUmGmAmAmAmAmAmGmUmGmGmCmAm |
| | | CmCmGmAmGmUmCmGmGmUmGmCmU*mU*mU*mU |
| 41 | TTR | mA*mA*mA*GGC*U*fG*fC*fU*fGAfUfGAC*fAfCCUmGUUUfUAGmAm |
| | sgRNA | GmCmUmAmGmAmAmAmUmAmGmCmAmAGUfUmAfAmAfAmUAmAmGmGmCmU |
| | | mAGUmCmCGUfUAmUmCAmAmCmUmUmGmAmAmAmAmAmGmUmGmGmCmAmC |
| | | mCmGmAmGmUmCmGmGmUmGmCmU*mU*mU*mU |
| 42 | TTR | mA*mC*mA*CAAAUACCAGUCCAGCGGUUUUAGAmGmCmUmAmGmAmAmAmU |
| | sgRNA | mAmGmCAAGUUAAAAUAAGGCUAGUCCGUUAUCACGAAAGGGCACCGAGUCG |
| | | GmU*mG*mC*mU |
| 43 | TTR | AAAGGCUGCUGAUGACACCUGUUUUAGAGCUAGAAAUAGCAAGUUAAAAUAA |
| | sgRNA | GGCUAGUCCGUUAUCAACUUGGCACCGAGUCGGUGC |
| 44 | TTR | mA*mA*mA*GGCUGCUGAUGACACCUGUUUUAGAGCUAGAAAUAGCAAGUUA |
| | sgRNA | AAAUAAGGCUAGUCCGUUAUCAACUUGGCACCGAGUCGG*mU*mG*mC |
| | modified | |
| 45 | TTR | AAAGGCUGCUGAUGACACCUGUUUUAGAGCUAGAAAUAGCAAGUUAAAAUAA |
| | sgRNA | GGCUAGUCCGUUAUCACGAAAGGGCACCGAGUCGGUGC |
| 46 | TTR | mA*mA*mA*GGCUGCUGAUGACACCUGUUUUAGAmGmCmUmAmGmAmAmAmU |
| | sgRNA | mAmGmCAAGUUAAAAUAAGGCUAGUCCGUUAUCACGAAAGGGCACCGAGUCG |
| | modified | G*mU*mG*mC |
| 47 | TTR | AAAGGCUGCUGAUGACACCUGUUUUAGAGCUAGAAAUAGCAAGUUAAAAUAA |
| | sgRNA | GGCUAGUCCGUUAUCAAAAAUGGCACCGAGUCGGUGC |
| 48 | TTR | mA*mA*mA*GGCUGCUGAUGACACCUGUUUUAGAmGmCmUmAmGmAmAmAmU |
| | sgRNA | mAmGmCAAGUUAAAAUAAGGCUAGUCCGUUAUCAAAAAUGGCACCGAGUCGG |
| | modified | *mU*mG*mC |
| 49 | TTR | AAAGGCUGCUGAUGACACCUGUUUUAGAGCUAGAAAUAGCAAGUUAAAAUAA |
| | sgRNA | GGCUAGUCCGUUAUCACAAGGGCACCGAGUCGGUGC |
| 50 | TTR′ | mA*mA*mA*GGCUGCUGAUGACACCUGUUUUAGAmGmCmUmAmGmAmAmAmU |
| | sgRNA | mAmGmCAAGUUAAAAUAAGGCUAGUCCGUUAUCACAAGGGCACCGAGUCGG* |
| | modified | mU*mG*mC |
| 51 | TTR | AAAGGCUGCUGAUGACACCUGUUUUAGAGCUAGAAAUAGCAAGUUAAAAUAA |
| | sgRNA | GGCUAGUCCGUUAUCAAAAUGGCACCGAGUCGGUGC |
| 52 | TTR | mA*mA*mA*GGCUGCUGAUGACACCUGUUUUAGAmGmCmUmAmGmAmAmAmU |
| | sgRNA | mAmGmCAAGUUAAAAUAAGGCUAGUCCGUUAUCAAAAUGGCACCGAGUCGG* |
| | modified | mU*mG*mC |
| 53 | TTR | AAAGGCUGCUGAUGACACCUGUUUUAGAGCGCGAAGCGCAAGUUAAAAUAAG |
| | sgRNA | GCUAGUCCGUUAUCAAAAUGGCACCGAGUCGGUGC |
| 54 | TTR | mA*mA*mA*GGCUGCUGAUGACACCUGUUUUAGAGCGCGAAGCGCAAGUUAA |
| | sgRNA | AAUAAGGCUAGUCCGUUAUCAAAAUGGCACCGAGUCGG*mU*mG*mC |
| | modified | |
| 55 | Generic | mN*mN*mN*NNNNNNNNNNNNNNNNNGUUUUAGAmGmCmUmAmGmAmAmAmU |
| | sgRNA | mAmGmCAAGUUAAAAUAAGGCUAGUCCGUUAUCACGAAAGGGCACCGAGUCG |
| | modified | GmU*mG*mC*mU |
| 56 | Generic | mN*mN*mN*mNNN*N*fN*fN*fN*fNNfNfNNN*fNfNNmGUUUfUAGmAm |
| | sgRNA | GmCmUmAmGmAmAmAmUmAmGmCmAmAGUfUmAfAmAfAmUAmAmGmGmCmU |
| | modified | mAGUmCmCGUfUAmUmCAmAmCmUmUmGmAmAmAmAmAmGmUmGmGmCmAmC |
| | | mCmGmAmGmUmCmGmGmUmGmCmU*mU*mU*mU |
| 57 | Generic | mN*mN*mN*NNN*N*fN*fN*fN*fNNfNfNNN*fNfNNNmGUUUfUAGmAm |
| | sgRNA | GmCmUmAmGmAmAmAmUmAmGmCmAmAGUfUmAfAmAfAmUAmAmGmGmCmU |
| | modified | mAGUmCmCGUfUAmUmCAmAmCmUmUmGmAmAmAmAmAmGmUmGmGmCmAmC |
| | | mCmGmAmGmUmCmGmGmUmGmCmU*mU*mU*mU |

* = PS linkage; ′m′ = 2′-O-Me nucleotide, ′f′ = 2′-F nucleotide

1. A method for treating amyloidosis associated with TTR (ATTR) in a human subject, comprising:
a. systemically administering to the human subject a LNP composition comprising an effective amount of:
i. an mRNA encoding a Cas nuclease, and
ii. a guide RNA that targets the TTR gene,
wherein the administration of the composition reduces serum TTR relative to baseline serum TTR.

2. The method of claim 1, wherein the ATTR is hereditary transthyretin amyloidosis.

3. The method of claim 1, wherein the ATTR is wild-type transthyretin amyloidosis.

4. The method of any one of claim 1 or 2, wherein the ATTR is hereditary transthyretin amyloidosis with polyneuropathy.

5. The method of any one of claim 1 or 2-4, wherein the ATTR is hereditary transthyretin amyloidosis with cardiomyopathy.

6. The method of claim 1 or 3, wherein the ATTR is wildtype transthyretin amyloidosis with cardiomyopathy.

7. The method of claim 1, 5, or 6, wherein the subject is classified under the New York Health Association (NYHA) classification as Class I, Class II, or Class III.

8. The method of any one of claims 1-7, wherein the subject has ATTRv-PN and/or ATTR-CM.

9. The method of any one of claims 1-8 wherein the LNP comprises (9Z, 12Z)-3-((4,4-bis(octyloxy)butanoyl)oxy)-2-((((3-(diethylamino)propoxy)carbonyl)oxy)methyl)propyl octadeca-9, 12-dienoate.

10. The method of any one of claims 1-9, wherein the LNP comprises a PEG lipid.

11. The method of claim 10, wherein the PEG lipid comprises dimyristoylglycerol (DMG).

12. The method of claim 11, wherein the PEG lipid comprises PEG-2k.

13. The method of any one of claims 1-12, wherein the LNP composition has an N/P ratio of about 5-7.

14. The method of any one of claims 1-13, wherein the guide RNA and Cas nuclease are present in a ratio ranging from about 5:1 to about 1:5 by weight.

15. The method of any one of claims 1-14, wherein the mRNA encodes a Class 2 Cas nuclease.

16. The method of any one of claims 1-15, wherein the mRNA encodes a Cas9 nuclease.

17. The method of any one of claims 1-16, wherein the mRNA encodes S. pyogenes Cas9.

18. The method of any one of claims 1-17, wherein the mRNA encoding the Cas nuclease is codon-optimized.

19. The method of any one of claims 1-18, wherein the effective amount of mRNA encoding a Cas nuclease and the guide RNA that targets the TTR gene is a combined dose of about 0.3 mg/kg to about 2 mg/kg.

20. The method of any one of claims 1-18, wherein the effective amount of mRNA encoding a Cas nuclease and the guide RNA that targets the TTR gene is a combined dose of about 0.3 mg/kg to about 1 mg/kg.

21. The method of any one of claims 1-18, wherein the effective amount of mRNA encoding a Cas nuclease and the guide RNA that targets the TTR gene is a combined dose of about 0.3 mg/kg.

22. The method of any one of claims 1-18, wherein the effective amount of mRNA encoding a Cas nuclease and the guide RNA that targets the TTR gene is a combined dose of about 0.7 mg/kg.

23. The method of any one of claims 1-18, wherein the effective amount of mRNA encoding a Cas nuclease and the guide RNA that targets the TTR gene is a combined dose of about 1.0 mg/kg.

24. The method of any one of claims 1-18, wherein the effective amount of mRNA encoding a Cas nuclease and the guide RNA that targets the TTR gene is a combined dose of about 25 mg to about 150 mg of total RNA.

25. The method of any one of claims 1-18, wherein the effective amount of mRNA encoding a Cas nuclease and the guide RNA that targets the TTR gene is a combined dose of about 25 mg to about 100 mg of total RNA.

26. The method of any one of claims 1-18, wherein the effective amount of mRNA encoding a Cas nuclease and the guide RNA that targets the TTR gene is a combined dose of about 50 mg to about 90 mg of total RNA.

27. The method of any one of claims 1-18, wherein the effective amount of mRNA encoding a Cas nuclease and the guide RNA that targets the TTR gene is a combined dose of about 60 mg of total RNA.

28. The method of any one of claims 1-18, wherein the effective amount of mRNA encoding a Cas nuclease and the guide RNA that targets the TTR gene is a combined dose of about 70 mg of total RNA.

29. The method of any one of claims 1-18, wherein the effective amount of mRNA encoding a Cas nuclease and the guide RNA that targets the TTR gene is a combined dose of about 80 mg of total RNA.

30. The method of any one of claims 1-18, wherein the effective amount of mRNA encoding a Cas nuclease and the guide RNA that targets the TTR gene is a combined dose of about 90 mg of total RNA.

31. The method of any one of claims 1-30, wherein administration of the composition reduces serum TTR by 60-70%, 70-80%, 80-90%, 90-95%, 95-98%, 98-99%, or 99-100% as compared to baseline serum TTR before administration of the composition.

32. The method of any one of claims 1-31, wherein the serum TTR levels are less than about 50 μg/mL after administration of the composition.

33. The method of any one of claims 1-31, wherein the serum TTR levels are less than about 40 μg/mL after administration of the composition.

34. The method of any one of claims 1-31, wherein the serum TTR levels are less than about 30 μg/mL after administration of the composition.

35. The method of any one of claims 1-31, wherein the serum TTR levels are less than about 20 μg/mL after administration of the composition.

36. The method of any one of claims 1-31, wherein the serum TTR levels are less than about 10 μg/mL after administration of the composition.

37. The method of any one of claims 1-36, further comprising administering a second dose of the LNP composition, wherein administration of the second dose reduces serum TTR levels by at least 80% relative to the baseline serum TTR level prior to administration of the first dose.

38. The method of any one of claims 1-36, further comprising administering a second dose of the LNP composition, wherein administration of the second dose reduces serum TTR levels by at least 80% relative to the baseline serum TTR level prior to administration of the second dose and after administration of the first dose.

39. The method of any one of claims 1-37, wherein the composition is administered with a second therapeutic.

40. The method of claim 39, where in the second therapeutic is diflunisal or tafamidis.

41. A method for in vivo editing of the transthyretin (TTR) gene in a human subject having amyloidosis associated with TTR (ATTR), comprising:
a. systemically administering to the human subject a lipid nano particle (LNP) composition comprising:
i. an mRNA encoding a Cas nuclease, and
ii. a guide RNA that targets the TTR gene; and
b. editing the gene at the site targeted by the guide RNA in a hepatocyte of the subject;
wherein the administration of the composition results in a change in a level of a biosafety metric in the subject that is acceptable as compared to a baseline level of the biosafety metric.

42. A method for in vivo editing of the transthyretin (TTR) gene in a human subject having ATTR, comprising:
a. systemically administering to the human subject a LNP composition comprising an effective amount of:
i. an mRNA encoding a Cas nuclease, and
ii. a guide RNA that targets the TTR gene; and
b. editing the TTR gene at the site targeted by the guide RNA in a hepatocyte of the subject;
wherein the administration of the composition results in a clinically significant improvement in a level of a clinical metric in the subject as compared to a baseline level.

43. A method for treating amyloidosis associated with TTR (ATTR) in a human subject, comprising:
a. systemically administering to the human subject a LNP composition comprising an effective amount of:
i. an mRNA encoding a Cas nuclease, and
ii. a guide RNA that targets the TTR gene,
wherein the administration of the composition results in a change in a level of a biosafety metric in the subject that is acceptable as compared to a baseline level.

44. A method for treating amyloidosis associated with TTR (ATTR) in a human subject, comprising:
a. systemically administering to the human subject a LNP composition comprising an effective amount of:
i. an mRNA encoding a Cas nuclease, and
ii. a guide RNA that targets the TTR gene,
wherein the administration of the composition results in a clinically significant improvement in a level of a clinical metric in the subject as compared to a baseline level of the clinical metric.

45. A method for treating amyloidosis associated with TTR (ATTR) in a human subject, comprising:
a. systemically administering to the human subject a LNP composition comprising an effective amount of:
i. an mRNA encoding a Cas nuclease, and
ii. a guide RNA that targets the TTR gene,
wherein the mRNA encoding a Cas nuclease and the guide RNA that targets the TTR gene are administered at a combined dose of about 25 to about 100 mg.

46. A method for in vivo editing of a gene in the liver of a human subject having a monogenic disorder, comprising:
a. systemically administering to the human subject a LNP composition comprising:
i. an mRNA encoding a Cas nuclease, and
ii. a guide RNA that targets a gene in the liver; and
b. editing the gene at the site targeted by the guide RNA in a hepatocyte of the subject;
wherein the administration of the composition results in a change in a level of a biosafety metric in the subject that is acceptable as compared to a baseline level of the biosafety metric.

47. A method for treating a human subject having a monogenic disorder, comprising:
a. systemically administering to the human subject a LNP composition comprising an effective amount of:
i. an mRNA encoding a Cas nuclease, and
ii. a guide RNA that targets a gene in the liver; and
b. editing the gene in the liver thereby treating the monogenic disorder,
wherein the treatment is safe and well-tolerated.

48. A method for treating a human subject having a monogenic disorder, comprising:
a. systemically administering to the human subject a LNP composition comprising an effective amount of:
i. an mRNA encoding a Cas nuclease, and
ii. a guide RNA that targets a gene in the liver;
b. determining a first level of a biosafety metric in the subject prior to administration;
c. determining a second level of the biosafety metric in the subject a period of time after administration; and
d. assessing the change between the first and the second levels of the biosafety metric,
wherein the administration of the composition results in a change in a level of a biosafety metric in the subject that is acceptable as compared to a baseline level, thereby treating ATTR.

49. The method of any one of claims 41-45 comprising selecting a human subject having amyloidosis associated with TTR (ATTR) prior to the systemic administration.

50. The method of any one of claims 46-48 comprising selecting a human subject having a monogenic disorder prior to the systemic administration.

51. The method of any one of claims 41-50, wherein the LNP comprises (9Z, 12Z)-3-((4,4-bis(octyloxy)butanoyl)oxy)-2-((((3-(diethylamino)propoxy)carbonyl)oxy)methyl)propyl octadeca-9, 12-dienoate.

52. The method of any one of claims 41-51, wherein the LNP comprises a PEG lipid.

53. The method of claim 52, wherein the PEG lipid comprises dimyristoylglycerol (DMG).

54. The method of claim 53, wherein the PEG lipid comprises PEG-2k.

55. The method of any one of claims 41-54, wherein the LNP composition has an N/P ratio of about 5-7.

56. The method of any one of claims 41-55, wherein the guide RNA and Cas nuclease are present in a ratio ranging from about 5:1 to about 1:5 by weight.

57. The method of any one of claims 41-56, wherein the mRNA encodes a Class 2 Cas nuclease.

58. The method of any one of claims 41-57, wherein the mRNA encodes a Cas9 nuclease.

59. The method of any one of claims 41-58, wherein the mRNA encodes S. pyogenes Cas9.

60. The method of any one of claims 41-59, wherein the mRNA encoding the Cas nuclease is codon-optimized.

61. The method of any one of claims 41-60, wherein the guide RNA comprises at least one modification.

62. The method of claim 61, wherein the at least one modification to the guide RNA includes a 2′-O-methyl modified nucleotide or a phosphorothioate bond between nucleotides.

63. The method of any one of claims 41-62, wherein the mRNA comprises at least one modification.

64. The method of any one of claims 46-48, wherein the monogenic disorder is ATTR.

65. The method of any one of claims 46-48, wherein the gene in the liver is TTR.

66. The method of any one of claims 41-65, wherein the ATTR is hereditary transthyretin amyloidosis.

67. The method of any one of claims 41-65, wherein the ATTR is wild-type transthyretin amyloidosis.

68. The method of any one of claims 41-66, wherein the ATTR is hereditary transthyretin amyloidosis with polyneuropathy.

69. The method of any one of claim 41-66, or 68 wherein the ATTR is hereditary transthyretin amyloidosis with cardiomyopathy.

70. The method of claim 67, wherein the ATTR is wildtype transthyretin amyloidosis with cardiomyopathy.

71. The method of claim 69 or 70, wherein the subject is classified under the New York Health Association (NYHA) classification as Class I, Class II, or Class III.

72. The method of any one of claims 41-65, wherein the subject has ATTRv-PN and/or ATTR-CM.

73. The method of any one of claims 41-67, wherein the biosafety metric is prothrombin.

74. The method of any one of claim 41, 43, or 46, wherein administration of the composition results in a change in a level of a biosafety metric in the subject that is acceptable as compared to a baseline level of the biosafety metric.

75. The method of any of claim 41, 43, 46, or 48, wherein the biosafety metric is activated partial thromboplastin time (aPTT).

76. The method of any one of claim 41, 43, 46, or 48, wherein the biosafety metric is fibrinogen.

77. The method of any one of claim 41, 43, 46, or 48, wherein the biosafety metric is alanine aminotransferase (ALT).

78. The method of any one of claim 41, 43, 46, or 48, wherein the biosafety metric is aspartate aminotransferase (AST).

79. The method of any one of claims 41-78, wherein the mRNA encoding a Cas nuclease and the guide RNA that targets the TTR gene are administered at a combined dose of about 0.3 mg/kg to about 2 mg/kg.

80. The method of any one of claims 41-78, wherein the mRNA encoding a Cas nuclease and the guide RNA that targets the TTR gene are administered at a combined dose of about 0.3 mg/kg to about 1 mg/kg.

81. The method of any one of claims 41-80, wherein the mRNA encoding a Cas nuclease and the guide RNA that targets the TTR gene are administered at a combined dose of about 0.3 mg/kg.

82. The method of any one of claims 41-80, wherein the mRNA encoding a Cas nuclease and the guide RNA that targets the TTR gene are administered at a combined dose of about 0.7 mg/kg.

83. The method of any one of claims 41-80, wherein the mRNA encoding a Cas nuclease and the guide RNA that targets the TTR gene are administered at a combined dose of about 1.0 mg/kg.

84. The method of any one of claims 41-78, wherein the mRNA encoding a Cas nuclease and the guide RNA that targets the TTR gene are administered at a combined dose of about 50 mg to 90 mg of total RNA.

85. The method of claim 84, further comprising administering a second dose of the LNP composition, wherein administration of the second dose reduces serum TTR level by at least 80% relative to the baseline serum TTR level.

86. The method of any one of claims 41-78, wherein the mRNA encoding a Cas nuclease and the guide RNA that targets the TTR gene are administered at a combined dose of about 15 mg to 100 mg of total RNA.

87. The method of any one of claims 41-78, wherein the mRNA encoding a Cas nuclease and the guide RNA that targets the TTR gene are administered at a combined dose of about 70 mg to about 90 mg of total RNA.

88. The method of claim 87, further comprising administering a second dose of the LNP composition, wherein administration of the second dose reduces serum TTR level by at least 80% relative to the baseline serum TTR level (i) prior to administration of the first dose or (2) prior to administration of the second dose and after administration of the first dose.

89. The method of any one of claims 41-78, wherein the mRNA encoding a Cas nuclease and the guide RNA that targets the TTR gene are administered at a combined dose of about 25 mg to about 27 mg of total RNA.

90. The method of any one of claims 41-78, wherein the mRNA encoding a Cas nuclease and the guide RNA that targets the TTR gene are administered at a combined dose of about 25 mg to about 150 mg of total RNA.

91. The method of any one of claims 41-78, wherein the mRNA encoding a Cas nuclease and the guide RNA that targets the TTR gene are administered at a combined dose of about 25 mg to about 100 mg of total RNA.

92. The method of any one of claims 41-78, wherein the mRNA encoding a Cas nuclease and the guide RNA that targets the TTR gene are administered at a combined dose of about 50 mg to about 90 mg of total RNA.

93. The method of any one of claims 41-78, wherein the mRNA encoding a Cas nuclease and the guide RNA that targets the TTR gene are at a combined dose of about 35 mg to 65 mg of total RNA.

94. The method of any one of claims 41-78, wherein the mRNA encoding a Cas nuclease and the guide RNA that targets the TTR gene are at a combined dose of about 40 mg of total RNA.

95. The method of any one of claims 41-78, wherein the mRNA encoding a Cas nuclease and the guide RNA that targets the TTR gene are administered at a combined dose of about 50 mg of total RNA.

96. The method of any one of claims 41-78, wherein the mRNA encoding a Cas nuclease and the guide RNA that targets the TTR gene are at a combined dose of about 60 mg of total RNA.

97. The method of any one of claims 41-78, wherein the mRNA encoding a Cas nuclease and the guide RNA that targets the TTR gene are at a combined dose of about 70 mg of total RNA.

98. The method of any one of claims 41-78, wherein the mRNA encoding a Cas nuclease and the guide RNA that targets the TTR gene are at a combined dose of about 80 mg of total RNA.

99. The method of any one of claims 41-78, wherein the mRNA encoding a Cas nuclease and the guide RNA that targets the TTR gene are at a combined dose of about 90 mg of total RNA.

100. The method of any one of claims 41-78, wherein the mRNA encoding a Cas nuclease and the guide RNA that targets the TTR gene are at a combined dose of about 100 mg of total RNA.

101. The method of any one of claims 84-100, wherein the total RNA about is within +5% of the combined mg dose.

102. The method of any one of claims 84-100, wherein the total RNA about is within ±10% of the combined mg dose.

103. The method of any one of claims 41-102, wherein the mRNA encoding a Cas nuclease and the guide RNA that targets the TTR gene are at a combined dose of 75 mg of total RNA.

104. The method of any one of claims 41-102, wherein the mRNA encoding a Cas nuclease and the guide RNA that targets the TTR gene are at a combined dose of 76 mg of total RNA.

105. The method of any one of claims 41-102, wherein the mRNA encoding a Cas nuclease and the guide RNA that targets the TTR gene are at a combined dose of 77 mg of total RNA.

106. The method of any one of claims 41-102, wherein the mRNA encoding a Cas nuclease and the guide RNA that targets the TTR gene are at a combined dose of 78 mg of total RNA.

107. The method of any one of claims 41-102, wherein the mRNA encoding a Cas nuclease and the guide RNA that targets the TTR gene are at a combined dose of 79 mg of total RNA.

108. The method of any one of claims 41-102, wherein the mRNA encoding a Cas nuclease and the guide RNA that targets the TTR gene are at a combined dose of 80 mg of total RNA.

109. The method of any one of claims 41-102, wherein the mRNA encoding a Cas nuclease and the guide RNA that targets the TTR gene are at a combined dose of 81 mg of total RNA.

110. The method of any one of claims 41-102, wherein the mRNA encoding a Cas nuclease and the guide RNA that targets the TTR gene are at a combined dose of 82 mg of total RNA.

111. The method of any one of claims 41-102, wherein the mRNA encoding a Cas nuclease and the guide RNA that targets the TTR gene are at a combined dose of 83 mg of total RNA.

112. The method of any one of claims 41-102, wherein the mRNA encoding a Cas nuclease and the guide RNA that targets the TTR gene are at a combined dose of 84 mg of total RNA.

113. The method of any one of claims 41-102, wherein the mRNA encoding a Cas nuclease and the guide RNA that targets the TTR gene are at a combined dose of 85 mg of total RNA.

114. The method of any one of claims 41-102, wherein the mRNA encoding a Cas nuclease and the guide RNA that targets the TTR gene are at a combined dose of 86 mg of total RNA.

115. The method of any one of claims 41-102, wherein the mRNA encoding a Cas nuclease and the guide RNA that targets the TTR gene are at a combined dose of 87 mg of total RNA.

116. The method of any one of claims 41-102, wherein the mRNA encoding a Cas nuclease and the guide RNA that targets the TTR gene are at a combined dose of 88 mg of total RNA.

117. The method of any one of claims 41-102, wherein the mRNA encoding a Cas nuclease and the guide RNA that targets the TTR gene are at a combined dose of 89 mg of total RNA.

118. The method of any one of claims 41-102, wherein the mRNA encoding a Cas nuclease and the guide RNA that targets the TTR gene are at a combined dose of 90 mg of total RNA.

119. The method of claim 42 or 44, wherein the clinical metric is serum TTR level.

120. The method of any one of claims 41-119, wherein administration of the composition reduces or knocks down expression of the TTR gene.

121. The method of any one of claims 41-120, wherein administration of the composition reduces or knocks down expression of the TTR gene by 60-70%, 70-80%, 80-90%, 90-95%, 95-98%, 98-99%, or 99-100% as compared to baseline before administration of the composition.

122. The method of any of claims 41-120, wherein administration of the composition reduces TTR serum level in the subject by at least 60% as compared to serum TTR level before administration of the composition.

123. The method of any of claims 41-120, wherein administration of the composition reduces TTR serum level in the subject by 60-70%, 70-80%, 80-90%, 90-95%, 95-98%, 98-99%, or 99-100% as compared to serum TTR level before administration of the composition.

124. The method of claim 123, wherein the serum TTR level is reduced by at least 60% relative to baseline serum TTR level.

125. The method of claim 123, wherein the serum TTR level is reduced by at least 80% relative to baseline serum TTR level.

126. The method of claim 123, wherein the serum TTR level is reduced by at least 90% relative to baseline serum TTR level.

127. The method of claim 123, wherein the serum TTR level is reduced by at least 95% relative to baseline serum TTR level.

128. The method of any one of claims 41-120, wherein the serum TTR levels are less than about 50 μg/mL after administration of the composition.

129. The method of any one of claims 41-120, wherein the serum TTR levels are less than about 40 μg/mL after administration of the composition.

130. The method of any one of claims 41-120, wherein the serum TTR levels are less than about 30 μg/mL after administration of the composition.

131. The method of any one of claims 41-120, wherein the serum TTR levels are less than about 20 μg/mL after administration of the composition.

132. The method of any one of claims 41-120, wherein the serum TTR levels are less than about 10 μg/mL after administration of the composition.

133. The method of any one of claims 41-132, wherein the composition is administered with a second therapeutic.

134. The method of claim 133, where in the second therapeutic is diflunisal or tafamidis.

135. A method for treating amyloidosis associated with TTR (ATTR) in a human subject, comprising administering to the subject an effective amount of a composition that reduces serum TTR level in the subject by at least 95% as compared to a baseline serum TTR level.

136. The method of any previous claim, comprising durably reducing expression of the gene, after a single administration of the composition.

137. The method of claim 136, wherein the gene is TTR gene.

138. The method of claim 136 or 137, wherein the composition comprises: (i) an mRNA encoding a Cas nuclease, and (ii) a guide RNA that targets the TTR gene.
